(19) United States
(12) Patent Application Publication
(10) Pub. No.: US 2015/0218636 A1
(43) Pub. Date: Aug. 6, 2015

(54) Mutations In Calmodulin Genes

(71) Applicants: Aarhus Universitet, Aarhus C (DK); Aalborg Universitet, Aalborg Ost (DK)

(72) Inventors: Michael Toft Overgaard, Gistrup (DK); Mette Nyegaard, Gistrup (DK); Anders Borglum, Hojbjerg (DK)

(21) Appl. No.: 14/391,266

(22) PCT Filed: Apr. 12, 2013
(86) PCT No.: PCT/EP2013/057726

§ 371 Date: Oct. 8, 2014

Related U.S. Application Data
(60) Provisional application No. 61/695,416, filed on Aug. 31, 2012.
(60) Provisional application No. 61/623,191, filed on Apr. 12, 2012.

(51) Int. Cl. C12Q 1/68; G01N 33/573; C12N 9/12

(52) U.S. Cl. CPC C12Q 1/6883; C12N 9/12; G01N 33/573; C12Y 207/11019; C12Q 2600/156; G01N 2333/4727; G01N 2333/91205; G01N 2500/02; G01N 2800/325

(57) ABSTRACT

The present invention relates to an isolated polynucleotide encoding at least a part of calmodulin and an isolated polypeptide comprising at least a part of a calmodulin protein, wherein the polynucleotide and the polypeptide comprise at least one mutation associated with a cardiac disorder. The present invention also relates to a method for determining whether an individual has an increased risk of contracting a cardiac disorder, a method for diagnosing a cardiac disorder, method for treatment of an individual having a cardiac disorder, method for identifying a compound, capable of enhancing the binding of calmodulin to ryanodine receptor 2 and use of such compound in a treatment of an individual having a cardiac disorder. The invention further provides a kit that can be used to detect specific mutations in calmodulin encoding genes.

### RELATED APPLICATIONS

This application is the U.S. National Stage of International Application No. PCT/EP2013/057726, filed Apr. 12, 2013, which designates the U.S., published in English, and claims the benefit of U.S. Provisional Application No. 61/695,416, filed Aug. 31, 2012 and U.S. Provisional Application No. 61/623,191, filed Apr. 12, 2012.

### INCORPORATION BY REFERENCE OF MATERIAL IN ASCII TEXT FILE

This application incorporates by reference the Sequence Listing contained in the following ASCII text file being submitted concurrently herewith:
a) File name: 44751023001SEQLISTING.txt; created Apr. 9, 2015, 109 KB in size.

### FIELD OF INVENTION

The present invention relates to an isolated polynucleotide encoding at least a part of calmodulin and an isolated polypeptide comprising at least a part of a calmodulin protein, wherein the polynucleotide and the polypeptide comprise at least one mutation associated with a cardiac disorder. The present invention also relates to a method for determining whether an individual has an increased risk of contracting a cardiac disorder, a method for diagnosing a cardiac disorder, method for treatment of an individual having a cardiac disorder, method for identifying a compound, capable of enhancing the binding of calmodulin to ryanodine receptor 2 and use of such compound in a treatment of an individual having a cardiac disorder. The invention further provides a kit that can be used to detect specific mutations in calmodulin encoding genes.

### BACKGROUND OF INVENTION

Cardiac arrest without warning, due to an electrical malfunction in the heart muscle wall, is a well-known and feared cardiac event. This can be due to several underlying functional diseases of the heart. One example is disturbances, congenital or acquired, in the conduct of electrical impulses in the heart leading to this fatal incidence, responsible for more than 7,000,000 deaths worldwide each year (300,000 in the United States). However, in 5% to 10% of these cases, Sudden Cardiac Death (SCD) occurs in the absence of congenital heart disease (CHD) or cardiomyopathy.

During the past two decades a group of genetically-inherited abnormalities (“channelopathies”) have been identified (Campuzano, O. et al., Genet Med. 2010 May; 12(5):260-7) such as the long QT syndrome (LQTS), short QT syndrome (SQTS), Brugada syndrome, and catecholaminergic VT (CPVT), which can precipitate SCD without overt structural changes in the heart.

Prototype cardiac channelopathy characterized by delayed re-polarization of the myocardium, QT prolongation, and increased clinical risk for syncope, seizures, and sudden cardiac death is frequently described in the literature and is now understood to be a collection of genetically distinct arrhythmogenic cardiovascular disorders resulting from mutations in fundamental cardiac ion channels that orchestrate the action potential of the human heart (Vincent et al., N. Engl. J. Med., 327:846-852 (1992); Moss and Robinson, Ann. N.Y. Acad. Sci., 644:103-111 (1992); and Ackerman, Mayo Clin. Proc., 73:250-269 (1998)). Abnormalities in potassium and sodium channels, in ankyrin B, and in the ryanodine2 receptor (RyR2) of the sarcoplasmic reticulum, responsible for release of the calcium required for cardiac muscle contraction, can disrupt the normal electrical processes of the heart to cause life-threatening ventricular arrhythmias. Therefore, genetic information is progressively entering clinical practice and is being integrated in the risk stratification schemes in patients affected by inherited arrhythmogenic disorders.

Today, hundreds of LQTS-causing mutations have been discovered and about 75 percent of clinically-robust LQTS can be genetically elucidated with pathogenic mutations identifiable in three genes encoding critical ion channel sub-units, (Splawski et al., Circulation, 102:1178-1185 (2000) and Tester et al., Heart Rhythm, 2:507-517 (2005)): KCNQ1/KVLQT1 (LQT1; Wang et al., Nat. Genet., 12:17-23 (1996)), KCNH2/HERG (LQT2; Curran et al., Cell, 80:795-803 (1995)), SCN5A (LQT3; Wang et al., Cell, 80:805-811 (1995)).

Idiopathic ventricular tachycardia (VT) is a cardiac arrhythmia that is seen in patients without structural heart disease. Depending on the ECG characteristics it may be classified into monomorphic VT and polymorphic VT, the latter comprising a number of uncommon, often malignant familial disorders, including catecholaminergic polymorphic VT (CPVT) 1. CPVT is characterized by episodic syncope and/or sudden cardiac arrest induced by exercise or acute emotion. The ECG is usually within normal limits at rest often displaying prominent U waves, but may give way to ventricular arrhythmias at times of adrenergic activation. The arrhythmias, typically bidirectional and/or polymorphic VT, may develop into ventricular fibrillation and sudden death, causing this disorder to have a high mortality rate (30-50% by the age of 30). CPVT often manifests in childhood, and a family history of juvenile sudden death and stress-induced syncope is present in approximately one third of the cases. It may present as sudden death in children without any prior signs or warning, and may cause up to 15% of unexplained sudden cardiac deaths in young people (Cardiovasc. Dis, 2008, 51, 23-30).

Compared to LQTS, catecholaminergic polymorphic ventricular tachycardia (CPVT) is a more recent addition to the compendium of cardiac channelopathies (Leenhardt et al., Circulation, 91:1512-1519 (1995); Schimpf, R. et al., Herz Kardiovaskuläre Erkrankungen, Volume 34, Issue 4, pp 281-288 (June 2009)). Genetically, CPVT1-associated mutations, residing in critical regions of the RyR2-encoded cardiac ryanodine receptor/calcium release channel, account for about 50 to 65 percent of CPVT, whereas a small minority of patients has type 2 CPVT secondary to mutations in CASQ2-encoded calsequestrin (Mohamed, U. et al., Journal of Cardiovascular Electrophysiology, 18(7):791-797 (2007), Lahat et al., Circulation, 103:2822-2827 (2001); Priori et al., Circulation, 103:196-200 (2001); Priori et al., Circulation, 106:69-74 (2002); Marks, Circulation, 106(1):8-10 (2002); Lahat et al., Circulation, 107(3):e29 (2003); and Postma et al., Circulation Research, 91:E21-E26 (2002)). Phenotypically, CPVT is characterized by exertional syncope or sudden death in a structurally normal heart. The resting electrocardiogram in CPVT is completely normal. The electrocardiographic signature of CPVT is either exercise- or catecholamine-induced ventricular dysrhythmia. CPVT1 closely mimics the phenotype of LQTS, particularly concealed LQT1.

Mutations in the ryanodine receptor 2 gene (RYR2) are known to cause dominantly inherited CPVT (Circulation, 2001, 103, 196-200) and more than 70 different mutations are currently known. A less common autosomal recessive form of the disorder (CPVT2) is caused by mutations in the calsequestrin-2 gene (CASQ2) (Am. J. Hum. Genet, 2001, 69, 1378-1384). Mutations in these genes together can explain a little more than half of all familial CPVT cases. Ankyrin-2 (ANK2) mutations have been demonstrated to cause type 4 long-QT syndrome, however, a mutation in ANK2 was reported in a single individual with polymorphic VT similar to CPVT (Proc. Natl. Acad. Sci. U.S.A., 2004, 101, 9137-9142).

Tightly controlled cycling of the intracellular calcium concentration is the basis for cardiac muscle contraction and determination of heart rhythm. Control of the calcium concentration is governed by a complex network of ion-channels, enzymes and calcium binding proteins. A key intracellular calcium sensor and mediator of calcium signalling events is calmodulin (CaM), a remarkably conserved protein which is 100% identical at the amino acid level across all vertebrate species. The presence of three independent genes in the human genome, CALM1, CALM2 and CALM3, all encoding ubiquitously expressed identical CaM protein molecules, further underscores the selection pressure against any amino acid changes in this classic calcium binding protein.

### SUMMARY OF INVENTION

The present invention relates to the surprising identification of mutations in one of the human genes encoding calmodulin. Three mammalian genes, CALM1, CALM2 and CALM3 all encode calmodulin protein having the same amino acid sequence. Due to the essential function of the calmodulin genes and the extremely high conservation it was not expected that amino acid mutations in the calmodulin protein could be tolerated. The identification of amino acid causing mutations in a human gene encoding calmodulin is therefore surprising. Mutations in the calmodulin gene can, as described herein, cause disorders such as for example cardiac disorders or such as severe cardiac arrhythmia and sudden cardiac death.

One aspect of the present invention relates to an isolated polynucleotide encoding calmodulin (CaM) or at least a part of calmodulin (CaM), wherein said polynucleotide comprises at least one mutation associated with a disorder. In a preferred embodiment the present invention relates to an isolated polynucleotide encoding calmodulin (CaM) or at least a part of calmodulin (CaM), wherein said polynucleotide comprises at least one mutation associated with a cardiac disorder.

In a preferred embodiment the isolated nucleotide sequence has at least 90% sequence identity with a polynucleotide selected from the group consisting of SEQ ID NO:1 (CALM1), SEQ ID NO:2 (CALM2) and SEQ ID NO:3 (CALM3) or part thereof.

Preferably, the at least one mutation in the polynucleotide sequence results in at least one mutation in the encoded polypeptide sequence and, preferably, results in the encoded mutated calmodulin having one or more altered functional property compared to wild type calmodulin.

Preferably the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

In one embodiment, the altered functional property exhibited by the mutated calmodulin comprises or consists of aberrant binding to the RYR2 receptor. Preferably, binding is reduced or is abolished, as compared to the binding exhibited by wild type calmodulin. However, in an alternative embodiment, the binding affinity for RYR2 is increased, as compared to the binding affinity exhibited by wild type calmodulin.

In another embodiment, the altered functional property exhibited by the mutated calmodulin comprises or consists of aberrant binding to calcium (Ca²⁺). Preferably, binding is reduced or is abolished, as compared to the binding exhibited by wild type calmodulin. However, in an alternative embodiment, the binding affinity for calcium (Ca²⁺) is increased, as compared to the binding affinity exhibited by wild type calmodulin.

In one embodiment, the altered functional property exhibited by the mutated calmodulin comprises or consists of both aberrant binding to the RYR2 receptor (which may be either binding which is reduced or is abolished, as compared to the binding exhibited by wild type calmodulin, or which is increased, as compared to the binding exhibited by wild type calmodulin), and aberrant binding to calcium (and which may be either binding which is reduced or is abolished, as compared to the binding exhibited by wild type calmodulin, or which is increased, as compared to the binding exhibited by wild type calmodulin).

It is preferred that the at least one mutation results in an amino acid substitution.

In a particular embodiment the isolated polynucleotide comprises a mutation that results in the amino acid substitution Asn97Ser. As demonstrated in the accompanying Examples, the amino acid substitution Asn97Ser in calmodulin results in the reduction or abolition in its binding with the RYR2 receptor, and/or the reduction or abolition in its binding affinity for the RYR2 receptor. The accompanying Examples also demonstrate that the amino acid substitution Asn97Ser in calmodulin also results in the reduction or abolition in its binding to calcium, and/or the reduction or abolition in its binding affinity for calcium.

In another particular embodiment the isolated polynucleotide comprises a mutation that results in the amino acid substitution Asn53Ile. As demonstrated in the accompanying Examples, the amino acid substitution Asn53Ile in calmodulin results in its aberrant binding to calcium (such as an increase or a reduction or abolition in its binding to calcium, and/or an increase or a reduction or abolition in its binding affinity for calcium). In particular, the Examples show that the C-domain of the Asn53Ile variant (located in the N-domain) has a slightly increased calcium binding affinity and binds to the RYR2-peptide tryptophan (Trp) residue at slightly lower calcium concentrations compared to wild type calmodulin.

In one embodiment, the isolated polynucleotide comprises mutations that result in the amino acid substitution Asn97Ser and Asn53Ile.

A second aspect of the present invention relates to an isolated polypeptide comprising calmodulin (CaM) or at least a part of a calmodulin protein, wherein said polypeptide comprises at least one mutation associated with a cardiac disorder.

In a preferred embodiment the isolated polypeptide has at least 90% sequence identity with SEQ ID NO:4 or part thereof. In a particular embodiment the polypeptide comprises a sequence at least 90% identical to at least 20 contiguous amino acids of SEQ ID NO:4.

Preferably, the at least one mutation in the polypeptide sequence results in the encoded mutated calmodulin having one or more altered functional property compared to wild type calmodulin.

In one embodiment, the altered functional property exhibited by the mutated calmodulin comprises or consists of aberrant binding to the RYR2 receptor. Preferably, binding is reduced or is abolished, as compared to the binding exhibited by wild type calmodulin. However, in an alternative embodiment, the binding affinity for RYR2 is increased, as compared to the binding affinity exhibited by wild type calmodulin.

In another embodiment, the altered functional property exhibited by the mutated calmodulin comprises or consists of aberrant binding to calcium (Ca²⁺). Preferably, binding is reduced or is abolished, as compared to the binding exhibited by wild type calmodulin. However, in an alternative embodiment, the binding affinity for calcium (Ca²⁺) is increased, as compared to the binding affinity exhibited by wild type calmodulin.

In one embodiment, the altered functional property exhibited by the mutated calmodulin comprises or consists of both aberrant binding to the RYR2 receptor (which may be either binding which is reduced or is abolished, as compared to the binding exhibited by wild type calmodulin, or which is increased, as compared to the binding exhibited by wild type calmodulin), and aberrant binding to calcium (and which may be either binding which is reduced or is abolished, as compared to the binding exhibited by wild type calmodulin, or which is increased, as compared to the binding exhibited by wild type calmodulin).

It is preferred that the at least one mutation results in an amino acid substitution.

In a particular embodiment the isolated polypeptide comprises the mutation Asn97Ser. As demonstrated in the accompanying Examples, the amino acid substitution Asn97Ser in calmodulin results in the reduction or abolition in its binding with the RYR2 receptor, and/or the reduction or abolition in its binding affinity for the RYR2 receptor. The accompanying Examples also demonstrate that the amino acid substitution Asn97Ser in calmodulin also results in the reduction or abolition in its binding to calcium, and/or the reduction or abolition in its binding affinity for calcium.

In another particular embodiment the isolated polypeptide comprises a mutation that results in the amino acid substitution Asn53Ile. As demonstrated in the accompanying Examples, the amino acid substitution Asn53Ile in calmodulin results in its aberrant binding to calcium (such as an increase or a reduction or abolition in its binding to calcium, and/or an increase or a reduction or abolition in its binding affinity for calcium). In particular, the Examples show that the C-domain of the Asn53Ile variant (located in the N-domain) has a slightly increased calcium binding affinity and binds to the RYR2-peptide tryptophan (Trp) residue at slightly lower calcium concentrations compared to wild type calmodulin.

In one embodiment, the isolated polypeptide comprises mutations that result in the amino acid substitution Asn97Ser and Asn53Ile.

The term “cardiac disorder” as referred to herein includes any adverse event associated with the heart including, without limitation, an exertion- or exercise-induced cardiac event, sudden cardiac death, cardiac arrest, ventricular fibrillation, ventricular tachycardia, ventricular extrasystoles, premature ventricular contractions, and ventricular bigeminy. The cardiac disorder as referred to herein may for example be heart arrhythmia. In a preferred embodiment the cardiac disorder is Ventricular tachycardia, such as Polymorphic Ventricular Tachycardia or in particular Catecholerminergic Polymorphic Ventricular Tachycardia (CPVT). In further preferred embodiments, the cardiac disorder is selected from the group consisting of: Sudden Infant Death Syndrome (SIDS); Sudden Unexpected Death Syndrome (SUDS); syncope; seizure; cardiac event.

The identification of mutations in a calmodulin encoding gene can be used in methods for determining whether an individual has an increased risk of contracting a cardiac disorder and in methods for diagnosing a disorder such as a cardiac disorder of an individual. Thus, an aspect of the invention relates to a method for determining whether or not an individual has an increased risk of contracting a disorder or sudden cardiac death, wherein said method comprises determining the presence or absence of at least one mutation in a calmodulin encoding gene or in a part of a calmodulin encoding gene, as defined herein, wherein the presence of said at least one mutation indicates an increased risk of contracting a disorder or sudden cardiac death. The calmodulin encoding gene may for example be selected from the group consisting of CALM1, CALM2 and CALM3. It is preferred that the disorder is a cardiac disorder.

In a specific embodiment the present invention relates to a method for determining whether an individual has an increased risk of contracting a cardiac disorder or sudden cardiac death, wherein said method comprises
determining the presence or absence of at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof in a sample from said individual and/or
determining the presence or absence of at least one mutation in the polypeptide having SEQ ID NO:4 or at least 90% sequence identity with SEQ ID NO:4 or part thereof in a sample from said individual,
wherein the presence of said at least one mutation indicates an increased risk of contracting a cardiac disorder, and preferably wherein the at least one mutation results in the mutated calmodulin having one or more altered functional property compared to wild type calmodulin.

As discussed above, it is preferred that the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

Preferably, the altered functional property exhibited by the mutated calmodulin comprises or consists of aberrant binding to the RYR2 receptor (which binding is preferably increased or is reduced or is abolished) as compared to the binding exhibited by wild type calmodulin and/or comprises or consists of aberrant binding to calcium (Ca²⁺) (which binding is preferably increased or is reduced or is abolished) as compared to the binding exhibited by wild type calmodulin.

In such cases, it is possible to prophylactically treat individuals having an increased risk of contracting a cardiac disorder or sudden cardiac death, thereby reducing and/or abolishing the risk of onset of a cardiac disorder or sudden cardiac death.

Therefore, in one embodiment, the method for determining whether an individual has an increased risk of contracting a cardiac disorder or sudden cardiac death, the method comprises the further step, performed after having identifying an individual with an increased risk, of prophylactically treating that individual to reduce and/or abolish the risk of onset of a cardiac disorder or sudden cardiac death.

Methods for prophylactically treating individuals to reduce and/or abolish the risk of onset of cardiac disorders or sudden cardiac death are well known to those skilled in the art of medicine and pharmacy. For example, such individuals can be treated using “beta-blocker” therapy, and/or a calcium channel blocker, and/or via implantation of a defibrillator.

A further aspect of the present invention relates to a method for diagnosing a cardiac disorder of an individual, wherein said method comprises
determining the presence or absence of at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof in a sample from said individual and/or
determining the presence or absence of at least one mutation in the polypeptide having SEQ ID NO:4 or at least 90% sequence identity with SEQ ID NO:4 or part thereof in a sample from said individual,
wherein the presence of said at least one mutation indicates a cardiac disorder or an increased risk of contracting a cardiac disorder, and preferably wherein the at least one mutation results in the mutated calmodulin having one or more altered functional property compared to wild type calmodulin.

As discussed above, it is preferred that the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

Preferably, the altered functional property exhibited by the mutated calmodulin comprises or consists of aberrant binding to the RYR2 receptor (which binding is preferably increased or is reduced or is abolished) as compared to the binding exhibited by wild type calmodulin and/or comprises or consists of aberrant binding to calcium (Ca²⁺) (which binding is preferably increased or is reduced or is abolished) as compared to the binding exhibited by wild type calmodulin.

In one embodiment, the method for diagnosing a cardiac disorder of an individual comprises the further step, performed after said diagnosis, of treating an individual identified as having a cardiac disorder.

Methods for treating such cardiac disorders are well known to those skilled in the art of medicine and pharmacy. For example, individuals having a cardiac disorder can be treated using “beta-blocker” therapy, and/or a calcium channel blocker, and/or via implantation of a defibrillator. For example, it is particularly preferred for individuals identified as having LQTS alone to be treated using “beta-blocker” therapy, and for individuals having CPVT, or very early onset severe LQTS, to be treated using a calcium channel blocker and/or using an implanted defibrillator.

In a specific embodiment the at least one mutation to be determined in
CALM1 (SEQ ID NO:1), CALM2 (SEQ ID NO:2) and/or CALM3 (SEQ ID NO:3) and/or
a polynucleotide having at least 90% sequence identity with SEQ ID NO:1, SEQ ID NO:2 and/or SEQ ID NO:3 or part thereof results in the amino acid substitution Asn53Ile and/or in the amino acid substitution Asn97Ser.

In another specific embodiment the least one mutation to be determined in the polypeptide having SEQ ID NO:4 or at least 90% sequence identity with SEQ ID NO:4 or part thereof is Asn53Ile and/or Asn97Ser.

A further aspect of the present invention relates to a method for treatment of an individual having a cardiac disorder associated with at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof, wherein said mutation results in the mutated calmodulin having one or more altered functional property compared to wild type calmodulin, said method comprising administering to said individual an agent capable of restoring and/or improving the altered functional property to the level in wild type calmodulin.

As discussed above, it is preferred that the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

In one embodiment, the altered functional property exhibited by the mutated calmodulin may comprise or consist of aberrant binding to the RYR2 receptor (which binding is preferably reduced or is abolished) as compared to the binding exhibited by wild type calmodulin—in that embodiment, said method comprises administering to said individual an agent capable of increasing the binding between calmodulin and RYR2 in a therapeutically effective amount, thereby treating said individual.

In another embodiment, the altered functional property exhibited by the mutated calmodulin may comprise or consist of aberrant binding to the RYR2 receptor (which binding is preferably increased) as compared to the binding exhibited by wild type calmodulin—in that embodiment, said method comprises administering to said individual an agent capable of decreasing the binding between calmodulin and RYR2 in a therapeutically effective amount, thereby treating said individual.

In a further embodiment, the altered functional property exhibited by the mutated calmodulin may comprise or consist of aberrant binding to calcium (Ca²⁺) (which binding is preferably reduced or is abolished) as compared to the binding exhibited by wild type calmodulin—in that embodiment, said method comprises administering to said individual an agent capable of increasing the binding between calmodulin and calcium (Ca²⁺) in a therapeutically effective amount, thereby treating said individual.

In a further embodiment, the altered functional property exhibited by the mutated calmodulin may comprise or consist of aberrant binding to calcium (Ca²⁺) (which binding is preferably increased) as compared to the binding exhibited by wild type calmodulin—in that embodiment, said method comprises administering to said individual an agent capable of decreasing the binding between calmodulin and calcium (Ca²⁺) in a therapeutically effective amount, thereby treating said individual.

In a particular embodiment the at least one mutation results in the amino acid substitution Asn97Ser and/or in the amino acid substitution Asn53Ile

The term “cardiac disorder” as referred to herein includes any adverse event associated with the heart including, without limitation, an exertion- or exercise-induced cardiac event, sudden cardiac death, cardiac arrest, ventricular fibrillation, ventricular tachycardia, ventricular extrasystoles, premature ventricular contractions, and ventricular bigeminy. The cardiac disorder may for example be heart arrhythmia. In one embodiment the cardiac disorder is Ventricular tachycardia or Polymorphic Ventricular Tachycardia. In a particular embodiment the cardiac disorder is Catecholerminergic Polymorphic Ventricular Tachycardia (CPVT). The cardiac disorder may in an embodiment be drug-induced arrhythmia. In further preferred embodiments, the cardiac disorder is selected from the group consisting of: Sudden Infant Death Syndrome (SIDS); Sudden Unexpected Death Syndrome (SUDS); syncope; seizure; cardiac event.

A further aspect of the present invention relates to a method for identifying a compound, capable of enhancing the binding of calmodulin to ryanodine receptor 2, wherein said calmodulin comprises at least one mutation that decreases the binding affinity to ryanodine receptor 2 (and which preferably reduces or abolishes binding to ryanodine receptor 2), said method comprising
providing a first sample comprising calmodulin protein or a fragment thereof having a mutation that decreases the binding affinity to ryanodine receptor 2 (and which preferably reduces or abolishes binding to ryanodine receptor 2), ryanodine receptor 2 or a fragment thereof and a test compound
measuring the amount of calmodulin protein bound to ryanodine receptor 2 protein in said first sample
providing a second sample comprising calmodulin protein or a fragment thereof having a mutation that decreases the binding affinity to ryanodine receptor 2 (and which preferably reduces or abolishes binding to ryanodine receptor 2), and ryanodine receptor 2 or a fragment thereof
measuring the amount of calmodulin protein bound to ryanodine receptor 2 protein in said second sample
comparing the amount of calmodulin protein bound to ryanodine receptor 2 protein in the first and second sample, whereby a higher amount of calmodulin protein bound to ryanodine receptor 2 protein in the first sample as compared to the second sample indicates that the test compound enhances binding of calmodulin protein to ryanodine receptor 2 protein.

Preferably, the mutation that decreases the binding affinity to ryanodine receptor 2 (and which preferably reduces or abolishes binding to ryanodine receptor 2) comprises the amino acid substitution Asn97Ser and, more preferably, comprises the amino acid substitutions Asn97Ser and Asn53Ile.

It is preferred that the calmodulin protein has at least 90% sequence identity with SEQ ID NO:4 or part thereof.

In a particular embodiment the at least one mutation in the amino acid sequence having SEQ ID NO:4 or at least 90% sequence identity with SEQ ID NO:4 or part thereof is Asn97Ser.

A still further aspect of the present invention relates to a method for identifying a compound capable of enhancing the binding between calmodulin and calcium (Ca²⁺), wherein said calmodulin comprises at least one mutation that decreases the binding affinity to calcium (Ca²⁺) (and which preferably reduces or abolishes binding to calcium (Ca²⁺)), said method comprising
providing a first sample comprising calmodulin protein or a fragment thereof having a mutation that decreases the binding affinity to calcium (Ca²⁺)(and which preferably reduces or abolishes binding to calcium (Ca²⁺)), and a test compound
measuring the amount of calmodulin protein bound to calcium (Ca²⁺) in said first sample
providing a second sample comprising calmodulin protein or a fragment thereof having a mutation that decreases the binding affinity to calcium (Ca²⁺)(and which preferably reduces or abolishes binding to calcium (Ca²⁺))
measuring the amount of calmodulin protein bound to calcium (Ca²⁺) in said second sample
comparing the amount of calmodulin protein bound to calcium (Ca²⁺) in the first and second sample, whereby a higher amount of calmodulin protein bound to calcium (Ca²⁺) in the first sample as compared to the second sample indicates that the test compound enhances binding of calmodulin protein to calcium (Ca²⁺).

Preferably, the mutation that decreases the binding affinity to calcium (Ca²⁺) (and which preferably reduces or abolishes binding to calcium (Ca²⁺)) comprises the amino acid substitution Asn53Ile and, more preferably, comprises the amino acid substitutions Asn53Ile and Asn97Ser.

It is preferred that the calmodulin protein has at least 90% sequence identity with SEQ ID NO:4 or part thereof.

In a particular embodiment the at least one mutation in the amino acid sequence having SEQ ID NO:4 or at least 90% sequence identity with SEQ ID NO:4 or part thereof is Asn53Ile.

Yet another aspect of the present invention relates to a compound identified by the methods described above for use in treatment of an individual having a cardiac disorder associated with at least one mutation in CALM1 (SEQ ID NO:1), CALM2 (SEQ ID NO:2) and/or CALM3 (SEQ ID NO:3) and/or in a nucleotide sequence having at least 90% sequence identity with SEQ ID NO:1, SEQ ID NO:2 and/or SEQ ID NO:3 or part thereof, wherein said mutation results in the mutated calmodulin having one or more altered functional property compared to wild type calmodulin.

As discussed above, it is preferred that the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

In one embodiment, the altered functional property exhibited by the mutated calmodulin may comprise or consist of aberrant binding to the RYR2 receptor (which binding is preferably increased or is reduced or is abolished) as compared to the binding exhibited by wild type calmodulin. In another embodiment, the altered functional property exhibited by the mutated calmodulin may comprise or consist of aberrant binding to calcium (Ca²⁺) (which binding is preferably increased or is reduced or is abolished) as compared to the binding exhibited by wild type calmodulin.

In a preferred embodiment SEQ ID NO:1, SEQ ID NO:2 and/or SEQ ID NO:3 and/or the nucleotide sequence having at least 90% sequence identity with SEQ ID NO:1, SEQ ID NO:2 and/or SEQ ID NO:3 or part thereof comprise a mutation that results in the amino acid substitution Asn97Ser and/or in the amino acid substitution Asn53Ile.

A further aspect of the present invention relates to a pharmaceutical composition for use in the treatment of an individual having a cardiac disorder associated with at least one mutation in CALM1 (SEQ ID NO:1), CALM2 (SEQ ID NO:2) and/or CALM3 (SEQ ID NO:3), wherein said mutation results in the mutated calmodulin having one or more altered functional property compared to wild type calmodulin, and wherein said composition comprises an agent capable of restoring and/or improving the altered functional property to the level in wild type calmodulin.

In one embodiment, the altered functional property exhibited by the mutated calmodulin may comprise or consist of aberrant binding to the RYR2 receptor (which binding is preferably reduced or is abolished) as compared to the binding exhibited by wild type calmodulin—in that embodiment, said composition comprises an agent capable of restoring and/or increasing the binding between calmodulin and RYR2.

In another embodiment, the altered functional property exhibited by the mutated calmodulin may comprise or consist of aberrant binding to the RYR2 receptor (which binding is preferably increased) as compared to the binding exhibited by wild type calmodulin—in that embodiment, said composition comprises an agent capable of restoring and/or decreasing the binding between calmodulin and RYR2.

In a further embodiment, the altered functional property exhibited by the mutated calmodulin may comprise or consist of aberrant binding to calcium (Ca²⁺) (which binding is preferably reduced or is abolished) as compared to the binding exhibited by wild type calmodulin—in that embodiment, said composition comprises an agent capable of restoring and/or increasing the binding between calmodulin and calcium (Ca²⁺).

In a further embodiment, the altered functional property exhibited by the mutated calmodulin may comprise or consist of aberrant binding to calcium (Ca²⁺) (which binding is preferably increased) as compared to the binding exhibited by wild type calmodulin—in that embodiment, said composition comprises an agent capable of restoring and/or decreasing the binding between calmodulin and calcium (Ca²⁺).

The term “cardiac disorder” as referred to herein includes any adverse event associated with the heart including, without limitation, an exertion- or exercise-induced cardiac event, sudden cardiac death, cardiac arrest, ventricular fibrillation, ventricular tachycardia, ventricular extrasystoles, premature ventricular contractions, and ventricular bigeminy. The cardiac disorder may for example be heart arrhythmia. In one embodiment the cardiac disorder is Ventricular tachycardia or Polymorphic Ventricular Tachycardia. In a particular embodiment the cardiac disorder is Catecholerminergic Polymorphic Ventricular Tachycardia (CPVT). The cardiac disorder may in an embodiment be drug-induced arrhythmia. In further preferred embodiments, the cardiac disorder is selected from the group consisting of: Sudden Infant Death Syndrome (SIDS); Sudden Unexpected Death Syndrome (SUDS); syncope; seizure; cardiac event.

An aspect of the present invention relates to a kit for detecting at least one mutation in a polynucleotide encoding calmodulin (CaM) or at least a part of calmodulin (CaM), wherein said kit comprises at least one oligonucleotide that is complementary to a sequence of said calmodulin encoding gene such that if the mutation is present in the polynucleotide, strand elongation from said oligonucleotide results in an extension product comprising said mutation.

In a preferred embodiment the polynucleotide has at least 90% sequence identity with a polynucleotide selected from the group consisting of SEQ ID NO:1, SEQ ID NO:2 and SEQ ID NO:3 or part thereof.

Preferably, the at least one mutation in the polypeptide sequence results in the encoded mutated calmodulin having one or more altered functional property compared to wild type calmodulin, as described herein.

In one embodiment, the altered functional property exhibited by the mutated calmodulin comprises or consists of aberrant binding to the RYR2 receptor, which binding is preferably increased or is reduced or is abolished, as compared to the binding exhibited by wild type calmodulin. In another embodiment, the altered functional property exhibited by the mutated calmodulin comprises or consists of aberrant binding to calcium (Ca²⁺), which binding is preferably increased or is reduced or is abolished, as compared to the binding exhibited by wild type calmodulin.

In one embodiment, the altered functional property exhibited by the mutated calmodulin comprises or consists of both aberrant binding to the RYR2 receptor (and, preferably, binding which is increased or is reduced or is abolished, as compared to the binding exhibited by wild type calmodulin) and aberrant binding to calcium (and, preferably, binding which is increased or is reduced or is abolished, as compared to the binding exhibited by wild type calmodulin). It is preferred that the at least one mutation results in an amino acid substitution—preferably, the at least one mutation comprises the amino acid substitution Asn97Ser and/or Asn53Ile.

### DESCRIPTION OF DRAWINGS

FIG. 1: Pedigree and electrocardiography

(A) Pedigree of Swedish family showing haplotypes in the region of maximum linkage on chromosome 14. The index patient is indicated with an arrow. (B) The ECG during rest and an arrhythmia event (while playing football) occurring during a 24 h ECG registration of the index patient II:6 of family 1. (C) Resting and exercise ECG's from a second unrelated patient of Iraqi origin. Both resting ECGs demonstrate prominent U waves in the anterior, while bidirectional ventricular ectopy is seen during physical activity or exercise.

FIG. 2: Genome-wide linkage analysis

A novel locus on chromosome 14q32 (lod score 3.9) harbouring approximately 100 genes.

FIG. 3: CALM1 gene structure and mutations

(A) Position of the Asn53Ile and Asn97Ser mutations identified in two different patients with ventricular tachycardia, and the sequencing electropherograms of exon 3 and 5 comparing patient and control subject. (B) Genotyping assay for the mutation identified in the Swedish family, with two peaks found for all subjects carrying the heterozygous mutation.

FIG. 4: Sequence alignment

Alignment of calmodulin amino acid sequences from different species with the Asn53Ile and Asn97Ser mutations indicated (red residues). Secondary structure elements are shown as green and orange shades (α-helix and β-strand, respectively) and the Ca²⁺ binding residues are framed. Dots indicate conserved residues, showing calmodulin is 100% conserved in all vertebrates and only displaying few changes to round worm and plants.

FIG. 5: Three dimensional structures of calmodulin

3D-structure models of calmodulin indicating the positions of the mutated residues Asn53 and Asn97 (shown in stick representation). (A) apo-calmodulin, (B) Ca²⁺ saturated calmodulin, and (C) Ca²⁺ bound calmodulin/RYR1 peptide complex. Calcium ions are shown as grey spheres, the RYR1 peptide in red. The two aromatic hydrophobic binding anchors in RYR1 are shown in stick representation. Asn53 is positioned on the solvent exposed side of α-helix C, not in contact with either Ca²⁺, peptide ligand, or other calmodulin domains. Asn97 is one of the Ca²⁺ coordinating residues of Ca²⁺ binding site Ill. The calmodulin N-domain is positioned on top and the C-domain at the bottom. The orientation of the calmodulin N-domain is rotated roughly 90 degrees counter clockwise around the vertical axis between the apo-, Ca²⁺-, and RYR1-complexed calmodulin structures.

FIG. 6: Calcium binding

The relative Ca²⁺ binding of the C-domain of calmodulin as a function of total Ca²⁺ concentration, determined as the change in the tyrosine fluorescence intensity. The data presents the average of three independent experiments (+/−SD), in most cases the error bars are smaller than the symbols. a: p<0.05, b: p<0.01, c: p<0.001, and d: p<0.0001 compared to native calmodulin.

FIG. 7: RYR2 binding

Calmodulin binding to RYR2 peptide (R3581-L3611). The RYR2 Trp3586 fluorescence emission spectrum, normalized to the maximal fluorescence intensity of the peptide alone, is shown without added calmodulin (RYR) and with a saturating amount of the indicated calmodulin variants added at (A) low intracellular (100 nM free Ca²⁺), (B) moderate (1 μM free Ca²⁺), and (C) saturating (200 μM Ca²⁺) concentration.

FIG. 8: SNP genome wide linkage analysis.

FIG. 9: Equilibrium titration curves (symbols) fitted the two-site Adair model (lines). Lobe specific Ca²⁺ binding curves for the N- and C-lobe of the three CaM variants, respectively, plotted as fractional saturation as a function of [Ca²⁺]_{free}.

FIG. 10: CaM N- and C-lobe Ca²⁺ dissociation measured by stopped-flow experiments. Left: CaM Ca²⁺ dissociations at 8° C. monitored by increase in Ca²⁺-probe Quin-2 fluorescence. Right: C-lobe dissociation monitored by decrease in tyrosine fluorescence at different temperatures (10, 15, 20, 25, 30 and 37° C.). Notice the alternate x-axis for the N97S.

FIG. 11: CaM C-lobe k_{off} values as a function of temperature. Bars correspond to the 95% confidence interval (symbol size covers this interval).

FIG. 12: Thermal and thermo-chemical denaturation of apo- and CaCaM variants, respectively. Top: Fractional unfolding of the apoCaM variants monitored as the normalized change in the θ_{MRW}^{222 nm} signal. Middle: Fractional unfolding of the apoCaM C-lobes monitored as the normalized increase in tyrosine fluorescence signal. Bottom: Fractional unfolding of CaCaM in the presence of 5 M urea monitored as the normalized change in the θ_{MRW}^{222 nm} signal.

FIG. 13: CaM variants structural integrity. Far UV CD spectra of CaM variants in the A) Ca²⁺ free and B) Ca²⁺ loaded form. C) Native gel-electrophoresis of CaM variants, analysed individually, or mixed with WT CaM prior to loading.

FIG. 14: Equilibrium Ca²⁺ titration curves (symbols) fitted by a two-site Adair model (lines). Fractional saturation of the tryptophan 340/356 nm emission ratio plotted as a function of [Ca⁺²]_{free}, illustrating Ca²⁺ binding of the C-lobe for the three CaM variants in complex with RYR2p.

### DETAILED DESCRIPTION OF THE INVENTION

### Definitions

The term “diagnose”, “diagnostic”, “diagnosis” or “diagnosing” as used herein refer to distinguishing or identifying a disease, syndrome or condition or distinguishing or identifying a person having a particular disease, syndrome or condition. Usually, a diagnosis of a disease or disorder is based on the evaluation of one or more factors and/or symptoms that are indicative of the disease. In this regard, the term means assessing whether or not an individual or a subject has a mutation in the CALM1, CALM2 and/or CALM3 gene.

As used herein, the term “predisposed” or “predisposition” refers to an increased likelihood that a patient may be afflicted with a disease.

The term “heterozygous” as used herein refers to a genotype of a diploid organism consisting of two different alleles at a locus or two different alleles of a gene. Heterozygous may also refer to a sample from an organism in which two different alleles at a locus may be detected. Methods, such as for example nucleotide sequencing, for determining whether a sample is heterozygous are well known in the art.

The term “homozygous” refers to a genotype of a diploid organism consisting of two identical alleles at a locus or two identical alleles of a gene. “Homozygous” may also refer to a sample from an organism in which two different alleles at a locus may be detected. Methods, such as for example nucleotide sequencing, for determining whether a sample is homozygous are well known in the art.

As used herein, the term “sample” or “biological sample” refers to any liquid or solid material obtained from a biological source, such a cell or tissue sample or bodily fluids. “Bodily fluids” include, but are not limited to, blood, serum, plasma, saliva, cerebrospinal fluid, pleural fluid, tears, lactal duct fluid, lymph, sputum, urine, saliva, amniotic fluid, and semen.

The terms ‘therapeutically effective amount’ means an amount that is sufficient to elicit a desired response.

The term ‘nucleotide’ as used herein refers to a monomer of RNA or DNA. A nucleotide is a ribose or a deoxyribose ring attached to both a base and a phosphate group. Both mono-, di-, and tri-phosphate nucleosides are referred to as nucleotides.

Nucleotides according to the invention includes ribonucleotides comprising a nucleobase selected from the group consisting of adenine (A), uracil (U), guanine (G), and cytosine (C), and deoxyribonucleotide comprising a nucleobase selected from the group consisting of adenine (A), thymine (T), guanine (G), and cytosine (C). Nucleobases are capable of associating specifically with one or more other nucleobases via hydrogen bonds. The specific interaction of one nucleobase with another nucleobase is generally termed “base-pairing”. The base pairing results in a specific hybridisation between predetermined and complementary nucleotides. Complementary nucleotides according to the present invention are nucleotides that comprise nucleobases that are capable of base-pairing. Of the naturally occurring nucleobases adenine (A) pairs with thymine (T) or uracil (U); and guanine (G) pairs with cytosine (C). Accordingly, e.g. a nucleotide comprising A is complementary to a nucleotide comprising either T or U, and a nucleotide comprising G is complementary to a nucleotide comprising C.

The term “polynucleotide” as used herein refers to a biopolymer composed of nucleotide monomers covalently bonded in a chain. DNA (deoxyribonucleic acid) and RNA (ribonucleic acid) are examples of polynucleotides.

The term “oligonucleotide” as used herein refers to a biopolymer composed of nucleotide monomers covalently bonded in a chain. DNA (deoxyribonucleic acid) and RNA (ribonucleic acid) are examples of oligonucleotides. The oligonucleotide as used herein is typically shorter than a polynucleotide. The oligonucleotide may for example be used to prime synthesis of nucleotide strands during PCR.

The term “gene” as used herein refers to its normal meaning, a nucleic acid sequence with a transcriptional capability, i.e., which can be transcribed into an RNA sequence (an expressed sequence) which in most cases, is translated into an amino acid sequence, along with the regulatory sequences that regulate expression or engage in the expression of expressed sequences. A gene is composed of introns that are the non-coding part of a gene and exons encoding the amino acid sequence of a protein

The term “coding region” as used herein refers to the coding region of a gene. The coding region is composed of exons, which encodes the amino acid sequence of the resulting protein.

The term “isolated”, when referring to a polynucleotide or a polypeptide refers to a naturally-occurring nucleic acid or a polypeptide (or fragments thereof) that is substantially free from the naturally-occurring molecules and cellular components with which it is naturally associated. For example, any nucleic acid or a polypeptide that has been produced synthetically is considered to be isolated. Nucleic acids that are recombinantly expressed, cloned, produced by a primer extension reaction (e.g., PCR), or otherwise excised from a genome are also considered to be isolated. Similarly, polypeptides that are recombinantly produced are also considered to be isolated. However, cDNA preparations, and the like, are not considered isolated because they do not contain naturally-occurring molecules.

As used herein the term “base mismatch” refers to a change in the nucleotides, such that when for example a primer anneals to a polynucleotide an abnormal base pairing of nucleotides is formed such as for example base pairing between G-G, C-C, A-A, T-T, A-G, A-C, T-G, or T-C. Normally guanine (G) binds to cytosine (C) and adenine (A) binds to thymine (T) in the formation of double stranded nucleic acids. The standard base pairing is A-T or G-C. Thus, base pairing between A-T or G-C is not a base mismatch.

As used herein the term “point mutation” refers to a mutation wherein a single nucleotide is exchanged for another. The point mutation may be an A>G mutation, an A>C mutation, an A>T mutation, a T>G mutation, a T>C mutation, a T>A mutation, a G>T mutation, a G>C mutation, n G>A mutation, a C>G mutation, a C>T mutation or a C>A mutation. A>G means that A is replaced with G.

The term “missense mutation” as used herein is a point mutation in which a single nucleotide is changed, resulting in a codon that codes for a different amino acid.

The term “dominant mutation” as used herein is a mutation in one allele that results in a phenotype, such as for example a cardiac disease, although the corresponding wild type allele is present. Thus a dominant mutation needs only to be present in one allele to result in a phenotype or disease.

The term “binding affinity” as used herein refers to the strength of binding between two molecules, for example between two proteins.

The term “annealing” as used herein refers to the pairing of complementary DNA or RNA sequences by hydrogen bonding to form a double-stranded polynucleotide. The term is for example used to describe the binding of a DNA probe, or the binding of a primer to a DNA strand during a polymerase chain reaction (PCR). The term is also used to describe the reformation (renaturation) of complementary nucleotide strands that were separated by heat (thermally denatured).

The term “amplicon” as used herein refers to a polynucleotide that is amplified. In the present invention, the polynucleotide that is amplified is the sequence between a competitive primer (which may for example be the 5′ primer) and a common primer (which may for example be the 3′ primer). The amplicon results from annealing of the extension products of a common primer and a competitive primer. Thus, the amplicon is a double stranded nucleotide and comprises the polynucleotide of the primers and the polynucleotide between the 5′ primer and 3′ primer. In a preferred embodiment the amplicon is a double stranded DNA molecule.

The term “polymerase” as used herein refers to an enzyme that catalyses the synthesis of a polynucleotide such as RNA or DNA against a nucleotide template strand by adding free nucleotides to the growing polynucleotide using base-pairing interactions. Thus, a polymerase catalyses the polymerization of nucleotides into a polynucleotide using an intact nucleotide strand as a template. In a preferred embodiment the polymerase is a DNA polymerase. A DNA polymerase is an enzyme that catalyzes the polymerization of deoxyribonucleotides using a DNA strand as a template. In a preferred embodiment the DNA polymerase is a Taq polymerase from Thermus aquaticus, which is a thermostable DNA polymerase having an optimum temperature for activity of about 70 to 80° C. Typically a temperature of 72° C. is used.

### Calmodulin Polynucleotide Comprising at Least One Mutation

Calmodulin (CaM) is a calcium-binding messenger protein that transduces calcium signals by binding calcium ions and then modifying its interactions with various target proteins. CaM mediates many crucial processes such as inflammation, metabolism, apoptosis, smooth muscle contraction, intracellular movement, short-term and long-term memory, and the immune response. CaM is expressed in many cell types and can have different subcellular locations, including the cytoplasm, within organelles, or associated with the plasma or organelle membranes. Many of the proteins that CaM binds are unable to bind calcium themselves, and as such use CaM as a calcium sensor and signal transducer. CaM can also make use of the calcium stores in the endoplasmic reticulum, and the sarcoplasmic reticulum.

The present invention relates to the surprising identification of mutations in one of the human genes encoding calmodulin. Three mammalian genes, CALM1, CALM2 and CALM3 all encode calmodulin protein having the same amino acid sequence. Calmodulin is a ubiquitous protein expressed in all eukaryotic cells, and it is extremely conserved. It shows 100% amino acid identity among vertebrates (J. Biochem., 126 (1999), pp. 572-577). Due to the essential function of the calmodulin genes and the extremely high conservation it was not expected that amino acid mutations in the calmodulin protein could be tolerated. The identification of amino acid causing mutations in a human gene encoding calmodulin is therefore surprising. Mutations in the calmodulin gene can, as described herein, cause cardiac disorders such as for example severe cardiac arrhythmia and sudden cardiac death.

Accordingly, the present invention directly allows for pre-symptomatic genetic diagnosis in families with severe cardiac disorders, enabling initiation of potentially life-saving treatment for children and young individuals carrying disease mutations. For example, the three calmodulin genes CALM1, CALM2 and CALM3 are candidates for genetic screening of patients with CPVT-like arrhythmia and unexplained sudden cardiac death.

One aspect of the present invention relates to an isolated polynucleotide encoding calmodulin (CaM) or at least a part of calmodulin (CaM), wherein said polynucleotide comprises at least one mutation associated with a disorder. In a preferred embodiment the present invention relates to an isolated polynucleotide encoding at least a part of calmodulin (CaM), wherein said polynucleotide comprises at least one mutation associated with a cardiac disorder.

Preferably, the at least one mutation in the polynucleotide sequence results in at least one mutation in the encoded polypeptide sequence and, preferably, results in the encoded mutated calmodulin having one or more altered functional property compared to wild type calmodulin.

As discussed above, it is preferred that the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

In one embodiment, the altered functional property exhibited by the mutated calmodulin comprises or consists of aberrant binding to the RYR2 receptor. Preferably, binding is reduced or is abolished, as compared to the binding exhibited by wild type calmodulin. However, in an alternative embodiment, the binding affinity for RYR2 is increased, as compared to the binding affinity exhibited by wild type calmodulin.

In another embodiment, the altered functional property exhibited by the mutated calmodulin comprises or consists of aberrant binding to calcium (Ca²⁺). Preferably, binding is reduced or is abolished, as compared to the binding exhibited by wild type calmodulin. However, in an alternative embodiment, the binding affinity for calcium (Ca²⁺) is increased, as compared to the binding affinity exhibited by wild type calmodulin.

In one embodiment, the altered functional property exhibited by the mutated calmodulin comprises or consists of both aberrant binding to the RYR2 receptor (which may be either binding which is reduced or is abolished, as compared to the binding exhibited by wild type calmodulin, or which is increased, as compared to the binding exhibited by wild type calmodulin), and aberrant binding to calcium (and which may be either binding which is reduced or is abolished, as compared to the binding exhibited by wild type calmodulin, or which is increased, as compared to the binding exhibited by wild type calmodulin).

In one embodiment the isolated polynucleotide has at least 85% sequence identity, such as at least 90% sequence identity, at least 95% sequence identity, such as at least 97% sequence identity, at least 98% sequence identity, such as at least 99% sequence identity with a polynucleotide selected from the group consisting of SEQ ID NO:1 (CALM1), SEQ ID NO:2 (CALM2) and SEQ ID NO:3 (CALM3) or part thereof.

In a particular embodiment the isolated polynucleotide has at least 85% sequence identity, such as at least 90% sequence identity, at least 95% sequence identity, such as at least 97% sequence identity, at least 98% sequence identity, such as at least 99% sequence identity with a polynucleotide selected from the group consisting of SEQ ID NO:1, SEQ ID NO:2 and SEQ ID NO:3.

It is preferred that the isolated polynucleotide has at least 90% sequence identity with a polynucleotide selected from the group consisting of SEQ ID NO:1, SEQ ID NO:2 and SEQ ID NO:3 or part thereof.

The isolated polynucleotide as described herein and above may for example comprise at least 10 nucleotides, such as for example at least 15 nucleotides, such as at least 20 nucleotides, at least 30 nucleotides, such as for example at least 40 nucleotides, such as at least 50 nucleotides, at least 60 nucleotides, such as for example at least 80 nucleotides, such as at least 100 nucleotides, at least 150 nucleotides, such as for example at least 200 nucleotides, such as at least 300 nucleotides, at least 400 nucleotides or such as for example at least 500 nucleotides of SEQ ID NO:1, SEQ ID NO:2 or SEQ ID NO:3. In one embodiment the isolated polynucleotide comprises the entire polynucleotide of SEQ ID NO:1, SEQ ID NO:2 or SEQ ID NO:3.

The isolated polynucleotide may in an embodiment comprise a sequence at least 85% identical, such as at least 90% identical, at least 95% identical, such as for example at least 97% identical, at least 98% identical, such as at least 99% identical to at least 10 contiguous nucleotides, such as at least 15 contiguous nucleotides, such as for example 30 contiguous nucleotides, at least 40 contiguous nucleotides, such as at least 50 contiguous nucleotides, such as for example 60 contiguous nucleotides at least 80 contiguous nucleotides, such as at least 100 contiguous nucleotides, such as for example 150 contiguous nucleotides, at least 200 contiguous nucleotides, such as at least 300 contiguous nucleotides or such as for example 400 contiguous nucleotides of a polynucleotide selected from the group consisting of SEQ ID NO:1, SEQ ID NO:2 and SEQ ID NO:3.

In a particular embodiment the isolated polynucleotide comprises a sequence at least 90% identical to at least 20 contiguous nucleotides of a polynucleotide selected from the group consisting of SEQ ID NO:1, SEQ ID NO:2 and SEQ ID NO:3.

In another particular embodiment the isolated polynucleotide has at least 90% sequence identity with the entire length of SEQ ID NO:1, SEQ ID NO:2 or SEQ ID NO:3.

It is preferred that the polynucleotide as described herein comprises at least a part of the coding region of SEQ ID NO:1, SEQ ID NO:2 or SEQ ID NO:3.

In one embodiment the isolated polynucleotide as described herein encodes at least 10 amino acids, such as at least 15 amino acids, such as for example at least 20 amino acids, such as at least 30 amino acids, such as for example at least 40 amino acids, such as at least 50 amino acids, such as for example at least 60 amino acids, such as at least 80 amino acids, such as for example at least 100 amino acids, such as at least 120 amino acids, such as for example at least 140 amino acids of calmodulin (SEQ ID NO:4).

In one embodiment the polynucleotide sequence as described herein is a cDNA sequence or an mRNA sequence encoding a encoding at least a part of calmodulin (CaM), wherein said polynucleotide comprises at least one mutation associated with a disorder. In a preferred embodiment the disorder is a cardiac disorder.

In one embodiment the isolated polynucleotide has at least 85% sequence identity, such as at least 90% sequence identity, at least 95% sequence identity, such as at least 97% sequence identity, at least 98% sequence identity, such as at least 99% sequence identity with a polynucleotide selected from the group consisting of SEQ ID NO:5 (CALM1 mRNA), SEQ ID NO:6 (CALM2 mRNA) and SEQ ID NO:7 (CALM3 mRNA) or part thereof.

In a particular embodiment the isolated polynucleotide has at least 85% sequence identity, such as at least 90% sequence identity, at least 95% sequence identity, such as at least 97% sequence identity, at least 98% sequence identity, such as at least 99% sequence identity with a polynucleotide selected from the group consisting of SEQ ID NO:5 (CALM1 mRNA), SEQ ID NO:6 (CALM2 mRNA) and SEQ ID NO:7 (CALM3 mRNA) or part thereof.

It is preferred that the isolated polynucleotide has at least 90% sequence identity with a polynucleotide selected from the group consisting of SEQ ID NO:5 (CALM1 mRNA), SEQ ID NO:6 (CALM2 mRNA) and SEQ ID NO:7 (CALM3 mRNA) or part thereof.

The isolated polynucleotide as described herein and above may for example comprise at least 10 nucleotides, such as for example at least 15 nucleotides, such as at least 20 nucleotides, at least 30 nucleotides, such as for example at least 40 nucleotides, such as at least 50 nucleotides, at least 60 nucleotides, such as for example at least 80 nucleotides, such as at least 100 nucleotides, at least 150 nucleotides, such as for example at least 200 nucleotides, such as at least 300 nucleotides, at least 400 nucleotides or such as for example at least 500 nucleotides of SEQ ID NO:5, SEQ ID NO:6 or SEQ ID NO:7. In one embodiment the isolated polynucleotide comprises the entire polynucleotide of SEQ ID NO:5, SEQ ID NO:6 or SEQ ID NO:7.

The isolated polynucleotide may in an embodiment comprise a sequence at least 85% identical, such as at least 90% identical, at least 95% identical, such as for example at least 97% identical, at least 98% identical, such as at least 99% identical to at least 10 contiguous nucleotides, such as at least 15 contiguous nucleotides, such as for example 30 contiguous nucleotides, at least 40 contiguous nucleotides, such as at least 50 contiguous nucleotides, such as for example 60 contiguous nucleotides at least 80 contiguous nucleotides, such as at least 100 contiguous nucleotides, such as for example 150 contiguous nucleotides, at least 200 contiguous nucleotides, such as at least 300 contiguous nucleotides or such as for example 400 contiguous nucleotides of a polynucleotide selected from the group consisting of SEQ ID NO:5, SEQ ID NO:6 and SEQ ID NO:7.

In a particular embodiment the isolated polynucleotide comprises a sequence at least 90% identical to at least 20 contiguous nucleotides of a polynucleotide selected from the group consisting of SEQ ID NO:5, SEQ ID NO:6 and SEQ ID NO:7.

In another particular embodiment the isolated polynucleotide has at least 90% sequence identity with the entire length of SEQ ID NO:5, SEQ ID NO:6 or SEQ ID NO:76.

It is preferred that the polynucleotide as described herein comprises at least a part of the coding region of SEQ ID NO:5, SEQ ID NO:6 or SEQ ID NO:7.

The polynucleotide as described herein may be either single stranded or double stranded. Thus, in the embodiments stated above, nucleotides can be exchanged with base pairs.

In one embodiment the isolated polynucleotide is a purified nucleic acid sequence. The polynucleotide may be purified according to methods known by the skilled person.

The nucleic acid (DNA or RNA) may be isolated from a sample according to any methods known to those skilled in the art. The samples may be selected from a tissue sample, or from body fluid samples such as samples selected from the group consisting of blood, plasma, serum, semen and urine. The samples may be obtained by standard procedures and may be used immediately or stored, under conditions appropriate for the type of biological sample, for later use.

For example, genomic DNA is typically extracted from biological samples such as peripheral blood samples, but can also be extracted from other biological samples, including tissues (e.g., mucosal scrapings of the lining of the mouth). Routine methods are available for extracting genomic DNA from a blood or tissue sample, including, for example, phenol extraction. Genomic DNA can also be extracted using commercially-available kits, such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), and the A.S.A.P.3 Genomic DNA isolation kit (Boehringer Mannheim, Indianapolis, Ind.).

The sample can be from a subject or an individual which includes any animal, preferably a mammal. It is preferred that the individual is a human. The biological sample may be obtained from an individual at any stage of life such as from a fetus, an infant, a child, young adult or an adult. Particularly preferred subjects are humans having an increased risk of contracting a cardiac disorder such as for example a hereditary cardiac disorder.

Numerous methods will be known to those skilled in the art of molecular biology that could be used to determine the presence of one or more mutation in the calmodulin polynucleotide and/or polypeptide sequence, as discussed below.

In one embodiment, the calmodulin polynucleotide sequence may be sequenced directly. Such direct determination of the calmodulin sequence within an individual could be performed using Polymerase Chain Reaction (PCR) and/or Multiple Displacement amplification (MDA) (Spits et al., 2006) to amplify nucleic acid (e.g., genomic DNA or cDNA) obtained from the individual to be tested. Once amplified, the nucleic acid can be sequenced and compared to wild-type calmodulin sequences to determine whether or not the nucleic acid contains a genetic mutation. The sequence can be determined using for example but not limited to Sanger Sequencing, Pyrosequencing (Ronaghi et al., 1999) and Next Generation Sequencing (also termed “targeted re-sequencing”) (Su et al., 2011; Metzker 2010).

Nucleic acid extracted from tissue or body fluid samples can be amplified using nucleic acid amplification techniques well known in the art. Many of these amplification methods can also be used to detect the presence of mutations simply by designing oligonucleotide primers or probes to interact with or hybridize to a particular nucleotide sequence. These techniques can for example include polymerase chain reaction (PCR), reverse transcriptase polymerase chain reaction (RT-PCR), nested PCR, ligase chain reaction.

In one embodiment, PCR is used to amplify a target or marker sequence of interest, such as for example a nucleotide sequence which is tested for a mutation. The skilled artisan is capable of designing and preparing primers that are appropriate for amplifying a target or marker sequence. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill. The primers must be sufficiently long to prime synthesis of extension products in the presence of a polymerase. The length of the primers may typically vary from about 8 nucleotides to 60 nucleotides.

In one type of PCR-based assay, a primer hybridizes to a region on a target nucleic acid molecule that overlaps a region of the gene or nucleic acid sequence comprising the mutation to be identified and only primes amplification of the allelic form to which the primer exhibits perfect complementarity (Gibbs, 1989, Nucleic Acid Res., 17:2427-2448). Typically, the primer's 3′-most nucleotide is aligned with and complementary to the region of the nucleic acid sequence comprising the mutation to be identified. This primer is used in conjunction with a second primer that hybridizes at a distal site. Amplification proceeds from the two primers, producing a detectable product that indicates which allelic form is present in the test sample. A control is usually performed with a second pair of primers, wherein one of the primers comprises a base-mismatch such that when the primer anneals to the nucleotide strand an abnormal nucleotide base-pairing is formed thereby inhibiting amplification. The method generally works most effectively when the mismatch is at the 3′-most position of the oligonucleotide (i.e., the 3′-most position of the oligonucleotide aligns with the region of the nucleotide sequence comprising the mutation to be detected) because this position is most destabilizing to elongation from the primer (see e.g. WO 93/22456).

In one example, a primer comprises a sequence substantially complementary to a segment of a mutation-containing target nucleic acid molecule except that the primer has a mismatched nucleotide in one of the three nucleotide positions at the 3′-most end of the primer, such that the mismatched nucleotide does not base pair with a particular allele at the mutation site. The mismatched nucleotide in the primer can be the first, second or the third nucleotide from the last nucleotide at the 3′-most position of the primer. In some examples, primers and/or probes are labelled with detectable labels.

The nucleic acid mutations of the present invention may be detected by DNA sequencing. Sequencing may be carried out by the dideoxy chain termination method of Sanger et al. (Proceedings of the National Academy of Sciences USA (1977), 74, 5463-5467) with modifications by Zimmermann et al. (Nucleic Acids Res. (1990), 18:1067). Sequencing by dideoxy chain termination method can be performed using Thermo Sequenase (Amersham Pharmacia, Piscataway, N.J.), Sequenase reagents from US Biochemicals or Sequatherm sequencing kit (Epicenter Technologies, Madison, Wis.). Sequencing may also be carried out by the “RR dRhodamine Terminator Cycle Sequencing Kit” from PE Applied Biosystems (product no. 403044, Weiterstadt, Germany), Taq DyeDeoxy™ Terminator Cycle Sequencing kit and method (Perkin-Elmer/Applied Biosystems) in two directions using an Applied Biosystems Model 373 A DNA or in the presence of dye terminators CEQ™ Dye Terminator Cycle Sequencing Kit, (Beckman 608000). Alternatively, sequencing can be performed by a method known as Pyrosequencing (Pyrosequencing, Westborough, Mass.). Detailed protocols for Pyrosequencing can be found in: Alderborn et al, Genome Res. (2000), 10: 1249-1265.

The sequencing of the nucleotides can be performed by massively parallel sequencing technologies also known as next-generation sequencing. Next-generation sequencing platforms are characterized by the ability to process millions of sequence reads in parallel rather than for example 96 at a time, as typically seen for capillary-based sequencing. The workflow to produce next-generation sequence-ready libraries is straightforward; DNA fragments are prepared for sequencing by ligating specific adaptor oligos to both ends of each DNA fragment. Importantly, relatively little input DNA (a few micrograms at most) is needed to produce a library. Currently available platforms for next-generation sequencing produce shorter read lengths (35-250 bp, depending on the platform), but longer reads will also be possible. (Mardis, E. R. The impact of next-generation sequencing technology on genetics, Trends Genet. 2008 March; 24(3):133-41).

In another embodiment, one or more mutation in the calmodulin polynucleotide sequence may be identified by sequencing the genome of an individual, and subsequently identifying mutations in the coding region of the calmodulin genes within that individual by comparison with a list of known sequence variations.

Mutations in the calmodulin sequence in an individual can also be determined from a list of variations identified using whole genome sequencing or exome sequencing/targeted re-sequencing, which include an initial enrichment of target (Su et al., 2011; Metzker 2010). Also, but not exclusively, any platforms that uses nanopores to analyze single molecules of DNA/RNA and proteins (Niedringhaus et al., 2011) can be used.

Importantly, a computer program or software can be used to identify and report calmodulin mutations to scientific and medical personnel. Any software that reports clinically and diagnostically important mutations from whole-genome or exome sequence data can be used to identify mutations in the calmodulin gene sequences.

In another embodiment, mutations in the calmodulin polynucleotide sequence may be identified by screening the calmodulin sequence for variations, followed by sequencing to identify the exact mutation,

Appropriate methods for screening the calmodulin sequence for variations include High Resolution Melting (Erali and Wittwer. 2010), Denaturing High Performance Liquid Chromatography (DHPLC; Liu et al., 1998), and Single-Stranded Conformational Polymorphism detection (SSCP: Schafer et al., 1998). In case an individual is found to contain a sequence variation in the calmodulin sequence, the specific mutation can subsequently be determined using standard polynucleotide sequencing approaches (such as those described above).

In another embodiment, one or more mutation in the calmodulin polynucleotide sequence may be identified by performing mutation analysis directed towards a single mutation or polynucleotide position—for example, toward a known mutation suspected of being present in the individual being tested. Such approaches may be appropriate when testing an individual that is related to an individual already known to possess a particular mutation—for example, individuals belonging to a family in which one or more family member has been identified as possessing a calmodulin mutation.

Appropriate methods for determining whether or not an individual has a specific mutation in the calmodulin sequence include: PCR/ligase detection reaction (Yi et al., 2011). Mass-Spectrometry applications (Rodi et al., 2002), allele-specific hybridization (Prince et al., 2001), allele-specific restriction digests, and mutation specific polymerase chain reactions.

In another embodiment, one or more mutation in the calmodulin polypeptide sequence may be identified by analyzing a calmodulin polypeptide or a fragment thereof in a sample from the individual (for example, in a sample of blood or heart tissue). Any appropriate method can be used to analyze calmodulin polypeptides including immunological, chromatographic, and spectroscopic methods.

For example, a mutation in a calmodulin sequence that results in expression of a mutant calmodulin polypeptide can be detected in a sample from a mammal using an antibody that recognizes the mutant calmodulin polypeptide but which does not recognise the wild type calmodulin polypeptide. Such an antibody can, for example, recognize a mutant calmodulin polypeptide that differs from a wild type calmodulin polypeptide by one or more amino acid residues, without recognizing a wild type polypeptide. Such antibodies can be naturally-occurring, recombinant, or synthetic.

In a preferred embodiment the mutations are detected by High Resolution Melting (HRM) analysis (see “Kit” section)

### Mutations

The isolated polynucleotide as described herein comprises at least one mutation, which is associated with a cardiac disease. I.e., an individual comprising the mutation may be predisposed to a cardiac disorder, have an increased risk of contracting a cardiac disorder or the individual may have contracted a cardiac disorder.

Preferably, the at least one mutation in the polynucleotide sequence results in at least one mutation in the encoded polypeptide sequence and, preferably, results in the encoded mutated calmodulin having one or more altered functional property compared to wild type calmodulin.

In one embodiment, the altered functional property exhibited by the mutated calmodulin comprises or consists of aberrant binding to the RYR2 receptor, which binding is preferably increased or is reduced or is abolished, as compared to the binding exhibited by wild type calmodulin.

In another embodiment, the altered functional property exhibited by the mutated calmodulin comprises or consists of aberrant binding to calcium (Ca²⁺), which binding is preferably increased or is reduced or is abolished, as compared to the binding exhibited by wild type calmodulin.

In one embodiment, the altered functional property exhibited by the mutated calmodulin comprises or consists of both aberrant binding to the RYR2 receptor (and, preferably, binding which is increased or is reduced or is abolished, as compared to the binding exhibited by wild type calmodulin), and aberrant binding to calcium (and, preferably, binding which is increased or is reduced or is abolished, as compared to the binding exhibited by wild type calmodulin).

The mutation can for example be present in the noncoding region of the calmodulin gene or the mutation. In an embodiment the mutation is a silent mutation that does not result in a change of the amino acid sequence of the polypeptide or protein. Such a mutation may for example change the expression level of the gene.

It is preferred that the mutation is present in the coding region of a calmodulin encoding gene, such as in the coding region of SEQ ID NO:1, SEQ ID NO:2 or SEQ ID NO:3. Thus, in a preferred embodiment the mutation is in an exon of a calmodulin encoding gene, preferably in an exon of SEQ ID NO:1, SEQ ID NO:2 or SEQ ID NO:3. In a particular embodiment the mutation is in exon 2 of SEQ ID NO:1, SEQ ID NO:2 or SEQ ID NO:3. In another preferred embodiment the mutation is in exon 4 of SEQ ID NO:1, SEQ ID NO:2 or SEQ ID NO:3.

In a particular embodiment the mutation is present in the coding region of a SEQ ID NO:1.

The mutation may be any kind of mutation. In one embodiment the at least one mutation is a deletion of one or more nucleotides, such as for example at least 1 nucleotide, such as at least 2 nucleotides, at least 3 nucleotides, such as for example at least 4 nucleotides, such as at least 5 nucleotide s, at least 6 nucleotides, such as for example at least 7 nucleotides, such as at least 8 nucleotides, at least 9 nucleotides, such as for example at least 10 nucleotides, such as at least 11 nucleotides, at least 12 nucleotides, such as for example at least 13 nucleotides, such as at least 14 nucleotides, at least 15 nucleotides or such as for example at least 20 nucleotides.

In another embodiment the at least one mutation is an insertion of one or more nucleotides, such as for example at least 1 nucleotide, such as at least 2 nucleotides, at least 3 nucleotides, such as for example at least 4 nucleotides, such as at least 5 nucleotides, at least 6 nucleotides, such as for example at least 7 nucleotides, such as at least 8 nucleotides, at least 9 nucleotides, such as for example at least 10 nucleotides, such as at least 11 nucleotides, at least 12 nucleotides, such as for example at least 13 nucleotides, such as at least 14 nucleotides, at least 15 nucleotides or such as for example at least 20 nucleotide.

In a preferred embodiment the at least one mutation is a point mutation. In a point mutation a single nucleotide is exchanged for another. The point mutation may be an A>G mutation, an A>C mutation, an A>T mutation, a T>G mutation, a T>C mutation, a T>A mutation, a G>T mutation, a G>C mutation, n G>A mutation, a C>G mutation, a C>T mutation or a C>A mutation. In a more preferred embodiment the point mutation is a missense mutation in which a single nucleotide is changed, resulting in a codon that code for a different amino acid.

Thus in a preferred embodiment the isolated polynucleotide according to the present invention comprises at least one mutation that results in an amino acid substitution.

In a particular embodiment the isolated polynucleotide comprises a mutation that results in the amino acid substitution Asn97Ser. As demonstrated in the accompanying Examples, the amino acid substitution Asn97Ser in calmodulin results in the reduction or abolition in its binding with the RYR2 receptor, and/or the reduction or abolition in its binding affinity for the RYR2 receptor. The accompanying Examples also demonstrate that the amino acid substitution Asn97Ser in calmodulin also results in the reduction or abolition in its binding to calcium, and/or the reduction or abolition in its binding affinity for calcium.

In another particular embodiment the isolated polynucleotide comprises a mutation that results in the amino acid substitution Asn53Ile. As demonstrated in the accompanying Examples, the amino acid substitution Asn53Ile in calmodulin results in its aberrant binding to calcium (such as an increase or a reduction or abolition in its binding to calcium, and/or an increase or a reduction or abolition in its binding affinity for calcium).

In one embodiment, the isolated polynucleotide comprises mutations that result in the amino acid substitution Asn97Ser and Asn53Ile.

Thus, in a preferred embodiment the isolated polynucleotide comprises a mutation that results in the amino acid substitution Asn53Ser. A specific example is the mutation A4403G, wherein the adenine at position 4403 of SEQ ID NO:1 is exchanged with guanine. The sequence comprising the mutation A4403G is SEQ ID NO: 8. The calmodulin amino acid sequence comprising the Asn53Ser is SEQ ID NO: 10.

In another preferred embodiment the isolated polynucleotide comprises a mutation that results in the amino acid substitution Asn97Ile. A specific example is the mutation A7404T, wherein the adenine at position 7404 of SEQ ID NO:1 is exchanged with thymine. The sequence comprising the mutation A7404T is SEQ ID NO: 11. The calmodulin amino acid sequence comprising the Asn97Ser is SEQ ID NO: 13.

Asn97Ser means that Asparagine at amino acid position 97 of SEQ ID NO:4 has been substituted with Serine (SEQ ID NO: 13). Asn53Ile means that Asparagine at amino acid position 53 of SEQ ID NO:4 has been substituted with Isoleucine (SEQ ID NO: 10).

The mutation as described herein may for example be recessive mutation. In a preferred embodiment the mutation is a dominant mutation, in particular an autosomal dominant mutation.

The dominant mutation may in one embodiment be a gain-of-function mutation that changes the gene product such that it gains a new function. In another embodiment the dominant mutation is a dominant negative mutation that has an altered gene product that acts antagonistically to the wild-type allele.

The mutation may in one embodiment be a hereditary mutation. Thus, the mutation may for example be a dominant hereditary mutation or an autosomal dominant hereditary mutation. In a preferred embodiment the hereditary mutation is a missense mutation.

In another embodiment the mutation is a de novo mutation or in particular a dominant de novo mutation. In a preferred embodiment the de novo mutation is a missense mutation.

The isolated polynucleotide as described herein may comprise one or more of the mutations as described herein, i.e. the polynucleotide may for example comprise a combination of two or more of the mutations as described herein.

### Calmodulin Polypeptide Comprising at Least One Mutation

Another aspect of the present invention relates to an isolated polypeptide comprising calmodulin (CaM) or at least a part of a calmodulin protein, wherein the polypeptide comprises at least one mutation associated with a cardiac disorder.

Preferably, the at least one mutation in the polypeptide sequence results in the encoded mutated calmodulin having one or more altered functional property compared to wild type calmodulin, as described herein.

In one embodiment, the altered functional property exhibited by the mutated calmodulin comprises or consists of aberrant binding to the RYR2 receptor, which binding is preferably increased or is reduced or is abolished, as compared to the binding exhibited by wild type calmodulin.

In another embodiment, the altered functional property exhibited by the mutated calmodulin comprises or consists of aberrant binding to calcium (Ca²⁺), which binding is preferably increased or is reduced or is abolished, as compared to the binding exhibited by wild type calmodulin.

In one embodiment, the altered functional property exhibited by the mutated calmodulin comprises or consists of both aberrant binding to the RYR2 receptor (and, preferably, binding which is increased or is reduced or is abolished, as compared to the binding exhibited by wild type calmodulin), and aberrant binding to calcium (and, preferably, binding which is increased or is reduced or is abolished, as compared to the binding exhibited by wild type calmodulin).

In a particular embodiment the isolated polypeptide has at least 85% sequence identity, such as at least 90% sequence identity, at least 95% sequence identity, such as at least 97% sequence identity, at least 98% sequence identity, such as at least 99% sequence identity with SEQ ID NO:4 or part thereof. It is preferred that the isolated polypeptide has at least 95% sequence identity with SEQ ID NO:4 or part thereof.

The isolated polypeptide as described herein may for example comprise at least 5 amino acids, such as for example at least 10 amino acids, such as at least 15 amino acids, at least 20 amino acids, such as for example at least 25 amino acids, such as at least 30 amino acids, at least 50 amino acids, such as for example at least 60 amino acids, such as at least 70 amino acids, at least 80 amino acids, such as for example at least 90 amino acids, such as at least 100 amino acids, at least 120 amino acids or such as for example at least 140 amino acids of SEQ ID NO: 4. In one embodiment the isolated polypeptide comprises the entire amino acid sequence of SEQ ID NO:4.

In an embodiment the isolated polypeptide as described herein has at least 85% sequence identity, such as at least 90% sequence identity, at least 95% sequence identity, such as at least 97% sequence identity, at least 98% sequence identity, such as at least 99% sequence identity to at least at least 10 contiguous amino acids, such as at least 15 contiguous amino acids, at least 20 contiguous amino acids, such as for example at least 25 contiguous amino acids, such as at least 30 contiguous amino acids, at least 50 contiguous amino acids, such as for example at least 60 contiguous amino acids, such as at least 70 contiguous amino acids, at least 80 contiguous amino acids, such as for example at least 90 contiguous amino acids, such as at least 100 contiguous amino acids, at least 120 contiguous amino acids or such as for example at least 140 contiguous amino acids of SEQ ID NO: 4. In a preferred embodiment the isolated polypeptide has at least 95% sequence identity to at least 20 contiguous amino acids of SEQ ID NO:4.

In one embodiment the isolated polypeptide is a purified amino acid sequence. The amino acid sequence may be purified according to methods known by the skilled person.

The isolated polypeptide as described herein comprises at least one mutation, which is associated with a cardiac disease. I.e., an individual comprising the mutation may be predisposed to a cardiac disorder, have an increased risk of contracting a cardiac disorder or the individual may have contracted a cardiac disorder.

The mutation can be any kind of amino acid mutation in a calmodulin protein or in SEQ ID NO:4 or part thereof. The mutation can for example be an amino acid deletion or an insertion. In a preferred embodiment the mutation is an amino acid substitution.

In a particular embodiment the isolated polypeptide comprises the mutation Asn97Ser. As demonstrated in the accompanying Examples, the amino acid substitution Asn97Ser in calmodulin results in the reduction or abolition in its binding with the RYR2 receptor, and/or the reduction or abolition in its binding affinity for the RYR2 receptor. The accompanying Examples also demonstrate that the amino acid substitution Asn97Ser in calmodulin also results in the reduction or abolition in its binding to calcium, and/or the reduction or abolition in its binding affinity for calcium.

In another particular embodiment the isolated polypeptide comprises a mutation that results in the amino acid substitution Asn53Ile. As demonstrated in the accompanying Examples, the amino acid substitution Asn53Ile in calmodulin results in its aberrant binding to calcium (such as an increase or a reduction or abolition in its binding to calcium, and/or an increase or a reduction or abolition in its binding affinity for calcium).

In one embodiment, the isolated polypeptide comprises mutations that result in the amino acid substitution Asn97Ser and Asn53Ile.

Thus, in a preferred embodiment the isolated polypeptide comprises the mutation Asn53Ile (SEQ ID NO: 10). In another preferred embodiment the isolated polypeptide comprises the mutation Asn97Ser (SEQ ID NO: 13).

Asn97Ser means that Asparagine at amino acid position 97 of SEQ ID NO:4 has been substituted with Serine, whereas Asn53Ile means that Asparagine at amino acid position 53 of SEQ ID NO:4 has been substituted with Isoleucine.

The isolated polypeptide as described herein may comprise one or more of the mutations as described herein, i.e. the isolated polypeptide may for example comprise a combination of two or more of the mutations as described herein.

### Cardiac Disorders

As described herein, the isolated polynucleotide encoding calmodulin (CaM) or at least a part of calmodulin (CaM) comprises at least one mutation associated with a cardiac disorder, and similarly, the isolated polypeptide comprising calmodulin (CaM) or at least a part of CaM comprises at least one mutation associated with a cardiac disorder.

The term “cardiac disorder” as referred to herein includes any adverse event associated with the heart including, without limitation, an exertion- or exercise-induced cardiac event, sudden cardiac death, cardiac arrest, ventricular fibrillation, ventricular tachycardia, ventricular extrasystoles, premature ventricular contractions, and ventricular bigeminy. The cardiac disorder may for example be heart arrhythmia. In one embodiment the cardiac disorder is Ventricular tachycardia or Polymorphic Ventricular Tachycardia. In a particular embodiment the cardiac disorder is Catecholerminergic Polymorphic Ventricular Tachycardia (CPVT). The cardiac disorder may in an embodiment be drug-induced arrhythmia. In further preferred embodiments, the cardiac disorder is selected from the group consisting of: Sudden Infant Death Syndrome (SIDS); Sudden Unexpected Death Syndrome (SUDS); syncope; seizure; cardiac event.

The cardiac disorder may in a particular embodiment be associated with sudden cardiac death or result in sudden cardiac death. If the mutation is found in sample from an infant, the infant may be susceptible to sudden infant death syndrome.

In a preferred embodiment the cardiac disorder is heart arrhythmia. Heart arrhythmia is an abnormal or irregular heart rhythm. The heartbeats may for example be too slow (bradycardia) or to rapid (tachycardia), or too early. When a single heartbeat occurs earlier than normal, it is called a premature contraction. In a specific embodiment the heart arrhythmia is drug-induced heart arrhythmia.

In another preferred embodiment the cardiac disorder is Ventricular tachycardia such as for example Polymorphic Ventricular Tachycardia. In a particular preferred the cardiac disorder is Catecholerminergic Polymorphic Ventricular Tachycardia (CPVT).

Catecholaminergic polymorphic ventricular tachycardia (CPVT) is an inherited disease causing arrhythmias and sudden death in the structurally normal heart. Affected patients have a typical pattern of stress-induced atrial (supraventricular tachycardia and atrial fibrillation) and ventricular (bidirectional/polymorphic ventricular tachycardia and ventricular fibrillation) arrhythmias. The resting electrocardiogram of CPVT patients is normal, while arrhythmias can be reproducibly triggered by sudden adrenergic activation (exercise or acute emotion).

The mutation Asn97Ser as described herein was identified in the CALM1 gene and found to segregate with CPVT in a large Swedish family with a severe dominantly inherited form of CPVT, whereas the mutation Asn53Ile was identified by the inventors as a de novo mutation in the CALM1 gene (see Example section).

### Methods

The identification of mutations in a calmodulin encoding gene and/or in a calmodulin protein can be used in methods for determining whether an individual has an increased risk of contracting a cardiac disorder and in methods for diagnosing a disorder such as a cardiac disorder of an individual.

Additionally, the identification of a mutated calmodulin having an altered functional property compared to wild type calmodulin can also be used in methods for determining whether an individual has an increased risk of contracting a cardiac disorder and in methods for diagnosing a disorder such as a cardiac disorder of an individual.

In one embodiment, the altered functional property exhibited by the mutated calmodulin comprises or consists of aberrant binding to the RYR2 receptor, which binding is preferably increased or is reduced or is abolished, as compared to the binding exhibited by wild type calmodulin. In another embodiment, the altered functional property exhibited by the mutated calmodulin comprises or consists of aberrant binding to calcium (Ca²⁺), which binding is preferably increased or is reduced or is abolished, as compared to the binding exhibited by wild type calmodulin. In a further embodiment, the altered functional property exhibited by the mutated calmodulin comprises or consists of both aberrant binding to the RYR2 receptor (and, preferably, binding which is increased or is reduced or is abolished, as compared to the binding exhibited by wild type calmodulin), and aberrant binding to calcium (and, preferably, binding which is increased or is reduced or is abolished, as compared to the binding exhibited by wild type calmodulin).

Methods suitable for determining aberrant binding of calmodulin to the RYR2 receptor (for example, increased or reduced or abolished binding) and/or aberrant binding to calcium (Ca²⁺) (for example, increased or reduced or abolished binding) will be well known to those skilled in the art and include, for example, the methods described in the accompanying Examples.

Thus, an aspect of the invention relates to a method for determining whether or not an individual has an increased risk of contracting a disorder or sudden cardiac death, wherein said method comprises determining the presence or absence of at least one mutation in a calmodulin encoding gene or in a part of a calmodulin encoding gene, wherein the presence of said at least one mutation indicates an increased risk of contracting a disorder or sudden cardiac death. Preferably, the at least one mutation results in the encoded mutated calmodulin having one or more altered functional property compared to wild type calmodulin (as described herein).

Another aspect of the invention relates to a method for determining whether or not an individual has an increased risk of contracting a disorder or sudden cardiac death, wherein said method comprises determining the presence or absence of calmodulin having one or more altered functional property compared to wild type calmodulin (as discussed above), wherein the presence of one or more altered property indicates an increased risk of contracting a disorder or sudden cardiac death.

The calmodulin encoding gene may for example be selected from the group consisting of CALM1, CALM2 and CALM3. It is preferred that the disorder is a cardiac disorder.

In a specific embodiment the present invention relates to a method for determining whether or not an individual has an increased risk of contracting a cardiac disorder or sudden cardiac death, wherein said method comprises
determining the presence or absence of at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof in a sample from said individual and/or
determining the presence or absence of at least one mutation in the polypeptide having SEQ ID NO:4 or at least 90% sequence identity with SEQ ID NO:4 or part thereof in a sample from said individual,
   wherein the presence of said at least one mutation indicates an increased risk of contracting a cardiac disorder or sudden cardiac death.

Preferably, the at least one mutation results in the encoded mutated calmodulin having one or more altered functional property compared to wild type calmodulin (as described herein).

As discussed above, it is preferred that the altered functional property exhibited by the mutated calmodulin comprises or consists of aberrant binding to the RYR2 receptor (which binding is preferably increased or is reduced or is abolished) as compared to the binding exhibited by wild type calmodulin and/or comprises or consists of aberrant binding to calcium (Ca²⁺) (which binding is preferably increased or is reduced or is abolished) as compared to the binding exhibited by wild type calmodulin.

In a particular embodiment the isolated polypeptide comprises the mutation Asn97Ser. As demonstrated in the accompanying Examples, the amino acid substitution Asn97Ser in calmodulin results in the reduction or abolition in its binding with the RYR2 receptor, and/or the reduction or abolition in its binding affinity for the RYR2 receptor. The accompanying Examples also demonstrate that the amino acid substitution Asn97Ser in calmodulin also results in the reduction or abolition in its binding to calcium, and/or the reduction or abolition in its binding affinity for calcium.

In another particular embodiment the isolated polypeptide comprises a mutation that results in the amino acid substitution Asn53Ile. As demonstrated in the accompanying Examples, the amino acid substitution Asn53Ile in calmodulin results in its aberrant binding to calcium (such as an increase or a reduction or abolition in its binding to calcium, and/or an increase or a reduction or abolition in its binding affinity for calcium).

In one embodiment, the isolated polypeptide comprises mutations that result in the amino acid substitution Asn97Ser and Asn53Ile.

The individual is in a preferred embodiment a mammal and in a most preferred embodiment the individual is a human. The individual may for example be an infant, a child, young adult or an adult. In an embodiment the individual is a human having a family history with an inherited form of a cardiac disorder such as for example CPVT. Thus, in one example the individual is a healthy individual suspected of contracting a cardiac disorder or having a mutation in CALM1, CALM2 and/or CALM3.

Another aspect of the present invention relates to a method for diagnosing a disorder of an individual, wherein said method comprises determining the presence or absence of at least one mutation in a calmodulin encoding gene or in a part of a calmodulin encoding gene, wherein the presence of said at least one mutation indicates a disorder or an increased risk of contracting a disorder. Preferably, the at least one mutation results in the encoded mutated calmodulin having one or more altered functional property compared to wild type calmodulin (as described herein).

Another aspect of the present invention relates to a method for diagnosing a disorder of an individual, wherein said method comprises determining the presence or absence of calmodulin having one or more altered functional property compared to wild type calmodulin (as discussed above), wherein the presence of one or more altered property indicates a disorder or an increased risk of contracting a disorder. Preferably, the one or more altered functional property are as discussed herein.

It is preferred that the disorder is a cardiac disorder. The calmodulin encoding gene may for example be selected from the group consisting of CALM1, CALM2 and CALM3.

The presence of one or more mutations in a calmodulin sequence can also be used to indicate whether the individual is more susceptible to drowning or Sudden Unexplained Death Syndrome than a corresponding individual that does not have mutations in a calmodulin sequence

In some cases, the presence or absence of one or more mutations in a calmodulin sequence can be used in combination with other information (e.g., results of other diagnostic tests) to determine whether or not an individual is susceptible to drowning or having syncope, a seizure, a cardiac event, Sudden Infant Death Syndrome (SIDS), and/or Sudden Unexpected Death Syndrome (SUDS). For example, the presence of absence of a mutation in a calmodulin sequence can be used in combination with results of an electrocardiogram, an echocardiogram, an exercise test (e.g., an electrocardiogram treadmill exercise test or a cardiopulmonary exercise test), or a medical examination. In some cases, information about the presence or absence of a mutation in a calmodulin sequence can be used together with a medical history, a family history, or information from a device that has recorded the electrical activity of the heart over a period of time (e.g., days).

The presence of one or more mutations in a calmodulin sequence can also be used to distinguish one condition (e.g., CPVT, or very early onset severe LQTS with possible polymorphic ventricular tachycardia) (Nyegaard et al 2012, Crotti et al 2013) from another condition (e.g., LQTS alone). For example, the presence of one or more than one mutation in a calmodulin sequence, that is associated with a change in calcium binding affinity, increase in calmodulin calcium binding off-rate, calmodulin/RYR2 complex calcium binding affinity, or calmodulin folding stability, can indicate that the mammal has CPVT rather than LQTS, particularly when the mammal is LQTS genotype-negative. A negative LQTS genotype can be a genotype that is negative for mutations in KCNQ1/KVLQT1, KCNH2/HERG, SCN5A, KCNE1/MirK, and KCNE2/MiRP1 sequences (Tester et al., Heart Rhythm, 2(10):1099-105 (2005)).

In a specific embodiment the present invention relates to a method for diagnosing a cardiac disorder of an individual, wherein said method comprises
determining the presence or absence of at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof in a sample from said individual and/or
determining the presence or absence of at least one mutation in the polypeptide having SEQ ID NO:4 or at least 90% sequence identity with SEQ ID NO:4 or part thereof in a sample from said individual, wherein the presence of said at least one mutation indicates a cardiac disorder or an increased risk of contracting a cardiac disorder, and preferably wherein the at least one mutation results in the mutated calmodulin having one or more altered functional property compared to wild type calmodulin.

As discussed above, it is preferred that the altered functional property exhibited by the mutated calmodulin comprises or consists of aberrant binding to the RYR2 receptor (which binding is preferably increased or is reduced or is abolished) as compared to the binding exhibited by wild type calmodulin and/or comprises or consists of aberrant binding to calcium (Ca²⁺) (which binding is preferably increased or is reduced or is abolished) as compared to the binding exhibited by wild type calmodulin.

In one embodiment, the method for diagnosing a cardiac disorder of an individual comprises the further step, performed after said diagnosis, of treating an individual identified as having a cardiac disorder.

Methods for treating such cardiac disorders are well known to those skilled in the art of medicine and pharmacy. For example, individuals having a cardiac disorder can be treated using “beta-blocker” therapy, and/or a calcium channel blocker, and/or via implantation of a defibrillator. For example, it is particularly preferred for individuals identified as having LQTS alone to be treated using “beta-blocker” therapy, and for individuals having CPVT, or very early onset severe LQTS, to be treated using a calcium channel blocker and/or using an implanted defibrillator.

The methods as described herein can be used in combination with other information (e.g., results of other diagnostic tests) to determine whether or not an individual is predisposed to a cardiac disorder. For example, the methods can be used in combination with results of an electrocardiogram, an echocardiogram, an exercise test (e.g., an electrocardiogram treadmill exercise test or a cardiopulmonary exercise test), or a medical examination. The methods can also be used together with a medical history or a family history, and/or information from a device that has recorded the electrical activity of the heart over a period of time (e.g., days).

The polynucleotide is as defined herein in the section “Calmodulin polynucleotide comprising at least one mutation is as defined in the section “Mutations”.

In particular, the mutation may comprise or consist of the amino acid substitution, Asn97Ser. As demonstrated in the accompanying Examples, that amino acid substitution in calmodulin results in the reduction or abolition in its binding with the RYR2 receptor, and/or the reduction or abolition in its binding affinity for the RYR2 receptor. The accompanying Examples also demonstrate that the amino acid substitution Asn97Ser in calmodulin also results in the reduction or abolition in its binding to calcium, and/or the reduction or abolition in its binding affinity for calcium.

Alternatively, the mutation may comprise or consist of the amino acid substitution Asn53Ile. As demonstrated in the accompanying Examples, the amino acid substitution Asn53Ile in calmodulin results in its aberrant binding to calcium (such as an increase or a reduction or abolition in its binding to calcium, and/or an increase or a reduction or abolition in its binding affinity for calcium).

In one embodiment, the mutation may comprise or consist of Asn97Ser and Asn53Ile.

In a preferred embodiment the at least one mutation to be determined in
CALM1 (SEQ ID NO:1), CALM2 (SEQ ID NO:2) and/or CALM3 (SEQ ID NO:3) and/or
a polynucleotide having at least 90% sequence identity with SEQ ID NO:1, SEQ ID NO:2 and/or SEQ ID NO:3 or part thereof
   results in the amino acid substitution Asn53Ile and/or in the amino acid substitution Asn97Ser.

In another preferred embodiment the at least one mutation to be determined in the polypeptide having SEQ ID NO:4 or at least 90% sequence identity with SEQ ID NO:4 or part thereof is Asn53Ile and/or Asn97Ser.

The method for determining whether or not an individual has an increased risk of contracting a cardiac disorder and the method for diagnosing a cardiac disorder of an individual may further comprise a step of determining whether the individual is homozygous or heterozygous with respect to a mutation in a calmodulin encoding gene. Methods, such as for example nucleotide sequencing, for determining the zygosity status are known to the skilled person.

As described above, the samples may for example be a tissue sample. In one embodiment the sample is a body fluid sample such as for example a plasma, serum, semen or urine sample. In a preferred embodiment the sample is a blood sample. The samples may be obtained by standard procedures and may be used immediately or stored, under conditions appropriate for the type of biological sample, for later use.

The sample can be from a subject or an individual which includes any animal, preferably a mammal. It is preferred that the individual is a human. The biological sample may be obtained from an individual at any stage of life such as from a fetus, an infant, a child, young adult or an adult. Particularly preferred subjects are humans having an increased risk of contracting a cardiac disorder such as for example a hereditary cardiac disorder.

Calmodulin is known to bind directly to ryanodine receptor 2 (RyR2) thereby modulating the function of RyR2. RyR2 is a Ca²⁺ release channel located in the sarcoplasmic reticulum (SR) and physiologic control of Ca²⁺ release from the SR is necessary for timely contraction and relaxation during the cardiac cycle. A destabilized interaction between RyR2 and calmodulin may lead to abnormal RyR2-mediated Ca²⁺ release, which is associated with cardiac arrhythmia.

Thus, the mutations found in the calmodulin encoding genes may in one embodiment result in aberrant binding between calmodulin and the RyR2 receptor. The aberrant binding may for example be a decreased binding affinity between calmodulin and the RyR2. The binding between calmodulin and RyR2 is dependent on Ca²⁺. Thus, in one embodiment the mutation in the calmodulin gene changes the binding of Ca²⁺ to calmodulin. The changed binding between calmodulin and Ca²⁺ can result in aberrant binding between calmodulin and RyR2.

In a preferred embodiment the aberrant binding is a reduced binding. In a preferred embodiment, the mutation that changes the binding of Ca²⁺ to calmodulin is located in the C-terminal end of the calmodulin protein such as for example amino acids 75 to amino acids 148 of SEQ ID NO: 4

In a specific embodiment the mutation in the calmodulin gene changes the binding of Ca²⁺ to calmodulin thereby causing an aberrant binding such as for example a reduced binding affinity between calmodulin and RyR2.

The binding between the mutated calmodulin protein and RyR2 can in an embodiment be aberrant both at low and/or high concentrations of Ca²⁺. In a specific, the binding between the mutated calmodulin protein and RyR2 is defective at low Ca²⁺-concentrations, whereas at high Ca²⁺-concentrations (e.g. at least 1 μM Ca²⁺) the binding between the mutated calmodulin protein and RyR2 is restored. In one embodiment the mutation in the calmodulin gene result in a decreased binding affinity between calmodulin and RyR2 at Ca²⁺-concentrations below 1 μM. The binding affinity between RyR2 and the calmodulin gene may in one embodiment be restored at Ca²⁺-concentrations above 1 μM.

An individual having a mutation in a calmodulin encoding gene that destabilizes the interaction between RyR2 and calmodulin may be treated with an agent that increases or enhances the binding affinity between calmodulin and RyR2.

A further aspect of the present invention relates to a method for treatment of an individual having a disorder associated with at least one mutation in a calmodulin encoding gene or in a part of a calmodulin encoding gene, wherein said mutation results in the mutated calmodulin having one or more altered functional property compared to wild type calmodulin, said method comprising administering to said individual an agent capable of restoring and/or improving the altered functional property to the level in wild type calmodulin.

As discussed above, it is preferred that the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

In one embodiment, the altered functional property exhibited by the mutated calmodulin may comprise or consist of aberrant binding to the RYR2 receptor (which binding is preferably reduced or is abolished) as compared to the binding exhibited by wild type calmodulin—in that embodiment, said method comprises administering to said individual an agent capable of increasing the binding between calmodulin and RYR2 in a therapeutically effective amount, thereby treating said individual.

In another embodiment, the altered functional property exhibited by the mutated calmodulin may comprise or consist of aberrant binding to the RYR2 receptor (which binding is preferably increased) as compared to the binding exhibited by wild type calmodulin—in that embodiment, said method comprises administering to said individual an agent capable of decreasing the binding between calmodulin and RYR2 in a therapeutically effective amount, thereby treating said individual.

In a further embodiment, the altered functional property exhibited by the mutated calmodulin may comprise or consist of aberrant binding to calcium (Ca²⁺) (which binding is preferably reduced or is abolished) as compared to the binding exhibited by wild type calmodulin—in that embodiment, said method comprises administering to said individual an agent capable of increasing the binding between calmodulin and calcium (Ca²⁺) in a therapeutically effective amount, thereby treating said individual.

In a further embodiment, the altered functional property exhibited by the mutated calmodulin may comprise or consist of aberrant binding to calcium (Ca²⁺) (which binding is preferably increased) as compared to the binding exhibited by wild type calmodulin—in that embodiment, said method comprises administering to said individual an agent capable of decreasing the binding between calmodulin and calcium (Ca²⁺) in a therapeutically effective amount, thereby treating said individual.

It is preferred that the disorder is a cardiac disorder. The calmodulin encoding gene may for example be selected from the group consisting of CALM1, CALM2 and CALM3.

In a specific embodiment the present invention relates to a method for treatment of an individual having a cardiac disorder associated with at least one mutation in
CALM1 (SEQ ID NO:1), CALM2 (SEQ ID NO:2) and/or CALM3 (SEQ ID NO:3) and/or
in a polynucleotide having at least 90% sequence identity with SEQ ID NO:1, SEQ ID NO:2 and/or SEQ ID NO:3 or part thereof,
   wherein said mutation results in the mutated calmodulin having one or more altered functional property compared to wild type calmodulin, said method comprising administering to said individual an agent capable of restoring and/or improving the altered functional property to the level in wild type calmodulin.

In one embodiment, the altered functional property exhibited by the mutated calmodulin may comprise or consist of aberrant binding to the RYR2 receptor (which binding is preferably reduced or is abolished) as compared to the binding exhibited by wild type calmodulin—in that embodiment, said method comprises administering to said individual an agent capable of increasing the binding between calmodulin and RYR2 in a therapeutically effective amount, thereby treating said individual. Such an agent could for example be dantrolene (Xu et al, Biochem Biophys Research Comm, 2010, 394(3):660-666).

In another embodiment, the altered functional property exhibited by the mutated calmodulin may comprise or consist of aberrant binding to the RYR2 receptor (which binding is preferably increased) as compared to the binding exhibited by wild type calmodulin—in that embodiment, said method comprises administering to said individual an agent capable of decreasing the binding between calmodulin and RYR2 in a therapeutically effective amount, thereby treating said individual. Such an agent could for example be dantrolene (Xu et al, Biochem Biophys Research Comm, 2010, 394(3):660-666).

In another embodiment, the altered functional property exhibited by the mutated calmodulin may comprise or consist of aberrant binding to calcium (Ca²⁺) (which binding is preferably reduced or is abolished) as compared to the binding exhibited by wild type calmodulin—in that embodiment, said method comprises administering to said individual an agent capable of increasing the binding between calmodulin and calcium (Ca²⁺) in a therapeutically effective amount, thereby treating said individual.

In another embodiment, the altered functional property exhibited by the mutated calmodulin may comprise or consist of aberrant binding to calcium (Ca²⁺) (which binding is preferably increased) as compared to the binding exhibited by wild type calmodulin—in that embodiment, said method comprises administering to said individual an agent capable of decreasing the binding between calmodulin and calcium (Ca²⁺) in a therapeutically effective amount, thereby treating said individual.

The polynucleotide is as defined herein in the section “Calmodulin polynucleotide comprising at least one mutation is as defined in the section “Mutations”.

In particular, the mutation may comprise or consist of the amino acid substitution, Asn97Ser. As demonstrated in the accompanying Examples, that amino acid substitution in calmodulin results in the reduction or abolition in its binding with the RYR2 receptor, and/or the reduction or abolition in its binding affinity for the RYR2 receptor. The accompanying Examples also demonstrate that the amino acid substitution Asn97Ser in calmodulin also results in the reduction or abolition in its binding to calcium, and/or the reduction or abolition in its binding affinity for calcium.

In addition, the mutation may comprise or consist of the amino acid substitution Asn53Ile. As demonstrated in the accompanying Examples, the amino acid substitution Asn53Ile in calmodulin results in its aberrant binding to calcium (such as an increase or a reduction or abolition in its binding to calcium, and/or an increase or a reduction or abolition in its binding affinity for calcium).

In one embodiment, the mutation may comprise or consist of Asn97Ser and Asn53Ile.

It is preferred that the mutation inhibits the binding of calmodulin to RyR2.

In a preferred embodiment the at least one mutation in
CALM1 (SEQ ID NO:1), CALM2 (SEQ ID NO:2) and/or CALM3 (SEQ ID NO:3) and/or
a polynucleotide having at least 90% sequence identity with SEQ ID NO:1, SEQ ID NO:2 and/or SEQ ID NO:3 or part thereof results in the amino acid substitution Asn97Ser.

In another preferred embodiment the at least one mutation in the polypeptide having SEQ ID NO:4 or at least 90% sequence identity with SEQ ID NO:4 or part thereof is Asn97Ser.

The cardiac disorder as mentioned in the methods herein is as defined in the section “Cardiac disorders”. Thus, the cardiac disorder may for example be heart arrhythmia. In a particular embodiment the cardiac disorder is Ventricular Tachycardia, such as for example Catecholerminergic Polymorphic Ventricular Tachycardia (CPVT).

### Compound for Use in Treatment of Cardiac Disorders

As described above, mutations in calmodulin may alter in one or more of the functional properties of calmodulin compared to wild type calmodulin.

In one aspect, the invention provides an agent capable of restoring and/or improving the altered functional property of the mutated calmodulin to the level in wild type calmodulin. As will be appreciated, such agents provide a means of treating individuals having a cardiac disorder (such as those disorders described herein) which are associated with altered calmodulin function caused by a mutation in calmodulin.

In one embodiment, the altered functional property exhibited by the mutated calmodulin comprises or consists of aberrant binding to the RYR2 receptor (which binding is preferably increased or is reduced or is abolished) as compared to the binding exhibited by wild type calmodulin.

In that embodiment, an agent capable of restoring binding between the mutated calmodulin and RYR2 may therefore be used in the treatment of an individual having a disorder, such as a cardiac disorder caused by the one or more mutation in calmodulin. For example, where the binding of the mutated calmodulin to RYR2 is reduced or abolished, an agent capable of establishing and/or enhancing and/or increasing binding between the mutated calmodulin and RYR2 may be used to treat the individual. Where the binding of the mutated calmodulin to RYR2 is increased, an agent capable of decreasing binding (for example, to wild type levels) between the mutated calmodulin and RYR2 may be used to treat the individual.

In a further embodiment, the one or more mutation in calmodulin inhibits the binding of calmodulin to RyR2. Preferably, the binding of calmodulin to RyR2 is decreased when the Ca²⁺ concentration is lower than 1 μM.

An agent capable of enhancing the binding of calmodulin to ryanodine receptor 2 may therefore be used in the treatment of an individual having a disorder such as a cardiac disorder caused by one or more mutations in the calmodulin that inhibit the binding of calmodulin to RyR2. Enhancing the binding of calmodulin to ryanodine receptor 2 means increasing the binding affinity between calmodulin and RyR2.

In another embodiment, the altered functional property exhibited by the mutated calmodulin may comprise or consist of aberrant binding to calcium (Ca²⁺) (which binding is preferably increased or is reduced or is abolished) as compared to the binding exhibited by wild type calmodulin.

In that embodiment, an agent capable of restoring binding between the mutated calmodulin and calcium (Ca²⁺) may therefore be used in the treatment of an individual having a disorder, such as a cardiac disorder caused by the one or more mutation in calmodulin. For example, where the binding of the mutated calmodulin to calcium (Ca²⁺) is reduced or abolished, an agent capable of establishing and/or enhancing and/or increasing binding between the mutated calmodulin and calcium (Ca²⁺) may be used to treat the individual. Where the binding of the mutated calmodulin to calcium (Ca²⁺) is increased, an agent capable of decreasing (for example, to wild type levels) binding between the mutated calmodulin and calcium (Ca²⁺) may be used to treat the individual.

Accordingly, a further aspect of the present invention relates to a method for identifying a compound, capable of enhancing the binding of calmodulin to ryanodine receptor 2, wherein said calmodulin comprises at least one mutation that decreases the binding affinity to ryanodine receptor 2 (and which preferably reduces or abolishes binding to ryanodine receptor 2), said method comprising
providing a first sample comprising calmodulin protein or a fragment thereof having a mutation that decreases the binding affinity to ryanodine receptor 2 (and which preferably reduces or abolishes binding to ryanodine receptor 2), ryanodine receptor 2 or a fragment thereof, and a test compound
measuring the amount of calmodulin protein bound to ryanodine receptor 2 in said first sample
providing a second sample comprising calmodulin protein or a fragment thereof having a mutation that decreases the binding affinity to ryanodine receptor 2 (and which preferably reduces or abolishes binding to ryanodine receptor 2), and ryanodine receptor 2 or a fragment thereof
measuring the amount of calmodulin protein bound to ryanodine receptor 2 protein in said second sample
comparing the amount of calmodulin protein bound to ryanodine receptor 2 protein in the first and second sample, whereby a higher amount of calmodulin protein bound to ryanodine receptor 2 protein in the first sample as compared to the second sample indicates that the test compound enhances binding of calmodulin protein to ryanodine receptor 2 protein.

Preferably, the mutation that decreases the binding affinity to ryanodine receptor 2 (and which preferably reduces or abolishes binding to ryanodine receptor 2) comprises the amino acid substitution Asn97Ser and, more preferably, comprises the amino acid substitutions Asn97Ser and Asn53Ile.

In a preferred embodiment the calmodulin protein has at least 90% sequence identity with SEQ ID NO:4 or part thereof. In another preferred embodiment the at least one mutation in the amino acid sequence having SEQ ID NO:4 or at least 90% sequence identity with SEQ ID NO:4 or part thereof is Asn97Ser.

The first sample comprises calmodulin protein having a mutation that decreases the binding affinity to ryanodine receptor 2 (and which preferably reduces or abolishes binding to ryanodine receptor 2) and a test compound. The test compound is the compound which is tested for its ability to improve the binding of the mutated calmodulin protein the RyR2. The calmodulin and RyR2 protein may for example be present in cell extracts or in a purified form. Thus, the sample may comprise cell extract comprising RyR2 protein and mutated calmodulin protein. In one embodiment the sample comprises purified calmodulin and RyR2 protein. Methods for purification of protein are well known in the art.

The affinity of calmodulin binding to RyR2 may be investigated by using different concentrations of calmodulin and RyR2 in the presence of different concentrations of test compound. The concentration of protein in the sample may for example be in the range of from 10 nM to 10 μM, such as for example in the range of from 100 nM to 10 μM, such as in the range of from 1 μM to 5 μM. The concentration of test compound in the sample may for example be in the range of from 1 nM to 1 mM.

It is appreciated that the first and the second sample further comprise Ca²⁺. The samples may comprise various concentrations of Ca²⁺, for example from 10 nM to 200 μM Ca²⁺.

A study of the affinity of calmodulin binding to RyR2 in the presence of various Ca²⁺-concentrations is described in detail in Example 4.

It is preferred that the first sample and the second sample comprises equivalent concentrations of calmodulin protein, RyR2 protein and Ca²⁺ such that the data obtained on the binding affinities from the first and second sample in the presence or absence of test compound, respectively, are comparable.

Measuring the amount of calmodulin protein bound to ryanodine receptor 2 protein in the first and the second samples can be performed by conventional methods known to the skilled person. The affinity of calmodulin binding to RyR2 can for example be assessed by measuring the intrinsic fluorescence of a specific amino acid (see Example section). Alternatively, the affinity of calmodulin binding to RyR2 can be assessed by using a conventional pull-down assay employing an anti-RyR2 antibody followed by detection of bound calmodulin using an anti-calmodulin antibody (Biochem Biophys Res Commun, 2010, 394(3): 660-666).

A still further aspect of the present invention relates to a method for identifying a compound, capable of enhancing the binding of calmodulin to calcium (Ca²⁺), wherein said calmodulin comprises at least one mutation that decreases the binding affinity to calcium (Ca²⁺) (and which preferably reduces or abolishes binding to calcium (Ca²⁺)), said method comprising
providing a first sample comprising calmodulin protein or a fragment thereof having a mutation that decreases the binding affinity to calcium (Ca²⁺)(and which preferably reduces or abolishes binding to calcium (Ca²⁺)), calcium (Ca²⁺) and a test compound
measuring the amount of calmodulin protein bound to calcium (Ca²⁺) in said first sample
providing a second sample comprising calmodulin protein or a fragment thereof having a mutation that decreases the binding affinity to calcium (Ca²⁺)(and which preferably reduces or abolishes binding to calcium (Ca²⁺)), and calcium (Ca²⁺)
measuring the amount of calmodulin protein bound to calcium (Ca²⁺) in said second sample
comparing the amount of calmodulin protein bound to calcium (Ca²⁺) in the first and second sample, whereby a higher amount of calmodulin protein bound to calcium (Ca²⁺) in the first sample as compared to the second sample indicates that the test compound enhances binding of calmodulin protein to calcium (Ca²⁺).

Preferably, the mutation that decreases the binding affinity to calcium (Ca²⁺) (and which preferably reduces or abolishes binding to calcium (Ca²⁺)) comprises the amino acid substitution Asn53Ile and, more preferably, comprises the amino acid substitutions Asn53Ile and Asn97Ser.

The present method further relates to a compound identified by the method as described above for use in treatment of an individual having a cardiac disorder associated with at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof, wherein said mutation decreases the binding affinity between calmodulin and ryanodine receptor 2.

In a preferred embodiment the at least one mutation in
CALM1 (SEQ ID NO:1), CALM2 (SEQ ID NO:2) and/or CALM3 (SEQ ID NO:3) and/or
a polynucleotide having at least 90% sequence identity with SEQ ID NO:1, SEQ ID NO:2 and/or SEQ ID NO:3 or part thereof
   results in the amino acid substitution Asn97Ser.

In another preferred embodiment the at least one mutation in the polypeptide having SEQ ID NO:4 or at least 90% sequence identity with SEQ ID NO:4 or part thereof is Asn97Ser.

### Pharmaceutical Composition

One aspect of the present invention relates to a pharmaceutical composition for use in the treatment of an individual having a cardiac disorder associated with at least one mutation in calmodulin (CaM) or at least a part thereof, wherein said mutation results in the mutated calmodulin having one or more altered functional property compared to wild type calmodulin, and wherein said composition comprises an agent capable of restoring and/or improving the altered functional property to the level in wild type calmodulin.

As discussed above, it is preferred that the one or more altered functional property is one or more property selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability.

In one embodiment, the altered functional property exhibited by the mutated calmodulin comprises or consists of aberrant binding to the RYR2 receptor. Preferably, binding is reduced or is abolished, as compared to the binding exhibited by wild type calmodulin. However, in an alternative embodiment, the binding affinity for RYR2 is increased, as compared to the binding affinity exhibited by wild type calmodulin.

In another embodiment, the altered functional property exhibited by the mutated calmodulin comprises or consists of aberrant binding to calcium (Ca²⁺). Preferably, binding is reduced or is abolished, as compared to the binding exhibited by wild type calmodulin. However, in an alternative embodiment, the binding affinity for calcium (Ca²⁺) is increased, as compared to the binding affinity exhibited by wild type calmodulin.

In one embodiment, the invention relates to a pharmaceutical composition for use in the treatment of an individual having a cardiac disorder associated with at least one mutation in CALM1 (SEQ ID NO:1), CALM2 (SEQ ID NO:2) and/or CALM3 (SEQ ID NO:3), wherein said composition comprises an agent capable of increasing the binding affinity between calmodulin and ryanodine receptor 2.

A pharmaceutical composition is a composition comprising one or more substances that have medicinal properties, together with a pharmaceutical acceptable carrier.

The compound as described above can be formulated as pharmaceutical compositions and administered to a mammalian host, such as a human patient in a variety of forms adapted to the chosen route of administration. Routes for administration include, for example, intravenous, intra-arterial, subcutaneous, intramuscular, intraperitoneal and other routes selected by one of skill in the art.

Solutions of the compound can for example be prepared in water or saline, and optionally mixed with a nontoxic surfactant. Formulations for intravenous or intra-arterial administration may include sterile aqueous solutions that may also contain buffers, liposomes, diluents and other suitable additives. The pharmaceutical composition may also comprise or include serum.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

The pharmaceutical composition as described herein comprises an agent capable of increasing the binding affinity between calmodulin and ryanodine receptor 2. In one preferred embodiment the agent is identified by the method for identifying a compound as described above.

The pharmaceutical composition is used in the treatment of an individual having a cardiac disease. The cardiac disease is described in the section “Cardiac diseases”. Thus, the cardiac disorder may for example be heart arrhythmia or for example drug induced heart arrhythmia. In a particular embodiment the cardiac disorder is Ventricular Tachycardia, such as for example Catecholerminergic Polymorphic Ventricular Tachycardia (CPVT).

### Kit

The present invention also provides a kit that can be used to detect mutations in calmodulin.

In one embodiment, the invention provides a kit that can be used to detect mutations in calmodulin encoding genes such as CALM1 (SEQ ID NO:1), CALM2 (SEQ ID NO:2) and/or CALM3 (SEQ ID NO:3).

Thus, in one aspect the invention provides a kit for detecting at least one mutation in a polynucleotide encoding calmodulin (CaM), or at least a part of calmodulin (CaM), wherein said kit comprises at least one oligonucleotide that is complementary to a sequence of said calmodulin encoding gene such that if the mutation is present in the polynucleotide, strand elongation from said oligonucleotide results in an extension product comprising said mutation.

The polynucleotide is defined in the section “Calmodulin polynucleotide comprising at least one mutation”. In a preferred embodiment the polynucleotide is a calmodulin encoding gene.

The oligonucleotide comprises a sequence that is complementary or almost complementary to a sequence of the polynucleotide such that the oligonucleotide binds to the polynucleotide thereby enabling strand elongation. The oligonucleotide is designed to bind or anneal in the presence of or to the region of the polynucleotide, which is tested for the presence of at least one mutation such that the resulting extension product comprises the sequence which is tested for the presence of at least one mutation. If the mutation is present in the sample, this will result in extension products comprising the mutation. Thus, the oligonucleotide can be used to prime extension of the region of the polynucleotide comprising the mutation to be detected.

In one embodiment the oligonucleotide comprises the mutation to be detected. The oligonucleotide therefore binds more specifically to genes having the mutation. It is preferred that the mutation is located in the 3′ end of the oligonucleotide.

In a specific embodiment the oligonucleotide binds specifically to a region of a calmodulin encoding gene comprising Asn97Ser. Thus in one embodiment the oligonucleotide comprises a sequence that is complementary to a nucleotide sequence of the polynucleotide, wherein said nucleotide sequence comprising a mutation resulting in the amino acid substitution Asn97Ser.

In another specific embodiment the oligonucleotide binds specifically to a region of a calmodulin encoding gene comprising Asn53Ile. Thus in one embodiment the oligonucleotide comprises a sequence that is complementary to a nucleotide sequence of the polynucleotide, wherein said nucleotide sequence comprising a mutation resulting in the amino acid substitution Asn53Ile.

In a particularly preferred embodiment, the kit comprises a first oligonucleotide which binds specifically to a region of a calmodulin encoding gene comprising Asn97Ser (for example, an oligonucleotide having a sequence that is complementary to a nucleotide sequence of the polynucleotide which results in the amino acid substitution Asn97Ser); and a second oligonucleotide which binds specifically to a region of a calmodulin encoding gene comprising Asn53Ile (for example, an oligonucleotide having a sequence that is complementary to a nucleotide sequence of the polynucleotide which results in the amino acid substitution Asn53Ile).

In a preferred embodiment the polynucleotide is selected from the group consisting of CALM1 (SEQ ID NO:1), CALM2 (SEQ ID NO:2) and CALM3 (SEQ ID NO:3). In a particular embodiment the polynucleotide is CALM1 (SEQ ID NO:1).

In one embodiment, the kit further comprises one or more polynucleotide template comprising part or all of a mutated calmodulin polynucleotide sequence, which may be used as a positive control with which to identify mutated calmodulin polynucleotides in a sample. For example, the one or more polynucleotide template may comprise a calmodulin polynucleotide sequence (for example, as defined by SEQ ID NO:1, SEQ ID NO:2 and/or SEQ ID NO:3) which has a mutation encoding a Asn97Ser and/or a Asn53Ile mutation.

In one embodiment the kit further comprises a primer that binds to the nucleotide strand complementary to the nucleotide strand to which the oligonucleotide comprising the mutation binds, such that the extension product of the primer comprises a region complementary to extension product of the oligonucleotide comprising the mutation.

The kit may further comprises a temperature resistant DNA polymerase, for example Taq DNA polymerase, nucleotides and cofactors to initiate amplification of DNA sequences. Thus, the kit may comprise all necessary reagents for rapid and sensitive detection of a low percentage of mutant DNA in a background of wild-type genomic DNA. The mutations can for example be detected by DNA sequencing analysis.

In one embodiment the kit is used to detect mutation by real time PCR or qPCR.

In this regard the kit may comprise an oligonucleotide comprising the mutation to be detected, which allows selectively amplification of sequences comprising the mutation. The oligonucleotide comprising the mutation to be detected can for example be covalently linked to a probe comprising a fluorophore and a quencher. When the oligonucleotide binds to the polynucleotide strand during PCR, the fluorophore and quencher become separated. This leads to an increase in fluorescence from the reaction tube.

The kit may also be used to detect mutations by melting analysis. Thus kit may also comprise components used for melting analysis, such as for example high resolution melting (HRM) analysis.

High Resolution Melting (HRM) analysis is a post-PCR analysis method used to identify variations in nucleic acid sequences. The method is based on detecting small differences in the melting temperature of PCR generated amplicons. The process is simply a precise warming of the sample or the reaction mixture from around 50° C. up to around 95° C. At some point during this process, the melting temperature of the amplicon is reached and the two strands of DNA separate, i.e. the amplicon denatures into single stranded DNA.

In HRM analysis a fluorescent dye is used that allows to monitor the process in real-time. The dyes that are used for HRM are known as intercalating dyes that bind specifically to double-stranded DNA and when they are bound they fluoresce brightly. When the double stranded DNA denatures into single stranded DNA the intercalating dyes dissociates from the DNA. When the intercalating dyes are not bound to DNA they only fluoresce at a low level. At the beginning of the HRM analysis there is a high level of fluorescence in the sample because of the double stranded amplicons. But as the temperature increases the two strands of the amplicon melt apart, the concentration of double stranded DNA decreases and the fluorescence is consequently reduced. The HRM machine has a camera that measures the fluorescence and the machine then plots the data as a graph known as a melt curve, showing the level of fluorescence versus temperature. When for example two amplicons with different melting temperatures are present in the sample this will give rise to two different melting curves.

In one embodiment the kit is used to detect mutations by co-amplification of lower denaturation temperature PCR (COLD-PCR). Thus kit may also comprise components used for melting analysis, such as for example COLD-PCR.

In full-COLD-PCR, a five-step PCR protocol is performed, which includes a standard denaturation step, a hybridization step, a critical denaturation step at a defined critical temperature (Tc), a primer annealing step and an extension step. The hybridization step (normally performed at 70° C.) is used during PCR cycling to allow hybridization of mutant and wild-type alleles. The resulting heteroduplexes, which melt at lower temperatures than homoduplexes can be selectively denatured using an amplicon-specific Tc and preferentially amplified throughout the course of PCR; conversely, the denaturation efficiency is reduced for homoduplex molecules, and consequently, the majority of the molecules will remain in a double-stranded homoduplex state throughout the course of thermocycling. The efficiency of amplification of the major alleles (typically wild-type) is therefore appreciably reduced. However, by decreasing the denaturation temperature to the T_{C}, mutations at any position along the sequence are preferentially enriched during COLD-PCR amplification

In fast COLD-PCR amplicons containing a mutation are selectively denatured. The underlying principle of COLD-PCR is that single nucleotide mutations may slightly alter the melting temperature of the double-stranded DNA if the mutation implies that number of hydrogen bonds in the amplicon is decreased, for instance, G>A mutations, C>T mutations, G>T mutations, or C>A mutations (melting temperature decreasing mutations). A single nucleotide melting temperature decreasing mutation anywhere along a double-stranded DNA sequence generates a small change in the melting temperature for that sequence, with mutated sequences melting at a lower temperature than wild-type sequences. COLD-PCR uses a critical temperature during the PCR process in order to enrich mutations of the amplified sequence. During the denaturation step in the PCR reaction, the temperature is set to this critical temperature that results in denaturation of only the amplicon containing the mutation. Thereby, mutation-containing sequences are preferentially denatured and available for primer binding during the annealing step and subsequent amplification.

In a further embodiment, the invention provides a kit that can be used to detect mutations in the calmodulin polypeptide, for example, the polypeptide of SEQ ID NO:4.

In that embodiment, the kit preferably contains a detectable agent capable of specifically binding to a calmodulin polypeptide sequence (such as SEQ ID NO:4) which comprises a mutation (and, preferably, a Asn97Ser and/or a Asn53Ile mutation). It is preferred that the detectable agent does not bind to the wild type calmodulin polypeptide sequence and/or binds more strongly to the mutated calmodulin polypeptide sequence than to the wild type calmodulin polypeptide sequence (for example, 5-fold more strongly, or 10-fold more strongly; or 100-fold more strongly), thereby allowing the mutated calmodulin sequence to be selectively detected.

Preferably, the detectable agent is an antibody. In a preferred embodiment, the antibody is a monoclonal antibody, such as a monoclonal IgG antibody.

In a further preferred embodiment, the antibody (such as a monoclonal antibody, or a monoclonal IgG antibody) is capable of specifically binding to a calmodulin polypeptide sequence (such as SEQ ID NO:4) which comprises a Asn97Ser and/or a Asn53Ile mutation.

In that embodiment of the invention, the kit preferably further comprises one or more polypeptide molecule comprising part or all of a mutated calmodulin polypeptide sequence, which may be used as a positive control with which to identify mutated calmodulin polypeptide sequences in a sample. For example, the one or more polypeptide molecule may comprise a calmodulin polypeptide sequence (for example, as defined by SEQ ID NO:4) which has a Asn97Ser and/or a Asn53Ile mutation.

In a still further embodiment, the invention provides a kit that can be used to detect one or more altered functional property in a mutated calmodulin polypeptide, as compared to wild type calmodulin.

Preferably, the one or more property is selected from the list comprising: aberrant binding to the RYR2 receptor; aberrant binding to calcium (Ca²⁺); aberrant calmodulin-calcium binding off-rate (such as an increase in the calmodulin-calcium binding off-rate); aberrant calmodulin/RYR2 complex calcium binding affinity; calmodulin folding stability mutations in the calmodulin polypeptide, for example, the polypeptide of SEQ ID NO:4.

In a preferred embodiment, the invention provides a kit for detecting decreased binding of calmodulin to the RYR2 receptor, as compared to wild type calmodulin. In that embodiment, the kit comprises RYR2 receptor (or a part thereof); wild type calmodulin polypeptide (such as a polypeptide of SEQ ID NO:4); and one or more polypeptide molecule comprising part or all of a mutated calmodulin polypeptide sequence exhibiting reduced binding to the RYR2 receptor (which may be used as a control). Preferably, the one or more polypeptide molecule comprises a calmodulin polypeptide sequence (for example, as defined by SEQ ID NO:4) which has a Asn97Ser and/or a Asn53Ile mutation.

In another preferred embodiment, the invention provides a kit for detecting decreased binding of calmodulin to calcium (Ca²⁺), as compared to wild type calmodulin. In that embodiment, the kit comprises wild type calmodulin polypeptide (such as a polypeptide of SEQ ID NO:4); and one or more polypeptide molecule comprising part or all of a mutated calmodulin polypeptide sequence exhibiting reduced binding to calcium (Ca²⁺)(which may be used as a control). Preferably, the one or more polypeptide molecule comprises a calmodulin polypeptide sequence (for example, as defined by SEQ ID NO:4) which has a Asn97Ser and/or a Asn53Ile mutation. Optionally, the kit may further comprise RYR2 receptor (or a part thereof).

### EXAMPLES

### A—Experimental Data

### Methods

### CALM1 Sequencing

Primers amplifying the coding regions, adjacent splice sites, and the 5′- and 3′-untranslated regions of the CALM1 gene were designed using Primer315 (Supplementary Table 1). After PCR using standard conditions and an annealing temperature of 60° C., the amplified products were purified and sequenced on both strands at MWG (Eurofins MWG Operon, Ebersberg, Germany). Sequence analysis was performed using Mutation Surveyor (Softgenetics, State College, Pa., USA). One affected (the index patient) and one unaffected family member from family 1 was sequenced. After mutation discovery, the co-segregation with disease was confirmed using a genotyping assay developed by TIB MOLBIOL (Berlin, Germany) (Supplementary Table 1) using a LightCycler 480 instrument and Roche (Roche, Hvidovre, Denmark), HPLC purified primers (MWG) and LightCycler 480 Genotyping Master (Roche) according to the manufactures description.

### Mutation Screening

To screen for new mutations in all coding exons of CALM1, five screening assays were developed based on High Resolution Melting (HRM) of amplified PCR product (Supplementary Table 1). Primers were designed using Primer315. The screening was performed on a Lightcycler 480 Instrument (Roche) using HPLC purified primers (MWG) and LightCycler 480 High Resolution Melting Master (Roche) according to the manufactures description. All samples were analysed in duplicates and samples with aberrant melting curves, as well as 10 samples with normal melting curves for control, were sequenced. All assay conditions are available on request.

The October 2011 release of the 1000 Genome was interrogated through the Project Browser #ICHG2011, based on Ensembl release 63 and the Interim 20101123 phase 1 variant calls, at http://browser.1000genomes.org, representing an integrated set of variant calls and INDELS from low coverage and exome sequencing data across 1092 individuals.

### Miscellaneous

The structural models of calmodulin were prepared with Pymol (Schrödinger, LLC) using PDB ID 1DMO, 1CLL, and 2BCX for apo-, Ca2+-saturated, and Ca2+-bound calmodulin/RYR1complex, respectively 16, 17, 18. The calmodulin numbering used is that of the mature processed calmodulin without the initiator methionine residue.

### Plasmid Constructs

The CALM1 cDNA was obtained by PCR amplification using a nested priming approach (primers listed in Supplementary Table 1) KOD high fidelity polymerase (EMD Chemicals, Gibbstown, USA) and an in house cDNA preparation from liver 19. The PCR product using the inner cloning primers was ligated into a modified pMAL_c2x vector (New England Biolabs, Ipswich, USA) containing an additional linker sequence encoding the Tobacco Etch Virus (TEV) protease cleavage site (ENLYFQG) immediately followed by an XhoI restriction site. The resulting vector, pMaI_CaM, encodes an N-terminal Maltose Binding Protein, followed by a TEV protease recognition site and full-length native CaM without the initial Met residue. TEV cleavage of the fusion protein leaves an N-terminal GLE tri-peptide sequence before the N-terminal Ala residue from calmodulin. Missense mutations were introduced using the mutation oligonucleotides listed in Supplementary Table 1 and QuickChange® Lightning Site-Directed Mutagenesis kit (QIAGEN Nordic, Copenhagen, Denmark) according to the manufacturer's instructions, resulting in the pMaI_CaM-N53I and pMaI_CaM-N97S expression constructs. All constructs were verified by Sanger sequencing of the calmodulin encoding part.

### Protein Expression and Purification

The calmodulin variants were expressed in Rosetta B (DE3) cells (EMD Chemicals), grown at 37° C. in 1L LB cultures in baffled shaker flasks, by induction with 1 mM IPTG at OD600 around 0.3-0.4 and 3 hours continued growth. Cells were lysed with lysozyme and three freeze/thaw cycles in Lysis Buffer (20 mM Tris, 50 mM NaCl, pH 7.5) containing broad-spectrum protease inhibitors (Life Technology) Benzonase nuclease (EMD Chemicals). All buffers and reagents were prepared using MilliQ water (Millipore, Billerica, USA) purified and de-ionized (>18.2Ω resistance), and using plastic vessels, in order to minimize Ca2+ contamination. All laboratory equipment was washed in 1 M HCl and MilliQ water. The total Ca2+ concentration of the utilized buffers were determined to be between 1 and 3 μM, quantified using the Quin-2 fluorogenic calcium indicator and calcium calibration standard solutions according to the manufacturer's instructions (Calcium Calibration Buffer Kit #1, Invitrogen). Proteins were purified by Amylose Resin (New England Biolabs) affinity chromatography according to the manufacturer's instructions, then dialysed against TEV cleavage buffer (50 mM Tris, 2 mM EDTA, 2 mM β-Mercaptoethanol, 1% (V/V) glycerol, pH 8.0), before proceeding with TEV cleavage overnight at 4° C. Following complete cleavage, confirmed by SDS-PAGE analysis, the cleavage mixture was subjected to Ion Exchange Chromatography using ÄKTA purifier Fast Protein Liquid Chromatography (FPLC) equipped with a Source 15Q 10/10 column (GE health care) equilibrated with solvent A (20 mM Tris, 50 mM NaCl, pH 7.5). A linear gradient was performed from 20 to 100% (V/V) of solvent B (20 mM Tris, 1 M NaCl, pH 7.5). Final polishing of calmodulin purity was achieved by size exclusion chromatography using a Superdex 75 (16/60) column (GE health care, USA), equilibrated with 20 mM Hepes, 100 mM KCl, 6.9 mM NaCl at pH 7.2. Protein concentrations were determined by absorption at 280 nm. The identity and integrity of each protein preparation was confirmed by intact mass and tryptic peptide fingerprinting MALDI-TOF mass spectrometry using a Bruker Reflex III mass spectrometer (Bruker-Daltronics).

### Example 1

### Identifying an Asn53Ile Mutation

### Swedish Multiplex Family

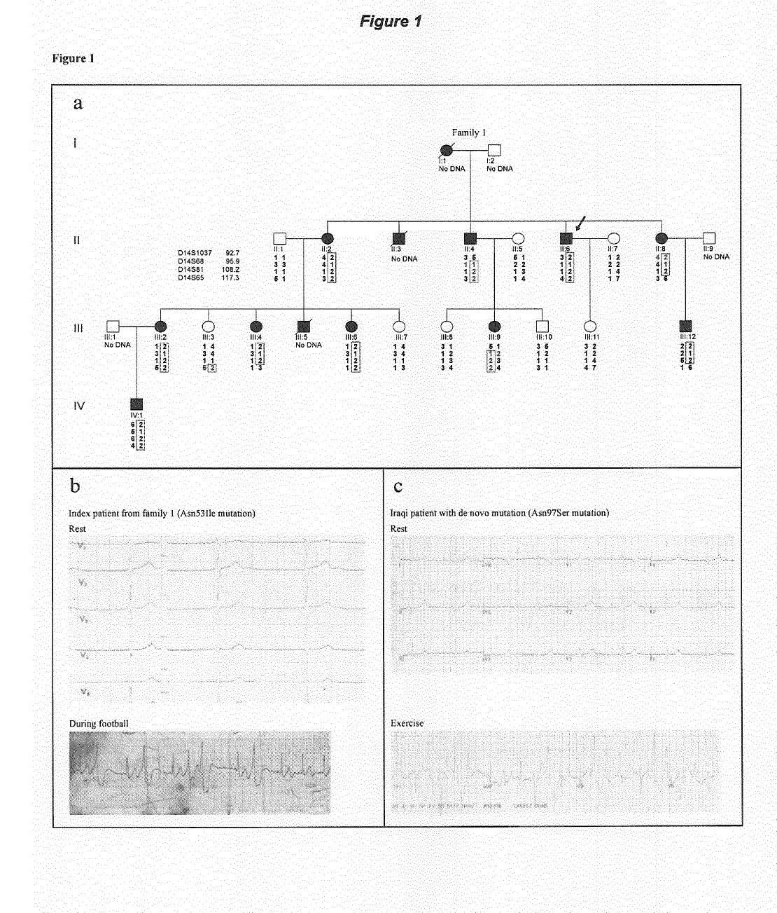

A Swedish multiplex family presented with a history of ventricular arrhythmias, syncopes and sudden death, predominantly in association with physical exercise or stress. The index case (II:6), a now 42-year old man of Swedish ethnic origin, presented with syncope while playing football at the age of 12 (FIG. 1). The ECG at admission (FIG. 1) showed sinus bradycardia (HR=45/min) and prominent U-wave in V2 and V3, but no evidence of QT prolongation (QTc=0.42 seconds). He had a history of loss of consciousness on at least four occasions during physical activity and once in connection with a fire alarm. A 24 h ECG registration revealed ventricular extrasystoles (VES), bigemini, and paired VES (FIG. 1) during football training, but no symptoms were reported. The patient was started on 61-adrenergic-receptor blocker treatment. At follow up, 24 h ECG and exercise test still showed some VES and bigemini with increasing load and heart rate. The index patient was later screened and found negative for mutations in a panel of arrhythmic genes including the CPVT genes RYR2 and CASQ2. An older brother (II:4), aged 23, had a history of repeated syncopes during exercise. During an exercise test he displayed polymorphic VT (ECG from that time not available). After β1-adrenergic-receptor blocker treatment a follow-up exercise test showed VES at high load The family history also included a brother (II:3) who drowned during a swimming competition at age 15 with prior episodes of syncope, and an older sister (II:2) who suffered fits or syncope. Following a later episode of ventricular fibrillation (VF) she was stabilized with β1-adrenergic-receptor blocker treatment and became asymptomatic. A younger sister (II:8) presented at the age of 7, with a history of repeated syncopes during intense physical activity.

One younger family member (III:5), with reported syncope from age 2.5, died suddenly at age 13, having been on β1-adrenergic-receptor blocker treatment for several years. Another family member (III:4) started having syncopes at age 4, was asymptomatic on β1-adrenergic-receptor blocker treatment and suffered cardiac arrest at age 16. After rapid defibrillation and resuscitation she recovered and had an ICD implanted. A younger sister (III:2) presented with syncope from age 6-7, and became asymptomatic on β1-adrenergic-receptor blocker (Pindolol) treatment. An older sister (III:6) presented with syncopes at age 3-4. Family member III:9, III:12, and IV:I were put on β1-adrenergic-receptor blocker after having suffered syncopes and attacks of dizziness. Based on information from the family, the subject I:1 had multiple syncopes in her youth and was on medication. She died 60 years old. Thus, the phenotypic picture of the family is characterized by CPVT-like features with symptoms including frequent syncopes and three cases of sudden death/cardiac arrest. The affected family members display no other apparent clinical manifestations.

### Linkage Analysis

Genome-wide linkage analysis was performed in the Swedish family using the Affymetrix GeneChip Human Mapping 50K SNP Array Xba240 chips. Twelve of the Swedish family members were included; nine affected (II:2, II:4, II:6, II:8, III:2, III:4, III:6, III:9, and III:12) and three unaffected (III:3, III:7, and III:8) (FIG. 1). The unaffected subjects were all older than 16 years at the time of inclusion. Genotypes were called by the Affymetrix Genotyping Console and uploaded to the BCSNP data management platform (BC Platforms, Espoo, Finland) (58.958 markers) for quality control filtering. First, markers with Mendelian errors were detected with MERLIN12 and removed from the dataset. This resulted in the removal of 113 markers. Next, PLINK13 was used to remove all monomorphic markers, and perform LD pruning using a sliding window of 50 SNPs and a r̂2 threshold of 0.5. This resulted in a dataset of 7199 SNPs in approximate linkage equilibrium with each other. Finally, MERLIN was used to identify unlike genotypes, resulting in the removal of 292 genotypes from the dataset. A multi parametric linkage analysis was performed with MERLIN using a 1 cM grid under the assumption of autosomal-dominant inheritance, full penetrance, and a disease gene frequency of 0.0001. SNP frequencies from the Affymetrix 100K frequencies (Caucasians) and genetic distances from the Affymetrix 100K Marshfield cM map file were used. Haplotypes were reconstructed with MERLIN and presented graphically with HaploPainter 14. A follow-up analysis with microsatellites was performed by including additionally six subjects from the family (one affected; IV:I, two unaffected; III:10, III:11, and three healthy married-in individuals; II:1, II:5, II:7). In total 18 individuals were genotyped with D1451037, D14568, D14581, D14565, D6S305, D6S386, and D6S1590 and analysed on an ABI310 Genetic analyser (Applied Biosystem, Foster City, US). Primer sequences were retrieved from the NCBI UniSTS database.

### Mapping of the Disease Locus and Identification of CALM1 Mutations

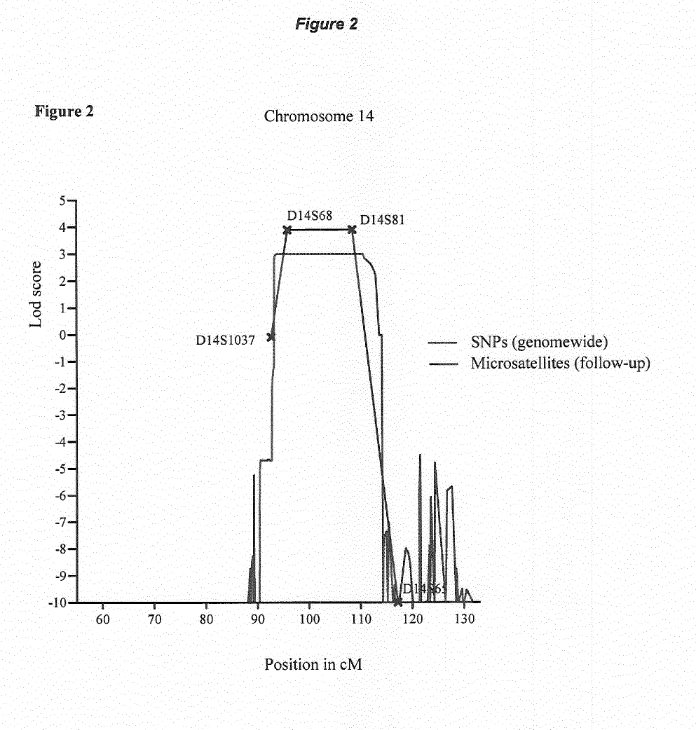

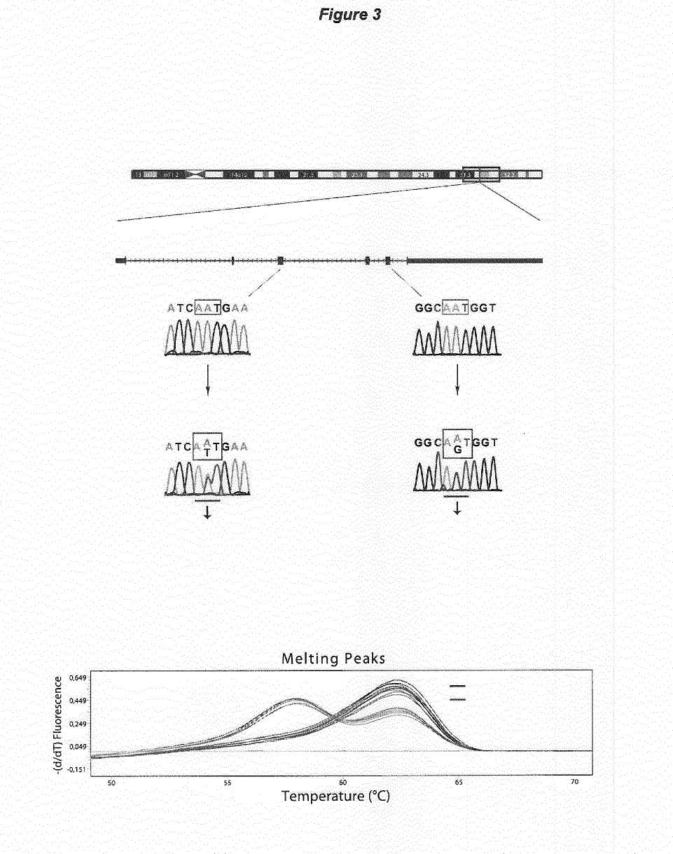

A genome-wide SNP-based linkage analysis was performed on twelve members from the large Swedish family (FIG. 1) and two possible linked loci on chromosomes 14q31-32 and 6q27-qter were identified, each with a maximum lodscore of 3.01 (Supplementary FIG. 1). Extensive follow-up analysis in these two regions with microsatellite markers including one additional newly diagnosed affected child (IV:1), and three unaffected children (II:1, II:5, II:7) excluded the locus on chromosome 6 and mapped the disease locus to chromosome 14 (lodscore 3.9) (FIG. 2). Haplotype analysis in the family confirmed the segregation of the chromosome 14 disease haplotype to all affected and none of the unaffected individuals (FIG. 1), suggesting 100% penetrance of the mutation in this family. The disease locus on chromosome 14 spanning 21 cM, ranged from rs323782 (86.6 Mb) to rs721403 (95.7 Mb) (hg19). Among the approximately 100 genes within in the linked region on chromosome 14, the CALM1gene was selected for sequencing due to the pivotal role of calmodulin in calcium signalling and heart contraction. The systematic sequencing of all five coding exons revealed a heterozygous missense mutation in exon 3 (corresponding to coding exon 2), resulting in an asparagine (Asn) to isoleucine (Ile) change (Asn53Ile) in the index patient (FIG. 3). A genotyping assay for the mutation (FIG. 3) confirmed that the Asn53Ile mutation co-segregated with the disease and was not found in any of the unaffected family members or in 500 Danish control individuals. Genotyping of 1200 control individuals (500 Danish medical student volunteers from Aarhus University, and 700 anonymous Danish blood donors) showed that the mutation was absent among these 2400 control chromosomes. This sample size provided an 80% power to detect a variant with minor allele frequency of 0.001 verifying that the mutation is highly unlikely to be a normal rare sequence variation.

### Example 2

### Iraqi De Novo Case

The index case, a 23-year old female of Iraqi ethnic origin, presented at age 4 with a successfully resuscitated, out-of-hospital cardiac arrest due to VF during running. She made a full neurological recovery and was stabilized on β1-adrenergic-receptor blocker treatment. An initial ECG and echocardiogram were within normal limits with no evidence of QT prolongation (not shown). Evaluation of her immediate family was unremarkable. An exercise ECG and electrophysiological study undertaken on full betablockade and right ventricular and coronary angiography were within normal limits. An initial diagnosis of idiopathic VF was made at the time. Follow-up ECGs demonstrated prominent U waves in the anterior leads, but no evidence of the long QT or Brugada syndromes (FIG. 1). At age 12 further investigation including signal-averaged ECG, echocardiography, cardiac MRI and cold pressor and procainamide tests were unremarkable. An exercise ECG off β1-adrenergic-receptor blocker demonstrated ventricular ectopy with couplets and triplets of varying morphology into recovery (FIG. 1). These appeared bidirectional at times. Based on this, a diagnosis of CPVT was made, but genetic testing for mutations in RYR2, CASQ2 and KCNJ2 proved negative. Follow-up genetic testing of other arrhythmic genes, KCNQ1, KCNH2, KCNE1, KCNE2, SCN5A also proved negative. She further fainted twice in her teens and at age 15 suffered a further cardiac arrest. After recovery, an ICD was implanted and the patient remained well, but was diagnosed with systemic lupus erythematosus (SLE). The index patient's mother, aged 62, was asymptomatic until developing heart failure secondary to adriamycin-induced cardiomyopathy from breast cancer treatment. She did not experience syncope or arrhythmia. An ECG demonstrated left bundle branch block with leftward axis deviation. Her father, aged 66, had suffered non-exertional syncopal episodes in stressful situations consistent with vasovagal syncope. His resting ECG was normal and exercise testing for atypical chest pain had induced ischemic changes without arrhythmia. An angiogram was reported as showing unobstructed coronaries and vasospasm.

### Discovery of a Second, De Novo, Mutation.

To systematically screen for novel CALM1 mutations, a HRM mutation detection assay was developed for all five coding exons. The assay was used to screen 63 patients referred for RYR2 mutation analysis at the Statens Serum Institute, Denmark, with 61 of these found to be RYR2 mutation negative. We identified a second heterozygous CALM1 missense mutation in a patient of Iraqi origin presenting atypic ventricular tachycardia and diagnosed with CPVT. This mutation was located in exon 5 and resulted in an asparagine (Asn) to serine (Ser) change (Asn97Ser) (FIG. 3). DNA sequencing of the family revealed that this mutation was absent in the mother and the father, both showing no signs of heart arrhythmias, demonstrating the presence of a de novo mutation in the patient. A microsatellite marker analysis confirmed the paternity relationship.

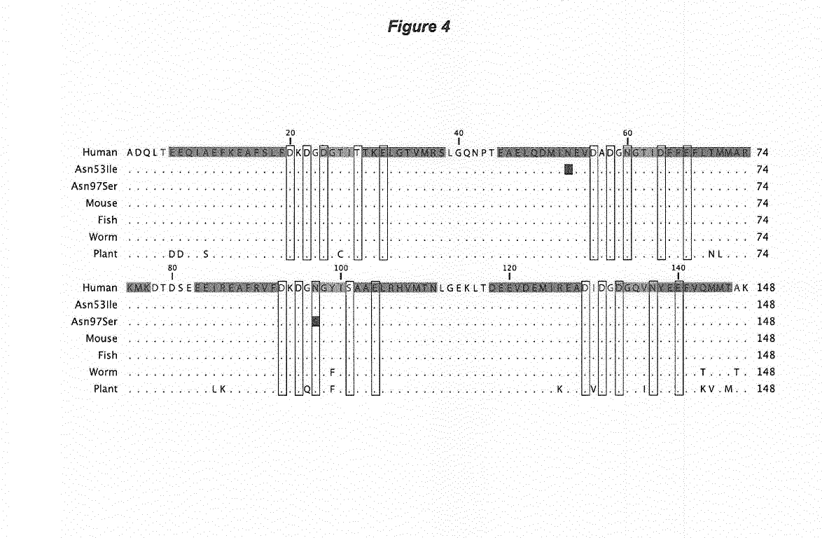

To assess if CALM1 missense mutations exist in the general population, a systematic HRM screen of all five CALM1 coding exons were performed in the 500 Danish control individuals. No missense mutations were identified among these 1000 control chromosomes. In contrast, three rare silent polymorphisms were identified (present among in total five control individuals) (Supplementary Table 2) stressing the selection pressure against missense mutations in this gene. We did not investigate a specific Iraqi population as controls for the Iraqi mutation, since this mutation was a de novo mutation, and the mutation rate is likely to be the same in all populations. To pursue the existence of missense mutations in other populations, we interrogated sequencing data from the 1000 genome project (October 2011 release based on 1092 individuals sequenced) and found no reported missense mutations in CALM1. A literature search also failed to present evidence for any previously identified calmodulin mutations. This is consistent with calmodulin being one of the most conserved molecules throughout evolution, being fully conserved in all vertebrate species and having evolved only slightly since the divergence from plants (FIG. 4).

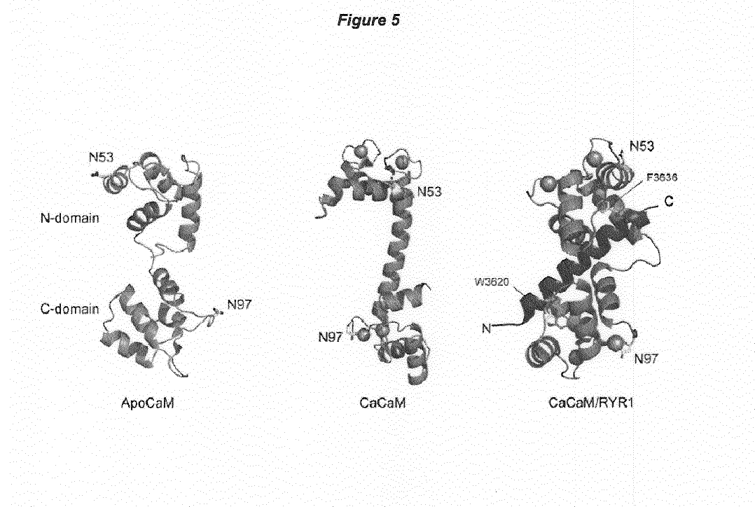

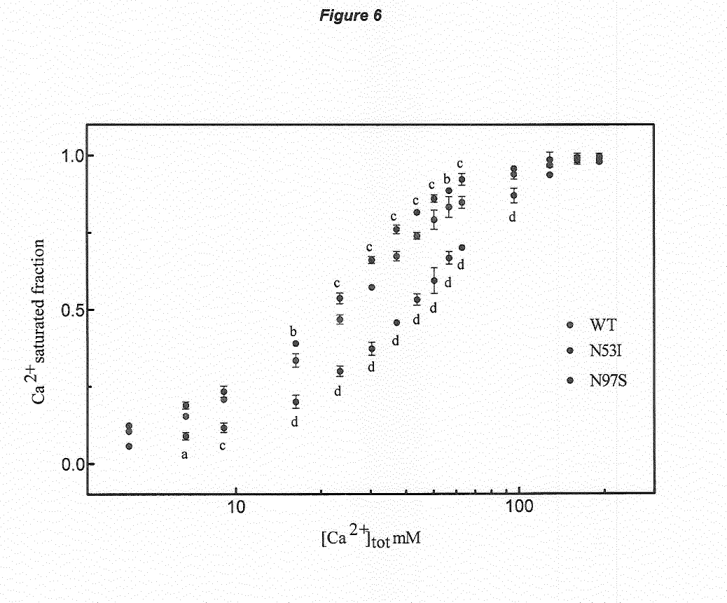

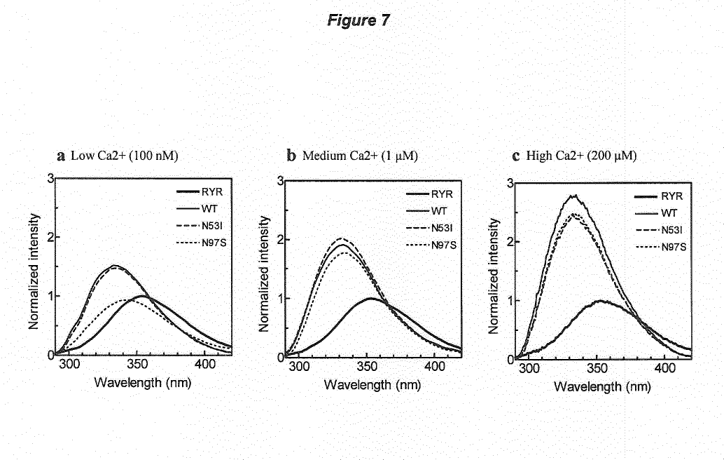

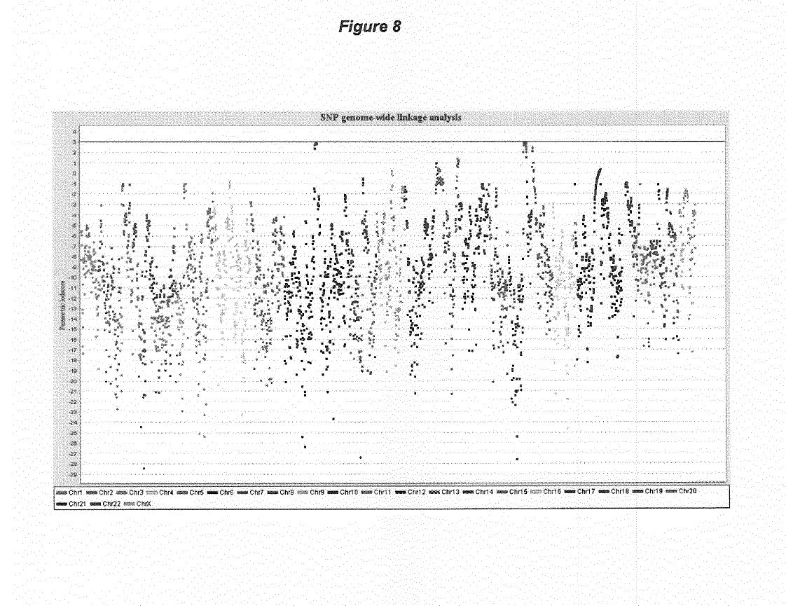

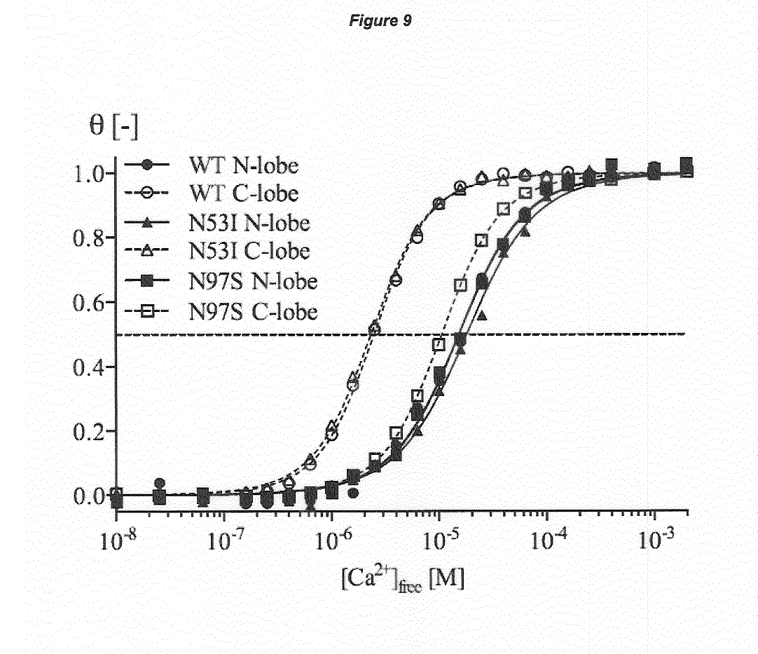

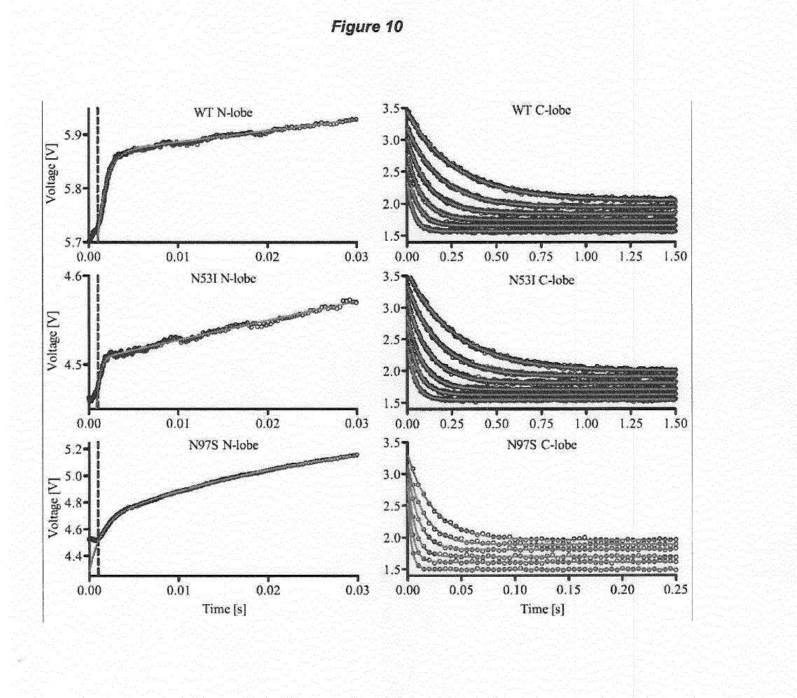

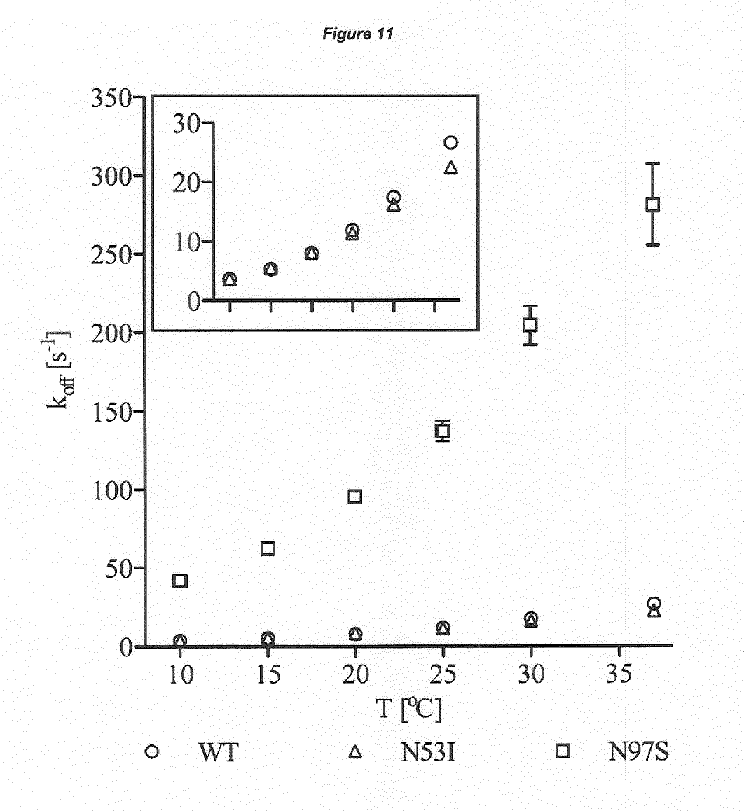

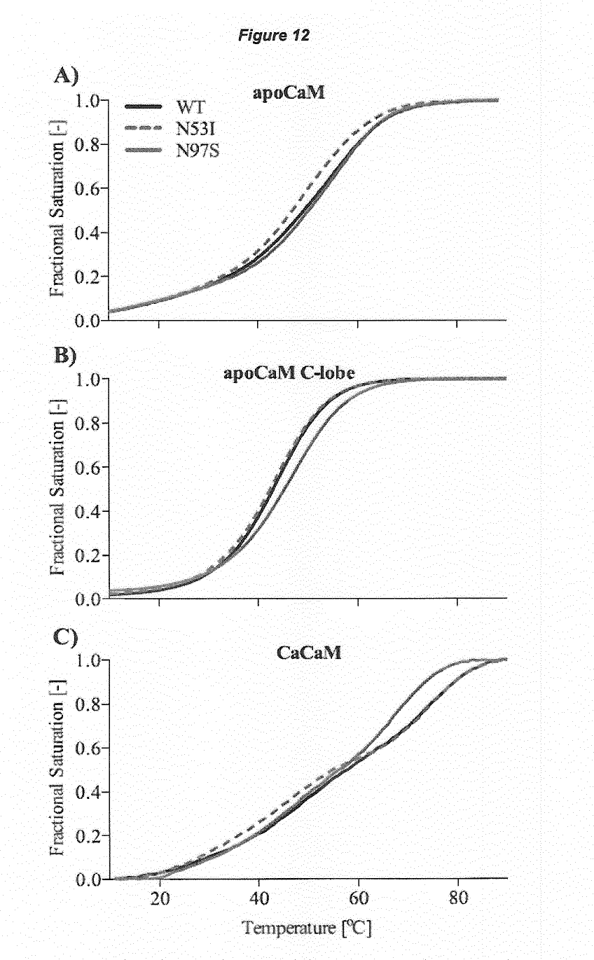

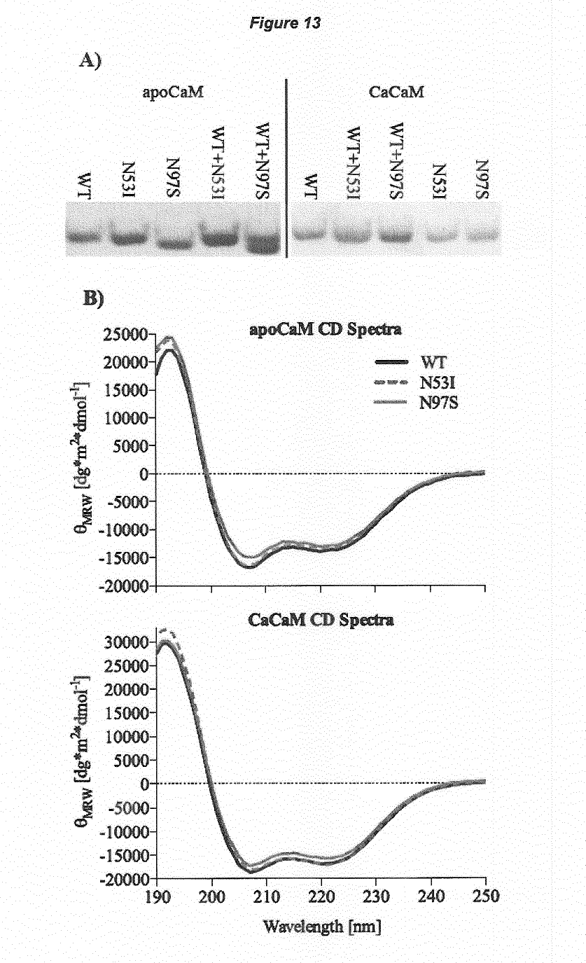

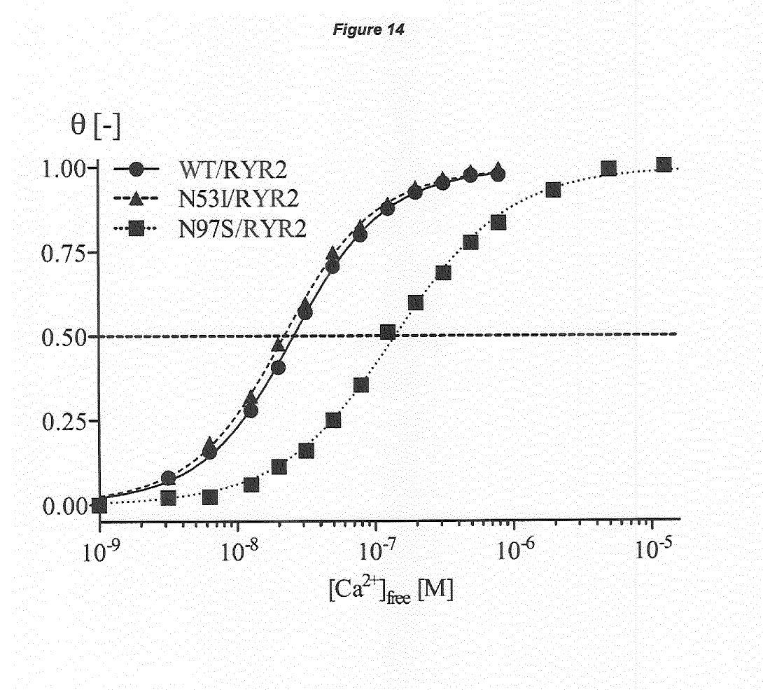

Calmodulin is an α-helical protein containing four classical Ca²⁺ binding EF-hands binding one calcium ion each. The two identified missense mutations are located in separate domains of the dumbbell shaped calmodulin molecule, with the Asn53 residue positioned on the solvent exposed surface of the first a-helix of Ca²⁺ binding site II in the N-domain, while the Asn97 residue is one of the Ca²⁺ binding residues of binding site III, located in the calmodulin C-domain (FIG. 5). Calmodulin binds to and regulates the activity of a large number of intracellular proteins, but in none of the published high resolution calmodulin structures available are the two mutated residues in direct contact with any bound protein or peptide (exemplified by the RYR1-peptide/calmodulin structure in FIG. 5).

### Example 3

### Binding of Calcium to Calmodulin

### Calcium Binding Assay

For calcium binding experiments, 15 μM calmodulin variants in gelfiltration buffer were titrated with CaCl₂ while the intrinsic tyrosine fluorescence was recorded on a FluoroMax 4 spectrofluorometer (HORIBA Jobin Yvon) equipped with a peltier temperature controller Emission scans, with 277 nm excitation, were measured from 290 to 400 nm at 1 nm increments, with 277 nm excitation, 5 nm band widths and 0.1 ms integration time. Temperature was kept at 37° C. Prior to each titration series, the cuvette was washed twice in 1% (V/V) Hellmanex II (HelmaAnalytics), twice in Milli-Q water, soaked in 1 M HCl and finally washed three times in Milli-Q water before drying with nitrogen. Fluorescence intensity signals at 320 nm (FI₃₂₀) for each titration point were normalized according to

$${θ}_{i} = \frac{{F}_{i}^{*} - {F}_{max}}{{F}_{min} - {F}_{max}}$$

where θᵢ is the normalized value for the i'th titration point and Fᵢ* the FI₃₂₀ for the i'th titration point corrected for the dilution of calmodulin during titration. Fₘₐₓ and Fₘᵢₙ were the highest and lowest FI₃₂₀ measured, respectively, within each titration data set. θᵢ values were averaged across triplicate measurements and plotted as a function of the total Ca²⁺ concentration. Error bars represent the standard deviation. The calcium binding data were analysed with Two-way Repeated Measurements ANOVA with Bonferroni multiple comparisons post hoc test using Graphpad Prism.

### Altered Calcium Binding for Calmodulin Mutations.

To functionally characterize the mutations, we first investigated the Ca²⁺ binding properties of native and mutated calmodulin. By monitoring the change in intrinsic tyrosine (Tyr) fluorescence that occurs as a consequence of the conformational change in calmodulin upon binding of Ca²⁺, the fractional Ca²⁺ saturation of the C-domain can be followed 22. We measured the C-domain Ca²⁺ binding as a function of the total Ca²⁺ concentration, and demonstrated a significant reduction in the Ca²⁺ affinity for the Asn97Ser mutation (FIG. 6), with an apparent half-saturation concentration of ˜40 μM compared to ˜25 μM total Ca²⁺ for native calmodulin. The Asn53Ile mutation, on the other hand, demonstrated a slight, but significantly increased C-domain Ca²⁺ saturation with an earlier half saturation of ˜21 μM. As the Asn53Ile mutation is located in the N-domain of calmodulin, we suggest this “earlier” C-domain Ca²⁺ saturation reflects a decreased N-domain Ca²⁺ affinity resulting in increased availability of free Ca²⁺ for the C-domain to bind at lower total Ca²⁺ concentration, or, less likely, an increased positive inter-domain co-operativity between N- and C-domains. Hence, we show that both mutations lead to altered calcium binding properties, and that the mutations impose different effects on the distribution of bound Ca²⁺ between the N- and C-domains of the mutated calmodulins.

### Example 4

### Binding of Calmodulin to RyR2

### RYR2 Binding Assay

A calmodulin binding peptide, corresponding to a fragment of RYR2 (R3581-SKKAVWHKLLSKQRKRAVVACFRMAPLYN-L3611) was obtained from Genscript (Piscataway, N.J., USA) and the integrity verified using MALDI-TOF MS. The calmodulin—RYR2 peptide binding was assessed by monitoring the intrinsic fluorescence emission of RYR2 Trp3586, with and without addition of saturating amounts of calmodulin variants. Fluorescence emission scans from 290 to 450 nm at 1 nm increments were collected using 280 nm excitation, band passes of 5 nm, and a temperature of 37° C. under four different free Ca²⁺ concentrations ([Ca²]free): no free Ca²⁺ ([Ca²]free=0 mM, 5 mM EGTA), resting cardiomyocyte conditions ([Ca²]free˜100 nM), excited conditions ([Ca²]free ˜1 μM), 20 and saturating Ca²⁺ conditions (200 μM total Ca²⁺), all in 20 mM HEPES; 100 mM KCl, pH 7.2. Equilibration time was set to 10 min before each data acquisition. The free Ca²⁺ concentration for excited and resting conditions were controlled using an EGTA-Ca2+ buffer system, as previously described 21. For all conditions, a concentration of 0.5 μM RYR2 peptide was used with none (RYR2 alone), 1 μM (1 μM [Ca²]free and 200 μM total Ca²⁺), or 3 μM (zero and 100 nM [Ca²]free) calmodulin variant added. Background emission scans were performed with identical concentrations of calmodulin without added RYR2 peptide. Data are presented as averaged (N=3), background subtracted and normalized to the maximum fluorescence intensity of the RYR2 fluorescence without added calmodulin.

### Aberrant RYR2 Binding at Low Calcium Concentration.

We next investigated the interaction between the calmodulin variants and a peptide from RYR2 encompassing the calmodulin binding domain (RYR2 residues R3581-L3611). In the high resolution 3D structure of calmodulin bound to the equivalent peptide from RYR1, the Ca²⁺ bound C-domain of calmodulin fully encloses the single tryptophan (Trp) residue of the RYR peptide (RYR1 Trp3620 corresponding to RYR2 Trp3586, FIG. 5). Since calmodulin itself does not contain any Trp residues, binding of calmodulin to the RYR2 peptide can be monitored by a large increase in fluorescence intensity of the RYR2 Trp3586 residue. When adding saturating amounts of calmodulin protein at low calcium concentrations (100 nM free Ca²⁺), a remarkable difference in RYR2 binding was demonstrated for Asn97Ser compared to Asn53Ile and native calmodulin. Asn97Ser only induced a slight blueshift and no increase in fluorescence intensity (FIG. 7), similar to the effect seen when calmodulin binds to the skeletal myocyte ryanodine receptor (RYR1) 23 in the absence of Ca²⁺. In marked contrast, little or no difference in RYR2 binding between Asn97Ser, Asn53Ile, and native calmodulin were observed at moderate (1 μM free) or saturating (200 μM total) calcium concentrations (FIG. 7). Thus, our data demonstrate that for the Asn97Ser mutation, the calmodulin-RYR2 interaction is defective at low intracellular Ca²⁺ and restored at moderate to high Ca²⁺ concentrations.

The release of sarcoplasmic reticulum (SR) calcium through RYR2 is one of the most critical molecular events during heart muscle contraction. RYR2 is the SR Ca²⁺-channel which upon a small increase in local intracellular Ca²⁺ concentration (from approximately 100 nanomolar to a few micromolar) switch from a closed to an open conformation, resulting in a large influx of Ca²⁺ from the SR storage, ultimately causing muscle contraction. The current molecular understanding of RYR2 associated VT is that RYR2 mutations render the tetrameric RYR2 complex “leaky”, thereby leading to increased local Ca²⁺ concentrations (Ca²⁺ sparks), untimely activation of nearby RYR2 clusters through calcium induced calcium release (CICR) and eventually arrhythmia. 27,28 As calmodulin binds directly to RYR2, and has been demonstrated to decrease the RYR2 open probability at all Ca²⁺ concentrations 29, the compromised calmodulin RYR2 interaction demonstrated here for the Ans97Ser mutation is likely to dominantly cause an increased RYR2 open probability at low diastolic Ca²⁺ concentrations. Hence, we suggest that calmodulin Asn97Ser mutation, similar to RYR2 mutations, leads to a gain-of-function mutation, which would explain how one mutated calmodulin allele out of six encoding identical proteins, is sufficient to cause a phenotype with a dominant inherited trait.

### B—Further Experimental Data

### Introduction

In eukaryotes changes in the concentration of free calcium ions ([Ca²⁺]_{free}) is a universal intra-cellular signal and the base for the function of excitable cells such as cardiomyocytes. A heartbeat is governed by Ca²⁺ signalling as a sarcolemma action potential is translated into a cardiomyocyte [Ca²⁺]_{free} transient wave that eventually couples the electrical signal to a chemical one for the contraction of myofilaments. The exact magnitude, spatial and temporal distribution of this [Ca²⁺]_{free} transient regulates heart rhythm and contractility. At the centre of this signalling is the Ca²⁺-sensing protein calmodulin (CaM), which conveys the spatially and temporally complex signals through interaction with hundreds of protein targets in Ca²⁺ signalling pathways.

CaM consists of two Ca²⁺ binding lobes, each with two EF-hands, separated by a linker in a dumbbell resembling conformation. Thus, CaM binds a total of four Ca²⁺ and may interact with protein targets both in the Ca²⁺ bound (CaCaM) and free (apoCaM) form. CaM Ca²⁺ binding and kinetics are sensitive to the interaction with protein targets, and may be regulated at the individual lobes. Furthermore, the binding states of the lobes also affect one another {Peersen:1997hx}. Combined with the CaM N- and C-lobe having markedly different Ca²⁺ binding properties, CaM may decipher Ca²⁺ signals in a highly complex manor. The two-lobe structure allows CaM to decode the frequency of Ca²⁺ oscillations into differential activation of some enzymes at the expense of others.

The pivotal role of CaM in transmitting the universal [Ca²⁺]_{free} signals onto a multitude of targets is reflected in its remarkable degree of evolutionary conservation. The three human CaM genes (CALM1-3) all encode the exact same protein and no amino acid changes in the 148 residue protein have been introduced since the appearance of vertebrates. For example, no missense mutations were found in a screen of CALM1-3 exons in 1500 humans, i.e in 9000 CaM encoding alleles, further illustrating that mutations in CaM are generally not tolerated (Nyegaard, M, et al, 2012).

In a recent study, however, we linked two novel mutations in CaM to catecholaminergic polymorphic ventricular tachycardia (CPVT) and sudden cardiac death (Nyegaard, M, et al, 2012). CPVT is an inherited heart disorder in which exercise or acute emotion can lead to syncope or sudden cardiac death without prior symptoms. Although diagnosed individuals are rare, CPVT is speculated as a significant cause of unexplained cardiac death among young people and has a very high mortality rate. Intriguingly and somewhat puzzling given the omnipresence of CaM, carriers of the CaM mutations showed no pathological symptoms other than CPVT. Mutations in the CaM regulated cardiac sarcoplasmic reticulum Ca²⁺ channel, the ryanodine receptor 2 (RYR2), and some of its accessory proteins, calsequestrin and triadin, have previously been linked to CPVT, making the Ca²⁺-CaM-RYR2 interaction a likely candidate for the molecular mechanism of CaM linked CPVT. The two CaM mutations, N53I and N97S, are localized in opposite Ca²⁺ binding lobes, and we previously found that Ca²⁺ affinities were qualitatively different to those of the wild type protein (WT) and that the N97S variant had diminished interaction with RYR2 (Nyegaard, M, et al, 2012).

In this Example, we further characterized the altered properties of the mutated CaM proteins as a step towards further understanding the molecular mechanism of CaM related CPVT.

### Experimental Procedures

### Materials

### Plasmid Constructs—

Two types of constructs were used.

1) Expression vector constructs from a previous study (Nyegaard, M, et al, 2012) were modified by removing a CaM N-terminal GLE augmentation using the QuickChange® Lightning Site-Directed Mutagenesis kit (QIAGEN Nordic, Copenhagen, Denmark). The resulting vectors (pMaI_CaMn, pMaI_CaMn-N53I and pMaI_CaMn-N97S) encode fusion proteins consisting of a Maltose Binding Protein variant, followed by a Tobacco Etch Virus (TEV) protease truncated cleavage site (ENLYFQ) and full-length CaM variants without the initial Met residue. The TEV protease recognition site variant adds no additional residues to CaM. All constructs were verified by Sanger sequencing of the CaM encoding part. 2) Mutated CaM genes (with no tags) were also produced by PCR using the synthetic gene for human CaM with codons optimised for expression in E. coli as a template. The mutants were cloned between NdeI and SacI in the PetSac vector (a derivative of Pet3) and plasmid preparations from single colonies confirmed by DNA sequencing.

### Protein Expression and Purification—

The plasmid constructs were used for two separate protein and purification procedures. CaM variants expressed in Rosetta B cells from pMAL_CaMn constructs were purified as previously described (Nyegaard, M, et al, 2012). CaM bound Ca²⁺ was removed by EDTA addition prior to final size-exclusion chromatography, and the final buffer (20 mM HEPES, 100 mM KCl at pH 7.2 (25° C.)) contained ˜1 uM Ca²⁺ as determined with the Quin-2 fluorogenic Ca²⁺ indicator and Calcium Calibration Buffer Kit #1 (Invitrogen). Protein concentrations were determined by absorption at 280 nm. The identity and integrity of each protein preparation was confirmed by MALDI-TOF mass spectrometry (Bruker Reflex III, Bruker-Daltronics) and sodium dodecyl sulfate-polyacrylamide gel electrophoresis (SDS-PAGE) analysis. This batch of proteins was used for Ca²⁺-buffered equilibrium titrations, stopped-flow, native polyacrylamide gel electrophoresis (PAGE), circular dichroism spectroscopy (CD), fluorescence emission scans and denaturation experiments—see below.

Each tag-free mutant was expressed in E. coli ER2566 using the PetSac expression constructs, and isolated using probe sonication in 20 mM imidazole, 20 mM NaCl, 1 mM EDTA, pH 7.0 (buffer A), centrifugation 10 min at 27000×g, pouring of the supernatant into boiling buffer, heating to 90° C., followed by rapid cooling on ice and removal of E. coli proteins by centrifugation as above. The CaM mutants were then purified by loading the supernatant onto a 3.4×20 cm DEAE cellulose ion exchange chromatography column equilibrated in buffer A. The protein was eluted over 1.4 L by a linear NaCl gradient from 20 to 500 mM NaCl in buffer A. Protein fractions were pooled, supplemented with 5 mM CaCl₂, pH raised to 7.5, and pumped onto a 0.4 L phenyl sepharose column equilibrated in 10 mM Tris/HCl, 1 mM CaCl₂, pH 7.5 (buffer B). The column was washed with 0.5 L buffer B, and CaM eluted in 10 mM Tris/HCl, 1 mM EDTA, pH 7.5. CaM fractions were again pooled, supplemented with 5 mM CaCl₂ and loaded onto a 2.2×20 cm DEAE Sephacel column in buffer B and eluted using a 1 L linear NaCl gradient from 0 to 400 mM NaCl in buffer B. CaM fractions were pooled, lyophilized, dissolved in 3 mL 0.1 M EDTA pH 8.0 and desalted on a 3.4×20 cm Sephadex G25 superfine column in H₂O. Preparations were loaded after 15 mL saturated NaCl (decalcified by chelex-100 resin). The protein travels through the NaCl zone during gel filtration and elutes free from both EDTA and Ca²⁺ as verified by NMR spectroscopy. This batch of proteins was used for equilibrium titrations in the presence of the 5,5-Br₂BAPTA [Ca²⁺]_{free} probe—see below.

### Ca²⁺ Equilibrium Titrations—

For determining lobe specific Ca²⁺ affinities, 15 M CaM with 0.75 M of either the fura-2 or the fura-6F Ca²⁺-probe (Invitrogen) in pH- and Ca²⁺-buffered solution (50 mM HEPES, 100 mM KCl, 0.5 mM EGTA and 2 mM NTA at pH 7.2 (25° C.)), were titrated with Ca²⁺ by changing [Ca²⁺]_{free} via volume replacement of the initial solution with a solution with the same composition plus 7 mM CaCl₂. The total Ca²⁺ concentration required for 24 titration points in the range 1 nM-2 mM were calculated using pCa-Calculator (Dweck, D et al, 2005). The fluorescence intensity was followed using a spectrofluorometer (HORIBA Jobin Yvon, FluoroMax®-4) with a Peltier element for temperature control. 0.8 mL protein solution in a 1 mL stir cuvette was kept at 25° C. and the CaM intrinsic protein fluorescence was measured as a partial phenylalanine and tyrosine emission spectrum, respectively (VanScyoc, W S, et al., 2002). Phenylalanine spectrum: 250 nm excitation and 265-275 nm emission with 7 nm bandwidths. Data points were collected at 1 nm increments with 3 spectra averaged. Tyrosine spectrum: 277 nm excitation and 314-326 emission with 5 and 7 nm bandwidths, respectively. Data points were collected at 2 nm increments with 2 spectra averaged. The [Ca²⁺]_{free} was measured using the two probes. Probe excitation spectra: 510 nm emission and 330-390 nm excitation with 3 nm bandwidths. Data points collected at 2 nm increments with 2 spectra averaged. Integration time was 0.2 s for all recordings. Measurements were done in quadruplicates. The phenylalanine and tyrosine spectra showed decreasing and increasing fluorescence intensities (FI), respectively, with increasing [Ca²⁺]_{free} as previously described (Vanscyoc, W S, et al., 2002). The fractional saturation of the CaM N-lobe (θ_{N}) was calculated by normalizing the phenylalanine 270 nm emission signal as a function of [Ca²⁺]_{free} (FI_{N}) for each replicate according to

$${θ}_{N} = \frac{{FI}_{N - max} - {FI}_{N}}{{FI}_{N - max} - {FI}_{N - min}}$$

with FI_{N-max} and FI_{N-min} measured as the average of the initial and last six titration points, respectively. The fractional saturation of the CaM C-lobe (sc) was calculated by normalizing the Tyr 320 nm emission signal as a function of [Ca²⁺]_{free} (FI_{C}) from each replicate according to

$${θ}_{C} = \frac{{FI}_{C} - {FI}_{C mi n}}{{FI}_{C - max} - {FI}_{C - min}}$$

with FI_{C-max} and FI_{C-min} measured as the average of the initial and last four titration points, respectively. The Fura-2 and Fura-6F probes allowed for measuring [Ca²⁺]_{free} in the range 63.1 nM-63.1 uM and these demonstrated >97% agreement with the pCa-Calculator determined values across the 15 titration points within the probes' range. As a very conservative estimate of the titration [Ca²⁺]_{free} accuracy, the relative standard deviations of the probe measured values were used to calculate a minimum standard deviation of the pCa-Calculator values used for plotting and fitting. The average relative standard deviation (5.0%) was applied to titration points outside the probe range. For evaluation of the thermodynamic parameters of the individual CaM lobes' Ca²⁺ binding, quadruplicate sets of θ_{N} and θ_{C} for each CaM variant were fitted to an Adair model of binding. For a heterogeneous two-site binding, as shown in scheme below, the Adair model takes the form

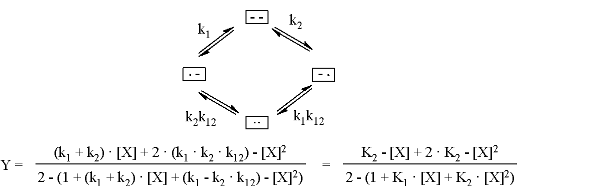

where Y indicates average fractional saturation of the macromolecule with ligand X and equilibrium constants k are as shown in the binding scheme with k₁₂ accounting for cooperativity. K₂ may be viewed as the equilibrium constant for the binding of ligands to both sites; hence the free energy of Ca²⁺ binding to both sites (ΔG₂) may be calculated as

ΔG₂=−K·T·ln(K₂)

Fitting was performed by non-linear regression in Prism 5 (Graphpad, Version 5.0d). Using the approach above and the same buffers, Ca²⁺ titration of 1 uM the RYR2 calmodulin binding domain peptide (RYR2p, 3580-RSKKAVWHKLLSKQRKRAWACFRMAPLYNL-3612) with 10 uM of CaM variants were performed. For each titration point a tryptophan emission spectrum was recorded: excitation at 295 nm and 320-370 nm emission both with 5 nm bandwidths and 3 spectra averaged. Integration time was set at 0.2 s and data points collected at 2 nm increments. The ratio of fluorescence intensity (FI) at 340 nm (CaCaM bound FI maximum) to that at 356 nm (free RYR2p FI maximum) was used to follow the binding of Ca²⁺ to the RYR2p bound CaM C-lobes. CaM C-lobe Ca²⁺ binding induces a conformational shift in the complex resulting in increased RYR2p tryptophan fluorescence. The ratio signal was normalized and fitted to the Adair equation as described above.

### CaM Ca²⁺ Dissociation Kinetics

To determine CaM Ca²⁺ dissociation rates (k_{off}) intrinsic and extrinsic fluorescence stopped-flow studies were performed using a stopped-flow instrument (SX20, Applied Photophysics) equipped with a 20 uL optical cell and an SQ.1 sequential mixer sample handling unit was used. Instrument dead-time was ˜1.6 ms. Temperature was controlled by a circulating water-bath and an internal temperature probe. An R6095 photo multiplier tube was mounted on the 10 mm viewport, with one of two filters in place for tyrosine (WG320, Schott) or Quin-2 (CG495, Schott) fluorescence. The drive syringes were 1.5 mL glass Hamilton syringes and the injection was set to 50 μL from each syringe for all experiments. CaM C-lobe k_{off} was measured using intrinsic protein tyrosine fluorescence and mixing a solution of 80 μM CaM in buffer (20 mM HEPES, 100 mM KCl and 640 μMCaCl2) with an equal volume of EDTA buffer (20 mM HEPES, 100 mM KCl and 4 mM EDTA). Measurements were performed at 10, 15, 20, 25, 30 and 37° C. with 4-5 measurements at each temperature. Excitation was at 277 nm with 4.6 nm slit width and 12.5 s averaging time. CaM N-lobe k_{off} was measured extrinsically with the Quin-2 Ca²⁺ probe (Invitrogen). 16 μM CaM in buffer (20 mM HEPES, 100 mKCl and 130 μM CaCl₂) was mixed with a Quin-2 solution (20 mM HEPES, 100 mKCl and 250 μM Quin-2). Measurements were performed at 8° C. with 20 measurements averaged over 50 ms using a logarithmic sampling approach. Excitation was at 330 nm with 7 nm slit width and 12.5 us averaging time. All buffers were pH 7.2 at 25° C. Using excess of Ca²⁺ chelators, EDTA and Quin-2, allows for analysing CaM Ca²⁺ dissociation kinetics as a pseudo-first order reaction, i.e.

$$- \frac{d [ CaM \cdot {Ca}^{2 +} ]}{d t} = k \cdot [ CaM \cdot {Ca}^{2 +} ] \cdot [ Chelator ] = - \frac{d [ CaM \cdot {Ca}^{2 +} ]}{d t} = {k}_{obs} \cdot [ CaM \cdot {Ca}^{2 +} ]$$

where the rate constant (k) becomes a observed rate constant (k_{obs}) being the product of the approximately constant chelator concentration ([Chelator]) and k. Assuming negligible Ca²⁺ dissociation from chelators and CaM dissociation as the rate limiting step k_{obs} equates to k_{off}.

k_{off} for the tyrosine fluorescence monitored CaM C-lobes were obtained by fitting the fluorescence intensity signals as a function of time (FI(t)) to a first-order decay

$$FI ( t ) = FI ( 0 ) + {FI}_{ss} - {FI}_{ss} \cdot {e}^{- {k}_{off} t}$$

, and k_{off} for the quin-2 fluorescence monitored CaM N- and C-lobe by fitting FI(t) to a first order two-phase increase function

$$FI ( t ) = FI ( 0 ) + {FI}_{ss} \cdot ( {e}^{- {k}_{off , N - {lobe}^{\cdot t}}} + {e}^{- {k}_{off , C - {lobe}^{\cdot t}}} )$$

, where Fₛₛ is the FI signal at steady state {Vogel:2002wl}. During N-lobe fitting routines the much slower C-lobe k_{off} value was entered as a constant, allowing proper fitting of the N-lobe k_{off} value. C-lobe k_{off} values were plotted as a function of T and also ln(k_{off}) as a function of 1/T. The latter was used for fitting to a linearized Arrhenius equation

$$k = A \cdot {e}^{\frac{- {E}_{a}}{R \cdot T}} ⇔ Ink = ln A + \frac{- {E}_{a}}{R} \cdot \frac{1}{T} ,$$

where A is the frequency factor, R the ideal gas constant and Eₐ the activation energy. Eₐ and InA are related to the transition state enthalpy (ΔH^{l}) and entropy (ΔS^{l}) via

$${E}_{a} = Δ {H}^{1} + R \cdot T$$

$$ln ( A ) = ln ( κ ) + ln ( \frac{- {k}_{B} \cdot T}{h} ) + \frac{Δ {S}^{1}}{R} ,$$

where k is the transmission factor, k_{B} and h the Boltzmann and Planck's constants, respectively {Winzor:2006hq}. ΔH^{l} and ΔS^{l} are assumed temperature independent. Since 0<k<1 and typically in the range 0.8-1, while ln(A) ˜25 and

$$ln ( \frac{- {k}_{B} \cdot T}{h} ) ∼ 30 ,$$

the ln(k) term is assumed neglibale in the calculation of ΔS^{l}. The standard transition state Gibbs free energy [(ΔG]^{o‡}) was calculated from

ΔG^{o‡}=ΔH^{l}−T·ΔS^{l}

, with T=298.15.

### Protein Stability

Thermal denaturation curves of apoCaM variants were obtained by monitoring the change in CD signals during heating of protein solutions. 25 μM CaM in buffer (10 mM HEPES, 100 mM KCl and 2 mM EDTA at pH 7.2) were placed in the spectropolarimeter (Chirascan Plus CD, Applied Photophysics) with a wire-thermometer inserted into the cuvette (Spectrosil 21/Q/0.5/CD 0.5 mm, Starna). CD signal at 222 nm (−helix signal) was recorded during thermal scan from 1-90° C. at a 1° C./min heating rate. Bandwidth was 1 nm and averaging time 15 s with measurements every 60 s. Measurements were done in triplicates and sequentially to avoid batch effects. Additional experiments with 0.5° C./min heating rate and 12.5 μM protein were performed to rule out heating rate and protein concentration effects. The 222 nm CD signal were subtracted buffer scans and converted to mean residue weight ellipticity (θ_{MBW}^{222 nm}. A three-state model was used for analyzing the unfolding of CaM. The model assumes unfolding of the two CaM domains with an intermediate state with one folded domain and one unfolded. The fractions of CaM in either the native (n), intermediate (i) or unfolded (u) state (Fₙ, Fᵢ, Fᵤ), respectively, is described by the equilibrium constants for the two transitions (Kᵢ and Kᵤ)

$${F}_{n} = \frac{1}{1 + {K}_{i} + {K}_{i} \cdot {K}_{u}} ⋀ {F}_{𝓁} = {K}_{i} \cdot {F}_{n} ⋀ {F}_{u} = {K}_{i} \cdot {K}_{u} \cdot {F}_{n}$$

Given the three-state model, the θ_{MRW}^{222 nm} signal as a function of temperature is

θ_{MRW}^{222 nm}=Fₙ(aₙ+bₙ·T)+Fᵢ(aᵢ+bᵢ·T)+Fᵤ(aᵤ+bᵤ·T)

where the constants a and b are the linear temperature dependencies of the signal contribution from the three different CaM states. Dependencies were determined using the y-intercept (aₙ, aᵢ, aᵤ) and the slope (bₙ, bᵢ, bᵤ) of θ_{M.RW}^{222 nm}(T) for the CaM variants individually. aₙ, bₙ and aᵤ, bᵤ were determined by linear regression in the ranges 1-15° C. and 78-88° C., respectively. aᵢ and bᵢ were calculated as the averages of the values for native and unfolded state (Masino, L, et al, 2000; Sorensen, B R, et al., 1998). Combining the modified Gibbs-Helmholtz equation with the Gibbs free energy expressed as a function of the equilibrium constant yields

$$K = {e}^{\frac{Δ {H}_{m} \cdot ( \frac{1}{{T}_{m}} \frac{1}{T} ) + Δ {C}_{p} \cdot ( {T}_{m} - T + T \cdot ln ( \frac{T}{{T}_{m}} ) )}{- RT}} ,$$

where ΔHₘ is the enthalpy of denaturation at Tₘ, ΔCₚ the heat capacity change, R the ideal gas constant and Tₘ the melting temperature of the specific transition and the equation applies to both transitions in the three-state model i.e. Kᵢ and Kᵤ. Fitting θ_{MRW}^{222 nm}(T) data to the model was done using non-linear regression in Prism 5 (Graphpad, Version 5.0d) and ΔCₚ fixed at 3.363 kJ/mol and 3.041 kJ/mol for the first and second transition, respectively, and ΔHₘ also considered independent of T {Masino:2000dm, Sorensen:1998fr}. The standard enthalpy (ΔH°), entropy (ΔS°) and Gibbs free energy (ΔG°) of denaturing the individual CaM domain were calculated according to

$$Δ {H}_{T} = Δ {H}_{{T}_{m}} + ( T - {T}_{m} ) \cdot Δ {C}_{p}$$

$$Δ {S}_{T} = \frac{Δ {H}_{{T}_{m}}}{{T}_{m}} + ln ( \frac{T}{{T}_{m}} ) \cdot Δ {C}_{p}$$

$$Δ {G}_{T} = Δ {H}_{T} + T \cdot Δ {S}_{T} ,$$

with T=298.15 K. For plotting purposes the θ_{MRW}^{222 nm}(T) signals and model fits were normalized as fractions of the magnitude of the total signal change. To investigate the protein stability of CaCaM variants, thermal denaturation in the presence of 5 M urea was performed using the same CD approach as described above. Data normalization was also performed in an identical manner, however no model could be fitted as the presence of urea excludes the use of conventional unfolding models. Thermal denaturation of apoCaM variants were also performed by monitoring of the intrinsic tyrosine fluorescence signal (Chirascan Plus CD, Applied Photophysics) as a signal for CaM C-lobe denaturation. A thermal scan from 10-90° C. was performed with a 1° C./min heating rate. Excitation and emission were at 277 nm and 320 nm with 5 and 9 nm slid widths, respectively. Integration time was 20 s and measurements done in a Hellma 115-QS 10 mm cuvette. The 320 nm fluorescence signals were subtracted buffer scans and normalized to fractions of the largest absolute value at 10° C. As only the unfolding of the one CaM lobe was monitored, a simplified two-state version of the model above was used for analysing the unfolding of the CaM C-lobes. Calculation of equilibrium constants and thermodynamic parameters were performed as for the three-state model.

### Protein Structure Analysis

The CaM variants were submitted to native PAGE. 20 uL of 13-18 uM CaM samples in loading buffer (0.75 M Tris, 15%(V/V) glycerol and 18 mg L⁻¹ bromophenol blue at pH 8.8) with either 2 mM EDTA or 0.5 mM CaCl₂ were loaded to custom cast 20% (W/V) acrylamide (37.5:1) gel and gel-electrophoresis done at 100 V constant for 5 h in native running buffer (0.19 M glycine and 0.025 M Tris at pH 8.8). Gels were stained with Coomassie Brilliant Blue G-250 and digitally scanned. PAGE were done both at ambient temperature and isothermally at 5° C. with identical results. Buffers and gels were prepared so as too minimize Ca²⁺ contamination (˜1 uM). In combination with prediction algorithms, CD was also used to probe protein secondary structure distribution. 25 μM of CaM variants in buffer (2 mM HEPES, 50 mM KCl at pH 7.2 (25° C.)) with either 0.5 mM or 2 mM EDTA in a cuvette (Suprasil 106-QS-0.1 mm, Hellma) were placed in the spectropolarimeter. CD spectra were recorded in the range 190-250 nm with 1 nm bandwidth, 3 s averaging time and 3 spectra averaged. Samples were prepared and measured in triplicate at 25° C. CD spectra were subtracted buffer blanks and the signal converted to mean residue weight ellipticity (θ_{MRW}). Distributions of secondary structure elements in the CaM variants were calculated using the CDpro (http://lamar.colostate.edu/˜sreeram/CDPro/main.html) run CDSSTR program with the SP43 reference set off proteins (Sreerama, N, et al., 2000). Secondary structure elements are divided into regular α-helix, distorted α-helix, regular β-strand, distorted β-strand, turns and unordered (Sreerama, N, et al., 2000).

### Results

CaM variants display divergent changes in Ca²⁺ affinities Intrinsic protein fluorescence with buffering of [Ca²⁺]_{free} was employed to investigating the Ca²⁺ affinities of the CaM variants (Vanscoyc, W S, et al., 2002; Dweck, D, et al., 2005). With increasing [Ca²⁺]_{free}, the lobe specific fluorescence signals reported on the saturation states of CaM N- and C-lobe, respectively. The normalized saturation curves indicated significant differences between the WT, N53I and N97S; The N53I titration curves showed reduced N-lobe Ca²⁺ affinity and minutely increased C-lobe affinity, whereas the N97S C-lobe curve showed markedly reduced affinity. These differences were quantified by curve fitting to a two-site Adair model. (FIG. 9A and Table 1). The ΔG₂ values, corresponding to the Gibbs free energy of binding (calculated of from the compounded equilibrium constant K2), confirmed the qualitative observations. The ΔG₂ values demonstrated that N53I had significantly lowered N-lobe affinity and increased C-lobe affinity, although with a relatively small magnitude for the latter. Furthermore, the ΔG₂ values for the N97S variant demonstrated no change in N-lobe affinity, but a significant change in its C-lobe affinity shifting it close to the N-lobe affinities. The K1 values report on the sum of the equilibrium constants of binding either one of the EF-hand sites in a single lobe, whereas the K2 value reports the product of the same two equilibrium constants and the contribution from any cooperative effect (see methods). In contrast to the N53I N-lobe K2 values, the K1 values were not significantly altered compared to the WT values. This indicated that the decreased N53I N-lobe affinity was due to diminished cooperativity between the two N-lobe EF-hands. The same observation was made for the N53I C-lobe. In the case of the N97S C-lobe, both the K1 and K2 values were altered, indicating an alteration of the C-domain EF-hands' single site affinities and possibly also their cooperativity.

The calculated Gibbs free energies of CaM binding four Ca²⁺ ions and either of the lobes binding two (ΔG₂ and ΔGₜₒₜ, respectively) for the WT CaM were in good agreement with values determined in equivalent studies (Table 1) {Linse:1991wo, VanScyoc:2002jm}. The N97S variant had clearly altered ΔG₂ and ΔGₜₒₜ values for its C-lobe, which in both cases translated to an altered Gibbs free energy of Ca²⁺ saturating for the entire N97S CaM molecule. On the other hand, the individual the N53I lobes were subject to opposing changes in Ca²⁺ affinities, therefore the N53I ΔG₂ and ΔGₜₒₜ values for the total molecule were not significantly different from that of the WT protein.

CaM Variants have Altered Ca²⁺ Dissociation Rates

We determined the CaM Ca²⁺ dissociation rates (k_{off}) for the CaM variants by stopped-flow experiments at different temperatures. The C-lobe k_{off} were determined using the change in intrinsic protein tyrosine fluorescence upon Ca²⁺ release and the N-lobe (and C-lobe) k_{off} by employing the fast fluorescent Ca²⁺ probe, quin-2. Measurements of N-lobe k_{off} with quin-2 also allows for simultaneous measurement of C-lobe k_{off}. The CaM N-lobe k_{off} was in the range of the instrument dead time even at 15° C., why these were evaluated at the lowest temperature obtainable, 8° C. (FIG. 10 and Table 2). The N53I N-lobe showed a two-fold increase in k_{off} relative to the WT N-lobe i.e. at the instrument the upper limit of measurement (circa 2000 s⁻¹). Conversely, the N97S N-lobe had a markedly reduced k_{off}. Interestingly, the equilibrium titrations using the Ca²⁺-buffer system, and corresponding to the experimental conditions for stopped-flow analysis, did not show a significant change in N97S N-lobe Ca²⁺ affinity (Table 1, top).

C-lobe k_{off} values were assayed at 10, 15, 20, 25, 30 and 37° C. and all showed typical Arrhenius equation behaviour (FIGS. 10 and 11). The N53I C-lobe showed significantly reduced k_{off} only at 37° C., which was consistent with an altered temperature dependence of k_{off} i.e. changed Eₐ. Transition state theory analysis further indicated that the N53I C-lobe ΔH^{l} and ΔS^{l} were significantly altered (Table 2). Nevertheless, the net effect on ΔG^{o‡}was not significant as changes in ΔH^{l} and ΔS^{l} were energetically opposing. As expected from the equilibrium titrations, the most dramatic effect on k_{off} was seen for the N97S C-lobe which demonstrated a ˜10 fold increase. The N97S C-lobe transition state parameters showed a significant lowering of the ΔG^{o‡}, and this was mainly due to a less favourable entropic contribution. CaM Ca²⁺ association rates were not assayed as these are too fast for current methods.

### CaM Mutations Affect Protein Stability

Thermal denaturation of apo- and Ca²⁺ bound CaM variants were monitored by CD and tyrosine fluorescence. The apoCaM N53I and N97S variants were destabilized and stabilized, respectively, relative to the WT protein (FIG. 12A). Fitting the unfolding process by a three state model, demonstrated that the apoN53I N-lobe was destabilized primarily due to lowered enthalpy of unfolding (ΔH°, albeit with a contribution from a slightly less favourable folding entropy (ΔS° (Table 3). In addition, the apoN53I C-lobe had a lowered melting temperature (Tₘ), a destabilization most likely due to a shift in ΔS. The apoN97S thermal denaturation curve was overall very similar to the WT, but with the first part shifted towards higher Tₘ. In keeping with the qualitative observation, fitting of apoN97S demonstrated a significant increase in Tₘ for the C-lobe, attributable to an increased ΔH°. No significant changes were observed for the apoN97S N-lobe. Thermal denaturation of apoCaM monitored by tyrosine fluorescence and subsequent curve fitting determined Tₘ for the WT, N53I and N97S C-lobes to be 43.0, 42.4 and 45.3° C., respectively (FIG. 11B). These Tₘ values were in good agreement with those determined for the C-lobe unfolding by fitting to the CD monitored denaturation, and also consistent with apoN53I and apoN97S C-lobe destabilization and stabilization, respectively.

Heat denaturation in the presence of 5 M urea was applied to denature the highly stable CaCaM (FIG. 11C). Urea denaturation reverses the order of CaM lobe unfolding i.e. the N-lobe unfolds at lower temperature than the C-lobe (Masino, L, et al., 2000). Thus the left shift in the initial part of the CaN53I denaturation curve relative to the WT indicated that the CaN53I N-lobe was destabilized. Similarly the left shift in the latter part of the CaN97S denaturation curve was consistent with a destabilized CaN97S C-lobe. The dual effects of urea and heating on CaCaM elude thermodynamic evaluation by model fitting. However, addition of the individual apoCaM lobes' Gibbs free energy of folding and the corresponding free energy of binding two Ca²⁺, gave an estimate of the Gibbs free energy of folding for the CaCaM variants (Table 4). Interestingly, these values were in agreement with the CaCaM denaturation curves and consistent with the CaM mutations also affecting the folding stability of the Ca²⁺ loaded forms.

### N53I and N97S Mutations Affect CaM Structure

Using native gel electrophoresis, we investigated the effects of CaM mutations on the protein hydrodynamic radius. There were no apparent changes in the absolute mobility of the CaN53I and CaN97S relative to the WT CaCaM. However, the apoN97S showed an increased absolute mobility that could not be attributed to binding of residual Ca²⁺ in the gel nor thermal effects (FIG. 13A). Hence, apoN97S CaM appeared more compact than WT. No difference in electrophoretic mobility was observed for apoN53I.

Differences in secondary structure distributions in the CaM variants were investigated by CD (FIG. 13B). In both the apo and Ca²⁺ loaded form, the N53I was highly similar to the CaWT except for a signal increase at the 194 nm maximum. Both the apo- and CaN97S showed an altered CD spectrum with increased signal intensities at the 208 and 222 nm minima. In addition, the apoN97S displayed increased signal intensity at the 194 nm maximum. Consistent with the differences at the 208 and 222 nm minima between the N97S and WT CD spectra, prediction of the secondary structure distributions using the CDSSTR program indicated a small decrease in the apo- and CaN97S α-helix content. The equivalent analysis for the N53I variant indicated a decreased α-helix and unordered contents in the apo and Ca²⁺ loaded forms, respectively. CD signal intensities across CaM variants were identical in the 245-250 nm range.

### Differences in Ca²⁺ Affinities of CPVT-CaM/RYR2 CaMBD Complexes

The effect of CaM mutations on the CaM mediated Ca²⁺ sensing of RYR2 was investigated using a 31 residue peptide (RYR2_CaMBD) corresponding to the RYR2 CaM binding domain. ApoCaM binds RYR2_CaMBD and upon Ca²⁺ binding to the C-lobe, the complex (CaM/RYR2_CaMBD) undergoes a structural shift, where the Ca²⁺ bound C-lobe almost fully encompasses the RYR2_CaMBD single Trp residue, markedly affecting the fluorescence (Maximciuc, A A, et al., 2006; Nyegaard, M, et al., 2012). The apoCaM/RYR2_CaMBD complex was titrated with Ca²⁺ and the change in tryptophan fluorescence signal monitored, and taken as a measure of the Ca²⁺ binding of the CaM C-lobes in complex (FIG. 14). The Ca²⁺ affinity of all CaM variants' C-lobes were markedly increased (˜100 fold) in the CaM/RYR2_CaMBD complex compared to free CaM. The RYR2_CaMBD bound N53I and N97S C-lobes demonstrated Ca²⁺ affinity differences mirroring those of the free variants. Fitting to the two-site Adair model demonstrated a minute but significant increase in the K2 value for the N53I/RYR2_CaMBD C-lobe, i.e. an increased Ca²⁺ affinity (Table 5). Interestingly, the magnitude of this K2 increase was greater than that observed for the free proteins (compared in Tables 1 and 5). The N97S/RYR2_CaMBD C-lobe showed a marked decrease in Ca²⁺ affinity owing to significantly decreased K1 and K2 values (Table 5). As for the N53I/RYR2_CaMBD C-lobe, the magnitude of the changes in Ca²⁺ affinity for the N97S/RYR2_CaMBD C-lobe were attenuated compared to the free protein.

### TABLES

TABLE 1
Thermodynamic parameters for the CaM Ca²⁺ binding as calculated from the two
equilibrium titration methods. Results from [Ca²⁺]_{free}-buffered titrations. Calculated equilibrium
constants K1-2 and ΔG₂ values for either CaM lobe binding two Ca²⁺ ions and that of total
CaM. Errors in parenthesis indicate 95% confidence intervals (+/−). Previously published
values for the WT variant provided. The magnitudes of statistically significant changes to WT
CaM were calculated as the ratio of dissociation constants (ΔK1, ΔK2 [—]).

| | | | | ratio to | | ΔG₂ [kJ/mol] | |
|---|---|---|---|---|---|---|---|
| | | log values [—] | | WT [—] | | | Vanscoyc, WS, et |
| | | K1 | K2 | ΔK1 | ΔK2 | This study | al., 2002) |
| N-lobe | WT | 4.75 (0.10) | 9.59 (0.05) | | | −54.71 (0.27) | −56.07 (0.49) |
| | N53I | 4.68 (0.13) | 9.44 (0.07) | | 0.71 | −53.86 (0.37) | |
| | N97S | 4.68 (0.11) | 9.61 (0.05) | | | −54.85 (0.26) | |
| C-lobe | WT | 5.23 (0.07) | 11.21 (0.02) | | | −63.95 (0.11) | −64.02 (0.57) |
| | N53I | 5.34 (0.05) | 11.27 (0.02) | | 1.14 | −64.27 (0.09) | |
| | N97S | 4.73 (0.05) | 9.95 (0.02) | 0.31 | 0.06 | −56.77 (0.10) | |
| CaM | WT | | | | | −118.66 (0.29) | −120.08 (0.75) |
| | N53I | | | | | −118.13 (0.38) | |
| | N97S | | | | | −111.62 (0.27) | |

TABLE 2
CaM N- and C-lobe koff values with N- and C-lobe rates measured at 8° C. and 25° C.,
respectively. The transition state theory thermodynamic parameters for the CaM C-lobe
Ca²⁺ dissociation calculated from fitting k_{off}(T) to the Arrhenius equation.
Values are in kJ/mol where nothing else is stated.

| | | k_{off} [s⁻¹] | Eₐ | ΔH^{\|} | ΔS^{\|} | ΔG^{\|} |
|---|---|---|---|---|---|---|
| CaM N-lobe | WT | 1000 (44) | | | | |
| | N53I | 2406 (371) | | | | |
| | N97S | 758 (23) | | | | |
| | WT | 11.8 (0.19) | 54.75 (0.72) | 52.27 (0.72) | −40.80 (0.49) | 64.44 (0.72) |
| CaM C-lobe | N53I | 11.4 (0.18) | 49.31 (1.47) | 46.83 (1.47) | −59.59 (1.59) | 64.60 (1.47) |
| | N97S | 137.5 (12.55) | 52.46 (2.37) | 49.98 (2.37) | −28.33 (1.04) | 58.43 (2.37) |

TABLE 3
Thermodynamic parameters for the thermal denaturation of the apoCaM
variants as calculated from the data presented in FIG 9. The
denaturation temperature (Tₘ) and enthalpy (ΔH(Tₘ)) was used to
calculate the standard enthalpy (ΔH°), entropy (ΔS°) and
Gibbs free energy (ΔG°) of the individual CaM lobe denaturation.
Errors in parenthesis (+/−) correspond to the 95% confidence intervals.

| | | | ΔH° | ΔS° | ΔG° |
|---|---|---|---|---|---|
| | | Tₘ [°C.] | [kJ/mol] | [kJ/mol] | [kJ/mol] |
| CaM | WT | 59.25 (0.09) | 216.64 (4.92) | 0.65 (0.02) | 22.33 (4.92) |
| N- | N53I | 56.45 (0.10) | 188.51 (4.92) | 0.57 (0.02) | 17.99 (4.29) |
| lobe | N97S | 60.05 (0.09) | 214.85 (4.39) | 0.64 (0.01) | 22.61 (4.39) |
| CaM | WT | 44.95 (0.12) | 156.11 (2.10) | 0.28 (0.01) | 9.79 (2.10) |
| C- | N53I | 43.55 (0.13) | 157.45 (1.99) | 0.31 (0.01) | 9.22 (1.99) |
| lobe | N97S | 47.45 (0.11) | 163.77 (1.96) | 0.29 (0.01) | 11.47 (1.96) |
| CaM | WT | | | | 32.12 (5.35) |
| | N53I | | | | 27.22 (4.73) |
| | N97S | | | | 34.08 (4.81) |

TABLE 4
Estimates of the Gibbs free energy of folding (ΔG_{folding}) for the CaCaM
variants based on values determined for apoCaM variants and
their free energy of binding two Ca²⁺ ions. Errors
in parenthesis indicate 95% confidence intervals (+/−)

| | | ΔG_{folding} [kJ/mol] | |
|---|---|---|---|
| | | N-lobe | C-lobe |
| CaCaM | WT | −77.04 (4.93) | −73.74 (2.10) |
| | N53I | −71.85 (4.31) | −73.49 (1.99) |
| | N97S | −77.46 (4.40) | −68.24 (1.96) |

TABLE 5
CaM C-lobe Adair values from CaM:RYR2 Ca²⁺ titrations. Errors
in parenthesis indicate 95% confidence intervals (+/−). The magnitudes
of statistically significant changes to WT CaM were calculated as the
ratio of dissociation constants (ΔK1, ΔK2 [—]).

| | | | | ratio to | | |
|---|---|---|---|---|---|---|
| | | log values [—] | | WT [—] | | |
| | | K1 | K2 | ΔK1 | ΔK2 | ΔG₂ [kJ/mol] |
| CaM | WT | 7.62 (0.13) | 15.18 (0.06) | | | −86.58 (0.37) |
| C- | N53I | 7.70 (0.12) | 15.31 (0.06) | | 1.37 | −87.35 (0.36) |
| lobe | N97S | 7.11 (0.10) | 13.70 (0.08) | 0.31 | 0.03 | −78.18 (0.44) |

### REFERENCES

Crotti L et al., (2013) Calmodulin mutations associated with recurrent cardiac arrest in infants. Circulation. 127(9):1009-17
Spits et al., (2006) Whole-genome multiple displacement amplification from single cells. Nature Protocols 1, 1965-1970.
Ronaghi, M., Nygren, M., Lundeberg, J., and Nyren, P. (1999) Analyses of secondary structures in DNA by pyrosequencing. Anal. Biochem. 267, 65-71
Su et al., (2011) Next-generation sequencing and its applications in molecular diagnostics. Expert Rev Mol Diagn. April; 11(3).
Metzker. (2010) Applications of next-generation Sequencing technologies—the next generation. Nature Reviews Genetics 11, 31-46.
Niedringhaus et al., (2011). Landscape of next-generation sequencing technologies. Anal Chem. June 15; 83(12):4327-41.
Erg and Wittwer. (2010) High resolution melting analysis for gene scanning. Methods April; 50(4):250-61.
Liu W, Smith D I, Rechtzigel K J, Thibodeau S N, James C D. (1998) Denaturing high performance liquid chromatography (DHPLC) used in the detection of germline and somatic mutations. Nucleic Acids Res. 1998 Mar. 15; 26(6):1396-400.
Schafer A J, Hawkins J R. (1998) DNA variation and the future of human genetics. Nat Biotechnol. January; 16(1):33-9.
Yi et al., (2011) Development of a approach for the detection of low-abundant DNA point mutations. Cell Biochem Biophys. December; 61(3):629-36.
Rodi et al., (2002) A strategy for the rapid discovery of disease markers using the MassARRAY system. Biotechniques June; Suppl: 62-6, 68-9.
Prince et al., (2011) Robust and accurate single nucleotide polymorphism genotyping by dynamic allele-specific hybridization (DASH): design criteria and assay validation.
Genome Res. January; 11(1):152-62.
Dweck D, Reyes-Alfonso A Jr, Potter J D. Expanding the range of free calcium regulation in biological solutions.
Anal Biochem. 2005 Dec. 15; 347(2):303-15.
VanScyoc W S, Sorensen B R, Rusinova E, Laws W R, Ross J B, Shea M A. Calcium binding to calmodulin mutants monitored by domain-specific intrinsic phenylalanine and tyrosine fluorescence.
Biophys J. 2002 November; 83(5):2767-80.
Masino L, Martin S R, Bayley P M.
Ligand binding and thermodynamic stability of a multidomain protein, calmodulin.
Protein Sci. 2000 August; 9(8):1519-29.
Sorensen B R, Shea M A.
Interactions between domains of apo calmodulin alter calcium binding and stability. Biochemistry. 1998 Mar. 24; 37(12):4244-53.
Sreerama N, Woody R W.
Estimation of protein secondary structure from circular dichroism spectra: comparison of CONTIN, SELCON, and CDSSTR methods with an expanded reference set.
Anal Biochem. 2000 Dec. 15; 287(2):252-60.
Maximciuc A A, Putkey J A, Shamoo Y, Mackenzie K R.
Complex of calmodulin with a ryanodine receptor target reveals a novel, flexible binding mode.
Structure. 2006 October; 14(10):1547-56.

Sequences

SEQ ID NO: 1
Calmodulin 1 (CALM1) gene sequence (from UCSC
genome browser). Exon sequence in upper case.
>hg19_knownGene_uc001xy1.2 range =
chr14: 90863327-90874619 5′pad = 0 3′pad = 0
strand = + repeatMasking = none
GATACGGCGCACCATATATATATCGCGGGGCGCAGACTCGCGCTCCGGCA
GTGGTGCTGGGAGTGTCGTGGACGCCGTGCCGTTACTCGTAGTCAGGCGG
CGGCGCAGGCGGCGGCGGCGGCATAGCGCACAGCGCGCCTTAGCAGCAGC
AGCAGCAGCAGCGGCATCGGAGGTACCCCCGCCGTCGCAGCCCCCGCGCT
GGTGCAGCCACCCTCGCTCCCTCTGCTCTTCCTCCCTTCGCTCGCACCAT
Ggtaggtcgggagtggcaaatgccggcgtagcagctgcccgagatttctt
cccagatttctagttgttttgtttgttttttgtttgtttttggttcttgg
aggtttttcttttctgagtgttacgcagcagctgcgcttaaaggaggttg
cattttggatttgcatctcggcgacctctgccagggagcttcatttattg
gttccccttggagctggacttggtcgtaggccgtccacgggcaggggctc
cggccgcaactgcagcgggggtttctgcatccaatccccctgccccccgc
ccagccccgcacccactgcatccactagcgccgcacccgggctgcctgca
gcgcagcgtttcggcctgggagccgggcggggccgggcactagacccccc
cccccggcccgcccctccccaccccgcttctccgccggcgcgaaggtggc
aggtcgggcgggcagtggagaatgaatgggctggagctggccggtggcgc
acattgttccggccgggtgttgaggggcgcagtcagcgcccgccacctcc
ccactttggccggccctgctgggcgccctccctcggtcgctctcccctcc
ttcttcccggggggcgcggcgcgggcgtgggctgggaaggaaggagccgg
ggaagggtggggttgggggcaggaaggcgaggggttgggggcggagaggg
cggaagcggcggccgggccgccctgcgcccgggcggggccctgcggtgtg
gccgtggcttgttcctgccgctttcgcaccctgcggccccccacccagtg
cagcagtgcgggcgggcgtgagcctcggtgcaccaggaggcacttcccgc
gggaggcgctgggctcgcgctaattggggcggggggggggggcggcgggg
gaggagggaactggcgcgcggcttggtttccattagagacgcaaagtttc
tgctccgggaggaggcggcggcgccgcgggctcgtcgcctgggggagcag
aagcgggtgggaggtgcgggtggccttggcctcagccctggtgcgcgggg
gccgggggtggtgaccctcctggccgaggaggggcggcgtccagacgccc
gctcgggggccgccttcccccccacgcctgcccccgggcacgcgccctgc
ccggtccctcgccccgcgccacttccagtccgcagagagatgccctccac
gtttctgctttctctgcagcctctagattgccagatgcgactgtgcgcct
cgctgggtgtgttttccacagccccttcctcctcggcgtgcagggctgac
atcaccgactgcgtttctggtttggcgggtggggagatggttccccgcag
ggttctggtacacctttgcccccagggctagcgccatttgggggaggagg
ttttcgttgtcgagaaagttggatgctcctggtaacccctctaacaagag
agttctgtagcgaggtgggactgttctccccataaggtgacagtttctct
tgcgaggtgtggcagcgcttcctgttgtacaagacagatgttgccttggc
gttacgtaaatcatcgtgtctccgtcatttaaagaaagccaatttttagt
gattgaggtagaaagaaagatccgtttataatttgtaaaaacaaattttc
acccagaatcaatatattggaacaccattcctactgttaaagttttcact
taagagtataaacttcatcagctttctattaggacttattttgtaattgg
cttcttaggcatccttctttaaaagagaaatccacgttagctctccttga
ggtctcgagttccctcggctggaggcacaggttcagtggagaccaaataa
tgcaggtgaattaccttcgtggccattactgcctccaacgaagtgtgttt
attaagaacagttcttatgtcattcttaaggtaggtagggttaatactct
ccagcaaatttagtagatactctttgccagaaaagagaggagtatatata
gtttgataattattgtgtagttttctgtgtacttaatttttgcagttttg
taacacttcatttgtaagatggtaccattttttcctggcttctgaatcat
aggatagtttgacccagggcattagccattgtaatggtaggcttttaaca
aataactgcctaatttaaaggattggaaagcatttgttacatggaaatga
agttggtggcgtacccagttgctgtatctttattttttctacttaattat
ttctcataaaatggatataaaagcctgttaatccaacccaatgccattat
gtaacgccagtttggagatttcgagggcctggagcagtgcgcaaggtgcg
ctgaaagcctgcccctggatgagatccttatcctggctgtgatggcagtg
gcagtgggctgggtcccttgttgagtggaaagggggactgcggtgtccat
ggtgcagtaggtggcgctcttctgtcttagagcctgccgccactgcagct
ggtgccaaggggccttctgccactagaggtgccatttttcacatgatgaa
cttagcctagttagatcgcagagcaagctgtaagccatgggcccagaaaa
gaaaacttgaagtgagcagatgttgtcacttccttgtaatcctttgttaa
aatagcataaggagttttctttattctatttactttcattaaatgaccgt
gctacaggtttcaaaggattttaagattgatttttgaaagatcacaatat
taaaagtataactggaaaacctatgttgaaatcaaccaaacatgtcgtgg
actgaatgataaccttttctttcttcatatagGCTGATCAGCTGACCGAA
GAACAGATTGCTGgtaagttgacaactccaaggagtccccagaaggccag
aactaggcactgactcagttttggtgactcctctgttcctccccgctaca
gtctgggcagttttctaagaatttatttaaataagaacagtaagcagaaa
cactgaggtcagatgttattcttgccagtactttatagatgaggtgaaag
gaagtaaaactaaggatgcccacatgttaaactctggagaatttgaccat
gtttcacaatgtgcaaagtttgcgtatgattaattgtactgagcctgcta
ctcagcggtttagtttacaattcttatgccatgggtctttcagtaatctg
ccacgaaagcttgtgctcgctatcctaaaataaatggaaatgggtgaata
tgagtgttaggaccactgtagtaattgggaagaaagttacattagttaaa
ctctgttgcccaggctggtctctaactcctgggctcaagcaatcctcctg
cctcagcctcctgagtagctgggactacaggcatgtgccaccacgtctgg
cagattttagctttttaatattcctggaggacttgttttgagactgtttc
tcgttaggaaaccaggaatgcttctgaaatattctaaaagtcatgtggag
agagtttacctgggaatgtacatttctagtaaccattttatttgttatga
aacaagggattcttatggctttagaaatgtaacaggaagggatttgaagg
gggcacatggaccaatcttgtcagattggatttagtcccttgaacctggg
aggcaggggttgtagtgagctgagattgcaccactgcactccaatctcgg
tgacagagcgagactccattgtttaaaaaaaaaaaaaaagattggattta
ggactaatttaagcatgttccagcttagccgccttgaaacctttgggaat
attgtggtgtgtggcactgtttattgggagcagtgtttgctttatgggct
gctgtatgaaggccagtccaacaggactattgtggtcattatttcagtag
ataaagaccagacttctgatacgttgcacaacttgaatggctggctttgg
caagcccccggcaagtgtgtattgtgactgggttggataaagacattgat
tctaacgggtcaacttttgttttcagAATTCAAGGAAGCCTTCTCCCTAT
TTGATAAAGATGGCGATGGCACCATCACAACAAAGGAACTTGGAACTGTC
ATGAGGTCACTGGGTCAGAACCCAACAGAAGCTGAATTGCAGGATATGAT
CAATGAAGTGGATGCTGATGgtaagagctttaaaaccatgaatgagggcc
attgttgtgtaattcaagttcagacatgttacaggattgtctttcaggtc
cccagagcaaagcaaatgtgcaaagatcctttctgtggttgccccagggc
cattgacaattaaaatagaagatgatgggccttgcgtccatcctgcttag
tgtctagaatgttttctgcatgggatcactattgttttcttcctgcttgg
tgcgacctagagctcaaatctatttttttttttttttttggagacggagt
ctcgccctgtcgcccaggctggagtggcactggcgcgatctcggctcact
gcaacctctgcctcttgggttccagcgattctcctgcgtcagccttctga
gtagctggaattacaggcgtgtgtcgccacgcccagttagtgttttgtat
ctttagtagagatggggtttcaccatgttggccaggctggtctcaaactc
ctgacctcgtgatccgccctccccggcctcccaaagtgctgggattacag
gcgtgaaccactgctcctggccgagctcaaagcttttatcaactggccca
tgagtctgcactgagtcttgaggggggaggtgaaattaaatagccataga
aagtgctttttaacaaacttactgtgtttaaagaggaggaggaaccccca
gatgaagtaggtgacgagcactcttagaagttaccataaaagtgagtaca
gtgtgagctgtagatgtgtttgctgcagaggagcatgtgaggtttggagg
cggatgtgtggtgactccaggggatagatttgcagaacctaacggaaagg
gaagctgtaaggtgcagggccagagggaaccagcagtaaccctgatagcg
gtctgtcatctgttcctctcgactctacagcagcggacaacagaactttg
attgctgatttccatcagtaagcaggctttgaagcacacttccccacccc
taaaaaaaaaccacgtattttggtaaatcctatatatattctaatgtact
gtatgacagtatagaacatgatttttaaaagatgagttgggaggagaaaa
ggataaaagaaaaaataaaagaagcattaagaataaacaattcggatcta
gattttactttctagatgattgactcgagggtggtgtagtaaaatcgctt
gtctggtcacaaacatttggcagcagagcttttgattaggttctttgaca
aagccttcagcacgttagagtggttttcactaatagtgttttggaaagaa
aaggttgtccatagttctctagtttgctaagatgatcagctacccaggaa
cgtggagtaacttcctcttgtttgtgggagccccgggaatctgtgcctgg
ggaggggagaagtctgttaggctcttggattgtgtggaagaaggagaagt
tgtgccaggctacagaatcctgtgtttgcactgagaaaacaggatggtac
ctgaccttctctgcatggctgtgagatagcttaaaataatttcttttgtt
tttgatgaatatgaacaatatcttaaaatttttgaggctaaaaaagtctt
gaagggatccctgaggtattttctttgaaaggtactggtgaaaatgagta
acttaacctaagggtttttctttctaattttatttccatttagttcaatg
acactgttagtctggagtgcttgtctttgggggtattcatctcttagttt
taaagaggagttgtttggagtactggccgtagaacagattgttctgacag
ttccctaagtgttactagtctgagctgtgagaatgctcctgagcttttcc
cttaatgggaaataaagatactgagttggaagaaaacaggtggctaacca
tcatagcgtggccaagaaatgatcctggagaagacttggtaagacttcat
ggcccatgcatggcataacagaatcaatgttcctctctcataatcttttc
tcctctgaaacactttatacacttaacctgcagctcagttctaggccttt
tttgtgttactgctgtcactaaccaaggcagagtgagacctgagtgattt
ccctaactcagggatggcagtcgggggcgctttcttccctcggagtggaa
agattcagcctgcggagtggtgtatgctatttttctcttgaactgtacag
cccttcatgacccttccatgggcttgaatccagatgtgcagtttcctttg
tataattaaatactatcctgggcactgatgatgagtttgaaattatgtga
aattgccctgtgaagtgtttgaacgttttagacctgcagatgattgaacc
tagtaagatagtctgcccctttgtcctagtacatgtttaccgttccgtac
agtggttctgaaatgattactgcagagcagcgttaatggagtgcttactt
tacatgagctttttgttttttaattcgagGTAATGGCACCATTGACTTCC
CCGAATTTTTGACTATGATGGCTAGAAAAATGAAAGATACAGATAGTGAA
GAAGAAATCCGTGAGGCATTCCGAGTCTTTGACAAGgtaatccagcatct
acatagcagatggtacttaagtatggcttcttccgctttcacttctaaaa
gctataataatgttatagacagaagacttaaatctaactgcctgagcctc
tgatctcactttcaaaaatcctccttatggtaaccgtatcaggggagggt
aggcataataaataggaattttggaccatgtttcttgactgttactttga
attgttgtgagcttttgcaaatcctgttttctgcattagctgtttgcatg
tatttagtaggttagaggtgggaactagagatcagagaattgtttatggc
agcagagttagcagtaacttgagagggcatagctaagtcaaagacctact
tccccacactacatcattagcaataacaattgctgaatgttcacagGATG
GCAATGGTTATATCAGTGCAGCAGAACTACGTCACGTCATGACAAACTTA
GGAGAAAAACTAACAGATGAAGAAGTAGATGAAATGATCAGAGAAGCAGA
TATTGATGGAGACGGACAAGTCAACTATGAAGgtaaaactaaattctctg
agctcagtgtttcatagtcttacctttagatctgtaagcaagccaactgc
ttcactagacagcctttgactttattttatgtacagtaaagatgttgtgt
tcattaaagctgttttcaaagataaccaaaagttactattatatttgtct
tttcagAATTCGTACAGATGATGACTGCAAAATGAAGACCTACTTTCAAC
TCCTTTTTCCCCCCTCTAGAAGAATCAAATTGAATCTTTTACTTACCTCT
TGCAAAAAAAAGAAAAAAGAAAAAAGTTCATTTATTCATTCTGTTTCTAT
ATAGCAAAACTGAATGTCAAAAGTACCTTCTGTCCACACACACAAAATCT
GCATGTATTGGTTGGTGGTCCTGTCCCCTAAAGATCAAGCTACACATCAG
TTTTACAATATAAATACTTGTACTACCTTAATGATAAGGACTCCTTAAAG
TTCCATTTGCTAATGATTAATACACTGTTTGGGCTGGCCAGTTTTTCATG
CATGCAGCTTGACGATTGAGCACAGTCAGGCCTTTGTATTAAAAATGAAA
AATGAAAAAACAAATTCAAAACCTATTCAAATGGGTTCTAGTTCAATTTG
TTTAGTATAAATTGTCATAGCTGGTTTACTGAAAACAAACACATTTAAAA
TTGGTTTACCTCAGGATGACGTGCAGAAAAATGGGTGAAGGATAAACCGT
TGAGACGTGGCCCCACTGGTAGGATGGTCCTCTTGTACTTCGTGTGCTCC
GACCCATGGTGACGATGACACACCCTGGTGGCATGCCCGTGTATGTTGGT
TTAGCGTTGTCTGCATTGTTCTAGAGTGAAACAGGTGTCAGGCTGTCACT
GTTCACACAAATTTTTAATAAGAAACATTTACCAAGGGAGCATCTTTGGA
CTCTCTGTTTTTAAAACCTTCTGAACCATGACTTGGAGCCGGCAGAGTAG
GCTGTGGCTGTGGACTTCAGCACAACCATCAACATTGCTGTTCAAAGAAA
TTACAGTTTACGTCCATTCCAAGTTGTAAATGCTAGTCTTTTTTTTTTTT
TTTCCAATAAAAAGACCATTAACTTAAAGTGGTGTTAAATGCTTTGTAAA
GCTGAGATCTAAATGGGGACAAGGCAGGTGGAGGGGAGGCCAGTGTACAT
GTAAATGCCCACAGCCCAGCATTGGGTTTCCCTCCCAAGGCCCCAGCACC
AACCTCTGAGCCCAAGACCTTGCCTGAAAACAAGCAGATACCGATTGCTT
CATCCTATTTATGGACATGTAGGTCTAGTTGCATTTTCACTGGGGGGAGG
GGGGAAGGTGAATTATGGTAACTTTTAATGATCTATTCAGGCAGTAGAGC
TCTTAAGGAAAAAAAAAAACCCACTTTCTCTCAAGCATGTATTTAGGGGT
TGTTCTCAATTGTGCTGCTGATTACCTGTCTTATGTAACTACTTGAGACC
ATCTGCAAGAGACATGATTTAGTGTGTCTGTAATTCAATCTTCGCTGTGT
GTGGTAGAAGCAGTAGTCACTTTTGTAAGCCAGTCTCTTCATGCCTAAAA
GACACTACCAGTCACCTTTGATTCGCGACTTTTAATTTATGATTATACTT
AGCCTCCTCCTCCTTTTTTTTTTTTTCCCAAGTTGACTTGACTTTGCTTT
TTTCCCCCCAAGTAGAACTAATGCTAGCTTCCAGCTTGAAAGTAAAACTC
CAGTGTGGAGTGAATTTTGTGTCTAATTATAAACCTGTAACCAAAACTCA
GACATCTGGTACTGGTCTTTGCATTGAGATTGGTCCCTGTAAAACCCCCT
TTAAAAGCATATTGCATTTAGTACAGAGCTCTTTTTTGAAATGAAGGCTG
GAGATGTGCATTTTTCACGGTGTTAACTGGTTGTATCTTATTAGCAAGGA
GATTGGGGTTTTGAGTGTTTGCGTGGGTGGTTTCAATTTGCCAGGGAACA
GTGGCAGGCTGCTAGCAAGGCAGTGAGAAGCTCTTGGCAGCCAAATGGGT
GCATTCAGGGCTGATTTATAGAGACCCTTGGCTTCTCCTTCTCCTACTCC
CTGTCTTTCTGGCATTTTGTAGCTTGTTAGATTTTCTGCCAGAGGGGTGG
GTCAGAGCAGTGGAGGGGAGACATCGCCCATGTGCTTCTGCTACTGGTCC
TTGGGCTGGGTGGTTGGTAGAGGAGATGTTGACACTATGAGCTAAGGGTT
GGCTTTTGTAATTACCTGAATCTGAAAGGAATGCCTAAGGTTACCTTGGG
GTTTCTCTTCTGGTGAGATAGGGTTCCTGGTTTGAGTAAGTTAATGTCCT
GGATATTTCTTGTGGCAGGGGGTGGTCAAAGAGCCTGATTGCTGACCCAG
TCTCAGGCCTGTGGTCGATGACCTCTCGGTAGTTTCAAAGGGGGCTGGAG
GGGGATATTTGACTTGTTTTTTCGAAATGTAGCCTTCTAACCCTCAAGTC
TTTAGAAGCTGGGTGGACTCTTAGTGGTCCTGCAGCGTATCCTAAAAGAC
TACCTTTGAAACAGGATTCTTGTATGGCCAGGATCCTGTCTGGGAACCAG
AAACCCTACACCCTCCCCCTCCAGGGAATGCTGAGTTCCAGTTTTGAGCA
GAGGTGAGGCAGAATCCACTGTAGCCTTCCGCCCTGGTATTTGGGGGGAT
GACCAGCCCAGGCGTTGGGTGTTAGTCTGCATGAGTTTGTGAGAGGAAAT
AGCTGGGTGTCCTGGCAGTGCCCTTGAAGTTGGTTAGGACCTTCCTGTAA
ACTCTTGCCCCTACTTCTAACTACTCTATAAATATATACATATATTTATA
TATAAAGTGATTAGTTGAACTGGCATCCTGCTTTAGCCTGAGACTTGCCA
TAAGAAACTGCTGAGTACTTGGCAAACCCTTTCATAGTTTTGTTCTCCAT
CTGTTTGGGGTAGGTGTTGAGCGAGGCAAATGGATCTCGATATTTCAGAT
GGGCTTTTGATGCACTGTTGCCAAGGAAGGCTTTTTCTGATTTTTTGACA
AATGAATTTTTGCACACTTTCATTGGTGTCTTTCGGCAACTTACACACAT
TGAAAATGAGCTATTGTACATATTTTTATATTCTCTTTATAAATGCATGT
CTGATTGTACTTGTAACAATATTGTAATGAACGGCTGTGCAGTAGGCCCA
GCGCTGCTGTGTCTCGTCAGAGGAATAGCTTACCACGAACCCCTCAGCAT
ACTGGGAATCTCTTCCTGAACAACGAATGTAAATTTGGTCAAGTCTACTC
TTCCGTTCATTCAATTATTTTAAGCATTTGAATTATTTATTGTATATCCT
AAATATATTTCTCCTTTGGCAGTGACTAGATTTCCACTAATGTGTCTTAA
TCTATCCCTCCAGCTGGCAGTTACTGTTTTTTTAATCCCCTGAAGTTGTC
CTGTAGGAGACAGAAATTCTTTGCTGTCTGTATCCCTTGGAGTAAGAAGG
TAGTGGCATGGGTGGAGTGTGTGTTCTTTCTCCAAATCTATTATGATGTT
TATTAAACACTTCTGTAGCAAAGATGGTGGTAGTTCTTTTGTTACTGAAG
TTGCCCTTCACCATGGCTATTTGAAAAGGAGATGTACTTGGACGTTTCTG
TAAATCTTGAGATAAACTGTTTGGAGATTTAACCACCTCTCTGATGGGGG
ACCAACTCTATGGAAATTGTAAATACGTTTTATTTATAAACCTGGCACTG
TATTCAATAAACATTTCTGCAGCCTTTCATCTCTAACTGCGAA
SEQ ID NO: 2
Calmodulin 2 (CALM2) gene sequence (from UCSC
genome browser). Exon sequence in upper case.
>hg19_knownGene_uc002rvt.2 range =
chr2: 47387221-47403740 5′pad = 0 3′pad = 0
strand = − repeatMasking = none
CTCGTTTGCGATGTTCCGTTATCTGGATGCGGCGGAGGGATCTGGCGGAG
GGAGGTGTTTATGAGGCGCTGGGGGCGGAGGAGGCGAATTAGTCCGAGTG
GAGAGAGCGAGCTGAGTGGTTGTGTGGTCGCGTCTCGGAAACCGGTAGCG
CTTGCAGCATGgtgagtgcatcgctgagctgccggcgctgggcctgtggg
gggaagagactggagcgaactgactaaacaaaggaaactcgcctccctta
caccttcaaggagtggtttctgccagagggatggcgcgaaagaggccagg
gatgccggcgacggttgtgtcgcaggtgctcccccacccccattctcttc
aggggggcttcctctagtgagttgagggccggacgcacagttcccgaggt
ggtgaagctcctcgggggcttggctggaggaggatgggtccgggatgagg
gacagcccagtgcacgggaaggagagaaagtgaaggaagggagagaggga
gcgtagagagccttctgtcgggcgtctggaaaggtcaagcagggggcgaa
ttcaatccacccgaccgacggttagtgcgggcgccaggaagcgcctttgt
ccgcttgcggccgccatgcgcccagggggcgggaggagcgcgcggggccc
tggggcgagcgtcggcgtcgcagttagcccgcgtcctccgcgtgccggga
tccagggggcgcgcgggcgggcgggcggcgcgctgtgtgggccgagcccg
cgcggcgcagggagcggccggtgggggcggggcgggcgagcgcctcgcgg
gacccggagccgccgccccgctcggccccgctcgagctccagctgctctg
ctggcacctgcaggactgcggagatctccttggcgctgcggggccattgg
cggggatgggcgggggaggggggcctcgacggtttcccatccccctctgg
caaccctaatcttccttctccatctgggccctggggcgtcttccaccacc
caggccggatgctttaaaaaaaattgctttttaaagtgtctgtcgttgaa
agaaattagtcttacccttgaagtcaggggcgcgtcctcgcaatacacat
cttgttagggaaagtgttcggctccagctagggttctacaaggcgtttct
tgttcaccgccggagggaagcaggctccggagtgactgccttctgaaagt
cggtcttgtaacaattggatggatgcctttgaagagcccctgtccctatt
ctatgcttgaaaacagcgtgcagtcctaatgttcaagaaccacgaccaca
taaaaacattgctccccttctgctgctttgaaaacgacccctaaattccg
tgtagaagttgccaggtcgtcttgacgtacacttcgtttgtatgatgttt
gtctgtcaaatactgtgatggaagagtgtatgcgggggaggagcagggaa
tttttaaaagcattttccggtcacctcagactgggagatcatgttctttc
ctgaaaaaaaaaaaaaaaaacacctaatgagggcggtgatctgtgatact
tactttgtggttcctcctgccaagtggttacagcgatgggtgtgtccagg
aggcaaggtgacatcttaaacaggagttagggatccttgccaactgaaac
ctccagcctgtatgggtgtggggagacgatttcaccttaaataaaactag
cgttcattgacgtttgatggatttgcttcatcttgatgcttcaaatgtta
ttctgaatttgaatgtgctggttacatttgctttcttaaagatgtagtta
caggcatatgtaataaacattacaagttgtggagtaagctttttttttta
aataaacgaagggtaaagttttctttaatcagttttgataattgaatcag
taattcgaatagattttttcttggtagagttggtatctacgcatttgtgg
tcccttgcccagtgttaagcttttgtagttactgtttgcatcactggtgc
tttgggggttttctgttaaattgacagctcttactataagagacaaagtc
gaaaaaaaaaatcttaataaaaattgacagatatcattatttgtgacaga
tacagtaaatttgaaaattgccttcacataactttataattagaggactg
agattttacattaggaatccaattaaaaaaaattagccggacataatggc
ctgtgcgtgtagtcctggctagtcaatgggctgaggcaggaggatcatct
tgggcccgggagttggagactgtagtgagctatgatcacaccactgcact
ccctgggcaacagagtgagaccctgtctctcaaaaaataaaaaaaattag
gaccccaagttaaagtataaaaaattttatacggaagtaaagtaagattg
ctcatgtaactcttgagtttacatgtaatcaacatatgctcattgaaaac
gggattgcttcaagaggactttgagtgcagggtgattaggtaagtaaaag
atgtaaaaaggtagaaaatttttgtcacttgagtctaaataattgttctt
ataagtgccaacgcctgtttctgttaggctcagaagatcaaaggatttgg
ctcttttaaaatatagaaagctctagcttcagctagaatttaggccttta
gtaatagccctaatttttatgaagccattttgttccagtgatcttttggt
gagagatgctatgtaagtactattcttcagaattaggtgtctttttaccc
taatgaaataatttagattgcttttgatacaggtaaaacaaatatcctgg
cttccataattgtagaaaaaacttcatataggaatccttgttgtatcaaa
gtagcacctgatgggaatgaacagacaggaatggatgaaggatagcagtt
tgcgttccatttcaagcctatgggctcacacatttattcagataagaaca
ccacctttcactagataaactccaacagtattcatgcatacttttgaatg
gcatgtaggaaatgtttgataggtacataatgtattcacttcaggtcact
aatgtaatacggggtcgtgctccttagtgttgacagatcacctatggttc
tccaaaatgaacattctagtacaggaggtctagggaggaacctgagagta
tactaatgcctaggaactttctctggagtggcaagagcagtgggaagaat
tatgtcaatagctacagaaataagggagtaagaacaagtcatctctctag
tgaattcttcttcactttactgagataaacatacatgttaatgagcttga
gttttcccaaaagtataattcttctggttcttctaagaaaatggcactcc
ctggaaacaaggaagaaccaaatttattcgcctttgtagcagttgggaaa
gttagtgctaggaagtcttcttgatttatagtaggctttaatctggatat
tgctggtaaaagtttattctaaaacctgaactctggataagtaatacaaa
aagcttctcaaccttccaagcaaaattgagagctttcaggttatgtgagt
aatttggtctcttgggtgcttaattcattccttgaagctcatttttgtga
tctcttccaagattgcatttgcttggaggtagggagttagacaagatggt
atgaggtccctaaattttgactttccaagcaaaattggacagtggttcct
aaattgctaacatcctcgtttcttcctaaggcttctcatgtttcatatat
agtagccttcccaaagtcccatttcccaccccccccccccccccaaccca
tgtagagagaacgaacctgtctcccttcctgtacagagtacgggatcctt
caactttcacacaggctgcagtgtctgccacacatttagctcaacttttt
tttagccttaaagtgatgtccgctgcatctgtcgctgggttgcaccttgt
ggatttagtttgcataaattttctcagcttaaacaaagttaacattgaat
agagtaagcttaccataaagggcttaataaatgccatgcatgtctacatt
cggtgtggaaattgagctagtcaggttgatatttaacattgtaggttctt
tgttaatttatatgaaataatggttatcatttaactctttaggttagctt
tgtacatagcatctcactttgcacaacaaccctgcaaggtaagtattgtt
attcttgtgctacaaatgaagttgactgagaggaggagtaccacgtccaa
ggtcacacagctattaaatggcagggctgggatactggcctgtgactcag
aacttgatgctttccccccacgccacgcatgccaggttgcccttcctttc
agaaatggtggaagtcctgcaaaatgcaataaactgaagtaatgtagctt
ctattaatacaaagtaaataactcagatttactggattttaaaccttatt
ccttgggtaaacaatctgtgactgacttcacaccaaatatttgttggcgg
aggatttggactttagggataaaagtggatacattttttattttacaaac
tctgtatttgaacttaattattggctcttcaattttacgttaccagcttt
tttttttttttttttttttaatgaatttgatttacatcatggtcaaacaa
aaattgttgagcagggaaaataaactgctttctggattccttcttgaatt
ttctcatgtgccctagagaaaatgtgttccacattaaggtgttacttttt
ccaggggtgtgttcatttaaaaagaatgaagccaggcaatgtttattttt
cttttacctataaataaatgaatggattaatcattgtatacttgactccc
atgttggtagggattttagataggaggctatttcttgtctgtgcttctca
ataccccataagcagttgcttcatggatgtatatactaataagcagtgaa
agaaagtgcatgttcaaagaatacaacaaggagtctggatattttgcaat
catctttatatattacggtgctctgaattaaaagctaaaagttactgggt
atgtctgacaccttagtgctttatctttgttctactaattttctgtgccc
caatcccacttaaccctagcctcattccttatctgtaagataggggataa
taccactgtaaggttattattaagattgaataaggataaaatttataatg
ggttttagcaaatggcagaaaatattttctgaagaaaaccaagtgctatt
aaaaaaacatcacaagccttgggcttactttgggattttaaaaaccaaga
gaaaatggatggctgaactttcaaacatttggtaaatattatagtactgt
agttcagagctctggattctttgcattttgcctgctgggtgagaaggaat
aaaagtttgtgcctttttttttttttaatcactttaatttcaaaacaatg
tgtttaaccatttgtgggagtaattttcattttgtgagcctgaagcattt
tgattcagtgggaatttctggtgatttatatctggaatagaagtgagctt
aagtttagctattctaacgttgaaaaaggaagcaatgtttctattggatt
ctaaagtatattttcaaaaatattctgaagtatttgtatatcttaaactt
ggagttaagacagcttagctttgaagataagagaaactagatgtgtgcat
tttctatccagatgtgtttgttgctggaactaaatgaaacagtacatggt
aacccttgaaaggttttaaacttgtttctgtaactgctaatctacatact
ctcaagtcactaaccttcctctttgatctctttgtagGCTGACCAACTGA
CTGAAGAGCAGATTGCAGgtgagaaatactcagctagattgtacccatta
gattcttattataaattgaatagccaacgtcagaataagagacttgtatg
aaataattgactttggtatatgtcatggatactaacatatgggtataata
taatacagtaattcaacctgctttggcaagtgggattgtaaacttgccga
tggaagatgtgcagggctaattgtagttaaagacatgtattttagtctgc
gtggatatactttggagccttgtggtgtagtggtggtctttgttttgttt
tctgaagactaccttagtataaaacaggaattctgggctagctaacacgc
tagaaaaggaggccaagaagtaactaaggcaacttgaaatggagaatcaa
aaaaggaacaattgaatttaccctagttaaaattagctaacattttaacc
taagaattgcatatataatcagtcagggaataaccatttctcaagtgaaa
aaatttaagaagttggcattctattctataaatgtcactgtagcaatgtg
gatttttctcttaaaaaacagtgacagctgttgaaatgaagccgcttaaa
tcttgtcccttttagtttgctatggaagtatgcaaaattattacataaaa
cctgatgtcatgttctctttatgccatgccatagaacattaatacttttg
atttatccttgtacattgagatggctggttaatgtcaatgttggggaaac
ttttttttccccccttgccggagttgttttttttttttattgtagtgttg
aaatgctttgcaaactattcctttgttttcattagacacttttattagca
tgagcaccaagcagtttatttgccacagttttcacagtaagcaggaaggg
tttgataagagctcaaaagcacttgagcactttttcttaggctggagaaa
actgagaagcccagttggaactatatagcccatttaaagcaagctcccca
cttccataagcatatagaagtttgggggataccagaattacactagtcac
ctcaaatactatttgataggactgtaggtagaaagctattagtattaaag
acaaatttaagcaggttttatttttatttattgtgtaatttgacttaaac
ttagtttttactttctgaggttggtaaattacatgtattgactgttatga
acaaaaagcaaacagttttgggaataattttttttttgttgagttacact
gttttaaaggtatcacctatttgatctcatgtctcctcaaaaaaaataga
ctgggcctgatggctggtaatcttatcactttgggaggctgaggcagcag
gatcacttgagcccaggagtttgagaccagcctgggcaacatagtgagac
cccatctctacaaagtaaatgtatcgtcaaaatgtttttctgaagtagtt
ttttttataaatatactgtctccttcccaaattgtctcaggctgtctaat
actttcactcttaaactctgaccttctgtaatacatttcttaatattccc
agtcaggtacaggtaccaactgtatgcagtacagcatgttcactttaggg
tagaaaacgttactcagtgtgccattgcatagttgatggtctgctctctt
aaacaactctttgaaaagaaagtttgttggccgggcgcagtggctcacgc
ctgtaattccagcactttgggaggccgaggcggatcacttgaggtcagga
gttcatgaccagcctggccaacgtggtgaaaccccatctctcctaaaaat
acaaaaattagccaggcatggtggcgtgcacctgtaatcccagccactca
ggaggctgatgcaggagaatcacttgaacctgggaggcagaggttgcagt
gaggcaagatcatgccactgcactccagcctgggcaacagagcgagactt
tgcctcaaaaaaaaaaaagtttgtgtctattgaatataagaggatccctc
ttcagtgattgatagcatttttattttttaatttattttgagacagtttc
actcttgttgcccaggctggagtgcagtggctcaacttctgtctcccggg
ttcaagctgttctgcttcagtctcccgagtagctgggattagaggcatgc
accacctggctatttttgtatttttagtagagatggggtttctccatgtt
ggtcaggctggtctcaaactcgcaacctcaggtgatccacccgcctcagc
ctcctaaagtgctgggattacaggtgtgaatcaccatgcctggcctggta
gaattcttataaagaatacttgtagttcatcaaaactgtgacattcagga
cctgttctctttattgtctttaagttaaatcctatcttttcttggctttc
atcataaaattaggtttccaatgggagtagagggaaaaaaatctgttctt
aggttctcttcttatcctgccttttcctagaccacagatttctttaaagc
cattccttggacctccaggtttcagttaagatttagggcagaggaaccaa
aacttctatgctactaaaataaaaatcatgtgcggtcagggacctttttg
ttctactttgtaggcccggtacctgccacatagtaatcattgaatattcc
ttgaataaattttattctcaatgagtgtgtaacacaaaaaagttctagag
cactactatcctaagatttctgcctactctccaagtcccttcctcccaaa
tttggtatatttcagtgtttacctttgagatgaaggcaattttttaatct
ggtaaattgtaattgttggtggctgggcatggtggcgcatgtctaatccc
aacactttgggaggctgaggcagtgggattgcttgagcccaggagttcaa
gaccagtctgggcaacacagcaagaccctgtctcaaagaaaagaaaaaaa
aatttttttttttttttttttagttgtaagtgctggtgaccgctaacatc
cttgtgtagatgaagttactaaattttttttttttttttttttttttttt
ttttttttgagacggagcttcactctcttgcccaggctggagtgcagtag
catgatcttggctcactgcaacctttgctgtccaacttcaagcaattctc
ctggctcagtctcccaagtagctgggattacaggcgtgcaccaccacacc
cggctagtttttgtatttttaggagagatggggtttcaccatgttggcca
ggctggtcttgaactgctgacgtcaggtgatccgcttaccttgacctccc
aaagtgttgggattacaggcatgagccactgctcccggcctaaaattttt
tatattacttggtcgtataaagcattccagtctagtatgacatcactaac
aatttcattttgttgataactctgccatgatgtcattagttgttgctgtt
tgacttttggaagactgataggaaaaatgtacagtattttttccttaaga
agtctgtacctaaactttcattaaattttcaaggtccaaatttgtttgag
aaaaatattaatattactcaatgtatagcaacaactgcatgaagcgccaa
aagagttccagtacatgatctttaagggattattaagttcagaattttgt
gaagaaatagaagttaataaacagtcatttgatttgttttgtaattgtct
tgtgataacctattttaataaaatttttaaaacatgttattttaataaaa
ttatatgtttaggtaataaactaattagggaagggccttggggttgctaa
tgaacaaataaaacttgagtaatagatttttctgaaagtttcagtttggt
cagctaaaatggattttggtaggttccttatatctatcaattatgtagaa
gatagctgttctgtggcattcaggacctgttctctttattgtgtgtttag
gttttgactcttggatttaatttcaggtcattcctgggagttacagttta
gaattatatgcttctatacataaagattacgctagttctgaagtacagga
aaactgtaaaggcaggtgaattaaaattttactttggggtttattatttt
gagtttttacaggtattttttactctttagtataaaaatacaagtaaaaa
tttggaataaaccttgacctttaaaagaaatgttatttgttcctttcttg
aatgaataacaaatggtattagcatagatacggttttattttctgcattt
tacttattctagcaaacattttaagtgttattagatttttaaatttctcg
aaagttatacatagtcttacactatgcatcttccagtatttagctttttg
ttaaatcatgtaatagaaaggtgattactgctaattaaacattgggaatt
tatgcacctaatggtggtgttaaaagatacccatagcagagaatgtaata
taaacaaatacctcaaagtcctttaattttcttgtaacctaaattaggtg
aaaggtgcaatgtgtctgaactccaatatatgtatttctagatatttcta
cattctgtttaaaaacagtaaaattgaaatgtctttatcaattgtgctat
ttgtaggtggagactccctgatctatttttgtcattaggttgatctaggt
tagcactgtatactatcttcataaaatcaattagtgtggtatgaaccttc
tgattggtattagatgagttcctctataaaccagtaatatttaaataaga
aagaatgcagttaggaggcaattgtaatagccctggcaaaggacgatgga
gccttgaaccaaaatactggttgtagagatggaggaaaaatcaggcagta
tttaaatgatagaattcgagacctgttgacagatttcagactgaaagagt
taaaagtggctcttgggcttttgctagtacctttagggttgcctgggagt
atagaattcattttcatatgttttgcttgtcatcagccaggggagctata
tcctgtagacagccagaaataaagacatttattgagcgagatgggtagac
taaagatatcttcgttgtaagtgggtatagatgaagtggccaaggagtgg
aaaaaaaggataaaattggtcacatctagacataagagacaggagtaaga
gcctttggcagtatatggtaggaggaaaatcaaagtactgttacgaaggc
caagaaaaattttgagagtttttccatgcagttttggatgctgcaaaaaa
atgagatagtctttatcttgacatttattaagtatgtatttgttggacat
agttctaagtgtttgacacatactaatttttttatggggggtgaccaggt
gaataccccttgtgggcatggtatactgtgtcaccccacttactgggcac
atttatgtcatttcacagaaatgaggaagctgaggcagaaaaggtttagt
agcttgtccatattgcattgccagaagaagaaacagagctaggaaattct
aacccagtagtcaagttacaaagtctcattttagtttctatgccatatgg
cttcacattgaagcatgcttgatggattgggtgactgactagtagtcatt
ggtaaactcagcattttcaggtgatgagttggagctggatgtcattttaa
aatagtggattgtttggagagaggaaattggtatgagtagtagtgccttc
caaaatttggggagggataagggcaagcatttaaaatataactaaggttt
caatttagttttaggctaagcgatgtgaagatatttatgtggtaatgggc
aaattagtggaagaatagttgagtccagggaagaggtaatacggaatgaa
actggaaggtgtacgcgaattggagttagggataagcaggcagggaataa
attggccttggaccgtaaagaaggtacaccatttcttacacatgtgtcac
ataaattttcaaatgtatggctatgttgggaaagcttaatggcccaatga
acttggaaactttttgtattcatttgctgatagggtctaatattttctgg
gaaatgacttacttggaaaatactagctttattatttactgattggtata
actttgcagtatcaaagttagtctgttttagtttctttctttcttttttt
ttttttgagacaagtctcactctgttgcccaagctggagtgcagtggtgt
gatctcggctcactgcaacctccgcctctcgggttcaagcagttctgctg
ctgcagcctccccagtagctgggactacaggcgcacgccgccacacccag
cttgtgtgtgtgtttgtgttttaatagagacgggggactacaggcccacg
ctgccacacccagcgtgtgtgtgtgtgtgtgtgtgtgtgtgtgtgtgtgt
gtgttttagtagagacgggggactacaggcacacgccgccacacccagct
tgtgtgtgtgtgtgttttagtagagacggggtttcttttctttttcttct
ttttttttttttttgagacggagtctcgctctgtcgcccaggctggagtg
cagtggcgcaatatcagctcactgcaagctctgcctcccaggttcacgcc
attctcctgcctcagcctcccgagtagctgggactataggcgcccgccac
cacgcccggctaatttttttgtatttttagtagagatggggtttcaccgt
gttagccaggatggtctcgatctcctgacctcatgatccacccgcctcag
cctcccaaagtgctgggattacaggcttgagccaccgcgcccggcctgag
acggggtttcaccctgttgttcaggctggtctggaactcaccctgagctc
aggcagtctgcccaccttggcctcccaaagtgctaggattacaggtgtga
gccactgcacctggcttgttttagtttatctgtaaatggtgaatgaagcc
aggtagtctggagtgttgcaccagcaggaggtttccatgtaatctagtga
tgtattagaaggaacatatagccgggcacagtggctcatgccctgtaatc
ccagcactttgggaggcagagacaggcggatcacctgaggttgggagttc
gaggccagcctgaccaacgtggagaaaccccatctctactaaaaatagaa
aattagctgggcgtggtggtgcatgcctgtaattccagctactcgggagg
ctgaggcaggagaattgcttgatcctgggaggaggaggttgcggtgggct
gagattgcaccattgcactccagcctggacaacaagagcgaaagtccatc
tcaaaaaaaaaaacaacagcaggaaggaacatactatcacaacaagatgt
tgaggaataatcagtttcactatataaggttggggttttaatattccaaa
gaaagctcaaaatgatctttgttctcagtgacatgcatttagctaaaact
ggacatttatatcttgatcagcccattgttatagactctttatatagtcc
cttctcctgccttactagaaagggaaagtagcctgttttaatttagtagg
tgattgatttagacatagtggctaactctaaacacagtatacagaaacag
tttaattactctgtagaaatacgtatttgggtaccagtagtcttattttt
gttttgtgagtgtaggttaggtctgtaattttgaattatgttccacagag
cagcttcagtgacagaggagccagtcatttggggatttggttcttttcct
ctgtaaacctaggataattttacttaggtcagcatgaaagtacagtggta
tgattggtcaaagatatatttgaaatattatcataaggtatcataaagat
tggttaatctttaggagtcaaagtcacatttcaattgctacctccattgg
tatttttccgttaaaagtttttgggtttttctgagacgtggggtcttaca
gtttggccaggctgaagtgcagtggctgttcactggcacgatcccaccac
tgagtaggctttaagagttaatacgtttcatttaggttacattttattat
taatctgtgtatttggattgtggtctttctttcaaacagAATTCAAAGAA
GCTTTTTCACTATTTGACAAAGATGGTGATGGAACTATAACAACAAAGGA
ATTGGGAACTGTAATGAGATCTCTTGGGCAGAATCCCACAGAAGCAGAGT
TACAGGACATGATTAATGAAGTAGATGCTGATGgtaagtcttcaattttg
tggggctgggaggggttgaagaagtgatacttagatgtttccactaaacc
tttgctgtttgcttatatgtttccactaaagctttgatgtttccactaaa
ccatttcagGTAATGGCACAATTGACTTCCCTGAATTTCTGACAATGATG
GCAAGAAAAATGAAAGACACAGACAGTGAAGAAGAAATTAGAGAAGCATT
CCGTGTGTTTGATAAGgtacgtcaaggactggatatgttaaattttgagg
atgaaatgaagataccaggactcattatgaagacttgtgtttttttggtt
gtttttttttttttttttttttttttttttgatgatttaccaacatattt
taattaccaagcaaaagctttttaagaattcagtatagactttctaatat
atgtattgcgttcatcttgtcaaaggaggtgtcacttgactttgggacta
ggaaataatgatttcaatgtaggacattttgcttggctcttaatgacatt
tgtaggtcaactgtgtagatttgtatgcatttctaaagtatgtttttccc
ccaatgtgaaatttagtagtaaacttaataaatttccattccaactctca
agtctgtaatttgactcagaaactatactaaattttttgctagGATGGCA
ATGGCTATATTAGTGCTGCAGAACTTCGCCATGTGATGACAAACCTTGGA
GAGAAGTTAACAGATGAAGAAGTTGATGAAATGATCAGGGAAGCAGATAT
TGATGGTGATGGTCAAGTAAACTATGAAGgtaaatgtttcagtctcacgc
ctcattcttcactttgatttttaagataagaggctctttgttagggaact
gatcgaaattagtgacccttcccctgaaattaggttgctaaaacagttac
ttacatttaaccttcttaaaattcagagtgttgtctgcagactaggtaac
attgctctgacttgagaacttaatttagaaatatttactatcaggttcta
tccctacacctggaatcagcattttaataggattcttgggtgattcatgt
ataaagtttgagaagcactgttcagtgtttgtgtgattgtggaacaatgt
ggaaagcaaacttattccaagtttcctaaaatgaaattaatgtgactttc
tttccccgcactgtattgtcttttttctttaattggctttagtctgaaac
tgaagtttacatgtgcttcaaaagtatcgtatctactaacttttaagatg
gccatatcaggaagcaaggcatatgaaactgaaggattttgagtaaaact
ctgtatttttttcatgatgtaactacttaacttagaaaagagcataaaat
gtaatgttataaaaattaaacctagcccatagcattgaatttacaatatt
gtcttgtaatccagcagacttaaaattcaagtctaaactaagagccaaga
atgctcttttttatattggcctatctaaagcgttaatttggctcttacta
ataaacctgaatagttatggttaatgaagttccagcctgggcaataaact
agcttccctctgtacttaatggtaattctgaggaaagagaggaatggaaa
gagtactcagtaattgagtaaatgtgattttttttttttttttttttttt
tggtcttttgctattgactcattttttgtgtacttcttctttttcagAGT
TTGTACAAATGATGACAGCAAAGTGAAGACCTTGTACAGAATGTGTTAAA
TTTCTTGTACAAAATTGTTTATTTGCCTTTTCTTTGTTTGTAACTTATCT
GTAAAAGGTTTCTCCCTACTGTCAAAAAAATATGCATGTATAGTAATTAG
GACTTCATTCCTCCATGTTTTCTTCCCTTATCTTACTGTCATTGTCCTAA
AACCTTATTTTAGAAAATTGATCAAGTAACATGTTGCATGTGGCTTACTC
TGGATATATCTAAGCCCTTCTGCACATCTAAACTTAGATGGAGTTGGTCA
AATGAGGGAACATCTGGGTTATGCCTTTTTTAAAGTAGTTTTCTTTAGGA
ACTGTCAGCATGTTGTTGTTGAAGTGTGGAGTTGTAACTCTGCGTGGACT
ATGGACAGTCAACAATATGTACTTAAAAGTTGCACTATTGCAAAACGGGT
GTATTATCCAGGTACTCGTACACTATTTTTTTGTACTGCTGGTCCTGTAC
CAGAAACATTTTCTTTTATTGTTACTTGCTTTTTAAACTTTGTTTAGCCA
CTTAAAATCTGCTTATGGCACAATTTGCCTCAAAATCCATTCCAAGTTGT
ATATTTGTTTTCCAATAAAAAAATTACAATTTACCCAATGGTTGCTCTGC
ATCTGAGTCATTTAACTGTTGAAGTCTAATAATTTTGAAAATAAAATATG
GCATTGGTTTCTGCTTGGTA
SEQ ID NO: 3
Calmodulin 3 (CALM3) gene sequence (from UCSC
genome browser). Exon sequence in upper case.
>hg19_knownGene_uc002pew.3 range =
chr19: 47104512-47114039 5′pad = 0 3′pad = 0
strand = + repeatMasking = none
GGCGGGGCGCGCGCGGCGGCCGTTGAGGGACCGTTGGGGCGGGAGGCGGC
GGCGGCGGCGGCGCGCGCTGCGGGCAGTGAGTGTGGAGGCGCGGACGCGC
GGCGGAGCTGGAACTGCTGCAGCTGCTGCCGCCGCCGGAGGAACCTTGAT
CCCCGTGCTCCGGACACCCCGGGCCTCGCCATGgtgagtgaggctggggg
gtcgccgaggctgcgggctctgaggcgggcttaacggggcaggacccctg
agggggcgacagagcccagagtggggggcgtccgggcccggcgagagcct
cgggacccttttctacccgcgttgtcgggggctgttgaacccagagcggg
acgtctggatcaccagaggtttccagaagcgactttagcaccaaatggga
tgtttaagtccacaaatgggtatctgagcccctaaaggggacatttgaac
cccggatggaggatcgggaccctcagtgggacccctcaaaaggattttgg
acccaggatggggcgttgggtttcaggatggaggtgtttggaccccaggg
gacgaaataagaaagatgggctggggaggggagctattcgggccctgatg
aaggcgtttggtcctgagaggatgcggggaacgggggacgaagggaggct
gggctgctcctggatggggtggtggagggtagctgggcccgggcggggag
gggcgacccctgggctcctgtggagcagaggagagggtcaaggatgggcg
gtcacttctacagatgagactcccgagggtgggggagggtgggggagggt
ggtctccagagcccagcactgcagaggaggaggagggtgaggtgcaagct
tatgggaggaagagcctggggtgggtggcagcggagattagaagatactc
ttgaaggcttccgggacggagaaggatcagggtcgtggaggtggtgaaaa
gggatggtgagagtggggctcgccagtagtcgggggacagggacccttgg
ggttaatttggaagtagctagaaaaggttccgaggttactagtgggttaa
gactgttgctctagaaagacagaatttttttaacggggtgggagtgttgt
gtttgggtccaagagaagatggatttccaaggtgaggggtgggggaggac
ttgggcttgggtggttgggaggtgaggtggaaggagaaaatgaatggcag
tcaggggggcctaggtttgggggtggatgtgggtagcaccctgccagtct
ccagggacaggccaggacaaagacggctcagtgtggatggggagtgggct
gcagggcccggaggaagcttttctaccgtcctccttcctcaggaagaacg
cggctgggtggaagacaccggggcagtgggagcaggtgggcggagctgtt
cgggcccctattgcgcactgaagcctcatgaaatggggatgggagatcaa
agtttggggtgcctcgaatgagccagctttttcaagcctagaagcctcac
tctgtcgctgcccttttcaggccaagagcactgccttttcccagcaggag
cgccggttttctcatgatccccagccccacgcaccatgctgcacctcgtc
acttgtgccaaatgacagtgcagtacagtgtggcgtcagtgacaatcaga
tgcctattcccgtgccctcggggtgggggctgcagagcttgatcagagag
tgcctctggggagaccccttgatcggcctgagggccacttgttccattca
gggccctcgctgagcgtgtctgttaattcacagggctttgagagaaaacc
agcagactagttctttcctactccctgccacccacaacacccacagcccc
accaggctgagcctgggaaccgtgtgcagccatcctccctggcaaggctg
gggagaacaggcagccgctgctgtgtatcagatctcatacttgctgacac
agacctgatggctcctcttgccgctaccccattttcccccagcagaacct
ggctaaaaggggccctgaagcactagcaggccccctacctccagaatagg
acccaagctttggccacaactagtttctctttctgtttgtaatcagcaag
gtagaggtgggacaagtcactggtaactggccttggactttggcctgagc
tctgtggctcctccttggcccgggtgaggggccctggccttggtttctga
tctcatctaacactgctctctggcattggctgggggctggggcaaggcag
agaaatcacaccctcttccctcccccacctcatggttttttttcttgtcc
agccccttgctggagcctaagatgagggataaggaagcaggagaccagct
gagttattttggaaacagtttttgttgaaagtttagtcttcaggtctaga
aagggaagaaaccttgagcaaggatcaatgatatcttcttaggcggtcgg
ttttctgggcggcaaccttgcttgagatctgtacctttccttgaccaagc
agggaagtgggtctccaacatgggagggagagcatctgacaggccctcca
tgcagagccacgcctatttgcagcaaccctgtgacagaactgaacatggc
agtgcctcatctgcatcttccagtcttttggtggagactggcaaacggtg
gccttcagagccgctcggggcagtctgcagcctgtcacagcctcctcgcc
cccacccccagccccttacagtgctgcccaaaggtccggggagctggact
tcttgcagggcggaacagtgggctgggaaggccctgacctggctcgcttt
ttccattctaggccaggggaaagaatgaaaagcagatgggatggcagcca
cctccccacactgctgccctcctctctgttctttccctgaccctccctgg
gcattgcctggtctcggtaaccagaagcccatcctcccttctcactttcc
ctaagcctcttcctcaggtgtcaggacctaagggatggctccaggctttt
ttgtccatggtgggaggtggggggcaggtggatcaagaggctcatgggcc
tggggctaccaggttggcatttagaagcccaaccaccctgtcttttttga
aggcaatgcagaagaatcttcatttggcctccccacctcccctttacact
ttggacttttccagaatgtgcatgtgtgccgttttctcccttcctctttc
cccctccctaagccctctcccctctgtgatcatcattttgattacccctc
ccattctgcctatttggggcttgcaagttcttcttccgttaggttttttt
tttttttttctaacttttttagatcagtgattgttaatatttgggacggg
gatgggggtaggggaggcaggcagtggtttgttcctagcttctggaacct
ctccgtagaatactgcaaattcacataatagtctgcttctatttctgagg
ctctcagactcgagactagaacatgctatagacttggagcccctttccct
gattgtgaaaggaaggggaacagacccaccatggtcccgggatggtggtg
ccttcttcacagcatcaacaaaaaccagggaaaatgtgtcttttggctgc
taaagctaaatgttgggattcagcctccccaaccccccagccccgctcct
gccccataggatcactatgcctggctgatgcccagcacaccagtgcagga
gaagccagtccccgcatctcctccccacccccaaaccccgcatgcagcaa
gggctgcctgccacccactggccagtccccagagtgcccaagctggaaag
ggttaaaaactgcagtgcaagccagcttcagtcctctgctgctgcccgct
gcagccctctccctgacgtttgtcccttctgcccaaaagaaacaaaacag
aatggccctgtgccctcactgtgcgcagggggtggagcgcctgcctccag
cctgaccagagttctttaggtgcttgagcaactcgggccgccggttagga
gaatttttgccacttgggtggactctgcctgagccaaccgcctccaaata
ggtgacacagaaactagcagagatagccccagacatcgtccctctgccct
acccaaggactggcctgcttcagtttggagccttctagagcttcttgatg
aggcctccagggcaatcgttcccttcactcggcctctggggctctcactg
atgagatgcaaacctgtgccaggcctcaccagcgctgctggtttggggct
ggggcaggcttttctcccgcccctgtggctcccacatgctgagttgagat
tggaagcccgtgggtctgggcagcttcatcgggccgtgcgtccagcaggc
ctgagtgtccagcccaccctggtacccagctttctgaatcaccagacttt
atcatcaggcgctggctctgcctcagaccctgactctggcatgctcctcc
ctggcccggcgggaatggcacgtggagctggcctcactggggccgagggt
ctgggctgagtgaggaagatgcgtccgtgctgtccggcctggtgactgac
tttccccttcatgcttttacagGCTGACCAGCTGACTGAGGAGCAGATTG
CAGgtgagtctgctatccccctcatcttagctcaggaaagcctccacatc
cccagccaaatcttagccactgaggagtgacatctgatgggtgaacctgt
gtatcccttgtgtcacctaacactatgccttgtgcctagaatgctgataa
atgtgtgtaagagcacaccagtgccccaatgacacgaagccccatggccc
tgagacaggcctttaccagacaccaaaggctgtgttcctctcctgggcct
gggtcatctaggggcttgatatcagtggctgcaggacgagcccaactctc
tctcccagcttcagccaggttggcttagaaggtgagctccctgcctccct
gtggggcctgcagagctcctgtcccactttaggagccgcccaggagctgg
ctcgtcactcccacctctgcgcttgcctgcgctttgcacttcctttcgca
tgtgcctgcgcctgcctgttctgctggcggaagtgcctccccagtgcttt
gcatgggcagctccttcccgtcccttagctcagatgtcccctcggggagg
ccttccctgacccccatctcaagtacacagccttcctttactctccttca
catcactttattttcttcataacctttctcgctgtctgaaattattttct
tatgtcttcacctgttgtatctcctgttactagatgtgagttccattgag
tttgggacttggttttgttggtccctgctatagccttaatgcctgcctag
aacgtagtaggtcctccgcaaacttttgctggctaaacaaacatctctct
ctcagcaagctagaagaaatccctggatcccatctaattcatttttctaa
cccctccaaaaccccagaagggtcagctacaggaactattgtgtaattac
agatccgtgcctgcctacccgcccatactgagctgcccgaggacagagag
caactcttggtttatctccatagccaggggccagcacacagtagaccaga
gtagcgatccagagaatgggcctctgctccccaccagctacccagccccc
agctctgctgtcaggttcaaccggctgtgtaacaagtactaaaggcccag
cttctgcggtgggaggcaggaacggacttgccttctctcctttctcccct
caacccgcatcacagagtcacctttgcagcttcaaaattttggccctggg
tggctatggagacccctgaggaaagccagaagatggcctggtttagtgat
catgagctcggggtgccccatgacccactcactacctgccctccccaaga
acaacaaggcctcagcacagggaggtgccagcctccccaaagagactctt
gccatgtccccagggcccagaggctcagggccacagcttcccagctgtgc
atgtccctcccaatgcacctgccacccatctcccaactctggccttgtgc
aacataaacggtctgcttttcaccctgtccccattagcaaactgtccacc
gcctcaccccagcccacaccctctgggaataggctgccggacatctgttt
ttggagggcttgggggtagaggagcaccaggtgccaaaatgggcccagtg
ggagggcaagagccatgtgcttttctccctgcccctgatgcggccccagc
tgtggccttcccactcagcactgctagctcctcatcctcctggcttggca
gccgtcccctcgctttccatccccccatcaaccccacgcactgtccgtca
gccatgttctgctgccgaccacccccacgctcaccacacgcagtggctca
gctcctttggaagctggtagcacccacggcaaggaacagggctgccagtc
agaagggagtggatgcctgcatttcctcttcaggccggccagaggcattc
tgcagcctcccctcctcgtcccagccagccaaccctggctaacacactca
gacaaagaatcctttcggctctcattcactccatgactttggttcatttc
atcatttcaagggagaagtcaaagccagggaccaaacagaaatgtagaat
catctaaggacagctgtcagaccattttccccactgtcttcactggctgg
ccagaaaaatagcttcacctagcacagtgttgatgttcaataactgttga
atgaaggttggaagggctttgccctgagcactgaggagagagagctggtt
gcgtgggacttgaagtctgtctcagtttctttaggtgggactgatgccct
tggccttcctccagggaaggcatccagcatccagaggtaaggattctcct
gtggaccttgtgacctctgactcctcccccttcttcccccagAGTTCAAG
GAGGCCTTCTCCCTCTTTGACAAGGATGGAGATGGCACTATCACCACCAA
GGAGTTGGGGACAGTGATGAGATCCCTGGGACAGAACCCCACTGAAGCAG
AGCTGCAGGATATGATCAATGAGGTGGATGCAGATGgtgagccccacaga
gcgcgtgggcagccctgctcctggtcaccccgagtgactgcagggagcct
ctctcagggtgatggatgagcccgtgtctctcagggcccaggccaagagc
attctccatcctttccccaccttccagGGAACGGGACCATTGACTTCCCG
GAGTTCCTGACCATGATGGCCAGAAAGATGAAGGACACAGACAGTGAGGA
GGAGATCCGAGAGGCGTTCCGTGTCTTTGACAAGgtaagcagccctctcc
aggggcggctctgagactgacgccagccttcaggcagacaggcggaactg
gagccacggagctaccacttccaggaggtccgggtcccggtgccagcctc
attgccaacctgctctgccacctcaggcagcctccctcactgtgctcact
gctgagggatggtgatgacagccacccctctcactgcctctctccccacc
gggagaagtgcccagtgaaaggctttatccccaacccccagGATGGGAAT
GGCTACATCAGCGCCGCAGAGCTGCGTCACGTAATGACGAACCTGGGGGA
GAAGCTGACCGATGAGGAGGTGGATGAGATGATCAGGGAGGCTGACATCG
ATGGAGATGGCCAGGTCAATTATGAAGgtgagtcaaggccaggcttgatc
tctggaacaagaagagaatcgcagcttcaggaacaagcccccagatccct
cttgttggggagggccgctcgccacttagcctgcccgcctgacctcctct
ctctctgcttcactccacagAGTTTGTACAGATGATGACTGCAAAGTGAA
GGCCCCCCGGGCAGCTGGCGATGCCCGTTCTCTTGATCTCTCTCTTCTCG
CGCGCGCACTCTCTCTTCAACACTCCCCTGCGTACCCCGGTTCTAGCAAA
CACCAATTGATTGACTGAGAATCTGATAAAGCAACAAAAGATTTGTCCCA
AGCTGCATGATTGCTCTTTCTCCTTCTTCCCTGAGTCTCTCTCCATGCCC
CTCATCTCTTCCTTTTGCCCTCGCCTCTTCCATCCATGTCTTCCAAGGCC
TGATGCATTCATAAGTTGAAGCCCTCCCCAGATCCCCTTGGGGAGCCTCT
GCCCTCCTCCAGCCCGGATGGCTCTCCTCCATTTTGGTTTGTTTCCTCTT
GTTTGTCATCTTATTTTGGGTGCTGGGGTGGCTGCCAGCCCTGTCCCGGG
ACCTGCTGGGAGGGACAAGAGGCCCTCCCCCAGGCAGAAGAGCATGCCCT
TTGCCGTTGCATGCAACCAGCCCTGTGATTCCACGTGCAGATCCCAGCAG
CCTGTTGGGGCAGGGGTGCCAAGAGAGGCATTCCAGAAGGACTGAGGGGG
CGTTGAGGAATTGTGGCGTTGACTGGATGTGGCCCAGGAGGGGGTCGAGG
GGGCCAACTCACAGAAGGGGACTGACAGTGGGCAACACTCACATCCCACT
GGCTGCTGTTCTGAAACCATCTGATTGGCTTTCTGAGGTTTGGCTGGGTG
GGGACTGCTCATTTGGCCACTCTGCAAATTGGACTTGCCCGCGTTCCTGA
AGCGCTCTCGAGCTGTTCTGTAAATACCTGGTGCTAACATCCCATGCCGC
TCCCTCCTCACGATGCACCCACCGCCCTGAGGGCCCGTCCTAGGAATGGA
TGTGGGGATGGTCGCTTTGTAATGTGCTGGTTCTCTTTTTTTTTCTTTCC
CCTCTATGGCCCTTAAGACTTTCATTTTGTTCAGAACCATGCTGGGCTAG
CTAAAGGGTGGGGAGAGGGAAGATGGGCCCCACCACGCTCTCAAGAGAAC
GCACCTGCAATAAAACAGTCTTGTCGGCCAGCTGCCCAGGGGACGGCAGC
TACAGCAGCCTCTGCGTCCTGGTCCGCCAGCACCTCCCGCTTCTCCGTGG
TGACTTGGCGCCGCTTCCTCACATCTGTGCTCCGTGCCCTCTTCCCTGCC
TCTTCCCTCGCCCACCTGCCTGCCCCCATACTCCCCCAGCGGAGAGCATG
ATCCGTGCCCTTGCTTCTGACTTTCGCCTCTGGGACAAGTAAGTCAATGT
GGGCAGTTCAGTCGTCTGGGTTTTTTCCCCTTTTCTGTTCATTTCATCTG
GCTCCCCCCACCACCTCCCCACCCCACCCCCCACCCCCTGCTTCCCCTCA
CTGCCCAGGTCGATCAAGTGGCTTTTCCTGGGACCTGCCCAGCTTTGAGA
ATCTCTTCTCATCCACCCTCTGGCACCCAGCCTCTGAGGGAAGGAGGGAT
GGGGCATAGTGGGAGACCCAGCCAAGAGCTGAGGGTAAGGGCAGGTAGGC
GTGAGGCTGTGGACATTTTCGGAATGTTTTGGTTTTGTTTTTTTTAAACC
GGGCAATATTGTGTTCAGTTCAAGCTGTGAAGAAAAATATATATCAATGT
TTTCCAATAAAATACAGTGACTACCTGA
SEQ ID NO: 4
Calmodulin amino acid sequence.
ADQLTEEQIAEFKEAFSLFDKDGDGTITTKELGTVMRSLGQNPTEAELQD
MINEVDADGNGTIDFPEFLTMMARKMKDTDSEEEIREAFRVFDKDGNGYI
SAAELRHVMTNLGEKLTDEEVDEMIREADIDGDGQVNYEEFVQMMTAK
SEQ ID NO: 5
Mature mRNA sequence (CALM1)
>uc001xyl.2 (CALM1) length = 4268
gauacggcgcaccauauauauaucgcggggcgcagacucgcgcuccggca
guggugcugggagugucguggacgccgugccguuacucguagucaggcgg
cggcgcaggcggcggcggcggcauagcgcacagcgcgccuuagcagcagc
agcagcagcagcggcaucggagguacccccgccgucgcagcccccgcgcu
ggugcagccacccucgcucccucugcucuuccucccuucgcucgcaccau
ggcugaucagcugaccgaagaacagauugcugaauucaaggaagccuucu
cccuauuugauaaagauggcgauggcaccaucacaacaaaggaacuugga
acugucaugaggucacugggucagaacccaacagaagcugaauugcagga
uaugaucaaugaaguggaugcugaugguaauggcaccauugacuuccccg
aauuuuugacuaugauggcuagaaaaaugaaagauacagauagugaagaa
gaaauccgugaggcauuccgagucuuugacaaggauggcaaugguuauau
cagugcagcagaacuacgucacgucaugacaaacuuaggagaaaaacuaa
cagaugaagaaguagaugaaaugaucagagaagcagauauugauggagac
ggacaagucaacuaugaagaauucguacagaugaugacugcaaaaugaag
accuacuuucaacuccuuuuuccccccucuagaagaaucaaauugaaucu
uuuacuuaccucuugcaaaaaaaagaaaaaagaaaaaaguucauuuauuc
auucuguuucuauauagcaaaacugaaugucaaaaguaccuucuguccac
acacacaaaaucugcauguauugguuggugguccuguccccuaaagauca
agcuacacaucaguuuuacaauauaaauacuuguacuaccuuaaugauaa
ggacuccuuaaaguuccauuugcuaaugauuaauacacuguuugggcugg
ccaguuuuucaugcaugcagcuugacgauugagcacagucaggccuuugu
auuaaaaaugaaaaaugaaaaaacaaauucaaaaccuauucaaauggguu
cuaguucaauuuguuuaguauaaauugucauagcugguuuacugaaaaca
aacacauuuaaaauugguuuaccucaggaugacgugcagaaaaaugggug
aaggauaaaccguugagacguggccccacugguaggaugguccucuugua
cuucgugugcuccgacccauggugacgaugacacacccugguggcaugcc
cguguauguugguuuagcguugucugcauuguucuagagugaaacaggug
ucaggcugucacuguucacacaaauuuuuaauaagaaacauuuaccaagg
gagcaucuuuggacucucuguuuuuaaaaccuucugaaccaugacuugga
gccggcagaguaggcuguggcuguggacuucagcacaaccaucaacauug
cuguucaaagaaauuacaguuuacguccauuccaaguuguaaaugcuagu
cuuuuuuuuuuuuuuuccaauaaaaagaccauuaacuuaaagugguguua
aaugcuuuguaaagcugagaucuaaauggggacaaggcagguggagggga
ggccaguguacauguaaaugcccacagcccagcauuggguuucccuccca
aggccccagcaccaaccucugagcccaagaccuugccugaaaacaagcag
auaccgauugcuucauccuauuuauggacauguaggucuaguugcauuuu
cacuggggggaggggggaaggugaauuaugguaacuuuuaaugaucuauu
caggcaguagagcucuuaaggaaaaaaaaaaacccacuuucucucaagca
uguauuuagggguuguucucaauugugcugcugauuaccugucuuaugua
acuacuugagaccaucugcaagagacaugauuuagugugucuguaauuca
aucuucgcugugugugguagaagcaguagucacuuuuguaagccagucuc
uucaugccuaaaagacacuaccagucaccuuugauucgcgacuuuuaauu
uaugauuauacuuagccuccuccuccuuuuuuuuuuuuucccaaguugac
uugacuuugcuuuuuuccccccaaguagaacuaaugcuagcuuccagcuu
gaaaguaaaacuccaguguggagugaauuuugugucuaauuauaaaccug
uaaccaaaacucagacaucugguacuggucuuugcauugagauugguccc
uguaaaacccccuuuaaaagcauauugcauuuaguacagagcucuuuuuu
gaaaugaaggcuggagaugugcauuuuucacgguguuaacugguuguauc
uuauuagcaaggagauugggguuuugaguguuugcgugggugguuucaau
uugccagggaacaguggcaggcugcuagcaaggcagugagaagcucuugg
cagccaaaugggugcauucagggcugauuuauagagacccuuggcuucuc
cuucuccuacucccugucuuucuggcauuuuguagcuuguuagauuuucu
gccagaggggugggucagagcaguggaggggagacaucgcccaugugcuu
cugcuacugguccuugggcugggugguugguagaggagauguugacacua
ugagcuaaggguuggcuuuuguaauuaccugaaucugaaaggaaugccua
agguuaccuugggguuucucuucuggugagauaggguuccugguuugagu
aaguuaauguccuggauauuucuuguggcaggggguggucaaagagccug
auugcugacccagucucaggccuguggucgaugaccucucgguaguuuca
aagggggcuggagggggauauuugacuuguuuuuucgaaauguagccuuc
uaacccucaagucuuuagaagcuggguggacucuuagugguccugcagcg
uauccuaaaagacuaccuuugaaacaggauucuuguauggccaggauccu
gucugggaaccagaaacccuacacccucccccuccagggaaugcugaguu
ccaguuuugagcagaggugaggcagaauccacuguagccuuccgcccugg
uauuuggggggaugaccagcccaggcguuggguguuagucugcaugaguu
ugugagaggaaauagcuggguguccuggcagugcccuugaaguugguuag
gaccuuccuguaaacucuugccccuacuucuaacuacucuauaaauauau
acauauauuuauauauaaagugauuaguugaacuggcauccugcuuuagc
cugagacuugccauaagaaacugcugaguacuuggcaaacccuuucauag
uuuuguucuccaucuguuugggguagguguugagcgaggcaaauggaucu
cgauauuucagaugggcuuuugaugcacuguugccaaggaaggcuuuuuc
ugauuuuuugacaaaugaauuuuugcacacuuucauuggugucuuucggc
aacuuacacacauugaaaaugagcuauuguacauauuuuuauauucucuu
uauaaaugcaugucugauuguacuuguaacaauauuguaaugaacggcug
ugcaguaggcccagcgcugcugugucucgucagaggaauagcuuaccacg
aaccccucagcauacugggaaucucuuccugaacaacgaauguaaauuug
gucaagucuacucuuccguucauucaauuauuuuaagcauuugaauuauu
uauuguauauccuaaauauauuucuccuuuggcagugacuagauuuccac
uaaugugucuuaaucuaucccuccagcuggcaguuacuguuuuuuuaauc
cccugaaguuguccuguaggagacagaaauucuuugcugucuguaucccu
uggaguaagaagguaguggcauggguggaguguguguucuuucuccaaau
cuauuaugauguuuauuaaacacuucuguagcaaagauggugguaguucu
uuuguuacugaaguugcccuucaccauggcuauuugaaaaggagauguac
uuggacguuucuguaaaucuugagauaaacuguuuggagauuuaaccacc
ucucugaugggggaccaacucuauggaaauuguaaauacguuuuauuuau
aaaccuggcacuguauucaauaaacauuucugcagccuuucaucucuaac
ugcgaaaaaaaaaaaaaa
SEQ ID NO: 6
Mature mRNA sequence (CALM2)
>uc002rvt.2 (CALM2) length = 1309
cucguuugcgauguuccguuaucuggaugcggcggagggaucuggcggag
ggagguguuuaugaggcgcugggggcggaggaggcgaauuaguccgagug
gagagagcgagcugagugguuguguggucgcgucucggaaaccgguagcg
cuugcagcauggcugaccaacugacugaagagcagauugcagaauucaaa
gaagcuuuuucacuauuugacaaagauggugauggaacuauaacaacaaa
ggaauugggaacuguaaugagaucucuugggcagaaucccacagaagcag
aguuacaggacaugauuaaugaaguagaugcugaugguaauggcacaauu
gacuucccugaauuucugacaaugauggcaagaaaaaugaaagacacaga
cagugaagaagaaauuagagaagcauuccguguguuugauaaggauggca
auggcuauauuagugcugcagaacuucgccaugugaugacaaaccuugga
gagaaguuaacagaugaagaaguugaugaaaugaucagggaagcagauau
ugauggugauggucaaguaaacuaugaagaguuuguacaaaugaugacag
caaagugaagaccuuguacagaauguguuaaauuucuuguacaaaauugu
uuauuugccuuuucuuuguuuguaacuuaucuguaaaagguuucucccua
cugucaaaaaaauaugcauguauaguaauuaggacuucauuccuccaugu
uuucuucccuuaucuuacugucauuguccuaaaaccuuauuuuagaaaau
ugaucaaguaacauguugcauguggcuuacucuggauauaucuaagcccu
ucugcacaucuaaacuuagauggaguuggucaaaugagggaacaucuggg
uuaugccuuuuuuaaaguaguuuucuuuaggaacugucagcauguuguug
uugaaguguggaguuguaacucugcguggacuauggacagucaacaauau
guacuuaaaaguugcacuauugcaaaacggguguauuauccagguacucg
uacacuauuuuuuuguacugcugguccuguaccagaaacauuuucuuuua
uuguuacuugcuuuuuaaacuuuguuuagccacuuaaaaucugcuuaugg
cacaauuugccucaaaauccauuccaaguuguauauuuguuuuccaauaa
aaaaauuacaauuuacccaaugguugcucugcaucugagucauuuaacug
uugaagucuaauaauuuugaaaauaaaauauggcauugguuucugcuugg
uaaaaaaaa
SEQ ID NO: 7
Mature mRNA sequence (CALM3)
>uc002pew.3 (CALM3) length = 2277
ggcggggcgcgcgcggcggccguugagggaccguuggggcgggaggcggc
ggcggcggcggcgcgcgcugcgggcagugaguguggaggcgcggacgcgc
ggcggagcuggaacugcugcagcugcugccgccgccggaggaaccuugau
ccccgugcuccggacaccccgggccucgccauggcugaccagcugacuga
ggagcagauugcagaguucaaggaggccuucucccucuuugacaaggaug
gagauggcacuaucaccaccaaggaguuggggacagugaugagaucccug
ggacagaaccccacugaagcagagcugcaggauaugaucaaugaggugga
ugcagaugggaacgggaccauugacuucccggaguuccugaccaugaugg
ccagaaagaugaaggacacagacagugaggaggagauccgagaggcguuc
cgugucuuugacaaggaugggaauggcuacaucagcgccgcagagcugcg
ucacguaaugacgaaccugggggagaagcugaccgaugaggagguggaug
agaugaucagggaggcugacaucgauggagauggccaggucaauuaugaa
gaguuuguacagaugaugacugcaaagugaaggccccccgggcagcuggc
gaugcccguucucuugaucucucucuucucgcgcgcgcacucucucuuca
acacuccccugcguaccccgguucuagcaaacaccaauugauugacugag
aaucugauaaagcaacaaaagauuugucccaagcugcaugauugcucuuu
cuccuucuucccugagucucucuccaugccccucaucucuuccuuuugcc
cucgccucuuccauccaugucuuccaaggccugaugcauucauaaguuga
agcccuccccagauccccuuggggagccucugcccuccuccagcccggau
ggcucuccuccauuuugguuuguuuccucuuguuugucaucuuauuuugg
gugcugggguggcugccagcccugucccgggaccugcugggagggacaag
aggcccucccccaggcagaagagcaugcccuuugccguugcaugcaacca
gcccugugauuccacgugcagaucccagcagccuguuggggcaggggugc
caagagaggcauuccagaaggacugagggggcguugaggaauuguggcgu
ugacuggauguggcccaggagggggucgagggggccaacucacagaaggg
gacugacagugggcaacacucacaucccacuggcugcuguucugaaacca
ucugauuggcuuucugagguuuggcuggguggggacugcucauuuggcca
cucugcaaauuggacuugcccgcguuccugaagcgcucucgagcuguucu
guaaauaccuggugcuaacaucccaugccgcucccuccucacgaugcacc
caccgcccugagggcccguccuaggaauggauguggggauggucgcuuug
uaaugugcugguucucuuuuuuuuucuuuccccucuauggcccuuaagac
uuucauuuuguucagaaccaugcugggcuagcuaaaggguggggagaggg
aagaugggccccaccacgcucucaagagaacgcaccugcaauaaaacagu
cuugucggccagcugcccaggggacggcagcuacagcagccucugcgucc
ugguccgccagcaccucccgcuucuccguggugacuuggcgccgcuuccu
cacaucugugcuccgugcccucuucccugccucuucccucgcccaccugc
cugcccccauacucccccagcggagagcaugauccgugcccuugcuucug
acuuucgccucugggacaaguaagucaaugugggcaguucagucgucugg
guuuuuuccccuuuucuguucauuucaucuggcuccccccaccaccuccc
caccccaccccccacccccugcuuccccucacugcccaggucgaucaagu
ggcuuuuccugggaccugcccagcuuugagaaucucuucucauccacccu
cuggcacccagccucugagggaaggagggauggggcauagugggagaccc
agccaagagcugaggguaagggcagguaggcgugaggcuguggacauuuu
cggaauguuuugguuuuguuuuuuuuaaaccgggcaauauuguguucagu
ucaagcugugaagaaaaauauauaucaauguuuuccaauaaaauacagug
acuaccugaaaaaaaaaaaaaaaaaaa
SEQ ID NO: 8
Calmodulin 1 (CALM1) gene sequence comprising
the mutation A4403T. Exon sequence in
upper case.
>hg19_knownGene_uc001xyl.2 range =
chr14: 90863327-90874619 5′pad = 0 3′pad = 0
strand = + repeatMasking = none
GATACGGCGCACCATATATATATCGCGGGGCGCAGACTCGCGCTCCGGCA
GTGGTGCTGGGAGTGTCGTGGACGCCGTGCCGTTACTCGTAGTCAGGCGG
CGGCGCAGGCGGCGGCGGCGGCATAGCGCACAGCGCGCCTTAGCAGCAGC
AGCAGCAGCAGCGGCATCGGAGGTACCCCCGCCGTCGCAGCCCCCGCGCT
GGTGCAGCCACCCTCGCTCCCTCTGCTCTTCCTCCCTTCGCTCGCACCAT
Ggtaggtcgggagtggcaaatgccggcgtagcagctgcccgagatttctt
cccagatttctagttgttttgtttgttttttgtttgtttttggttcttgg
aggtttttcttttctgagtgttacgcagcagctgcgcttaaaggaggttg
cattttggatttgcatctcggcgacctctgccagggagcttcatttattg
gttccccttggagctggacttggtcgtaggccgtccacgggcaggggctc
cggccgcaactgcagcgggggtttctgcatccaatccccctgccccccgc
ccagccccgcacccactgcatccactagcgccgcacccgggctgcctgca
gcgcagcgtttcggcctgggagccgggcggggccgggcactagacccccc
cccccggcccgcccctccccaccccgcttctccgccggcgcgaaggtggc
aggtcgggcgggcagtggagaatgaatgggctggagctggccggtggcgc
acattgttccggccgggtgttgaggggcgcagtcagcgcccgccacctcc
ccactttggccggccctgctgggcgccctccctcggtcgctctcccctcc
ttcttcccggggggcgcggcgcgggcgtgggctgggaaggaaggagccgg
ggaagggtggggttgggggcaggaaggcgaggggttgggggcggagaggg
cggaagcggcggccgggccgccctgcgcccgggcggggccctgcggtgtg
gccgtggcttgttcctgccgctttcgcaccctgcggccccccacccagtg
cagcagtgcgggcgggcgtgagcctcggtgcaccaggaggcacttcccgc
gggaggcgctgggctcgcgctaattggggcggggggggggggcggcgggg
gaggagggaactggcgcgcggcttggtttccattagagacgcaaagtttc
tgctccgggaggaggcggcggcgccgcgggctcgtcgcctgggggagcag
aagcgggtgggaggtgcgggtggccttggcctcagccctggtgcgcgggg
gccgggggtggtgaccctcctggccgaggaggggcggcgtccagacgccc
gctcgggggccgccttcccccccacgcctgcccccgggcacgcgccctgc
ccggtccctcgccccgcgccacttccagtccgcagagagatgccctccac
gtttctgctttctctgcagcctctagattgccagatgcgactgtgcgcct
cgctgggtgtgttttccacagccccttcctcctcggcgtgcagggctgac
atcaccgactgcgtttctggtttggcgggtggggagatggttccccgcag
ggttctggtacacctttgcccccagggctagcgccatttgggggaggagg
ttttcgttgtcgagaaagttggatgctcctggtaacccctctaacaagag
agttctgtagcgaggtgggactgttctccccataaggtgacagtttctct
tgcgaggtgtggcagcgcttcctgttgtacaagacagatgttgccttggc
gttacgtaaatcatcgtgtctccgtcatttaaagaaagccaatttttagt
gattgaggtagaaagaaagatccgtttataatttgtaaaaacaaattttc
acccagaatcaatatattggaacaccattcctactgttaaagttttcact
taagagtataaacttcatcagctttctattaggacttattttgtaattgg
cttcttaggcatccttctttaaaagagaaatccacgttagctctccttga
ggtctcgagttccctcggctggaggcacaggttcagtggagaccaaataa
tgcaggtgaattaccttcgtggccattactgcctccaacgaagtgtgttt
attaagaacagttcttatgtcattcttaaggtaggtagggttaatactct
ccagcaaatttagtagatactctttgccagaaaagagaggagtatatata
gtttgataattattgtgtagttttctgtgtacttaatttttgcagttttg
taacacttcatttgtaagatggtaccattttttcctggcttctgaatcat
aggatagtttgacccagggcattagccattgtaatggtaggcttttaaca
aataactgcctaatttaaaggattggaaagcatttgttacatggaaatga
agttggtggcgtacccagttgctgtatctttattttttctacttaattat
ttctcataaaatggatataaaagcctgttaatccaacccaatgccattat
gtaacgccagtttggagatttcgagggcctggagcagtgcgcaaggtgcg
ctgaaagcctgcccctggatgagatccttatcctggctgtgatggcagtg
gcagtgggctgggtcccttgttgagtggaaagggggactgcggtgtccat
ggtgcagtaggtggcgctcttctgtcttagagcctgccgccactgcagct
ggtgccaaggggccttctgccactagaggtgccatttttcacatgatgaa
cttagcctagttagatcgcagagcaagctgtaagccatgggcccagaaaa
gaaaacttgaagtgagcagatgttgtcacttccttgtaatcctttgttaa
aatagcataaggagttttctttattctatttactttcattaaatgaccgt
gctacaggtttcaaaggattttaagattgatttttgaaagatcacaatat
taaaagtataactggaaaacctatgttgaaatcaaccaaacatgtcgtgg
actgaatgataaccttttctttcttcatatagGCTGATCAGCTGACCGAA
GAACAGATTGCTGgtaagttgacaactccaaggagtccccagaaggccag
aactaggcactgactcagttttggtgactcctctgttcctccccgctaca
gtctgggcagttttctaagaatttatttaaataagaacagtaagcagaaa
cactgaggtcagatgttattcttgccagtactttatagatgaggtgaaag
gaagtaaaactaaggatgcccacatgttaaactctggagaatttgaccat
gtttcacaatgtgcaaagtttgcgtatgattaattgtactgagcctgcta
ctcagcggtttagtttacaattcttatgccatgggtctttcagtaatctg
ccacgaaagcttgtgctcgctatcctaaaataaatggaaatgggtgaata
tgagtgttaggaccactgtagtaattgggaagaaagttacattagttaaa
ctctgttgcccaggctggtctctaactcctgggctcaagcaatcctcctg
cctcagcctcctgagtagctgggactacaggcatgtgccaccacgtctgg
cagattttagctttttaatattcctggaggacttgttttgagactgtttc
tcgttaggaaaccaggaatgcttctgaaatattctaaaagtcatgtggag
agagtttacctgggaatgtacatttctagtaaccattttatttgttatga
aacaagggattcttatggctttagaaatgtaacaggaagggatttgaagg
gggcacatggaccaatcttgtcagattggatttagtcccttgaacctggg
aggcaggggttgtagtgagctgagattgcaccactgcactccaatctcgg
tgacagagcgagactccattgtttaaaaaaaaaaaaaaagattggattta
ggactaatttaagcatgttccagcttagccgccttgaaacctttgggaat
attgtggtgtgtggcactgtttattgggagcagtgtttgctttatgggct
gctgtatgaaggccagtccaacaggactattgtggtcattatttcagtag
ataaagaccagacttctgatacgttgcacaacttgaatggctggctttgg
caagcccccggcaagtgtgtattgtgactgggttggataaagacattgat
tctaacgggtcaacttttgttttcagAATTCAAGGAAGCCTTCTCCCTAT
TTGATAAAGATGGCGATGGCACCATCACAACAAAGGAACTTGGAACTGTC
ATGAGGTCACTGGGTCAGAACCCAACAGAAGCTGAATTGCAGGATATGAT
CATTGAAGTGGATGCTGATGgtaagagctttaaaaccatgaatgagggcc
attgttgtgtaattcaagttcagacatgttacaggattgtctttcaggtc
cccagagcaaagcaaatgtgcaaagatcctttctgtggttgccccagggc
cattgacaattaaaatagaagatgatgggccttgcgtccatcctgcttag
tgtctagaatgttttctgcatgggatcactattgttttcttcctgcttgg
tgcgacctagagctcaaatctatttttttttttttttttggagacggagt
ctcgccctgtcgcccaggctggagtggcactggcgcgatctcggctcact
gcaacctctgcctcttgggttccagcgattctcctgcgtcagccttctga
gtagctggaattacaggcgtgtgtcgccacgcccagttagtgttttgtat
ctttagtagagatggggtttcaccatgttggccaggctggtctcaaactc
ctgacctcgtgatccgccctccccggcctcccaaagtgctgggattacag
gcgtgaaccactgctcctggccgagctcaaagcttttatcaactggccca
tgagtctgcactgagtcttgaggggggaggtgaaattaaatagccataga
aagtgctttttaacaaacttactgtgtttaaagaggaggaggaaccccca
gatgaagtaggtgacgagcactcttagaagttaccataaaagtgagtaca
gtgtgagctgtagatgtgtttgctgcagaggagcatgtgaggtttggagg
cggatgtgtggtgactccaggggatagatttgcagaacctaacggaaagg
gaagctgtaaggtgcagggccagagggaaccagcagtaaccctgatagcg
gtctgtcatctgttcctctcgactctacagcagcggacaacagaactttg
attgctgatttccatcagtaagcaggctttgaagcacacttccccacccc
taaaaaaaaaccacgtattttggtaaatcctatatatattctaatgtact
gtatgacagtatagaacatgatttttaaaagatgagttgggaggagaaaa
ggataaaagaaaaaataaaagaagcattaagaataaacaattcggatcta
gattttactttctagatgattgactcgagggtggtgtagtaaaatcgctt
gtctggtcacaaacatttggcagcagagcttttgattaggttctttgaca
aagccttcagcacgttagagtggttttcactaatagtgttttggaaagaa
aaggttgtccatagttctctagtttgctaagatgatcagctacccaggaa
cgtggagtaacttcctcttgtttgtgggagccccgggaatctgtgcctgg
ggaggggagaagtctgttaggctcttggattgtgtggaagaaggagaagt
tgtgccaggctacagaatcctgtgtttgcactgagaaaacaggatggtac
ctgaccttctctgcatggctgtgagatagcttaaaataatttcttttgtt
tttgatgaatatgaacaatatcttaaaatttttgaggctaaaaaagtctt
gaagggatccctgaggtattttctttgaaaggtactggtgaaaatgagta
acttaacctaagggtttttctttctaattttatttccatttagttcaatg
acactgttagtctggagtgcttgtctttgggggtattcatctcttagttt
taaagaggagttgtttggagtactggccgtagaacagattgttctgacag
ttccctaagtgttactagtctgagctgtgagaatgctcctgagcttttcc
cttaatgggaaataaagatactgagttggaagaaaacaggtggctaacca
tcatagcgtggccaagaaatgatcctggagaagacttggtaagacttcat
ggcccatgcatggcataacagaatcaatgttcctctctcataatcttttc
tcctctgaaacactttatacacttaacctgcagctcagttctaggccttt
tttgtgttactgctgtcactaaccaaggcagagtgagacctgagtgattt
ccctaactcagggatggcagtcgggggcgctttcttccctcggagtggaa
agattcagcctgcggagtggtgtatgctatttttctcttgaactgtacag
cccttcatgacccttccatgggcttgaatccagatgtgcagtttcctttg
tataattaaatactatcctgggcactgatgatgagtttgaaattatgtga
aattgccctgtgaagtgtttgaacgttttagacctgcagatgattgaacc
tagtaagatagtctgcccctttgtcctagtacatgtttaccgttccgtac
agtggttctgaaatgattactgcagagcagcgttaatggagtgcttactt
tacatgagctttttgttttttaattcgagGTAATGGCACCATTGACTTCC
CCGAATTTTTGACTATGATGGCTAGAAAAATGAAAGATACAGATAGTGAA
GAAGAAATCCGTGAGGCATTCCGAGTCTTTGACAAGgtaatccagcatct
acatagcagatggtacttaagtatggcttcttccgctttcacttctaaaa
gctataataatgttatagacagaagacttaaatctaactgcctgagcctc
tgatctcactttcaaaaatcctccttatggtaaccgtatcaggggagggt
aggcataataaataggaattttggaccatgtttcttgactgttactttga
attgttgtgagcttttgcaaatcctgttttctgcattagctgtttgcatg
tatttagtaggttagaggtgggaactagagatcagagaattgtttatggc
agcagagttagcagtaacttgagagggcatagctaagtcaaagacctact
tccccacactacatcattagcaataacaattgctgaatgttcacagGATG
GCAATGGTTATATCAGTGCAGCAGAACTACGTCACGTCATGACAAACTTA
GGAGAAAAACTAACAGATGAAGAAGTAGATGAAATGATCAGAGAAGCAGA
TATTGATGGAGACGGACAAGTCAACTATGAAGgtaaaactaaattctctg
agctcagtgtttcatagtcttacctttagatctgtaagcaagccaactgc
ttcactagacagcctttgactttattttatgtacagtaaagatgttgtgt
tcattaaagctgttttcaaagataaccaaaagttactattatatttgtct
tttcagAATTCGTACAGATGATGACTGCAAAATGAAGACCTACTTTCAAC
TCCTTTTTCCCCCCTCTAGAAGAATCAAATTGAATCTTTTACTTACCTCT
TGCAAAAAAAAGAAAAAAGAAAAAAGTTCATTTATTCATTCTGTTTCTAT
ATAGCAAAACTGAATGTCAAAAGTACCTTCTGTCCACACACACAAAATCT
GCATGTATTGGTTGGTGGTCCTGTCCCCTAAAGATCAAGCTACACATCAG
TTTTACAATATAAATACTTGTACTACCTTAATGATAAGGACTCCTTAAAG
TTCCATTTGCTAATGATTAATACACTGTTTGGGCTGGCCAGTTTTTCATG
CATGCAGCTTGACGATTGAGCACAGTCAGGCCTTTGTATTAAAAATGAAA
AATGAAAAAACAAATTCAAAACCTATTCAAATGGGTTCTAGTTCAATTTG
TTTAGTATAAATTGTCATAGCTGGTTTACTGAAAACAAACACATTTAAAA
TTGGTTTACCTCAGGATGACGTGCAGAAAAATGGGTGAAGGATAAACCGT
TGAGACGTGGCCCCACTGGTAGGATGGTCCTCTTGTACTTCGTGTGCTCC
GACCCATGGTGACGATGACACACCCTGGTGGCATGCCCGTGTATGTTGGT
TTAGCGTTGTCTGCATTGTTCTAGAGTGAAACAGGTGTCAGGCTGTCACT
GTTCACACAAATTTTTAATAAGAAACATTTACCAAGGGAGCATCTTTGGA
CTCTCTGTTTTTAAAACCTTCTGAACCATGACTTGGAGCCGGCAGAGTAG
GCTGTGGCTGTGGACTTCAGCACAACCATCAACATTGCTGTTCAAAGAAA
TTACAGTTTACGTCCATTCCAAGTTGTAAATGCTAGTCTTTTTTTTTTTT
TTTCCAATAAAAAGACCATTAACTTAAAGTGGTGTTAAATGCTTTGTAAA
GCTGAGATCTAAATGGGGACAAGGCAGGTGGAGGGGAGGCCAGTGTACAT
GTAAATGCCCACAGCCCAGCATTGGGTTTCCCTCCCAAGGCCCCAGCACC
AACCTCTGAGCCCAAGACCTTGCCTGAAAACAAGCAGATACCGATTGCTT
CATCCTATTTATGGACATGTAGGTCTAGTTGCATTTTCACTGGGGGGAGG
GGGGAAGGTGAATTATGGTAACTTTTAATGATCTATTCAGGCAGTAGAGC
TCTTAAGGAAAAAAAAAAACCCACTTTCTCTCAAGCATGTATTTAGGGGT
TGTTCTCAATTGTGCTGCTGATTACCTGTCTTATGTAACTACTTGAGACC
ATCTGCAAGAGACATGATTTAGTGTGTCTGTAATTCAATCTTCGCTGTGT
GTGGTAGAAGCAGTAGTCACTTTTGTAAGCCAGTCTCTTCATGCCTAAAA
GACACTACCAGTCACCTTTGATTCGCGACTTTTAATTTATGATTATACTT
AGCCTCCTCCTCCTTTTTTTTTTTTTCCCAAGTTGACTTGACTTTGCTTT
TTTCCCCCCAAGTAGAACTAATGCTAGCTTCCAGCTTGAAAGTAAAACTC
CAGTGTGGAGTGAATTTTGTGTCTAATTATAAACCTGTAACCAAAACTCA
GACATCTGGTACTGGTCTTTGCATTGAGATTGGTCCCTGTAAAACCCCCT
TTAAAAGCATATTGCATTTAGTACAGAGCTCTTTTTTGAAATGAAGGCTG
GAGATGTGCATTTTTCACGGTGTTAACTGGTTGTATCTTATTAGCAAGGA
GATTGGGGTTTTGAGTGTTTGCGTGGGTGGTTTCAATTTGCCAGGGAACA
GTGGCAGGCTGCTAGCAAGGCAGTGAGAAGCTCTTGGCAGCCAAATGGGT
GCATTCAGGGCTGATTTATAGAGACCCTTGGCTTCTCCTTCTCCTACTCC
CTGTCTTTCTGGCATTTTGTAGCTTGTTAGATTTTCTGCCAGAGGGGTGG
GTCAGAGCAGTGGAGGGGAGACATCGCCCATGTGCTTCTGCTACTGGTCC
TTGGGCTGGGTGGTTGGTAGAGGAGATGTTGACACTATGAGCTAAGGGTT
GGCTTTTGTAATTACCTGAATCTGAAAGGAATGCCTAAGGTTACCTTGGG
GTTTCTCTTCTGGTGAGATAGGGTTCCTGGTTTGAGTAAGTTAATGTCCT
GGATATTTCTTGTGGCAGGGGGTGGTCAAAGAGCCTGATTGCTGACCCAG
TCTCAGGCCTGTGGTCGATGACCTCTCGGTAGTTTCAAAGGGGGCTGGAG
GGGGATATTTGACTTGTTTTTTCGAAATGTAGCCTTCTAACCCTCAAGTC
TTTAGAAGCTGGGTGGACTCTTAGTGGTCCTGCAGCGTATCCTAAAAGAC
TACCTTTGAAACAGGATTCTTGTATGGCCAGGATCCTGTCTGGGAACCAG
AAACCCTACACCCTCCCCCTCCAGGGAATGCTGAGTTCCAGTTTTGAGCA
GAGGTGAGGCAGAATCCACTGTAGCCTTCCGCCCTGGTATTTGGGGGGAT
GACCAGCCCAGGCGTTGGGTGTTAGTCTGCATGAGTTTGTGAGAGGAAAT
AGCTGGGTGTCCTGGCAGTGCCCTTGAAGTTGGTTAGGACCTTCCTGTAA
ACTCTTGCCCCTACTTCTAACTACTCTATAAATATATACATATATTTATA
TATAAAGTGATTAGTTGAACTGGCATCCTGCTTTAGCCTGAGACTTGCCA
TAAGAAACTGCTGAGTACTTGGCAAACCCTTTCATAGTTTTGTTCTCCAT
CTGTTTGGGGTAGGTGTTGAGCGAGGCAAATGGATCTCGATATTTCAGAT
GGGCTTTTGATGCACTGTTGCCAAGGAAGGCTTTTTCTGATTTTTTGACA
AATGAATTTTTGCACACTTTCATTGGTGTCTTTCGGCAACTTACACACAT
TGAAAATGAGCTATTGTACATATTTTTATATTCTCTTTATAAATGCATGT
CTGATTGTACTTGTAACAATATTGTAATGAACGGCTGTGCAGTAGGCCCA
GCGCTGCTGTGTCTCGTCAGAGGAATAGCTTACCACGAACCCCTCAGCAT
ACTGGGAATCTCTTCCTGAACAACGAATGTAAATTTGGTCAAGTCTACTC
TTCCGTTCATTCAATTATTTTAAGCATTTGAATTATTTATTGTATATCCT
AAATATATTTCTCCTTTGGCAGTGACTAGATTTCCACTAATGTGTCTTAA
TCTATCCCTCCAGCTGGCAGTTACTGTTTTTTTAATCCCCTGAAGTTGTC
CTGTAGGAGACAGAAATTCTTTGCTGTCTGTATCCCTTGGAGTAAGAAGG
TAGTGGCATGGGTGGAGTGTGTGTTCTTTCTCCAAATCTATTATGATGTT
TATTAAACACTTCTGTAGCAAAGATGGTGGTAGTTCTTTTGTTACTGAAG
TTGCCCTTCACCATGGCTATTTGAAAAGGAGATGTACTTGGACGTTTCTG
TAAATCTTGAGATAAACTGTTTGGAGATTTAACCACCTCTCTGATGGGGG
ACCAACTCTATGGAAATTGTAAATACGTTTTATTTATAAACCTGGCACTG
TATTCAATAAACATTTCTGCAGCCTTTCATCTCTAACTGCGAA
SEQ ID NO: 9
Mature mRNA sequence (CALM1)
comprising the mutation A409T
>uc001xyl.2 (CALM1) length = 4268
gauacggcgcaccauauauauaucgcggggcgcagacucgcgcuccggca
guggugcugggagugucguggacgccgugccguuacucguagucaggcgg
cggcgcaggcggcggcggcggcauagcgcacagcgcgccuuagcagcagc
agcagcagcagcggcaucggagguacccccgccgucgcagcccccgcgcu
ggugcagccacccucgcucccucugcucuuccucccuucgcucgcaccau
ggcugaucagcugaccgaagaacagauugcugaauucaaggaagccuucu
cccuauuugauaaagauggcgauggcaccaucacaacaaaggaacuugga
acugucaugaggucacugggucagaacccaacagaagcugaauugcagga
uaugaucauugaaguggaugcugaugguaauggcaccauugacuuccccg
aauuuuugacuaugauggcuagaaaaaugaaagauacagauagugaagaa
gaaauccgugaggcauuccgagucuuugacaaggauggcaaugguuauau
cagugcagcagaacuacgucacgucaugacaaacuuaggagaaaaacuaa
cagaugaagaaguagaugaaaugaucagagaagcagauauugauggagac
ggacaagucaacuaugaagaauucguacagaugaugacugcaaaaugaag
accuacuuucaacuccuuuuuccccccucuagaagaaucaaauugaaucu
uuuacuuaccucuugcaaaaaaaagaaaaaagaaaaaaguucauuuauuc
auucuguuucuauauagcaaaacugaaugucaaaaguaccuucuguccac
acacacaaaaucugcauguauugguuggugguccuguccccuaaagauca
agcuacacaucaguuuuacaauauaaauacuuguacuaccuuaaugauaa
ggacuccuuaaaguuccauuugcuaaugauuaauacacuguuugggcugg
ccaguuuuucaugcaugcagcuugacgauugagcacagucaggccuuugu
auuaaaaaugaaaaaugaaaaaacaaauucaaaaccuauucaaauggguu
cuaguucaauuuguuuaguauaaauugucauagcugguuuacugaaaaca
aacacauuuaaaauugguuuaccucaggaugacgugcagaaaaaugggug
aaggauaaaccguugagacguggccccacugguaggaugguccucuugua
cuucgugugcuccgacccauggugacgaugacacacccugguggcaugcc
cguguauguugguuuagcguugucugcauuguucuagagugaaacaggug
ucaggcugucacuguucacacaaauuuuuaauaagaaacauuuaccaagg
gagcaucuuuggacucucuguuuuuaaaaccuucugaaccaugacuugga
gccggcagaguaggcuguggcuguggacuucagcacaaccaucaacauug
cuguucaaagaaauuacaguuuacguccauuccaaguuguaaaugcuagu
cuuuuuuuuuuuuuuuccaauaaaaagaccauuaacuuaaagugguguua
aaugcuuuguaaagcugagaucuaaauggggacaaggcagguggagggga
ggccaguguacauguaaaugcccacagcccagcauuggguuucccuccca
aggccccagcaccaaccucugagcccaagaccuugccugaaaacaagcag
auaccgauugcuucauccuauuuauggacauguaggucuaguugcauuuu
cacuggggggaggggggaaggugaauuaugguaacuuuuaaugaucuauu
caggcaguagagcucuuaaggaaaaaaaaaaacccacuuucucucaagca
uguauuuagggguuguucucaauugugcugcugauuaccugucuuaugua
acuacuugagaccaucugcaagagacaugauuuagugugucuguaauuca
aucuucgcugugugugguagaagcaguagucacuuuuguaagccagucuc
uucaugccuaaaagacacuaccagucaccuuugauucgcgacuuuuaauu
uaugauuauacuuagccuccuccuccuuuuuuuuuuuuucccaaguugac
uugacuuugcuuuuuuccccccaaguagaacuaaugcuagcuuccagcuu
gaaaguaaaacuccaguguggagugaauuuugugucuaauuauaaaccug
uaaccaaaacucagacaucugguacuggucuuugcauugagauugguccc
uguaaaacccccuuuaaaagcauauugcauuuaguacagagcucuuuuuu
gaaaugaaggcuggagaugugcauuuuucacgguguuaacugguuguauc
uuauuagcaaggagauugggguuuugaguguuugcgugggugguuucaau
uugccagggaacaguggcaggcugcuagcaaggcagugagaagcucuugg
cagccaaaugggugcauucagggcugauuuauagagacccuuggcuucuc
cuucuccuacucccugucuuucuggcauuuuguagcuuguuagauuuucu
gccagaggggugggucagagcaguggaggggagacaucgcccaugugcuu
cugcuacugguccuugggcugggugguugguagaggagauguugacacua
ugagcuaaggguuggcuuuuguaauuaccugaaucugaaaggaaugccua
agguuaccuugggguuucucuucuggugagauaggguuccugguuugagu
aaguuaauguccuggauauuucuuguggcaggggguggucaaagagccug
auugcugacccagucucaggccuguggucgaugaccucucgguaguuuca
aagggggcuggagggggauauuugacuuguuuuuucgaaauguagccuuc
uaacccucaagucuuuagaagcuggguggacucuuagugguccugcagcg
uauccuaaaagacuaccuuugaaacaggauucuuguauggccaggauccu
gucugggaaccagaaacccuacacccucccccuccagggaaugcugaguu
ccaguuuugagcagaggugaggcagaauccacuguagccuuccgcccugg
uauuuggggggaugaccagcccaggcguuggguguuagucugcaugaguu
ugugagaggaaauagcuggguguccuggcagugcccuugaaguugguuag
gaccuuccuguaaacucuugccccuacuucuaacuacucuauaaauauau
acauauauuuauauauaaagugauuaguugaacuggcauccugcuuuagc
cugagacuugccauaagaaacugcugaguacuuggcaaacccuuucauag
uuuuguucuccaucuguuugggguagguguugagcgaggcaaauggaucu
cgauauuucagaugggcuuuugaugcacuguugccaaggaaggcuuuuuc
ugauuuuuugacaaaugaauuuuugcacacuuucauuggugucuuucggc
aacuuacacacauugaaaaugagcuauuguacauauuuuuauauucucuu
uauaaaugcaugucugauuguacuuguaacaauauuguaaugaacggcug
ugcaguaggcccagcgcugcugugucucgucagaggaauagcuuaccacg
aaccccucagcauacugggaaucucuuccugaacaacgaauguaaauuug
gucaagucuacucuuccguucauucaauuauuuuaagcauuugaauuauu
uauuguauauccuaaauauauuucuccuuuggcagugacuagauuuccac
uaaugugucuuaaucuaucccuccagcuggcaguuacuguuuuuuuaauc
cccugaaguuguccuguaggagacagaaauucuuugcugucuguaucccu
uggaguaagaagguaguggcauggguggaguguguguucuuucuccaaau
cuauuaugauguuuauuaaacacuucuguagcaaagauggugguaguucu
uuuguuacugaaguugcccuucaccauggcuauuugaaaaggagauguac
uuggacguuucuguaaaucuugagauaaacuguuuggagauuuaaccacc
ucucugaugggggaccaacucuauggaaauuguaaauacguuuuauuuau
aaaccuggcacuguauucaauaaacauuucugcagccuuucaucucuaac
ugcgaaaaaaaaaaaaaa
SEQ ID NO: 10
Calmodulin amino acid sequence comprising
the mutation Asn53Ile.
ADQLTEEQIAEFKEAFSLFDKDGDGTITTKELGTVMRSLGQNPTEAELQD
MIIEVDADGNGTIDFPEFLTMMARKMKDTDSEEEIREAFRVFDKDGNGYI
SAAELRHVMTNLGEKLTDEEVDEMIREADIDGDGQVNYEEFVQMMTAK
SEQ ID NO: 11
Calmodulin 1 (CALM1) gene sequence comprising
the mutation A7404G. Exon sequence in upper case.
>hg19_knownGene_uc001xyl.2 range =
chr14: 90863327-90874619 5′pad = 0 3′pad = 0
strand = + repeatMasking = none
GATACGGCGCACCATATATATATCGCGGGGCGCAGACTCGCGCTCCGGCA
GTGGTGCTGGGAGTGTCGTGGACGCCGTGCCGTTACTCGTAGTCAGGCGG
CGGCGCAGGCGGCGGCGGCGGCATAGCGCACAGCGCGCCTTAGCAGCAGC
AGCAGCAGCAGCGGCATCGGAGGTACCCCCGCCGTCGCAGCCCCCGCGCT
GGTGCAGCCACCCTCGCTCCCTCTGCTCTTCCTCCCTTCGCTCGCACCAT
Ggtaggtcgggagtggcaaatgccggcgtagcagctgcccgagatttctt
cccagatttctagttgttttgtttgttttttgtttgtttttggttcttgg
aggtttttcttttctgagtgttacgcagcagctgcgcttaaaggaggttg
cattttggatttgcatctcggcgacctctgccagggagcttcatttattg
gttccccttggagctggacttggtcgtaggccgtccacgggcaggggctc
cggccgcaactgcagcgggggtttctgcatccaatccccctgccccccgc
ccagccccgcacccactgcatccactagcgccgcacccgggctgcctgca
gcgcagcgtttcggcctgggagccgggcggggccgggcactagacccccc
cccccggcccgcccctccccaccccgcttctccgccggcgcgaaggtggc
aggtcgggcgggcagtggagaatgaatgggctggagctggccggtggcgc
acattgttccggccgggtgttgaggggcgcagtcagcgcccgccacctcc
ccactttggccggccctgctgggcgccctccctcggtcgctctcccctcc
ttcttcccggggggcgcggcgcgggcgtgggctgggaaggaaggagccgg
ggaagggtggggttgggggcaggaaggcgaggggttgggggcggagaggg
cggaagcggcggccgggccgccctgcgcccgggcggggccctgcggtgtg
gccgtggcttgttcctgccgctttcgcaccctgcggccccccacccagtg
cagcagtgcgggcgggcgtgagcctcggtgcaccaggaggcacttcccgc
gggaggcgctgggctcgcgctaattggggcggggggggggggcggcgggg
gaggagggaactggcgcgcggcttggtttccattagagacgcaaagtttc
tgctccgggaggaggcggcggcgccgcgggctcgtcgcctgggggagcag
aagcgggtgggaggtgcgggtggccttggcctcagccctggtgcgcgggg
gccgggggtggtgaccctcctggccgaggaggggcggcgtccagacgccc
gctcgggggccgccttcccccccacgcctgcccccgggcacgcgccctgc
ccggtccctcgccccgcgccacttccagtccgcagagagatgccctccac
gtttctgctttctctgcagcctctagattgccagatgcgactgtgcgcct
cgctgggtgtgttttccacagccccttcctcctcggcgtgcagggctgac
atcaccgactgcgtttctggtttggcgggtggggagatggttccccgcag
ggttctggtacacctttgcccccagggctagcgccatttgggggaggagg
ttttcgttgtcgagaaagttggatgctcctggtaacccctctaacaagag
agttctgtagcgaggtgggactgttctccccataaggtgacagtttctct
tgcgaggtgtggcagcgcttcctgttgtacaagacagatgttgccttggc
gttacgtaaatcatcgtgtctccgtcatttaaagaaagccaatttttagt
gattgaggtagaaagaaagatccgtttataatttgtaaaaacaaattttc
acccagaatcaatatattggaacaccattcctactgttaaagttttcact
taagagtataaacttcatcagctttctattaggacttattttgtaattgg
cttcttaggcatccttctttaaaagagaaatccacgttagctctccttga
ggtctcgagttccctcggctggaggcacaggttcagtggagaccaaataa
tgcaggtgaattaccttcgtggccattactgcctccaacgaagtgtgttt
attaagaacagttcttatgtcattcttaaggtaggtagggttaatactct
ccagcaaatttagtagatactctttgccagaaaagagaggagtatatata
gtttgataattattgtgtagttttctgtgtacttaatttttgcagttttg
taacacttcatttgtaagatggtaccattttttcctggcttctgaatcat
aggatagtttgacccagggcattagccattgtaatggtaggcttttaaca
aataactgcctaatttaaaggattggaaagcatttgttacatggaaatga
agttggtggcgtacccagttgctgtatctttattttttctacttaattat
ttctcataaaatggatataaaagcctgttaatccaacccaatgccattat
gtaacgccagtttggagatttcgagggcctggagcagtgcgcaaggtgcg
ctgaaagcctgcccctggatgagatccttatcctggctgtgatggcagtg
gcagtgggctgggtcccttgttgagtggaaagggggactgcggtgtccat
ggtgcagtaggtggcgctcttctgtcttagagcctgccgccactgcagct
ggtgccaaggggccttctgccactagaggtgccatttttcacatgatgaa
cttagcctagttagatcgcagagcaagctgtaagccatgggcccagaaaa
gaaaacttgaagtgagcagatgttgtcacttccttgtaatcctttgttaa
aatagcataaggagttttctttattctatttactttcattaaatgaccgt
gctacaggtttcaaaggattttaagattgatttttgaaagatcacaatat
taaaagtataactggaaaacctatgttgaaatcaaccaaacatgtcgtgg
actgaatgataaccttttctttcttcatatagGCTGATCAGCTGACCGAA
GAACAGATTGCTGgtaagttgacaactccaaggagtccccagaaggccag
aactaggcactgactcagttttggtgactcctctgttcctccccgctaca
gtctgggcagttttctaagaatttatttaaataagaacagtaagcagaaa
cactgaggtcagatgttattcttgccagtactttatagatgaggtgaaag
gaagtaaaactaaggatgcccacatgttaaactctggagaatttgaccat
gtttcacaatgtgcaaagtttgcgtatgattaattgtactgagcctgcta
ctcagcggtttagtttacaattcttatgccatgggtctttcagtaatctg
ccacgaaagcttgtgctcgctatcctaaaataaatggaaatgggtgaata
tgagtgttaggaccactgtagtaattgggaagaaagttacattagttaaa
ctctgttgcccaggctggtctctaactcctgggctcaagcaatcctcctg
cctcagcctcctgagtagctgggactacaggcatgtgccaccacgtctgg
cagattttagctttttaatattcctggaggacttgttttgagactgtttc
tcgttaggaaaccaggaatgcttctgaaatattctaaaagtcatgtggag
agagtttacctgggaatgtacatttctagtaaccattttatttgttatga
aacaagggattcttatggctttagaaatgtaacaggaagggatttgaagg
gggcacatggaccaatcttgtcagattggatttagtcccttgaacctggg
aggcaggggttgtagtgagctgagattgcaccactgcactccaatctcgg
tgacagagcgagactccattgtttaaaaaaaaaaaaaaagattggattta
ggactaatttaagcatgttccagcttagccgccttgaaacctttgggaat
attgtggtgtgtggcactgtttattgggagcagtgtttgctttatgggct
gctgtatgaaggccagtccaacaggactattgtggtcattatttcagtag
ataaagaccagacttctgatacgttgcacaacttgaatggctggctttgg
caagcccccggcaagtgtgtattgtgactgggttggataaagacattgat
tctaacgggtcaacttttgttttcagAATTCAAGGAAGCCTTCTCCCTAT
TTGATAAAGATGGCGATGGCACCATCACAACAAAGGAACTTGGAACTGTC
ATGAGGTCACTGGGTCAGAACCCAACAGAAGCTGAATTGCAGGATATGAT
CAATGAAGTGGATGCTGATGgtaagagctttaaaaccatgaatgagggcc
attgttgtgtaattcaagttcagacatgttacaggattgtctttcaggtc
cccagagcaaagcaaatgtgcaaagatcctttctgtggttgccccagggc
cattgacaattaaaatagaagatgatgggccttgcgtccatcctgcttag
tgtctagaatgttttctgcatgggatcactattgttttcttcctgcttgg
tgcgacctagagctcaaatctatttttttttttttttttggagacggagt
ctcgccctgtcgcccaggctggagtggcactggcgcgatctcggctcact
gcaacctctgcctcttgggttccagcgattctcctgcgtcagccttctga
gtagctggaattacaggcgtgtgtcgccacgcccagttagtgttttgtat
ctttagtagagatggggtttcaccatgttggccaggctggtctcaaactc
ctgacctcgtgatccgccctccccggcctcccaaagtgctgggattacag
gcgtgaaccactgctcctggccgagctcaaagcttttatcaactggccca
tgagtctgcactgagtcttgaggggggaggtgaaattaaatagccataga
aagtgctttttaacaaacttactgtgtttaaagaggaggaggaaccccca
gatgaagtaggtgacgagcactcttagaagttaccataaaagtgagtaca
gtgtgagctgtagatgtgtttgctgcagaggagcatgtgaggtttggagg
cggatgtgtggtgactccaggggatagatttgcagaacctaacggaaagg
gaagctgtaaggtgcagggccagagggaaccagcagtaaccctgatagcg
gtctgtcatctgttcctctcgactctacagcagcggacaacagaactttg
attgctgatttccatcagtaagcaggctttgaagcacacttccccacccc
taaaaaaaaaccacgtattttggtaaatcctatatatattctaatgtact
gtatgacagtatagaacatgatttttaaaagatgagttgggaggagaaaa
ggataaaagaaaaaataaaagaagcattaagaataaacaattcggatcta
gattttactttctagatgattgactcgagggtggtgtagtaaaatcgctt
gtctggtcacaaacatttggcagcagagcttttgattaggttctttgaca
aagccttcagcacgttagagtggttttcactaatagtgttttggaaagaa
aaggttgtccatagttctctagtttgctaagatgatcagctacccaggaa
cgtggagtaacttcctcttgtttgtgggagccccgggaatctgtgcctgg
ggaggggagaagtctgttaggctcttggattgtgtggaagaaggagaagt
tgtgccaggctacagaatcctgtgtttgcactgagaaaacaggatggtac
ctgaccttctctgcatggctgtgagatagcttaaaataatttcttttgtt
tttgatgaatatgaacaatatcttaaaatttttgaggctaaaaaagtctt
gaagggatccctgaggtattttctttgaaaggtactggtgaaaatgagta
acttaacctaagggtttttctttctaattttatttccatttagttcaatg
acactgttagtctggagtgcttgtctttgggggtattcatctcttagttt
taaagaggagttgtttggagtactggccgtagaacagattgttctgacag
ttccctaagtgttactagtctgagctgtgagaatgctcctgagcttttcc
cttaatgggaaataaagatactgagttggaagaaaacaggtggctaacca
tcatagcgtggccaagaaatgatcctggagaagacttggtaagacttcat
ggcccatgcatggcataacagaatcaatgttcctctctcataatcttttc
tcctctgaaacactttatacacttaacctgcagctcagttctaggccttt
tttgtgttactgctgtcactaaccaaggcagagtgagacctgagtgattt
ccctaactcagggatggcagtcgggggcgctttcttccctcggagtggaa
agattcagcctgcggagtggtgtatgctatttttctcttgaactgtacag
cccttcatgacccttccatgggcttgaatccagatgtgcagtttcctttg
tataattaaatactatcctgggcactgatgatgagtttgaaattatgtga
aattgccctgtgaagtgtttgaacgttttagacctgcagatgattgaacc
tagtaagatagtctgcccctttgtcctagtacatgtttaccgttccgtac
agtggttctgaaatgattactgcagagcagcgttaatggagtgcttactt
tacatgagctttttgttttttaattcgagGTAATGGCACCATTGACTTCC
CCGAATTTTTGACTATGATGGCTAGAAAAATGAAAGATACAGATAGTGAA
GAAGAAATCCGTGAGGCATTCCGAGTCTTTGACAAGgtaatccagcatct
acatagcagatggtacttaagtatggcttcttccgctttcacttctaaaa
gctataataatgttatagacagaagacttaaatctaactgcctgagcctc
tgatctcactttcaaaaatcctccttatggtaaccgtatcaggggagggt
aggcataataaataggaattttggaccatgtttcttgactgttactttga
attgttgtgagcttttgcaaatcctgttttctgcattagctgtttgcatg
tatttagtaggttagaggtgggaactagagatcagagaattgtttatggc
agcagagttagcagtaacttgagagggcatagctaagtcaaagacctact
tccccacactacatcattagcaataacaattgctgaatgttcacagGATG
GCAGTGGTTATATCAGTGCAGCAGAACTACGTCACGTCATGACAAACTTA
GGAGAAAAACTAACAGATGAAGAAGTAGATGAAATGATCAGAGAAGCAGA
TATTGATGGAGACGGACAAGTCAACTATGAAGgtaaaactaaattctctg
agctcagtgtttcatagtcttacctttagatctgtaagcaagccaactgc
ttcactagacagcctttgactttattttatgtacagtaaagatgttgtgt
tcattaaagctgttttcaaagataaccaaaagttactattatatttgtct
tttcagAATTCGTACAGATGATGACTGCAAAATGAAGACCTACTTTCAAC
TCCTTTTTCCCCCCTCTAGAAGAATCAAATTGAATCTTTTACTTACCTCT
TGCAAAAAAAAGAAAAAAGAAAAAAGTTCATTTATTCATTCTGTTTCTAT
ATAGCAAAACTGAATGTCAAAAGTACCTTCTGTCCACACACACAAAATCT
GCATGTATTGGTTGGTGGTCCTGTCCCCTAAAGATCAAGCTACACATCAG
TTTTACAATATAAATACTTGTACTACCTTAATGATAAGGACTCCTTAAAG
TTCCATTTGCTAATGATTAATACACTGTTTGGGCTGGCCAGTTTTTCATG
CATGCAGCTTGACGATTGAGCACAGTCAGGCCTTTGTATTAAAAATGAAA
AATGAAAAAACAAATTCAAAACCTATTCAAATGGGTTCTAGTTCAATTTG
TTTAGTATAAATTGTCATAGCTGGTTTACTGAAAACAAACACATTTAAAA
TTGGTTTACCTCAGGATGACGTGCAGAAAAATGGGTGAAGGATAAACCGT
TGAGACGTGGCCCCACTGGTAGGATGGTCCTCTTGTACTTCGTGTGCTCC
GACCCATGGTGACGATGACACACCCTGGTGGCATGCCCGTGTATGTTGGT
TTAGCGTTGTCTGCATTGTTCTAGAGTGAAACAGGTGTCAGGCTGTCACT
GTTCACACAAATTTTTAATAAGAAACATTTACCAAGGGAGCATCTTTGGA
CTCTCTGTTTTTAAAACCTTCTGAACCATGACTTGGAGCCGGCAGAGTAG
GCTGTGGCTGTGGACTTCAGCACAACCATCAACATTGCTGTTCAAAGAAA
TTACAGTTTACGTCCATTCCAAGTTGTAAATGCTAGTCTTTTTTTTTTTT
TTTCCAATAAAAAGACCATTAACTTAAAGTGGTGTTAAATGCTTTGTAAA
GCTGAGATCTAAATGGGGACAAGGCAGGTGGAGGGGAGGCCAGTGTACAT
GTAAATGCCCACAGCCCAGCATTGGGTTTCCCTCCCAAGGCCCCAGCACC
AACCTCTGAGCCCAAGACCTTGCCTGAAAACAAGCAGATACCGATTGCTT
CATCCTATTTATGGACATGTAGGTCTAGTTGCATTTTCACTGGGGGGAGG
GGGGAAGGTGAATTATGGTAACTTTTAATGATCTATTCAGGCAGTAGAGC
TCTTAAGGAAAAAAAAAAACCCACTTTCTCTCAAGCATGTATTTAGGGGT
TGTTCTCAATTGTGCTGCTGATTACCTGTCTTATGTAACTACTTGAGACC
ATCTGCAAGAGACATGATTTAGTGTGTCTGTAATTCAATCTTCGCTGTGT
GTGGTAGAAGCAGTAGTCACTTTTGTAAGCCAGTCTCTTCATGCCTAAAA
GACACTACCAGTCACCTTTGATTCGCGACTTTTAATTTATGATTATACTT
AGCCTCCTCCTCCTTTTTTTTTTTTTCCCAAGTTGACTTGACTTTGCTTT
TTTCCCCCCAAGTAGAACTAATGCTAGCTTCCAGCTTGAAAGTAAAACTC
CAGTGTGGAGTGAATTTTGTGTCTAATTATAAACCTGTAACCAAAACTCA
GACATCTGGTACTGGTCTTTGCATTGAGATTGGTCCCTGTAAAACCCCCT
TTAAAAGCATATTGCATTTAGTACAGAGCTCTTTTTTGAAATGAAGGCTG
GAGATGTGCATTTTTCACGGTGTTAACTGGTTGTATCTTATTAGCAAGGA
GATTGGGGTTTTGAGTGTTTGCGTGGGTGGTTTCAATTTGCCAGGGAACA
GTGGCAGGCTGCTAGCAAGGCAGTGAGAAGCTCTTGGCAGCCAAATGGGT
GCATTCAGGGCTGATTTATAGAGACCCTTGGCTTCTCCTTCTCCTACTCC
CTGTCTTTCTGGCATTTTGTAGCTTGTTAGATTTTCTGCCAGAGGGGTGG
GTCAGAGCAGTGGAGGGGAGACATCGCCCATGTGCTTCTGCTACTGGTCC
TTGGGCTGGGTGGTTGGTAGAGGAGATGTTGACACTATGAGCTAAGGGTT
GGCTTTTGTAATTACCTGAATCTGAAAGGAATGCCTAAGGTTACCTTGGG
GTTTCTCTTCTGGTGAGATAGGGTTCCTGGTTTGAGTAAGTTAATGTCCT
GGATATTTCTTGTGGCAGGGGGTGGTCAAAGAGCCTGATTGCTGACCCAG
TCTCAGGCCTGTGGTCGATGACCTCTCGGTAGTTTCAAAGGGGGCTGGAG
GGGGATATTTGACTTGTTTTTTCGAAATGTAGCCTTCTAACCCTCAAGTC
TTTAGAAGCTGGGTGGACTCTTAGTGGTCCTGCAGCGTATCCTAAAAGAC
TACCTTTGAAACAGGATTCTTGTATGGCCAGGATCCTGTCTGGGAACCAG
AAACCCTACACCCTCCCCCTCCAGGGAATGCTGAGTTCCAGTTTTGAGCA
GAGGTGAGGCAGAATCCACTGTAGCCTTCCGCCCTGGTATTTGGGGGGAT
GACCAGCCCAGGCGTTGGGTGTTAGTCTGCATGAGTTTGTGAGAGGAAAT
AGCTGGGTGTCCTGGCAGTGCCCTTGAAGTTGGTTAGGACCTTCCTGTAA
ACTCTTGCCCCTACTTCTAACTACTCTATAAATATATACATATATTTATA
TATAAAGTGATTAGTTGAACTGGCATCCTGCTTTAGCCTGAGACTTGCCA
TAAGAAACTGCTGAGTACTTGGCAAACCCTTTCATAGTTTTGTTCTCCAT
CTGTTTGGGGTAGGTGTTGAGCGAGGCAAATGGATCTCGATATTTCAGAT
GGGCTTTTGATGCACTGTTGCCAAGGAAGGCTTTTTCTGATTTTTTGACA
AATGAATTTTTGCACACTTTCATTGGTGTCTTTCGGCAACTTACACACAT
TGAAAATGAGCTATTGTACATATTTTTATATTCTCTTTATAAATGCATGT
CTGATTGTACTTGTAACAATATTGTAATGAACGGCTGTGCAGTAGGCCCA
GCGCTGCTGTGTCTCGTCAGAGGAATAGCTTACCACGAACCCCTCAGCAT
ACTGGGAATCTCTTCCTGAACAACGAATGTAAATTTGGTCAAGTCTACTC
TTCCGTTCATTCAATTATTTTAAGCATTTGAATTATTTATTGTATATCCT
AAATATATTTCTCCTTTGGCAGTGACTAGATTTCCACTAATGTGTCTTAA
TCTATCCCTCCAGCTGGCAGTTACTGTTTTTTTAATCCCCTGAAGTTGTC
CTGTAGGAGACAGAAATTCTTTGCTGTCTGTATCCCTTGGAGTAAGAAGG
TAGTGGCATGGGTGGAGTGTGTGTTCTTTCTCCAAATCTATTATGATGTT
TATTAAACACTTCTGTAGCAAAGATGGTGGTAGTTCTTTTGTTACTGAAG
TTGCCCTTCACCATGGCTATTTGAAAAGGAGATGTACTTGGACGTTTCTG
TAAATCTTGAGATAAACTGTTTGGAGATTTAACCACCTCTCTGATGGGGG
ACCAACTCTATGGAAATTGTAAATACGTTTTATTTATAAACCTGGCACTG
TATTCAATAAACATTTCTGCAGCCTTTCATCTCTAACTGCGAA
SEQ ID NO: 12
Mature mRNA sequence (CALM1) comprising
the mutation A541G.
>uc001xyl.2 (CALM1) length = 4268
gauacggcgcaccauauauauaucgcggggcgcagacucgcgcuccggca
guggugcugggagugucguggacgccgugccguuacucguagucaggcgg
cggcgcaggcggcggcggcggcauagcgcacagcgcgccuuagcagcagc
agcagcagcagcggcaucggagguacccccgccgucgcagcccccgcgcu
ggugcagccacccucgcucccucugcucuuccucccuucgcucgcaccau
ggcugaucagcugaccgaagaacagauugcugaauucaaggaagccuucu
cccuauuugauaaagauggcgauggcaccaucacaacaaaggaacuugga
acugucaugaggucacugggucagaacccaacagaagcugaauugcagga
uaugaucaaugaaguggaugcugaugguaauggcaccauugacuuccccg
aauuuuugacuaugauggcuagaaaaaugaaagauacagauagugaagaa
gaaauccgugaggcauuccgagucuuugacaaggauggcagugguuauau
cagugcagcagaacuacgucacgucaugacaaacuuaggagaaaaacuaa
cagaugaagaaguagaugaaaugaucagagaagcagauauugauggagac
ggacaagucaacuaugaagaauucguacagaugaugacugcaaaaugaag
accuacuuucaacuccuuuuuccccccucuagaagaaucaaauugaaucu
uuuacuuaccucuugcaaaaaaaagaaaaaagaaaaaaguucauuuauuc
auucuguuucuauauagcaaaacugaaugucaaaaguaccuucuguccac
acacacaaaaucugcauguauugguuggugguccuguccccuaaagauca
agcuacacaucaguuuuacaauauaaauacuuguacuaccuuaaugauaa
ggacuccuuaaaguuccauuugcuaaugauuaauacacuguuugggcugg
ccaguuuuucaugcaugcagcuugacgauugagcacagucaggccuuugu
auuaaaaaugaaaaaugaaaaaacaaauucaaaaccuauucaaauggguu
cuaguucaauuuguuuaguauaaauugucauagcugguuuacugaaaaca
aacacauuuaaaauugguuuaccucaggaugacgugcagaaaaaugggug
aaggauaaaccguugagacguggccccacugguaggaugguccucuugua
cuucgugugcuccgacccauggugacgaugacacacccugguggcaugcc
cguguauguugguuuagcguugucugcauuguucuagagugaaacaggug
ucaggcugucacuguucacacaaauuuuuaauaagaaacauuuaccaagg
gagcaucuuuggacucucuguuuuuaaaaccuucugaaccaugacuugga
gccggcagaguaggcuguggcuguggacuucagcacaaccaucaacauug
cuguucaaagaaauuacaguuuacguccauuccaaguuguaaaugcuagu
cuuuuuuuuuuuuuuuccaauaaaaagaccauuaacuuaaagugguguua
aaugcuuuguaaagcugagaucuaaauggggacaaggcagguggagggga
ggccaguguacauguaaaugcccacagcccagcauuggguuucccuccca
aggccccagcaccaaccucugagcccaagaccuugccugaaaacaagcag
auaccgauugcuucauccuauuuauggacauguaggucuaguugcauuuu
cacuggggggaggggggaaggugaauuaugguaacuuuuaaugaucuauu
caggcaguagagcucuuaaggaaaaaaaaaaacccacuuucucucaagca
uguauuuagggguuguucucaauugugcugcugauuaccugucuuaugua
acuacuugagaccaucugcaagagacaugauuuagugugucuguaauuca
aucuucgcugugugugguagaagcaguagucacuuuuguaagccagucuc
uucaugccuaaaagacacuaccagucaccuuugauucgcgacuuuuaauu
uaugauuauacuuagccuccuccuccuuuuuuuuuuuuucccaaguugac
uugacuuugcuuuuuuccccccaaguagaacuaaugcuagcuuccagcuu
gaaaguaaaacuccaguguggagugaauuuugugucuaauuauaaaccug
uaaccaaaacucagacaucugguacuggucuuugcauugagauugguccc
uguaaaacccccuuuaaaagcauauugcauuuaguacagagcucuuuuuu
gaaaugaaggcuggagaugugcauuuuucacgguguuaacugguuguauc
uuauuagcaaggagauugggguuuugaguguuugcgugggugguuucaau
uugccagggaacaguggcaggcugcuagcaaggcagugagaagcucuugg
cagccaaaugggugcauucagggcugauuuauagagacccuuggcuucuc
cuucuccuacucccugucuuucuggcauuuuguagcuuguuagauuuucu
gccagaggggugggucagagcaguggaggggagacaucgcccaugugcuu
cugcuacugguccuugggcugggugguugguagaggagauguugacacua
ugagcuaaggguuggcuuuuguaauuaccugaaucugaaaggaaugccua
agguuaccuugggguuucucuucuggugagauaggguuccugguuugagu
aaguuaauguccuggauauuucuuguggcaggggguggucaaagagccug
auugcugacccagucucaggccuguggucgaugaccucucgguaguuuca
aagggggcuggagggggauauuugacuuguuuuuucgaaauguagccuuc
uaacccucaagucuuuagaagcuggguggacucuuagugguccugcagcg
uauccuaaaagacuaccuuugaaacaggauucuuguauggccaggauccu
gucugggaaccagaaacccuacacccucccccuccagggaaugcugaguu
ccaguuuugagcagaggugaggcagaauccacuguagccuuccgcccugg
uauuuggggggaugaccagcccaggcguuggguguuagucugcaugaguu
ugugagaggaaauagcuggguguccuggcagugcccuugaaguugguuag
gaccuuccuguaaacucuugccccuacuucuaacuacucuauaaauauau
acauauauuuauauauaaagugauuaguugaacuggcauccugcuuuagc
cugagacuugccauaagaaacugcugaguacuuggcaaacccuuucauag
uuuuguucuccaucuguuugggguagguguugagcgaggcaaauggaucu
cgauauuucagaugggcuuuugaugcacuguugccaaggaaggcuuuuuc
ugauuuuuugacaaaugaauuuuugcacacuuucauuggugucuuucggc
aacuuacacacauugaaaaugagcuauuguacauauuuuuauauucucuu
uauaaaugcaugucugauuguacuuguaacaauauuguaaugaacggcug
ugcaguaggcccagcgcugcugugucucgucagaggaauagcuuaccacg
aaccccucagcauacugggaaucucuuccugaacaacgaauguaaauuug
gucaagucuacucuuccguucauucaauuauuuuaagcauuugaauuauu
uauuguauauccuaaauauauuucuccuuuggcagugacuagauuuccac
uaaugugucuuaaucuaucccuccagcuggcaguuacuguuuuuuuaauc
cccugaaguuguccuguaggagacagaaauucuuugcugucuguaucccu
uggaguaagaagguaguggcauggguggaguguguguucuuucuccaaau
cuauuaugauguuuauuaaacacuucuguagcaaagauggugguaguucu
uuuguuacugaaguugcccuucaccauggcuauuugaaaaggagauguac
uuggacguuucuguaaaucuugagauaaacuguuuggagauuuaaccacc
ucucugaugggggaccaacucuauggaaauuguaaauacguuuuauuuau
aaaccuggcacuguauucaauaaacauuucugcagccuuucaucucuaac
ugcgaaaaaaaaaaaaaa
SEQ ID NO: 13
Calmodulin amino acid sequence comprising
the mutation Asn97Ser.
ADQLTEEQIAEFKEAFSLFDKDGDGTITTKELGTVMRSLGQNPTEAELQD
MIIEVDADGNGTIDFPEFLTMMARKMKDTDSEEEIREAFRVFDKDGSGYI
SAAELRHVMTNLGEKLTDEEVDEMIREADIDGDGQVNYEEFVQMMTAK

What is claimed is:

1. An isolated polynucleotide encoding calmodulin (CaM), or at least a part of calmodulin (CaM), wherein said polynucleotide comprises at least one mutation associated with a cardiac disorder.

2-15. (canceled)

16. An isolated polypeptide comprising calmodulin (CaM), or at least a part of a calmodulin protein (CaM), wherein said polypeptide comprises at least one mutation associated with a cardiac disorder.

17-35. (canceled)

36. A method for determining whether an individual has an increased risk of contracting a cardiac disorder or sudden cardiac death, wherein said method comprises
determining the presence or absence of at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof in a sample from said individual and/or
determining the presence or absence of at least one mutation in the polypeptide having SEQ ID NO:4 or at least 90% sequence identity with SEQ ID NO:4 or part thereof in a sample from said individual,
wherein the presence of said at least one mutation indicates an increased risk of contracting a cardiac disorder.

37. (canceled)

38. (canceled)

39. A method for diagnosing a cardiac disorder of an individual, wherein said method comprises
determining the presence or absence of at least one mutation in CALM1 (SEQ ID NO:1), CALM2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof in a sample from said individual and/or
determining the presence or absence of at least one mutation in the polypeptide having SEQ ID NO:4 or at least 90% sequence identity with SEQ ID NO:4 or part thereof in a sample from said individual,
wherein the presence of said at least one mutation indicates a cardiac disorder or an increased risk of contracting a cardiac disorder.

40-43. (canceled)

44. A method for treatment of an individual having a cardiac disorder associated with at least one mutation in CALM1 (SEQ ID NO:1), CALM 2 (SEQ ID NO:2) and/or CALM3 (SEQ ID NO:3) and/or in a polynucleotide having at least 90% sequence identity with SEQ ID NO:1, SEQ ID NO:2 and/or SEQ ID NO:3 or part thereof, wherein said mutation results in the mutated calmodulin having one or more altered functional property compared to wild type calmodulin, said method comprising administering to said individual an agent capable of restoring and/or improving the altered functional property to the level in wild type calmodulin.

45-57. (canceled)

58. A method for identifying a compound, capable of enhancing the binding of calmodulin to ryanodine receptor 2, wherein said calmodulin comprises at least one mutation that decreases the binding affinity to ryanodine receptor 2, said method comprising
providing a first sample comprising calmodulin protein or a fragment thereof having a mutation that decreases the binding affinity to ryanodine receptor 2, ryanodine receptor 2 or a fragment thereof and a test compound
measuring the amount of calmodulin protein bound to ryanodine receptor 2 protein in said first sample
providing a second sample comprising calmodulin protein or a fragment thereof having a mutation that decreases the binding affinity to ryanodine receptor 2 and ryanodine receptor 2 or a fragment thereof
measuring the amount of calmodulin protein bound to ryanodine receptor 2 protein in said second sample
comparing the amount of calmodulin protein bound to ryanodine receptor 2 protein in the first and second sample, whereby a higher amount of calmodulin protein bound to ryanodine receptor 2 protein in the first sample as compared to the second sample indicates that the test compound enhances binding of calmodulin protein to ryanodine receptor 2 protein.

59. (canceled)

60. A method for identifying a compound, capable of enhancing the binding of calmodulin to calcium (Ca²⁺), wherein said calmodulin comprises at least one mutation that decreases the binding affinity to calcium (Ca²⁺) (and which preferably reduces or abolishes binding to calcium (Ca²⁺)), said method comprising
providing a first sample comprising calmodulin protein or a fragment thereof having a mutation that decreases the binding affinity to calcium (Ca²⁺) (and which preferably reduces or abolishes binding to calcium (Ca²⁺)), calcium (Ca²⁺) and a test compound
measuring the amount of calmodulin protein bound to calcium (Ca²⁺) in said first sample
providing a second sample comprising calmodulin protein or a fragment thereof having a mutation that decreases the binding affinity to calcium (Ca²⁺) (and which preferably reduces or abolishes binding to calcium (Ca²⁺)), and calcium (Ca²⁺)
measuring the amount of calmodulin protein bound to calcium (Ca²⁺) in said second sample
comparing the amount of calmodulin protein bound to calcium (Ca²⁺) in the first and second sample, whereby a higher amount of calmodulin protein bound to calcium (Ca²⁺) in the first sample as compared to the second sample indicates that the test compound enhances binding of calmodulin protein to calcium (Ca2⁺).

61-72. (canceled)

73. A pharmaceutical composition for use in the treatment of an individual having a cardiac disorder associated with at least one mutation in CALM1 (SEQ ID NO:1), CALM2 (SEQ ID NO:2) and/or CALM 3 (SEQ ID NO:3, wherein said mutation results in the mutated calmodulin having one or more altered functional property compared to wild type calmodulin, and wherein said composition comprises an agent capable of restoring and/or improving the altered functional property to the level in wild type calmodulin.

74-83. (canceled)

84. A kit for detecting at least one mutation in a polynucleotide encoding calmodulin (CaM), or at least a part of calmodulin (CaM), wherein said kit comprises at least one oligonucleotide that is complementary to a sequence of said calmodulin encoding gene such that if the mutation is present in the polynucleotide, strand elongation from said oligonucleotide results in an extension product comprising said mutation.

85-96. (canceled)


SEQUENCE LISTING (19 sequences)

SEQ ID NO: 1 - DNA, 11293 bp, Homo sapiens
gatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccatggtaggtcgggagtggcaaatgccggcgtagcagctgcccgagatttcttcccagatttctagttgttttgtttgttttttgtttgtttttggttcttggaggtttttcttttctgagtgttacgcagcagctgcgcttaaaggaggttgcattttggatttgcatctcggcgacctctgccagggagcttcatttattggttccccttggagctggacttggtcgtaggccgtccacgggcaggggctccggccgcaactgcagcgggggtttctgcatccaatccccctgccccccgcccagccccgcacccactgcatccactagcgccgcacccgggctgcctgcagcgcagcgtttcggcctgggagccgggcggggccgggcactagacccccccccccggcccgcccctccccaccccgcttctccgccggcgcgaaggtggcaggtcgggcgggcagtggagaatgaatgggctggagctggccggtggcgcacattgttccggccgggtgttgaggggcgcagtcagcgcccgccacctccccactttggccggccctgctgggcgccctccctcggtcgctctcccctccttcttcccggggggcgcggcgcgggcgtgggctgggaaggaaggagccggggaagggtggggttgggggcaggaaggcgaggggttgggggcggagagggcggaagcggcggccgggccgccctgcgcccgggcggggccctgcggtgtggccgtggcttgttcctgccgctttcgcaccctgcggccccccacccagtgcagcagtgcgggcgggcgtgagcctcggtgcaccaggaggcacttcccgcgggaggcgctgggctcgcgctaattggggcggggggggggggcggcgggggaggagggaactggcgcgcggcttggtttccattagagacgcaaagtttctgctccgggaggaggcggcggcgccgcgggctcgtcgcctgggggagcagaagcgggtgggaggtgcgggtggccttggcctcagccctggtgcgcgggggccgggggtggtgaccctcctggccgaggaggggcggcgtccagacgcccgctcgggggccgccttcccccccacgcctgcccccgggcacgcgccctgcccggtccctcgccccgcgccacttccagtccgcagagagatgccctccacgtttctgctttctctgcagcctctagattgccagatgcgactgtgcgcctcgctgggtgtgttttccacagccccttcctcctcggcgtgcagggctgacatcaccgactgcgtttctggtttggcgggtggggagatggttccccgcagggttctggtacacctttgcccccagggctagcgccatttgggggaggaggttttcgttgtcgagaaagttggatgctcctggtaacccctctaacaagagagttctgtagcgaggtgggactgttctccccataaggtgacagtttctcttgcgaggtgtggcagcgcttcctgttgtacaagacagatgttgccttggcgttacgtaaatcatcgtgtctccgtcatttaaagaaagccaatttttagtgattgaggtagaaagaaagatccgtttataatttgtaaaaacaaattttcacccagaatcaatatattggaacaccattcctactgttaaagttttcacttaagagtataaacttcatcagctttctattaggacttattttgtaattggcttcttaggcatccttctttaaaagagaaatccacgttagctctccttgaggtctcgagttccctcggctggaggcacaggttcagtggagaccaaataatgcaggtgaattaccttcgtggccattactgcctccaacgaagtgtgtttattaagaacagttcttatgtcattcttaaggtaggtagggttaatactctccagcaaatttagtagatactctttgccagaaaagagaggagtatatatagtttgataattattgtgtagttttctgtgtacttaatttttgcagttttgtaacacttcatttgtaagatggtaccattttttcctggcttctgaatcataggatagtttgacccagggcattagccattgtaatggtaggcttttaacaaataactgcctaatttaaaggattggaaagcatttgttacatggaaatgaagttggtggcgtacccagttgctgtatctttattttttctacttaattatttctcataaaatggatataaaagcctgttaatccaacccaatgccattatgtaacgccagtttggagatttcgagggcctggagcagtgcgcaaggtgcgctgaaagcctgcccctggatgagatccttatcctggctgtgatggcagtggcagtgggctgggtcccttgttgagtggaaagggggactgcggtgtccatggtgcagtaggtggcgctcttctgtcttagagcctgccgccactgcagctggtgccaaggggccttctgccactagaggtgccatttttcacatgatgaacttagcctagttagatcgcagagcaagctgtaagccatgggcccagaaaagaaaacttgaagtgagcagatgttgtcacttccttgtaatcctttgttaaaatagcataaggagttttctttattctatttactttcattaaatgaccgtgctacaggtttcaaaggattttaagattgatttttgaaagatcacaatattaaaagtataactggaaaacctatgttgaaatcaaccaaacatgtcgtggactgaatgataaccttttctttcttcatataggctgatcagctgaccgaagaacagattgctggtaagttgacaactccaaggagtccccagaaggccagaactaggcactgactcagttttggtgactcctctgttcctccccgctacagtctgggcagttttctaagaatttatttaaataagaacagtaagcagaaacactgaggtcagatgttattcttgccagtactttatagatgaggtgaaaggaagtaaaactaaggatgcccacatgttaaactctggagaatttgaccatgtttcacaatgtgcaaagtttgcgtatgattaattgtactgagcctgctactcagcggtttagtttacaattcttatgccatgggtctttcagtaatctgccacgaaagcttgtgctcgctatcctaaaataaatggaaatgggtgaatatgagtgttaggaccactgtagtaattgggaagaaagttacattagttaaactctgttgcccaggctggtctctaactcctgggctcaagcaatcctcctgcctcagcctcctgagtagctgggactacaggcatgtgccaccacgtctggcagattttagctttttaatattcctggaggacttgttttgagactgtttctcgttaggaaaccaggaatgcttctgaaatattctaaaagtcatgtggagagagtttacctgggaatgtacatttctagtaaccattttatttgttatgaaacaagggattcttatggctttagaaatgtaacaggaagggatttgaagggggcacatggaccaatcttgtcagattggatttagtcccttgaacctgggaggcaggggttgtagtgagctgagattgcaccactgcactccaatctcggtgacagagcgagactccattgtttaaaaaaaaaaaaaaagattggatttaggactaatttaagcatgttccagcttagccgccttgaaacctttgggaatattgtggtgtgtggcactgtttattgggagcagtgtttgctttatgggctgctgtatgaaggccagtccaacaggactattgtggtcattatttcagtagataaagaccagacttctgatacgttgcacaacttgaatggctggctttggcaagcccccggcaagtgtgtattgtgactgggttggataaagacattgattctaacgggtcaacttttgttttcagaattcaaggaagccttctccctatttgataaagatggcgatggcaccatcacaacaaaggaacttggaactgtcatgaggtcactgggtcagaacccaacagaagctgaattgcaggatatgatcaatgaagtggatgctgatggtaagagctttaaaaccatgaatgagggccattgttgtgtaattcaagttcagacatgttacaggattgtctttcaggtccccagagcaaagcaaatgtgcaaagatcctttctgtggttgccccagggccattgacaattaaaatagaagatgatgggccttgcgtccatcctgcttagtgtctagaatgttttctgcatgggatcactattgttttcttcctgcttggtgcgacctagagctcaaatctatttttttttttttttttggagacggagtctcgccctgtcgcccaggctggagtggcactggcgcgatctcggctcactgcaacctctgcctcttgggttccagcgattctcctgcgtcagccttctgagtagctggaattacaggcgtgtgtcgccacgcccagttagtgttttgtatctttagtagagatggggtttcaccatgttggccaggctggtctcaaactcctgacctcgtgatccgccctccccggcctcccaaagtgctgggattacaggcgtgaaccactgctcctggccgagctcaaagcttttatcaactggcccatgagtctgcactgagtcttgaggggggaggtgaaattaaatagccatagaaagtgctttttaacaaacttactgtgtttaaagaggaggaggaacccccagatgaagtaggtgacgagcactcttagaagttaccataaaagtgagtacagtgtgagctgtagatgtgtttgctgcagaggagcatgtgaggtttggaggcggatgtgtggtgactccaggggatagatttgcagaacctaacggaaagggaagctgtaaggtgcagggccagagggaaccagcagtaaccctgatagcggtctgtcatctgttcctctcgactctacagcagcggacaacagaactttgattgctgatttccatcagtaagcaggctttgaagcacacttccccacccctaaaaaaaaaccacgtattttggtaaatcctatatatattctaatgtactgtatgacagtatagaacatgatttttaaaagatgagttgggaggagaaaaggataaaagaaaaaataaaagaagcattaagaataaacaattcggatctagattttactttctagatgattgactcgagggtggtgtagtaaaatcgcttgtctggtcacaaacatttggcagcagagcttttgattaggttctttgacaaagccttcagcacgttagagtggttttcactaatagtgttttggaaagaaaaggttgtccatagttctctagtttgctaagatgatcagctacccaggaacgtggagtaacttcctcttgtttgtgggagccccgggaatctgtgcctggggaggggagaagtctgttaggctcttggattgtgtggaagaaggagaagttgtgccaggctacagaatcctgtgtttgcactgagaaaacaggatggtacctgaccttctctgcatggctgtgagatagcttaaaataatttcttttgtttttgatgaatatgaacaatatcttaaaatttttgaggctaaaaaagtcttgaagggatccctgaggtattttctttgaaaggtactggtgaaaatgagtaacttaacctaagggtttttctttctaattttatttccatttagttcaatgacactgttagtctggagtgcttgtctttgggggtattcatctcttagttttaaagaggagttgtttggagtactggccgtagaacagattgttctgacagttccctaagtgttactagtctgagctgtgagaatgctcctgagcttttcccttaatgggaaataaagatactgagttggaagaaaacaggtggctaaccatcatagcgtggccaagaaatgatcctggagaagacttggtaagacttcatggcccatgcatggcataacagaatcaatgttcctctctcataatcttttctcctctgaaacactttatacacttaacctgcagctcagttctaggccttttttgtgttactgctgtcactaaccaaggcagagtgagacctgagtgatttccctaactcagggatggcagtcgggggcgctttcttccctcggagtggaaagattcagcctgcggagtggtgtatgctatttttctcttgaactgtacagcccttcatgacccttccatgggcttgaatccagatgtgcagtttcctttgtataattaaatactatcctgggcactgatgatgagtttgaaattatgtgaaattgccctgtgaagtgtttgaacgttttagacctgcagatgattgaacctagtaagatagtctgcccctttgtcctagtacatgtttaccgttccgtacagtggttctgaaatgattactgcagagcagcgttaatggagtgcttactttacatgagctttttgttttttaattcgaggtaatggcaccattgacttccccgaatttttgactatgatggctagaaaaatgaaagatacagatagtgaagaagaaatccgtgaggcattccgagtctttgacaaggtaatccagcatctacatagcagatggtacttaagtatggcttcttccgctttcacttctaaaagctataataatgttatagacagaagacttaaatctaactgcctgagcctctgatctcactttcaaaaatcctccttatggtaaccgtatcaggggagggtaggcataataaataggaattttggaccatgtttcttgactgttactttgaattgttgtgagcttttgcaaatcctgttttctgcattagctgtttgcatgtatttagtaggttagaggtgggaactagagatcagagaattgtttatggcagcagagttagcagtaacttgagagggcatagctaagtcaaagacctacttccccacactacatcattagcaataacaattgctgaatgttcacaggatggcaatggttatatcagtgcagcagaactacgtcacgtcatgacaaacttaggagaaaaactaacagatgaagaagtagatgaaatgatcagagaagcagatattgatggagacggacaagtcaactatgaaggtaaaactaaattctctgagctcagtgtttcatagtcttacctttagatctgtaagcaagccaactgcttcactagacagcctttgactttattttatgtacagtaaagatgttgtgttcattaaagctgttttcaaagataaccaaaagttactattatatttgtcttttcagaattcgtacagatgatgactgcaaaatgaagacctactttcaactcctttttcccccctctagaagaatcaaattgaatcttttacttacctcttgcaaaaaaaagaaaaaagaaaaaagttcatttattcattctgtttctatatagcaaaactgaatgtcaaaagtaccttctgtccacacacacaaaatctgcatgtattggttggtggtcctgtcccctaaagatcaagctacacatcagttttacaatataaatacttgtactaccttaatgataaggactccttaaagttccatttgctaatgattaatacactgtttgggctggccagtttttcatgcatgcagcttgacgattgagcacagtcaggcctttgtattaaaaatgaaaaatgaaaaaacaaattcaaaacctattcaaatgggttctagttcaatttgtttagtataaattgtcatagctggtttactgaaaacaaacacatttaaaattggtttacctcaggatgacgtgcagaaaaatgggtgaaggataaaccgttgagacgtggccccactggtaggatggtcctcttgtacttcgtgtgctccgacccatggtgacgatgacacaccctggtggcatgcccgtgtatgttggtttagcgttgtctgcattgttctagagtgaaacaggtgtcaggctgtcactgttcacacaaatttttaataagaaacatttaccaagggagcatctttggactctctgtttttaaaaccttctgaaccatgacttggagccggcagagtaggctgtggctgtggacttcagcacaaccatcaacattgctgttcaaagaaattacagtttacgtccattccaagttgtaaatgctagtctttttttttttttttccaataaaaagaccattaacttaaagtggtgttaaatgctttgtaaagctgagatctaaatggggacaaggcaggtggaggggaggccagtgtacatgtaaatgcccacagcccagcattgggtttccctcccaaggccccagcaccaacctctgagcccaagaccttgcctgaaaacaagcagataccgattgcttcatcctatttatggacatgtaggtctagttgcattttcactggggggaggggggaaggtgaattatggtaacttttaatgatctattcaggcagtagagctcttaaggaaaaaaaaaaacccactttctctcaagcatgtatttaggggttgttctcaattgtgctgctgattacctgtcttatgtaactacttgagaccatctgcaagagacatgatttagtgtgtctgtaattcaatcttcgctgtgtgtggtagaagcagtagtcacttttgtaagccagtctcttcatgcctaaaagacactaccagtcacctttgattcgcgacttttaatttatgattatacttagcctcctcctcctttttttttttttcccaagttgacttgactttgcttttttccccccaagtagaactaatgctagcttccagcttgaaagtaaaactccagtgtggagtgaattttgtgtctaattataaacctgtaaccaaaactcagacatctggtactggtctttgcattgagattggtccctgtaaaaccccctttaaaagcatattgcatttagtacagagctcttttttgaaatgaaggctggagatgtgcatttttcacggtgttaactggttgtatcttattagcaaggagattggggttttgagtgtttgcgtgggtggtttcaatttgccagggaacagtggcaggctgctagcaaggcagtgagaagctcttggcagccaaatgggtgcattcagggctgatttatagagacccttggcttctccttctcctactccctgtctttctggcattttgtagcttgttagattttctgccagaggggtgggtcagagcagtggaggggagacatcgcccatgtgcttctgctactggtccttgggctgggtggttggtagaggagatgttgacactatgagctaagggttggcttttgtaattacctgaatctgaaaggaatgcctaaggttaccttggggtttctcttctggtgagatagggttcctggtttgagtaagttaatgtcctggatatttcttgtggcagggggtggtcaaagagcctgattgctgacccagtctcaggcctgtggtcgatgacctctcggtagtttcaaagggggctggagggggatatttgacttgttttttcgaaatgtagccttctaaccctcaagtctttagaagctgggtggactcttagtggtcctgcagcgtatcctaaaagactacctttgaaacaggattcttgtatggccaggatcctgtctgggaaccagaaaccctacaccctccccctccagggaatgctgagttccagttttgagcagaggtgaggcagaatccactgtagccttccgccctggtatttggggggatgaccagcccaggcgttgggtgttagtctgcatgagtttgtgagaggaaatagctgggtgtcctggcagtgcccttgaagttggttaggaccttcctgtaaactcttgcccctacttctaactactctataaatatatacatatatttatatataaagtgattagttgaactggcatcctgctttagcctgagacttgccataagaaactgctgagtacttggcaaaccctttcatagttttgttctccatctgtttggggtaggtgttgagcgaggcaaatggatctcgatatttcagatgggcttttgatgcactgttgccaaggaaggctttttctgattttttgacaaatgaatttttgcacactttcattggtgtctttcggcaacttacacacattgaaaatgagctattgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcagttactgtttttttaatcccctgaagttgtcctgtaggagacagaaattctttgctgtctgtatcccttggagtaagaaggtagtggcatgggtggagtgtgtgttctttctccaaatctattatgatgtttattaaacacttctgtagcaaagatggtggtagttcttttgttactgaagttgcccttcaccatggctatttgaaaaggagatgtacttggacgtttctgtaaatcttgagataaactgtttggagatttaaccacctctctgatgggggaccaactctatggaaattgtaaatacgttttatttataaacctggcactgtattcaataaacatttctgcagcctttcatctctaactgcgaa

SEQ ID NO: 2 - DNA, 16520 bp, Homo sapiens
ctcgtttgcgatgttccgttatctggatgcggcggagggatctggcggagggaggtgtttatgaggcgctgggggcggaggaggcgaattagtccgagtggagagagcgagctgagtggttgtgtggtcgcgtctcggaaaccggtagcgcttgcagcatggtgagtgcatcgctgagctgccggcgctgggcctgtggggggaagagactggagcgaactgactaaacaaaggaaactcgcctcccttacaccttcaaggagtggtttctgccagagggatggcgcgaaagaggccagggatgccggcgacggttgtgtcgcaggtgctcccccacccccattctcttcaggggggcttcctctagtgagttgagggccggacgcacagttcccgaggtggtgaagctcctcgggggcttggctggaggaggatgggtccgggatgagggacagcccagtgcacgggaaggagagaaagtgaaggaagggagagagggagcgtagagagccttctgtcgggcgtctggaaaggtcaagcagggggcgaattcaatccacccgaccgacggttagtgcgggcgccaggaagcgcctttgtccgcttgcggccgccatgcgcccagggggcgggaggagcgcgcggggccctggggcgagcgtcggcgtcgcagttagcccgcgtcctccgcgtgccgggatccagggggcgcgcgggcgggcgggcggcgcgctgtgtgggccgagcccgcgcggcgcagggagcggccggtgggggcggggcgggcgagcgcctcgcgggacccggagccgccgccccgctcggccccgctcgagctccagctgctctgctggcacctgcaggactgcggagatctccttggcgctgcggggccattggcggggatgggcgggggaggggggcctcgacggtttcccatccccctctggcaaccctaatcttccttctccatctgggccctggggcgtcttccaccacccaggccggatgctttaaaaaaaattgctttttaaagtgtctgtcgttgaaagaaattagtcttacccttgaagtcaggggcgcgtcctcgcaatacacatcttgttagggaaagtgttcggctccagctagggttctacaaggcgtttcttgttcaccgccggagggaagcaggctccggagtgactgccttctgaaagtcggtcttgtaacaattggatggatgcctttgaagagcccctgtccctattctatgcttgaaaacagcgtgcagtcctaatgttcaagaaccacgaccacataaaaacattgctccccttctgctgctttgaaaacgacccctaaattccgtgtagaagttgccaggtcgtcttgacgtacacttcgtttgtatgatgtttgtctgtcaaatactgtgatggaagagtgtatgcgggggaggagcagggaatttttaaaagcattttccggtcacctcagactgggagatcatgttctttcctgaaaaaaaaaaaaaaaaacacctaatgagggcggtgatctgtgatacttactttgtggttcctcctgccaagtggttacagcgatgggtgtgtccaggaggcaaggtgacatcttaaacaggagttagggatccttgccaactgaaacctccagcctgtatgggtgtggggagacgatttcaccttaaataaaactagcgttcattgacgtttgatggatttgcttcatcttgatgcttcaaatgttattctgaatttgaatgtgctggttacatttgctttcttaaagatgtagttacaggcatatgtaataaacattacaagttgtggagtaagcttttttttttaaataaacgaagggtaaagttttctttaatcagttttgataattgaatcagtaattcgaatagattttttcttggtagagttggtatctacgcatttgtggtcccttgcccagtgttaagcttttgtagttactgtttgcatcactggtgctttgggggttttctgttaaattgacagctcttactataagagacaaagtcgaaaaaaaaaatcttaataaaaattgacagatatcattatttgtgacagatacagtaaatttgaaaattgccttcacataactttataattagaggactgagattttacattaggaatccaattaaaaaaaattagccggacataatggcctgtgcgtgtagtcctggctagtcaatgggctgaggcaggaggatcatcttgggcccgggagttggagactgtagtgagctatgatcacaccactgcactccctgggcaacagagtgagaccctgtctctcaaaaaataaaaaaaattaggaccccaagttaaagtataaaaaattttatacggaagtaaagtaagattgctcatgtaactcttgagtttacatgtaatcaacatatgctcattgaaaacgggattgcttcaagaggactttgagtgcagggtgattaggtaagtaaaagatgtaaaaaggtagaaaatttttgtcacttgagtctaaataattgttcttataagtgccaacgcctgtttctgttaggctcagaagatcaaaggatttggctcttttaaaatatagaaagctctagcttcagctagaatttaggcctttagtaatagccctaatttttatgaagccattttgttccagtgatcttttggtgagagatgctatgtaagtactattcttcagaattaggtgtctttttaccctaatgaaataatttagattgcttttgatacaggtaaaacaaatatcctggcttccataattgtagaaaaaacttcatataggaatccttgttgtatcaaagtagcacctgatgggaatgaacagacaggaatggatgaaggatagcagtttgcgttccatttcaagcctatgggctcacacatttattcagataagaacaccacctttcactagataaactccaacagtattcatgcatacttttgaatggcatgtaggaaatgtttgataggtacataatgtattcacttcaggtcactaatgtaatacggggtcgtgctccttagtgttgacagatcacctatggttctccaaaatgaacattctagtacaggaggtctagggaggaacctgagagtatactaatgcctaggaactttctctggagtggcaagagcagtgggaagaattatgtcaatagctacagaaataagggagtaagaacaagtcatctctctagtgaattcttcttcactttactgagataaacatacatgttaatgagcttgagttttcccaaaagtataattcttctggttcttctaagaaaatggcactccctggaaacaaggaagaaccaaatttattcgcctttgtagcagttgggaaagttagtgctaggaagtcttcttgatttatagtaggctttaatctggatattgctggtaaaagtttattctaaaacctgaactctggataagtaatacaaaaagcttctcaaccttccaagcaaaattgagagctttcaggttatgtgagtaatttggtctcttgggtgcttaattcattccttgaagctcatttttgtgatctcttccaagattgcatttgcttggaggtagggagttagacaagatggtatgaggtccctaaattttgactttccaagcaaaattggacagtggttcctaaattgctaacatcctcgtttcttcctaaggcttctcatgtttcatatatagtagccttcccaaagtcccatttcccaccccccccccccccccaacccatgtagagagaacgaacctgtctcccttcctgtacagagtacgggatccttcaactttcacacaggctgcagtgtctgccacacatttagctcaacttttttttagccttaaagtgatgtccgctgcatctgtcgctgggttgcaccttgtggatttagtttgcataaattttctcagcttaaacaaagttaacattgaatagagtaagcttaccataaagggcttaataaatgccatgcatgtctacattcggtgtggaaattgagctagtcaggttgatatttaacattgtaggttctttgttaatttatatgaaataatggttatcatttaactctttaggttagctttgtacatagcatctcactttgcacaacaaccctgcaaggtaagtattgttattcttgtgctacaaatgaagttgactgagaggaggagtaccacgtccaaggtcacacagctattaaatggcagggctgggatactggcctgtgactcagaacttgatgctttccccccacgccacgcatgccaggttgcccttcctttcagaaatggtggaagtcctgcaaaatgcaataaactgaagtaatgtagcttctattaatacaaagtaaataactcagatttactggattttaaaccttattccttgggtaaacaatctgtgactgacttcacaccaaatatttgttggcggaggatttggactttagggataaaagtggatacattttttattttacaaactctgtatttgaacttaattattggctcttcaattttacgttaccagcttttttttttttttttttttttaatgaatttgatttacatcatggtcaaacaaaaattgttgagcagggaaaataaactgctttctggattccttcttgaattttctcatgtgccctagagaaaatgtgttccacattaaggtgttactttttccaggggtgtgttcatttaaaaagaatgaagccaggcaatgtttatttttcttttacctataaataaatgaatggattaatcattgtatacttgactcccatgttggtagggattttagataggaggctatttcttgtctgtgcttctcaataccccataagcagttgcttcatggatgtatatactaataagcagtgaaagaaagtgcatgttcaaagaatacaacaaggagtctggatattttgcaatcatctttatatattacggtgctctgaattaaaagctaaaagttactgggtatgtctgacaccttagtgctttatctttgttctactaattttctgtgccccaatcccacttaaccctagcctcattccttatctgtaagataggggataataccactgtaaggttattattaagattgaataaggataaaatttataatgggttttagcaaatggcagaaaatattttctgaagaaaaccaagtgctattaaaaaaacatcacaagccttgggcttactttgggattttaaaaaccaagagaaaatggatggctgaactttcaaacatttggtaaatattatagtactgtagttcagagctctggattctttgcattttgcctgctgggtgagaaggaataaaagtttgtgcctttttttttttttaatcactttaatttcaaaacaatgtgtttaaccatttgtgggagtaattttcattttgtgagcctgaagcattttgattcagtgggaatttctggtgatttatatctggaatagaagtgagcttaagtttagctattctaacgttgaaaaaggaagcaatgtttctattggattctaaagtatattttcaaaaatattctgaagtatttgtatatcttaaacttggagttaagacagcttagctttgaagataagagaaactagatgtgtgcattttctatccagatgtgtttgttgctggaactaaatgaaacagtacatggtaacccttgaaaggttttaaacttgtttctgtaactgctaatctacatactctcaagtcactaaccttcctctttgatctctttgtaggctgaccaactgactgaagagcagattgcaggtgagaaatactcagctagattgtacccattagattcttattataaattgaatagccaacgtcagaataagagacttgtatgaaataattgactttggtatatgtcatggatactaacatatgggtataatataatacagtaattcaacctgctttggcaagtgggattgtaaacttgccgatggaagatgtgcagggctaattgtagttaaagacatgtattttagtctgcgtggatatactttggagccttgtggtgtagtggtggtctttgttttgttttctgaagactaccttagtataaaacaggaattctgggctagctaacacgctagaaaaggaggccaagaagtaactaaggcaacttgaaatggagaatcaaaaaaggaacaattgaatttaccctagttaaaattagctaacattttaacctaagaattgcatatataatcagtcagggaataaccatttctcaagtgaaaaaatttaagaagttggcattctattctataaatgtcactgtagcaatgtggatttttctcttaaaaaacagtgacagctgttgaaatgaagccgcttaaatcttgtcccttttagtttgctatggaagtatgcaaaattattacataaaacctgatgtcatgttctctttatgccatgccatagaacattaatacttttgatttatccttgtacattgagatggctggttaatgtcaatgttggggaaacttttttttccccccttgccggagttgttttttttttttattgtagtgttgaaatgctttgcaaactattcctttgttttcattagacacttttattagcatgagcaccaagcagtttatttgccacagttttcacagtaagcaggaagggtttgataagagctcaaaagcacttgagcactttttcttaggctggagaaaactgagaagcccagttggaactatatagcccatttaaagcaagctccccacttccataagcatatagaagtttgggggataccagaattacactagtcacctcaaatactatttgataggactgtaggtagaaagctattagtattaaagacaaatttaagcaggttttatttttatttattgtgtaatttgacttaaacttagtttttactttctgaggttggtaaattacatgtattgactgttatgaacaaaaagcaaacagttttgggaataattttttttttgttgagttacactgttttaaaggtatcacctatttgatctcatgtctcctcaaaaaaaatagactgggcctgatggctggtaatcttatcactttgggaggctgaggcagcaggatcacttgagcccaggagtttgagaccagcctgggcaacatagtgagaccccatctctacaaagtaaatgtatcgtcaaaatgtttttctgaagtagttttttttataaatatactgtctccttcccaaattgtctcaggctgtctaatactttcactcttaaactctgaccttctgtaatacatttcttaatattcccagtcaggtacaggtaccaactgtatgcagtacagcatgttcactttagggtagaaaacgttactcagtgtgccattgcatagttgatggtctgctctcttaaacaactctttgaaaagaaagtttgttggccgggcgcagtggctcacgcctgtaattccagcactttgggaggccgaggcggatcacttgaggtcaggagttcatgaccagcctggccaacgtggtgaaaccccatctctcctaaaaatacaaaaattagccaggcatggtggcgtgcacctgtaatcccagccactcaggaggctgatgcaggagaatcacttgaacctgggaggcagaggttgcagtgaggcaagatcatgccactgcactccagcctgggcaacagagcgagactttgcctcaaaaaaaaaaaagtttgtgtctattgaatataagaggatccctcttcagtgattgatagcatttttattttttaatttattttgagacagtttcactcttgttgcccaggctggagtgcagtggctcaacttctgtctcccgggttcaagctgttctgcttcagtctcccgagtagctgggattagaggcatgcaccacctggctatttttgtatttttagtagagatggggtttctccatgttggtcaggctggtctcaaactcgcaacctcaggtgatccacccgcctcagcctcctaaagtgctgggattacaggtgtgaatcaccatgcctggcctggtagaattcttataaagaatacttgtagttcatcaaaactgtgacattcaggacctgttctctttattgtctttaagttaaatcctatcttttcttggctttcatcataaaattaggtttccaatgggagtagagggaaaaaaatctgttcttaggttctcttcttatcctgccttttcctagaccacagatttctttaaagccattccttggacctccaggtttcagttaagatttagggcagaggaaccaaaacttctatgctactaaaataaaaatcatgtgcggtcagggacctttttgttctactttgtaggcccggtacctgccacatagtaatcattgaatattccttgaataaattttattctcaatgagtgtgtaacacaaaaaagttctagagcactactatcctaagatttctgcctactctccaagtcccttcctcccaaatttggtatatttcagtgtttacctttgagatgaaggcaattttttaatctggtaaattgtaattgttggtggctgggcatggtggcgcatgtctaatcccaacactttgggaggctgaggcagtgggattgcttgagcccaggagttcaagaccagtctgggcaacacagcaagaccctgtctcaaagaaaagaaaaaaaaatttttttttttttttttttagttgtaagtgctggtgaccgctaacatccttgtgtagatgaagttactaaatttttttttttttttttttttttttttttttttttgagacggagcttcactctcttgcccaggctggagtgcagtagcatgatcttggctcactgcaacctttgctgtccaacttcaagcaattctcctggctcagtctcccaagtagctgggattacaggcgtgcaccaccacacccggctagtttttgtatttttaggagagatggggtttcaccatgttggccaggctggtcttgaactgctgacgtcaggtgatccgcttaccttgacctcccaaagtgttgggattacaggcatgagccactgctcccggcctaaaattttttatattacttggtcgtataaagcattccagtctagtatgacatcactaacaatttcattttgttgataactctgccatgatgtcattagttgttgctgtttgacttttggaagactgataggaaaaatgtacagtattttttccttaagaagtctgtacctaaactttcattaaattttcaaggtccaaatttgtttgagaaaaatattaatattactcaatgtatagcaacaactgcatgaagcgccaaaagagttccagtacatgatctttaagggattattaagttcagaattttgtgaagaaatagaagttaataaacagtcatttgatttgttttgtaattgtcttgtgataacctattttaataaaatttttaaaacatgttattttaataaaattatatgtttaggtaataaactaattagggaagggccttggggttgctaatgaacaaataaaacttgagtaatagatttttctgaaagtttcagtttggtcagctaaaatggattttggtaggttccttatatctatcaattatgtagaagatagctgttctgtggcattcaggacctgttctctttattgtgtgtttaggttttgactcttggatttaatttcaggtcattcctgggagttacagtttagaattatatgcttctatacataaagattacgctagttctgaagtacaggaaaactgtaaaggcaggtgaattaaaattttactttggggtttattattttgagtttttacaggtattttttactctttagtataaaaatacaagtaaaaatttggaataaaccttgacctttaaaagaaatgttatttgttcctttcttgaatgaataacaaatggtattagcatagatacggttttattttctgcattttacttattctagcaaacattttaagtgttattagatttttaaatttctcgaaagttatacatagtcttacactatgcatcttccagtatttagctttttgttaaatcatgtaatagaaaggtgattactgctaattaaacattgggaatttatgcacctaatggtggtgttaaaagatacccatagcagagaatgtaatataaacaaatacctcaaagtcctttaattttcttgtaacctaaattaggtgaaaggtgcaatgtgtctgaactccaatatatgtatttctagatatttctacattctgtttaaaaacagtaaaattgaaatgtctttatcaattgtgctatttgtaggtggagactccctgatctatttttgtcattaggttgatctaggttagcactgtatactatcttcataaaatcaattagtgtggtatgaaccttctgattggtattagatgagttcctctataaaccagtaatatttaaataagaaagaatgcagttaggaggcaattgtaatagccctggcaaaggacgatggagccttgaaccaaaatactggttgtagagatggaggaaaaatcaggcagtatttaaatgatagaattcgagacctgttgacagatttcagactgaaagagttaaaagtggctcttgggcttttgctagtacctttagggttgcctgggagtatagaattcattttcatatgttttgcttgtcatcagccaggggagctatatcctgtagacagccagaaataaagacatttattgagcgagatgggtagactaaagatatcttcgttgtaagtgggtatagatgaagtggccaaggagtggaaaaaaaggataaaattggtcacatctagacataagagacaggagtaagagcctttggcagtatatggtaggaggaaaatcaaagtactgttacgaaggccaagaaaaattttgagagtttttccatgcagttttggatgctgcaaaaaaatgagatagtctttatcttgacatttattaagtatgtatttgttggacatagttctaagtgtttgacacatactaatttttttatggggggtgaccaggtgaataccccttgtgggcatggtatactgtgtcaccccacttactgggcacatttatgtcatttcacagaaatgaggaagctgaggcagaaaaggtttagtagcttgtccatattgcattgccagaagaagaaacagagctaggaaattctaacccagtagtcaagttacaaagtctcattttagtttctatgccatatggcttcacattgaagcatgcttgatggattgggtgactgactagtagtcattggtaaactcagcattttcaggtgatgagttggagctggatgtcattttaaaatagtggattgtttggagagaggaaattggtatgagtagtagtgccttccaaaatttggggagggataagggcaagcatttaaaatataactaaggtttcaatttagttttaggctaagcgatgtgaagatatttatgtggtaatgggcaaattagtggaagaatagttgagtccagggaagaggtaatacggaatgaaactggaaggtgtacgcgaattggagttagggataagcaggcagggaataaattggccttggaccgtaaagaaggtacaccatttcttacacatgtgtcacataaattttcaaatgtatggctatgttgggaaagcttaatggcccaatgaacttggaaactttttgtattcatttgctgatagggtctaatattttctgggaaatgacttacttggaaaatactagctttattatttactgattggtataactttgcagtatcaaagttagtctgttttagtttctttctttctttttttttttttgagacaagtctcactctgttgcccaagctggagtgcagtggtgtgatctcggctcactgcaacctccgcctctcgggttcaagcagttctgctgctgcagcctccccagtagctgggactacaggcgcacgccgccacacccagcttgtgtgtgtgtttgtgttttaatagagacgggggactacaggcccacgctgccacacccagcgtgtgtgtgtgtgtgtgtgtgtgtgtgtgtgtgtgtgtgttttagtagagacgggggactacaggcacacgccgccacacccagcttgtgtgtgtgtgtgttttagtagagacggggtttcttttctttttcttctttttttttttttttgagacggagtctcgctctgtcgcccaggctggagtgcagtggcgcaatatcagctcactgcaagctctgcctcccaggttcacgccattctcctgcctcagcctcccgagtagctgggactataggcgcccgccaccacgcccggctaatttttttgtatttttagtagagatggggtttcaccgtgttagccaggatggtctcgatctcctgacctcatgatccacccgcctcagcctcccaaagtgctgggattacaggcttgagccaccgcgcccggcctgagacggggtttcaccctgttgttcaggctggtctggaactcaccctgagctcaggcagtctgcccaccttggcctcccaaagtgctaggattacaggtgtgagccactgcacctggcttgttttagtttatctgtaaatggtgaatgaagccaggtagtctggagtgttgcaccagcaggaggtttccatgtaatctagtgatgtattagaaggaacatatagccgggcacagtggctcatgccctgtaatcccagcactttgggaggcagagacaggcggatcacctgaggttgggagttcgaggccagcctgaccaacgtggagaaaccccatctctactaaaaatagaaaattagctgggcgtggtggtgcatgcctgtaattccagctactcgggaggctgaggcaggagaattgcttgatcctgggaggaggaggttgcggtgggctgagattgcaccattgcactccagcctggacaacaagagcgaaagtccatctcaaaaaaaaaaacaacagcaggaaggaacatactatcacaacaagatgttgaggaataatcagtttcactatataaggttggggttttaatattccaaagaaagctcaaaatgatctttgttctcagtgacatgcatttagctaaaactggacatttatatcttgatcagcccattgttatagactctttatatagtcccttctcctgccttactagaaagggaaagtagcctgttttaatttagtaggtgattgatttagacatagtggctaactctaaacacagtatacagaaacagtttaattactctgtagaaatacgtatttgggtaccagtagtcttatttttgttttgtgagtgtaggttaggtctgtaattttgaattatgttccacagagcagcttcagtgacagaggagccagtcatttggggatttggttcttttcctctgtaaacctaggataattttacttaggtcagcatgaaagtacagtggtatgattggtcaaagatatatttgaaatattatcataaggtatcataaagattggttaatctttaggagtcaaagtcacatttcaattgctacctccattggtatttttccgttaaaagtttttgggtttttctgagacgtggggtcttacagtttggccaggctgaagtgcagtggctgttcactggcacgatcccaccactgagtaggctttaagagttaatacgtttcatttaggttacattttattattaatctgtgtatttggattgtggtctttctttcaaacagaattcaaagaagctttttcactatttgacaaagatggtgatggaactataacaacaaaggaattgggaactgtaatgagatctcttgggcagaatcccacagaagcagagttacaggacatgattaatgaagtagatgctgatggtaagtcttcaattttgtggggctgggaggggttgaagaagtgatacttagatgtttccactaaacctttgctgtttgcttatatgtttccactaaagctttgatgtttccactaaaccatttcaggtaatggcacaattgacttccctgaatttctgacaatgatggcaagaaaaatgaaagacacagacagtgaagaagaaattagagaagcattccgtgtgtttgataaggtacgtcaaggactggatatgttaaattttgaggatgaaatgaagataccaggactcattatgaagacttgtgtttttttggttgtttttttttttttttttttttttttttttgatgatttaccaacatattttaattaccaagcaaaagctttttaagaattcagtatagactttctaatatatgtattgcgttcatcttgtcaaaggaggtgtcacttgactttgggactaggaaataatgatttcaatgtaggacattttgcttggctcttaatgacatttgtaggtcaactgtgtagatttgtatgcatttctaaagtatgtttttcccccaatgtgaaatttagtagtaaacttaataaatttccattccaactctcaagtctgtaatttgactcagaaactatactaaattttttgctaggatggcaatggctatattagtgctgcagaacttcgccatgtgatgacaaaccttggagagaagttaacagatgaagaagttgatgaaatgatcagggaagcagatattgatggtgatggtcaagtaaactatgaaggtaaatgtttcagtctcacgcctcattcttcactttgatttttaagataagaggctctttgttagggaactgatcgaaattagtgacccttcccctgaaattaggttgctaaaacagttacttacatttaaccttcttaaaattcagagtgttgtctgcagactaggtaacattgctctgacttgagaacttaatttagaaatatttactatcaggttctatccctacacctggaatcagcattttaataggattcttgggtgattcatgtataaagtttgagaagcactgttcagtgtttgtgtgattgtggaacaatgtggaaagcaaacttattccaagtttcctaaaatgaaattaatgtgactttctttccccgcactgtattgtcttttttctttaattggctttagtctgaaactgaagtttacatgtgcttcaaaagtatcgtatctactaacttttaagatggccatatcaggaagcaaggcatatgaaactgaaggattttgagtaaaactctgtatttttttcatgatgtaactacttaacttagaaaagagcataaaatgtaatgttataaaaattaaacctagcccatagcattgaatttacaatattgtcttgtaatccagcagacttaaaattcaagtctaaactaagagccaagaatgctcttttttatattggcctatctaaagcgttaatttggctcttactaataaacctgaatagttatggttaatgaagttccagcctgggcaataaactagcttccctctgtacttaatggtaattctgaggaaagagaggaatggaaagagtactcagtaattgagtaaatgtgattttttttttttttttttttttttggtcttttgctattgactcattttttgtgtacttcttctttttcagagtttgtacaaatgatgacagcaaagtgaagaccttgtacagaatgtgttaaatttcttgtacaaaattgtttatttgccttttctttgtttgtaacttatctgtaaaaggtttctccctactgtcaaaaaaatatgcatgtatagtaattaggacttcattcctccatgttttcttcccttatcttactgtcattgtcctaaaaccttattttagaaaattgatcaagtaacatgttgcatgtggcttactctggatatatctaagcccttctgcacatctaaacttagatggagttggtcaaatgagggaacatctgggttatgccttttttaaagtagttttctttaggaactgtcagcatgttgttgttgaagtgtggagttgtaactctgcgtggactatggacagtcaacaatatgtacttaaaagttgcactattgcaaaacgggtgtattatccaggtactcgtacactatttttttgtactgctggtcctgtaccagaaacattttcttttattgttacttgctttttaaactttgtttagccacttaaaatctgcttatggcacaatttgcctcaaaatccattccaagttgtatatttgttttccaataaaaaaattacaatttacccaatggttgctctgcatctgagtcatttaactgttgaagtctaataattttgaaaataaaatatggcattggtttctgcttggta

SEQ ID NO: 3 - DNA, 9528 bp, Homo sapiens
ggcggggcgcgcgcggcggccgttgagggaccgttggggcgggaggcggcggcggcggcggcgcgcgctgcgggcagtgagtgtggaggcgcggacgcgcggcggagctggaactgctgcagctgctgccgccgccggaggaaccttgatccccgtgctccggacaccccgggcctcgccatggtgagtgaggctggggggtcgccgaggctgcgggctctgaggcgggcttaacggggcaggacccctgagggggcgacagagcccagagtggggggcgtccgggcccggcgagagcctcgggacccttttctacccgcgttgtcgggggctgttgaacccagagcgggacgtctggatcaccagaggtttccagaagcgactttagcaccaaatgggatgtttaagtccacaaatgggtatctgagcccctaaaggggacatttgaaccccggatggaggatcgggaccctcagtgggacccctcaaaaggattttggacccaggatggggcgttgggtttcaggatggaggtgtttggaccccaggggacgaaataagaaagatgggctggggaggggagctattcgggccctgatgaaggcgtttggtcctgagaggatgcggggaacgggggacgaagggaggctgggctgctcctggatggggtggtggagggtagctgggcccgggcggggaggggcgacccctgggctcctgtggagcagaggagagggtcaaggatgggcggtcacttctacagatgagactcccgagggtgggggagggtgggggagggtggtctccagagcccagcactgcagaggaggaggagggtgaggtgcaagcttatgggaggaagagcctggggtgggtggcagcggagattagaagatactcttgaaggcttccgggacggagaaggatcagggtcgtggaggtggtgaaaagggatggtgagagtggggctcgccagtagtcgggggacagggacccttggggttaatttggaagtagctagaaaaggttccgaggttactagtgggttaagactgttgctctagaaagacagaatttttttaacggggtgggagtgttgtgtttgggtccaagagaagatggatttccaaggtgaggggtgggggaggacttgggcttgggtggttgggaggtgaggtggaaggagaaaatgaatggcagtcaggggggcctaggtttgggggtggatgtgggtagcaccctgccagtctccagggacaggccaggacaaagacggctcagtgtggatggggagtgggctgcagggcccggaggaagcttttctaccgtcctccttcctcaggaagaacgcggctgggtggaagacaccggggcagtgggagcaggtgggcggagctgttcgggcccctattgcgcactgaagcctcatgaaatggggatgggagatcaaagtttggggtgcctcgaatgagccagctttttcaagcctagaagcctcactctgtcgctgcccttttcaggccaagagcactgccttttcccagcaggagcgccggttttctcatgatccccagccccacgcaccatgctgcacctcgtcacttgtgccaaatgacagtgcagtacagtgtggcgtcagtgacaatcagatgcctattcccgtgccctcggggtgggggctgcagagcttgatcagagagtgcctctggggagaccccttgatcggcctgagggccacttgttccattcagggccctcgctgagcgtgtctgttaattcacagggctttgagagaaaaccagcagactagttctttcctactccctgccacccacaacacccacagccccaccaggctgagcctgggaaccgtgtgcagccatcctccctggcaaggctggggagaacaggcagccgctgctgtgtatcagatctcatacttgctgacacagacctgatggctcctcttgccgctaccccattttcccccagcagaacctggctaaaaggggccctgaagcactagcaggccccctacctccagaataggacccaagctttggccacaactagtttctctttctgtttgtaatcagcaaggtagaggtgggacaagtcactggtaactggccttggactttggcctgagctctgtggctcctccttggcccgggtgaggggccctggccttggtttctgatctcatctaacactgctctctggcattggctgggggctggggcaaggcagagaaatcacaccctcttccctcccccacctcatggttttttttcttgtccagccccttgctggagcctaagatgagggataaggaagcaggagaccagctgagttattttggaaacagtttttgttgaaagtttagtcttcaggtctagaaagggaagaaaccttgagcaaggatcaatgatatcttcttaggcggtcggttttctgggcggcaaccttgcttgagatctgtacctttccttgaccaagcagggaagtgggtctccaacatgggagggagagcatctgacaggccctccatgcagagccacgcctatttgcagcaaccctgtgacagaactgaacatggcagtgcctcatctgcatcttccagtcttttggtggagactggcaaacggtggccttcagagccgctcggggcagtctgcagcctgtcacagcctcctcgcccccacccccagccccttacagtgctgcccaaaggtccggggagctggacttcttgcagggcggaacagtgggctgggaaggccctgacctggctcgctttttccattctaggccaggggaaagaatgaaaagcagatgggatggcagccacctccccacactgctgccctcctctctgttctttccctgaccctccctgggcattgcctggtctcggtaaccagaagcccatcctcccttctcactttccctaagcctcttcctcaggtgtcaggacctaagggatggctccaggcttttttgtccatggtgggaggtggggggcaggtggatcaagaggctcatgggcctggggctaccaggttggcatttagaagcccaaccaccctgtcttttttgaaggcaatgcagaagaatcttcatttggcctccccacctcccctttacactttggacttttccagaatgtgcatgtgtgccgttttctcccttcctctttccccctccctaagccctctcccctctgtgatcatcattttgattacccctcccattctgcctatttggggcttgcaagttcttcttccgttaggttttttttttttttttctaacttttttagatcagtgattgttaatatttgggacggggatgggggtaggggaggcaggcagtggtttgttcctagcttctggaacctctccgtagaatactgcaaattcacataatagtctgcttctatttctgaggctctcagactcgagactagaacatgctatagacttggagcccctttccctgattgtgaaaggaaggggaacagacccaccatggtcccgggatggtggtgccttcttcacagcatcaacaaaaaccagggaaaatgtgtcttttggctgctaaagctaaatgttgggattcagcctccccaaccccccagccccgctcctgccccataggatcactatgcctggctgatgcccagcacaccagtgcaggagaagccagtccccgcatctcctccccacccccaaaccccgcatgcagcaagggctgcctgccacccactggccagtccccagagtgcccaagctggaaagggttaaaaactgcagtgcaagccagcttcagtcctctgctgctgcccgctgcagccctctccctgacgtttgtcccttctgcccaaaagaaacaaaacagaatggccctgtgccctcactgtgcgcagggggtggagcgcctgcctccagcctgaccagagttctttaggtgcttgagcaactcgggccgccggttaggagaatttttgccacttgggtggactctgcctgagccaaccgcctccaaataggtgacacagaaactagcagagatagccccagacatcgtccctctgccctacccaaggactggcctgcttcagtttggagccttctagagcttcttgatgaggcctccagggcaatcgttcccttcactcggcctctggggctctcactgatgagatgcaaacctgtgccaggcctcaccagcgctgctggtttggggctggggcaggcttttctcccgcccctgtggctcccacatgctgagttgagattggaagcccgtgggtctgggcagcttcatcgggccgtgcgtccagcaggcctgagtgtccagcccaccctggtacccagctttctgaatcaccagactttatcatcaggcgctggctctgcctcagaccctgactctggcatgctcctccctggcccggcgggaatggcacgtggagctggcctcactggggccgagggtctgggctgagtgaggaagatgcgtccgtgctgtccggcctggtgactgactttccccttcatgcttttacaggctgaccagctgactgaggagcagattgcaggtgagtctgctatccccctcatcttagctcaggaaagcctccacatccccagccaaatcttagccactgaggagtgacatctgatgggtgaacctgtgtatcccttgtgtcacctaacactatgccttgtgcctagaatgctgataaatgtgtgtaagagcacaccagtgccccaatgacacgaagccccatggccctgagacaggcctttaccagacaccaaaggctgtgttcctctcctgggcctgggtcatctaggggcttgatatcagtggctgcaggacgagcccaactctctctcccagcttcagccaggttggcttagaaggtgagctccctgcctccctgtggggcctgcagagctcctgtcccactttaggagccgcccaggagctggctcgtcactcccacctctgcgcttgcctgcgctttgcacttcctttcgcatgtgcctgcgcctgcctgttctgctggcggaagtgcctccccagtgctttgcatgggcagctccttcccgtcccttagctcagatgtcccctcggggaggccttccctgacccccatctcaagtacacagccttcctttactctccttcacatcactttattttcttcataacctttctcgctgtctgaaattattttcttatgtcttcacctgttgtatctcctgttactagatgtgagttccattgagtttgggacttggttttgttggtccctgctatagccttaatgcctgcctagaacgtagtaggtcctccgcaaacttttgctggctaaacaaacatctctctctcagcaagctagaagaaatccctggatcccatctaattcatttttctaacccctccaaaaccccagaagggtcagctacaggaactattgtgtaattacagatccgtgcctgcctacccgcccatactgagctgcccgaggacagagagcaactcttggtttatctccatagccaggggccagcacacagtagaccagagtagcgatccagagaatgggcctctgctccccaccagctacccagcccccagctctgctgtcaggttcaaccggctgtgtaacaagtactaaaggcccagcttctgcggtgggaggcaggaacggacttgccttctctcctttctcccctcaacccgcatcacagagtcacctttgcagcttcaaaattttggccctgggtggctatggagacccctgaggaaagccagaagatggcctggtttagtgatcatgagctcggggtgccccatgacccactcactacctgccctccccaagaacaacaaggcctcagcacagggaggtgccagcctccccaaagagactcttgccatgtccccagggcccagaggctcagggccacagcttcccagctgtgcatgtccctcccaatgcacctgccacccatctcccaactctggccttgtgcaacataaacggtctgcttttcaccctgtccccattagcaaactgtccaccgcctcaccccagcccacaccctctgggaataggctgccggacatctgtttttggagggcttgggggtagaggagcaccaggtgccaaaatgggcccagtgggagggcaagagccatgtgcttttctccctgcccctgatgcggccccagctgtggccttcccactcagcactgctagctcctcatcctcctggcttggcagccgtcccctcgctttccatccccccatcaaccccacgcactgtccgtcagccatgttctgctgccgaccacccccacgctcaccacacgcagtggctcagctcctttggaagctggtagcacccacggcaaggaacagggctgccagtcagaagggagtggatgcctgcatttcctcttcaggccggccagaggcattctgcagcctcccctcctcgtcccagccagccaaccctggctaacacactcagacaaagaatcctttcggctctcattcactccatgactttggttcatttcatcatttcaagggagaagtcaaagccagggaccaaacagaaatgtagaatcatctaaggacagctgtcagaccattttccccactgtcttcactggctggccagaaaaatagcttcacctagcacagtgttgatgttcaataactgttgaatgaaggttggaagggctttgccctgagcactgaggagagagagctggttgcgtgggacttgaagtctgtctcagtttctttaggtgggactgatgcccttggccttcctccagggaaggcatccagcatccagaggtaaggattctcctgtggaccttgtgacctctgactcctcccccttcttcccccagagttcaaggaggccttctccctctttgacaaggatggagatggcactatcaccaccaaggagttggggacagtgatgagatccctgggacagaaccccactgaagcagagctgcaggatatgatcaatgaggtggatgcagatggtgagccccacagagcgcgtgggcagccctgctcctggtcaccccgagtgactgcagggagcctctctcagggtgatggatgagcccgtgtctctcagggcccaggccaagagcattctccatcctttccccaccttccagggaacgggaccattgacttcccggagttcctgaccatgatggccagaaagatgaaggacacagacagtgaggaggagatccgagaggcgttccgtgtctttgacaaggtaagcagccctctccaggggcggctctgagactgacgccagccttcaggcagacaggcggaactggagccacggagctaccacttccaggaggtccgggtcccggtgccagcctcattgccaacctgctctgccacctcaggcagcctccctcactgtgctcactgctgagggatggtgatgacagccacccctctcactgcctctctccccaccgggagaagtgcccagtgaaaggctttatccccaacccccaggatgggaatggctacatcagcgccgcagagctgcgtcacgtaatgacgaacctgggggagaagctgaccgatgaggaggtggatgagatgatcagggaggctgacatcgatggagatggccaggtcaattatgaaggtgagtcaaggccaggcttgatctctggaacaagaagagaatcgcagcttcaggaacaagcccccagatccctcttgttggggagggccgctcgccacttagcctgcccgcctgacctcctctctctctgcttcactccacagagtttgtacagatgatgactgcaaagtgaaggccccccgggcagctggcgatgcccgttctcttgatctctctcttctcgcgcgcgcactctctcttcaacactcccctgcgtaccccggttctagcaaacaccaattgattgactgagaatctgataaagcaacaaaagatttgtcccaagctgcatgattgctctttctccttcttccctgagtctctctccatgcccctcatctcttccttttgccctcgcctcttccatccatgtcttccaaggcctgatgcattcataagttgaagccctccccagatccccttggggagcctctgccctcctccagcccggatggctctcctccattttggtttgtttcctcttgtttgtcatcttattttgggtgctggggtggctgccagccctgtcccgggacctgctgggagggacaagaggccctcccccaggcagaagagcatgccctttgccgttgcatgcaaccagccctgtgattccacgtgcagatcccagcagcctgttggggcaggggtgccaagagaggcattccagaaggactgagggggcgttgaggaattgtggcgttgactggatgtggcccaggagggggtcgagggggccaactcacagaaggggactgacagtgggcaacactcacatcccactggctgctgttctgaaaccatctgattggctttctgaggtttggctgggtggggactgctcatttggccactctgcaaattggacttgcccgcgttcctgaagcgctctcgagctgttctgtaaatacctggtgctaacatcccatgccgctccctcctcacgatgcacccaccgccctgagggcccgtcctaggaatggatgtggggatggtcgctttgtaatgtgctggttctctttttttttctttcccctctatggcccttaagactttcattttgttcagaaccatgctgggctagctaaagggtggggagagggaagatgggccccaccacgctctcaagagaacgcacctgcaataaaacagtcttgtcggccagctgcccaggggacggcagctacagcagcctctgcgtcctggtccgccagcacctcccgcttctccgtggtgacttggcgccgcttcctcacatctgtgctccgtgccctcttccctgcctcttccctcgcccacctgcctgcccccatactcccccagcggagagcatgatccgtgcccttgcttctgactttcgcctctgggacaagtaagtcaatgtgggcagttcagtcgtctgggttttttccccttttctgttcatttcatctggctccccccaccacctccccaccccaccccccaccccctgcttcccctcactgcccaggtcgatcaagtggcttttcctgggacctgcccagctttgagaatctcttctcatccaccctctggcacccagcctctgagggaaggagggatggggcatagtgggagacccagccaagagctgagggtaagggcaggtaggcgtgaggctgtggacattttcggaatgttttggttttgttttttttaaaccgggcaatattgtgttcagttcaagctgtgaagaaaaatatatatcaatgttttccaataaaatacagtgactacctga

SEQ ID NO: 4 - protein, 148 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 5 - RNA, 4268 nt, Homo sapiens
gauacggcgcaccauauauauaucgcggggcgcagacucgcgcuccggcaguggugcugggagugucguggacgccgugccguuacucguagucaggcggcggcgcaggcggcggcggcggcauagcgcacagcgcgccuuagcagcagcagcagcagcagcggcaucggagguacccccgccgucgcagcccccgcgcuggugcagccacccucgcucccucugcucuuccucccuucgcucgcaccauggcugaucagcugaccgaagaacagauugcugaauucaaggaagccuucucccuauuugauaaagauggcgauggcaccaucacaacaaaggaacuuggaacugucaugaggucacugggucagaacccaacagaagcugaauugcaggauaugaucaaugaaguggaugcugaugguaauggcaccauugacuuccccgaauuuuugacuaugauggcuagaaaaaugaaagauacagauagugaagaagaaauccgugaggcauuccgagucuuugacaaggauggcaaugguuauaucagugcagcagaacuacgucacgucaugacaaacuuaggagaaaaacuaacagaugaagaaguagaugaaaugaucagagaagcagauauugauggagacggacaagucaacuaugaagaauucguacagaugaugacugcaaaaugaagaccuacuuucaacuccuuuuuccccccucuagaagaaucaaauugaaucuuuuacuuaccucuugcaaaaaaaagaaaaaagaaaaaaguucauuuauucauucuguuucuauauagcaaaacugaaugucaaaaguaccuucuguccacacacacaaaaucugcauguauugguuggugguccuguccccuaaagaucaagcuacacaucaguuuuacaauauaaauacuuguacuaccuuaaugauaaggacuccuuaaaguuccauuugcuaaugauuaauacacuguuugggcuggccaguuuuucaugcaugcagcuugacgauugagcacagucaggccuuuguauuaaaaaugaaaaaugaaaaaacaaauucaaaaccuauucaaauggguucuaguucaauuuguuuaguauaaauugucauagcugguuuacugaaaacaaacacauuuaaaauugguuuaccucaggaugacgugcagaaaaaugggugaaggauaaaccguugagacguggccccacugguaggaugguccucuuguacuucgugugcuccgacccauggugacgaugacacacccugguggcaugcccguguauguugguuuagcguugucugcauuguucuagagugaaacaggugucaggcugucacuguucacacaaauuuuuaauaagaaacauuuaccaagggagcaucuuuggacucucuguuuuuaaaaccuucugaaccaugacuuggagccggcagaguaggcuguggcuguggacuucagcacaaccaucaacauugcuguucaaagaaauuacaguuuacguccauuccaaguuguaaaugcuagucuuuuuuuuuuuuuuuccaauaaaaagaccauuaacuuaaagugguguuaaaugcuuuguaaagcugagaucuaaauggggacaaggcagguggaggggaggccaguguacauguaaaugcccacagcccagcauuggguuucccucccaaggccccagcaccaaccucugagcccaagaccuugccugaaaacaagcagauaccgauugcuucauccuauuuauggacauguaggucuaguugcauuuucacuggggggaggggggaaggugaauuaugguaacuuuuaaugaucuauucaggcaguagagcucuuaaggaaaaaaaaaaacccacuuucucucaagcauguauuuagggguuguucucaauugugcugcugauuaccugucuuauguaacuacuugagaccaucugcaagagacaugauuuagugugucuguaauucaaucuucgcugugugugguagaagcaguagucacuuuuguaagccagucucuucaugccuaaaagacacuaccagucaccuuugauucgcgacuuuuaauuuaugauuauacuuagccuccuccuccuuuuuuuuuuuuucccaaguugacuugacuuugcuuuuuuccccccaaguagaacuaaugcuagcuuccagcuugaaaguaaaacuccaguguggagugaauuuugugucuaauuauaaaccuguaaccaaaacucagacaucugguacuggucuuugcauugagauuggucccuguaaaacccccuuuaaaagcauauugcauuuaguacagagcucuuuuuugaaaugaaggcuggagaugugcauuuuucacgguguuaacugguuguaucuuauuagcaaggagauugggguuuugaguguuugcgugggugguuucaauuugccagggaacaguggcaggcugcuagcaaggcagugagaagcucuuggcagccaaaugggugcauucagggcugauuuauagagacccuuggcuucuccuucuccuacucccugucuuucuggcauuuuguagcuuguuagauuuucugccagaggggugggucagagcaguggaggggagacaucgcccaugugcuucugcuacugguccuugggcugggugguugguagaggagauguugacacuaugagcuaaggguuggcuuuuguaauuaccugaaucugaaaggaaugccuaagguuaccuugggguuucucuucuggugagauaggguuccugguuugaguaaguuaauguccuggauauuucuuguggcaggggguggucaaagagccugauugcugacccagucucaggccuguggucgaugaccucucgguaguuucaaagggggcuggagggggauauuugacuuguuuuuucgaaauguagccuucuaacccucaagucuuuagaagcuggguggacucuuagugguccugcagcguauccuaaaagacuaccuuugaaacaggauucuuguauggccaggauccugucugggaaccagaaacccuacacccucccccuccagggaaugcugaguuccaguuuugagcagaggugaggcagaauccacuguagccuuccgcccugguauuuggggggaugaccagcccaggcguuggguguuagucugcaugaguuugugagaggaaauagcuggguguccuggcagugcccuugaaguugguuaggaccuuccuguaaacucuugccccuacuucuaacuacucuauaaauauauacauauauuuauauauaaagugauuaguugaacuggcauccugcuuuagccugagacuugccauaagaaacugcugaguacuuggcaaacccuuucauaguuuuguucuccaucuguuugggguagguguugagcgaggcaaauggaucucgauauuucagaugggcuuuugaugcacuguugccaaggaaggcuuuuucugauuuuuugacaaaugaauuuuugcacacuuucauuggugucuuucggcaacuuacacacauugaaaaugagcuauuguacauauuuuuauauucucuuuauaaaugcaugucugauuguacuuguaacaauauuguaaugaacggcugugcaguaggcccagcgcugcugugucucgucagaggaauagcuuaccacgaaccccucagcauacugggaaucucuuccugaacaacgaauguaaauuuggucaagucuacucuuccguucauucaauuauuuuaagcauuugaauuauuuauuguauauccuaaauauauuucuccuuuggcagugacuagauuuccacuaaugugucuuaaucuaucccuccagcuggcaguuacuguuuuuuuaauccccugaaguuguccuguaggagacagaaauucuuugcugucuguaucccuuggaguaagaagguaguggcauggguggaguguguguucuuucuccaaaucuauuaugauguuuauuaaacacuucuguagcaaagauggugguaguucuuuuguuacugaaguugcccuucaccauggcuauuugaaaaggagauguacuuggacguuucuguaaaucuugagauaaacuguuuggagauuuaaccaccucucugaugggggaccaacucuauggaaauuguaaauacguuuuauuuauaaaccuggcacuguauucaauaaacauuucugcagccuuucaucucuaacugcgaaaaaaaaaaaaaa

SEQ ID NO: 6 - RNA, 1309 nt, Homo sapiens
cucguuugcgauguuccguuaucuggaugcggcggagggaucuggcggagggagguguuuaugaggcgcugggggcggaggaggcgaauuaguccgaguggagagagcgagcugagugguuguguggucgcgucucggaaaccgguagcgcuugcagcauggcugaccaacugacugaagagcagauugcagaauucaaagaagcuuuuucacuauuugacaaagauggugauggaacuauaacaacaaaggaauugggaacuguaaugagaucucuugggcagaaucccacagaagcagaguuacaggacaugauuaaugaaguagaugcugaugguaauggcacaauugacuucccugaauuucugacaaugauggcaagaaaaaugaaagacacagacagugaagaagaaauuagagaagcauuccguguguuugauaaggauggcaauggcuauauuagugcugcagaacuucgccaugugaugacaaaccuuggagagaaguuaacagaugaagaaguugaugaaaugaucagggaagcagauauugauggugauggucaaguaaacuaugaagaguuuguacaaaugaugacagcaaagugaagaccuuguacagaauguguuaaauuucuuguacaaaauuguuuauuugccuuuucuuuguuuguaacuuaucuguaaaagguuucucccuacugucaaaaaaauaugcauguauaguaauuaggacuucauuccuccauguuuucuucccuuaucuuacugucauuguccuaaaaccuuauuuuagaaaauugaucaaguaacauguugcauguggcuuacucuggauauaucuaagcccuucugcacaucuaaacuuagauggaguuggucaaaugagggaacaucuggguuaugccuuuuuuaaaguaguuuucuuuaggaacugucagcauguuguuguugaaguguggaguuguaacucugcguggacuauggacagucaacaauauguacuuaaaaguugcacuauugcaaaacggguguauuauccagguacucguacacuauuuuuuuguacugcugguccuguaccagaaacauuuucuuuuauuguuacuugcuuuuuaaacuuuguuuagccacuuaaaaucugcuuauggcacaauuugccucaaaauccauuccaaguuguauauuuguuuuccaauaaaaaaauuacaauuuacccaaugguugcucugcaucugagucauuuaacuguugaagucuaauaauuuugaaaauaaaauauggcauugguuucugcuugguaaaaaaaa

SEQ ID NO: 7 - RNA, 2277 nt, Homo sapiens
ggcggggcgcgcgcggcggccguugagggaccguuggggcgggaggcggcggcggcggcggcgcgcgcugcgggcagugaguguggaggcgcggacgcgcggcggagcuggaacugcugcagcugcugccgccgccggaggaaccuugauccccgugcuccggacaccccgggccucgccauggcugaccagcugacugaggagcagauugcagaguucaaggaggccuucucccucuuugacaaggauggagauggcacuaucaccaccaaggaguuggggacagugaugagaucccugggacagaaccccacugaagcagagcugcaggauaugaucaaugagguggaugcagaugggaacgggaccauugacuucccggaguuccugaccaugauggccagaaagaugaaggacacagacagugaggaggagauccgagaggcguuccgugucuuugacaaggaugggaauggcuacaucagcgccgcagagcugcgucacguaaugacgaaccugggggagaagcugaccgaugaggagguggaugagaugaucagggaggcugacaucgauggagauggccaggucaauuaugaagaguuuguacagaugaugacugcaaagugaaggccccccgggcagcuggcgaugcccguucucuugaucucucucuucucgcgcgcgcacucucucuucaacacuccccugcguaccccgguucuagcaaacaccaauugauugacugagaaucugauaaagcaacaaaagauuugucccaagcugcaugauugcucuuucuccuucuucccugagucucucuccaugccccucaucucuuccuuuugcccucgccucuuccauccaugucuuccaaggccugaugcauucauaaguugaagcccuccccagauccccuuggggagccucugcccuccuccagcccggauggcucuccuccauuuugguuuguuuccucuuguuugucaucuuauuuugggugcugggguggcugccagcccugucccgggaccugcugggagggacaagaggcccucccccaggcagaagagcaugcccuuugccguugcaugcaaccagcccugugauuccacgugcagaucccagcagccuguuggggcaggggugccaagagaggcauuccagaaggacugagggggcguugaggaauuguggcguugacuggauguggcccaggagggggucgagggggccaacucacagaaggggacugacagugggcaacacucacaucccacuggcugcuguucugaaaccaucugauuggcuuucugagguuuggcuggguggggacugcucauuuggccacucugcaaauuggacuugcccgcguuccugaagcgcucucgagcuguucuguaaauaccuggugcuaacaucccaugccgcucccuccucacgaugcacccaccgcccugagggcccguccuaggaauggauguggggauggucgcuuuguaaugugcugguucucuuuuuuuuucuuuccccucuauggcccuuaagacuuucauuuuguucagaaccaugcugggcuagcuaaaggguggggagagggaagaugggccccaccacgcucucaagagaacgcaccugcaauaaaacagucuugucggccagcugcccaggggacggcagcuacagcagccucugcguccugguccgccagcaccucccgcuucuccguggugacuuggcgccgcuuccucacaucugugcuccgugcccucuucccugccucuucccucgcccaccugccugcccccauacucccccagcggagagcaugauccgugcccuugcuucugacuuucgccucugggacaaguaagucaaugugggcaguucagucgucuggguuuuuuccccuuuucuguucauuucaucuggcuccccccaccaccuccccaccccaccccccacccccugcuuccccucacugcccaggucgaucaaguggcuuuuccugggaccugcccagcuuugagaaucucuucucauccacccucuggcacccagccucugagggaaggagggauggggcauagugggagacccagccaagagcugaggguaagggcagguaggcgugaggcuguggacauuuucggaauguuuugguuuuguuuuuuuuaaaccgggcaauauuguguucaguucaagcugugaagaaaaauauauaucaauguuuuccaauaaaauacagugacuaccugaaaaaaaaaaaaaaaaaaa

SEQ ID NO: 8 - DNA, 11293 bp, Homo sapiens
gatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccatggtaggtcgggagtggcaaatgccggcgtagcagctgcccgagatttcttcccagatttctagttgttttgtttgttttttgtttgtttttggttcttggaggtttttcttttctgagtgttacgcagcagctgcgcttaaaggaggttgcattttggatttgcatctcggcgacctctgccagggagcttcatttattggttccccttggagctggacttggtcgtaggccgtccacgggcaggggctccggccgcaactgcagcgggggtttctgcatccaatccccctgccccccgcccagccccgcacccactgcatccactagcgccgcacccgggctgcctgcagcgcagcgtttcggcctgggagccgggcggggccgggcactagacccccccccccggcccgcccctccccaccccgcttctccgccggcgcgaaggtggcaggtcgggcgggcagtggagaatgaatgggctggagctggccggtggcgcacattgttccggccgggtgttgaggggcgcagtcagcgcccgccacctccccactttggccggccctgctgggcgccctccctcggtcgctctcccctccttcttcccggggggcgcggcgcgggcgtgggctgggaaggaaggagccggggaagggtggggttgggggcaggaaggcgaggggttgggggcggagagggcggaagcggcggccgggccgccctgcgcccgggcggggccctgcggtgtggccgtggcttgttcctgccgctttcgcaccctgcggccccccacccagtgcagcagtgcgggcgggcgtgagcctcggtgcaccaggaggcacttcccgcgggaggcgctgggctcgcgctaattggggcggggggggggggcggcgggggaggagggaactggcgcgcggcttggtttccattagagacgcaaagtttctgctccgggaggaggcggcggcgccgcgggctcgtcgcctgggggagcagaagcgggtgggaggtgcgggtggccttggcctcagccctggtgcgcgggggccgggggtggtgaccctcctggccgaggaggggcggcgtccagacgcccgctcgggggccgccttcccccccacgcctgcccccgggcacgcgccctgcccggtccctcgccccgcgccacttccagtccgcagagagatgccctccacgtttctgctttctctgcagcctctagattgccagatgcgactgtgcgcctcgctgggtgtgttttccacagccccttcctcctcggcgtgcagggctgacatcaccgactgcgtttctggtttggcgggtggggagatggttccccgcagggttctggtacacctttgcccccagggctagcgccatttgggggaggaggttttcgttgtcgagaaagttggatgctcctggtaacccctctaacaagagagttctgtagcgaggtgggactgttctccccataaggtgacagtttctcttgcgaggtgtggcagcgcttcctgttgtacaagacagatgttgccttggcgttacgtaaatcatcgtgtctccgtcatttaaagaaagccaatttttagtgattgaggtagaaagaaagatccgtttataatttgtaaaaacaaattttcacccagaatcaatatattggaacaccattcctactgttaaagttttcacttaagagtataaacttcatcagctttctattaggacttattttgtaattggcttcttaggcatccttctttaaaagagaaatccacgttagctctccttgaggtctcgagttccctcggctggaggcacaggttcagtggagaccaaataatgcaggtgaattaccttcgtggccattactgcctccaacgaagtgtgtttattaagaacagttcttatgtcattcttaaggtaggtagggttaatactctccagcaaatttagtagatactctttgccagaaaagagaggagtatatatagtttgataattattgtgtagttttctgtgtacttaatttttgcagttttgtaacacttcatttgtaagatggtaccattttttcctggcttctgaatcataggatagtttgacccagggcattagccattgtaatggtaggcttttaacaaataactgcctaatttaaaggattggaaagcatttgttacatggaaatgaagttggtggcgtacccagttgctgtatctttattttttctacttaattatttctcataaaatggatataaaagcctgttaatccaacccaatgccattatgtaacgccagtttggagatttcgagggcctggagcagtgcgcaaggtgcgctgaaagcctgcccctggatgagatccttatcctggctgtgatggcagtggcagtgggctgggtcccttgttgagtggaaagggggactgcggtgtccatggtgcagtaggtggcgctcttctgtcttagagcctgccgccactgcagctggtgccaaggggccttctgccactagaggtgccatttttcacatgatgaacttagcctagttagatcgcagagcaagctgtaagccatgggcccagaaaagaaaacttgaagtgagcagatgttgtcacttccttgtaatcctttgttaaaatagcataaggagttttctttattctatttactttcattaaatgaccgtgctacaggtttcaaaggattttaagattgatttttgaaagatcacaatattaaaagtataactggaaaacctatgttgaaatcaaccaaacatgtcgtggactgaatgataaccttttctttcttcatataggctgatcagctgaccgaagaacagattgctggtaagttgacaactccaaggagtccccagaaggccagaactaggcactgactcagttttggtgactcctctgttcctccccgctacagtctgggcagttttctaagaatttatttaaataagaacagtaagcagaaacactgaggtcagatgttattcttgccagtactttatagatgaggtgaaaggaagtaaaactaaggatgcccacatgttaaactctggagaatttgaccatgtttcacaatgtgcaaagtttgcgtatgattaattgtactgagcctgctactcagcggtttagtttacaattcttatgccatgggtctttcagtaatctgccacgaaagcttgtgctcgctatcctaaaataaatggaaatgggtgaatatgagtgttaggaccactgtagtaattgggaagaaagttacattagttaaactctgttgcccaggctggtctctaactcctgggctcaagcaatcctcctgcctcagcctcctgagtagctgggactacaggcatgtgccaccacgtctggcagattttagctttttaatattcctggaggacttgttttgagactgtttctcgttaggaaaccaggaatgcttctgaaatattctaaaagtcatgtggagagagtttacctgggaatgtacatttctagtaaccattttatttgttatgaaacaagggattcttatggctttagaaatgtaacaggaagggatttgaagggggcacatggaccaatcttgtcagattggatttagtcccttgaacctgggaggcaggggttgtagtgagctgagattgcaccactgcactccaatctcggtgacagagcgagactccattgtttaaaaaaaaaaaaaaagattggatttaggactaatttaagcatgttccagcttagccgccttgaaacctttgggaatattgtggtgtgtggcactgtttattgggagcagtgtttgctttatgggctgctgtatgaaggccagtccaacaggactattgtggtcattatttcagtagataaagaccagacttctgatacgttgcacaacttgaatggctggctttggcaagcccccggcaagtgtgtattgtgactgggttggataaagacattgattctaacgggtcaacttttgttttcagaattcaaggaagccttctccctatttgataaagatggcgatggcaccatcacaacaaaggaacttggaactgtcatgaggtcactgggtcagaacccaacagaagctgaattgcaggatatgatcattgaagtggatgctgatggtaagagctttaaaaccatgaatgagggccattgttgtgtaattcaagttcagacatgttacaggattgtctttcaggtccccagagcaaagcaaatgtgcaaagatcctttctgtggttgccccagggccattgacaattaaaatagaagatgatgggccttgcgtccatcctgcttagtgtctagaatgttttctgcatgggatcactattgttttcttcctgcttggtgcgacctagagctcaaatctatttttttttttttttttggagacggagtctcgccctgtcgcccaggctggagtggcactggcgcgatctcggctcactgcaacctctgcctcttgggttccagcgattctcctgcgtcagccttctgagtagctggaattacaggcgtgtgtcgccacgcccagttagtgttttgtatctttagtagagatggggtttcaccatgttggccaggctggtctcaaactcctgacctcgtgatccgccctccccggcctcccaaagtgctgggattacaggcgtgaaccactgctcctggccgagctcaaagcttttatcaactggcccatgagtctgcactgagtcttgaggggggaggtgaaattaaatagccatagaaagtgctttttaacaaacttactgtgtttaaagaggaggaggaacccccagatgaagtaggtgacgagcactcttagaagttaccataaaagtgagtacagtgtgagctgtagatgtgtttgctgcagaggagcatgtgaggtttggaggcggatgtgtggtgactccaggggatagatttgcagaacctaacggaaagggaagctgtaaggtgcagggccagagggaaccagcagtaaccctgatagcggtctgtcatctgttcctctcgactctacagcagcggacaacagaactttgattgctgatttccatcagtaagcaggctttgaagcacacttccccacccctaaaaaaaaaccacgtattttggtaaatcctatatatattctaatgtactgtatgacagtatagaacatgatttttaaaagatgagttgggaggagaaaaggataaaagaaaaaataaaagaagcattaagaataaacaattcggatctagattttactttctagatgattgactcgagggtggtgtagtaaaatcgcttgtctggtcacaaacatttggcagcagagcttttgattaggttctttgacaaagccttcagcacgttagagtggttttcactaatagtgttttggaaagaaaaggttgtccatagttctctagtttgctaagatgatcagctacccaggaacgtggagtaacttcctcttgtttgtgggagccccgggaatctgtgcctggggaggggagaagtctgttaggctcttggattgtgtggaagaaggagaagttgtgccaggctacagaatcctgtgtttgcactgagaaaacaggatggtacctgaccttctctgcatggctgtgagatagcttaaaataatttcttttgtttttgatgaatatgaacaatatcttaaaatttttgaggctaaaaaagtcttgaagggatccctgaggtattttctttgaaaggtactggtgaaaatgagtaacttaacctaagggtttttctttctaattttatttccatttagttcaatgacactgttagtctggagtgcttgtctttgggggtattcatctcttagttttaaagaggagttgtttggagtactggccgtagaacagattgttctgacagttccctaagtgttactagtctgagctgtgagaatgctcctgagcttttcccttaatgggaaataaagatactgagttggaagaaaacaggtggctaaccatcatagcgtggccaagaaatgatcctggagaagacttggtaagacttcatggcccatgcatggcataacagaatcaatgttcctctctcataatcttttctcctctgaaacactttatacacttaacctgcagctcagttctaggccttttttgtgttactgctgtcactaaccaaggcagagtgagacctgagtgatttccctaactcagggatggcagtcgggggcgctttcttccctcggagtggaaagattcagcctgcggagtggtgtatgctatttttctcttgaactgtacagcccttcatgacccttccatgggcttgaatccagatgtgcagtttcctttgtataattaaatactatcctgggcactgatgatgagtttgaaattatgtgaaattgccctgtgaagtgtttgaacgttttagacctgcagatgattgaacctagtaagatagtctgcccctttgtcctagtacatgtttaccgttccgtacagtggttctgaaatgattactgcagagcagcgttaatggagtgcttactttacatgagctttttgttttttaattcgaggtaatggcaccattgacttccccgaatttttgactatgatggctagaaaaatgaaagatacagatagtgaagaagaaatccgtgaggcattccgagtctttgacaaggtaatccagcatctacatagcagatggtacttaagtatggcttcttccgctttcacttctaaaagctataataatgttatagacagaagacttaaatctaactgcctgagcctctgatctcactttcaaaaatcctccttatggtaaccgtatcaggggagggtaggcataataaataggaattttggaccatgtttcttgactgttactttgaattgttgtgagcttttgcaaatcctgttttctgcattagctgtttgcatgtatttagtaggttagaggtgggaactagagatcagagaattgtttatggcagcagagttagcagtaacttgagagggcatagctaagtcaaagacctacttccccacactacatcattagcaataacaattgctgaatgttcacaggatggcaatggttatatcagtgcagcagaactacgtcacgtcatgacaaacttaggagaaaaactaacagatgaagaagtagatgaaatgatcagagaagcagatattgatggagacggacaagtcaactatgaaggtaaaactaaattctctgagctcagtgtttcatagtcttacctttagatctgtaagcaagccaactgcttcactagacagcctttgactttattttatgtacagtaaagatgttgtgttcattaaagctgttttcaaagataaccaaaagttactattatatttgtcttttcagaattcgtacagatgatgactgcaaaatgaagacctactttcaactcctttttcccccctctagaagaatcaaattgaatcttttacttacctcttgcaaaaaaaagaaaaaagaaaaaagttcatttattcattctgtttctatatagcaaaactgaatgtcaaaagtaccttctgtccacacacacaaaatctgcatgtattggttggtggtcctgtcccctaaagatcaagctacacatcagttttacaatataaatacttgtactaccttaatgataaggactccttaaagttccatttgctaatgattaatacactgtttgggctggccagtttttcatgcatgcagcttgacgattgagcacagtcaggcctttgtattaaaaatgaaaaatgaaaaaacaaattcaaaacctattcaaatgggttctagttcaatttgtttagtataaattgtcatagctggtttactgaaaacaaacacatttaaaattggtttacctcaggatgacgtgcagaaaaatgggtgaaggataaaccgttgagacgtggccccactggtaggatggtcctcttgtacttcgtgtgctccgacccatggtgacgatgacacaccctggtggcatgcccgtgtatgttggtttagcgttgtctgcattgttctagagtgaaacaggtgtcaggctgtcactgttcacacaaatttttaataagaaacatttaccaagggagcatctttggactctctgtttttaaaaccttctgaaccatgacttggagccggcagagtaggctgtggctgtggacttcagcacaaccatcaacattgctgttcaaagaaattacagtttacgtccattccaagttgtaaatgctagtctttttttttttttttccaataaaaagaccattaacttaaagtggtgttaaatgctttgtaaagctgagatctaaatggggacaaggcaggtggaggggaggccagtgtacatgtaaatgcccacagcccagcattgggtttccctcccaaggccccagcaccaacctctgagcccaagaccttgcctgaaaacaagcagataccgattgcttcatcctatttatggacatgtaggtctagttgcattttcactggggggaggggggaaggtgaattatggtaacttttaatgatctattcaggcagtagagctcttaaggaaaaaaaaaaacccactttctctcaagcatgtatttaggggttgttctcaattgtgctgctgattacctgtcttatgtaactacttgagaccatctgcaagagacatgatttagtgtgtctgtaattcaatcttcgctgtgtgtggtagaagcagtagtcacttttgtaagccagtctcttcatgcctaaaagacactaccagtcacctttgattcgcgacttttaatttatgattatacttagcctcctcctcctttttttttttttcccaagttgacttgactttgcttttttccccccaagtagaactaatgctagcttccagcttgaaagtaaaactccagtgtggagtgaattttgtgtctaattataaacctgtaaccaaaactcagacatctggtactggtctttgcattgagattggtccctgtaaaaccccctttaaaagcatattgcatttagtacagagctcttttttgaaatgaaggctggagatgtgcatttttcacggtgttaactggttgtatcttattagcaaggagattggggttttgagtgtttgcgtgggtggtttcaatttgccagggaacagtggcaggctgctagcaaggcagtgagaagctcttggcagccaaatgggtgcattcagggctgatttatagagacccttggcttctccttctcctactccctgtctttctggcattttgtagcttgttagattttctgccagaggggtgggtcagagcagtggaggggagacatcgcccatgtgcttctgctactggtccttgggctgggtggttggtagaggagatgttgacactatgagctaagggttggcttttgtaattacctgaatctgaaaggaatgcctaaggttaccttggggtttctcttctggtgagatagggttcctggtttgagtaagttaatgtcctggatatttcttgtggcagggggtggtcaaagagcctgattgctgacccagtctcaggcctgtggtcgatgacctctcggtagtttcaaagggggctggagggggatatttgacttgttttttcgaaatgtagccttctaaccctcaagtctttagaagctgggtggactcttagtggtcctgcagcgtatcctaaaagactacctttgaaacaggattcttgtatggccaggatcctgtctgggaaccagaaaccctacaccctccccctccagggaatgctgagttccagttttgagcagaggtgaggcagaatccactgtagccttccgccctggtatttggggggatgaccagcccaggcgttgggtgttagtctgcatgagtttgtgagaggaaatagctgggtgtcctggcagtgcccttgaagttggttaggaccttcctgtaaactcttgcccctacttctaactactctataaatatatacatatatttatatataaagtgattagttgaactggcatcctgctttagcctgagacttgccataagaaactgctgagtacttggcaaaccctttcatagttttgttctccatctgtttggggtaggtgttgagcgaggcaaatggatctcgatatttcagatgggcttttgatgcactgttgccaaggaaggctttttctgattttttgacaaatgaatttttgcacactttcattggtgtctttcggcaacttacacacattgaaaatgagctattgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcagttactgtttttttaatcccctgaagttgtcctgtaggagacagaaattctttgctgtctgtatcccttggagtaagaaggtagtggcatgggtggagtgtgtgttctttctccaaatctattatgatgtttattaaacacttctgtagcaaagatggtggtagttcttttgttactgaagttgcccttcaccatggctatttgaaaaggagatgtacttggacgtttctgtaaatcttgagataaactgtttggagatttaaccacctctctgatgggggaccaactctatggaaattgtaaatacgttttatttataaacctggcactgtattcaataaacatttctgcagcctttcatctctaactgcgaa

SEQ ID NO: 9 - RNA, 4268 nt, Homo sapiens
gauacggcgcaccauauauauaucgcggggcgcagacucgcgcuccggcaguggugcugggagugucguggacgccgugccguuacucguagucaggcggcggcgcaggcggcggcggcggcauagcgcacagcgcgccuuagcagcagcagcagcagcagcggcaucggagguacccccgccgucgcagcccccgcgcuggugcagccacccucgcucccucugcucuuccucccuucgcucgcaccauggcugaucagcugaccgaagaacagauugcugaauucaaggaagccuucucccuauuugauaaagauggcgauggcaccaucacaacaaaggaacuuggaacugucaugaggucacugggucagaacccaacagaagcugaauugcaggauaugaucauugaaguggaugcugaugguaauggcaccauugacuuccccgaauuuuugacuaugauggcuagaaaaaugaaagauacagauagugaagaagaaauccgugaggcauuccgagucuuugacaaggauggcaaugguuauaucagugcagcagaacuacgucacgucaugacaaacuuaggagaaaaacuaacagaugaagaaguagaugaaaugaucagagaagcagauauugauggagacggacaagucaacuaugaagaauucguacagaugaugacugcaaaaugaagaccuacuuucaacuccuuuuuccccccucuagaagaaucaaauugaaucuuuuacuuaccucuugcaaaaaaaagaaaaaagaaaaaaguucauuuauucauucuguuucuauauagcaaaacugaaugucaaaaguaccuucuguccacacacacaaaaucugcauguauugguuggugguccuguccccuaaagaucaagcuacacaucaguuuuacaauauaaauacuuguacuaccuuaaugauaaggacuccuuaaaguuccauuugcuaaugauuaauacacuguuugggcuggccaguuuuucaugcaugcagcuugacgauugagcacagucaggccuuuguauuaaaaaugaaaaaugaaaaaacaaauucaaaaccuauucaaauggguucuaguucaauuuguuuaguauaaauugucauagcugguuuacugaaaacaaacacauuuaaaauugguuuaccucaggaugacgugcagaaaaaugggugaaggauaaaccguugagacguggccccacugguaggaugguccucuuguacuucgugugcuccgacccauggugacgaugacacacccugguggcaugcccguguauguugguuuagcguugucugcauuguucuagagugaaacaggugucaggcugucacuguucacacaaauuuuuaauaagaaacauuuaccaagggagcaucuuuggacucucuguuuuuaaaaccuucugaaccaugacuuggagccggcagaguaggcuguggcuguggacuucagcacaaccaucaacauugcuguucaaagaaauuacaguuuacguccauuccaaguuguaaaugcuagucuuuuuuuuuuuuuuuccaauaaaaagaccauuaacuuaaagugguguuaaaugcuuuguaaagcugagaucuaaauggggacaaggcagguggaggggaggccaguguacauguaaaugcccacagcccagcauuggguuucccucccaaggccccagcaccaaccucugagcccaagaccuugccugaaaacaagcagauaccgauugcuucauccuauuuauggacauguaggucuaguugcauuuucacuggggggaggggggaaggugaauuaugguaacuuuuaaugaucuauucaggcaguagagcucuuaaggaaaaaaaaaaacccacuuucucucaagcauguauuuagggguuguucucaauugugcugcugauuaccugucuuauguaacuacuugagaccaucugcaagagacaugauuuagugugucuguaauucaaucuucgcugugugugguagaagcaguagucacuuuuguaagccagucucuucaugccuaaaagacacuaccagucaccuuugauucgcgacuuuuaauuuaugauuauacuuagccuccuccuccuuuuuuuuuuuuucccaaguugacuugacuuugcuuuuuuccccccaaguagaacuaaugcuagcuuccagcuugaaaguaaaacuccaguguggagugaauuuugugucuaauuauaaaccuguaaccaaaacucagacaucugguacuggucuuugcauugagauuggucccuguaaaacccccuuuaaaagcauauugcauuuaguacagagcucuuuuuugaaaugaaggcuggagaugugcauuuuucacgguguuaacugguuguaucuuauuagcaaggagauugggguuuugaguguuugcgugggugguuucaauuugccagggaacaguggcaggcugcuagcaaggcagugagaagcucuuggcagccaaaugggugcauucagggcugauuuauagagacccuuggcuucuccuucuccuacucccugucuuucuggcauuuuguagcuuguuagauuuucugccagaggggugggucagagcaguggaggggagacaucgcccaugugcuucugcuacugguccuugggcugggugguugguagaggagauguugacacuaugagcuaaggguuggcuuuuguaauuaccugaaucugaaaggaaugccuaagguuaccuugggguuucucuucuggugagauaggguuccugguuugaguaaguuaauguccuggauauuucuuguggcaggggguggucaaagagccugauugcugacccagucucaggccuguggucgaugaccucucgguaguuucaaagggggcuggagggggauauuugacuuguuuuuucgaaauguagccuucuaacccucaagucuuuagaagcuggguggacucuuagugguccugcagcguauccuaaaagacuaccuuugaaacaggauucuuguauggccaggauccugucugggaaccagaaacccuacacccucccccuccagggaaugcugaguuccaguuuugagcagaggugaggcagaauccacuguagccuuccgcccugguauuuggggggaugaccagcccaggcguuggguguuagucugcaugaguuugugagaggaaauagcuggguguccuggcagugcccuugaaguugguuaggaccuuccuguaaacucuugccccuacuucuaacuacucuauaaauauauacauauauuuauauauaaagugauuaguugaacuggcauccugcuuuagccugagacuugccauaagaaacugcugaguacuuggcaaacccuuucauaguuuuguucuccaucuguuugggguagguguugagcgaggcaaauggaucucgauauuucagaugggcuuuugaugcacuguugccaaggaaggcuuuuucugauuuuuugacaaaugaauuuuugcacacuuucauuggugucuuucggcaacuuacacacauugaaaaugagcuauuguacauauuuuuauauucucuuuauaaaugcaugucugauuguacuuguaacaauauuguaaugaacggcugugcaguaggcccagcgcugcugugucucgucagaggaauagcuuaccacgaaccccucagcauacugggaaucucuuccugaacaacgaauguaaauuuggucaagucuacucuuccguucauucaauuauuuuaagcauuugaauuauuuauuguauauccuaaauauauuucuccuuuggcagugacuagauuuccacuaaugugucuuaaucuaucccuccagcuggcaguuacuguuuuuuuaauccccugaaguuguccuguaggagacagaaauucuuugcugucuguaucccuuggaguaagaagguaguggcauggguggaguguguguucuuucuccaaaucuauuaugauguuuauuaaacacuucuguagcaaagauggugguaguucuuuuguuacugaaguugcccuucaccauggcuauuugaaaaggagauguacuuggacguuucuguaaaucuugagauaaacuguuuggagauuuaaccaccucucugaugggggaccaacucuauggaaauuguaaauacguuuuauuuauaaaccuggcacuguauucaauaaacauuucugcagccuuucaucucuaacugcgaaaaaaaaaaaaaa

SEQ ID NO: 10 - protein, 148 aa, Homo sapiens
ADQLTEEQIAEFKEAFSLFDKDGDGTITTKELGTVMRSLGQNPTEAELQDMIIEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 11 - DNA, 11293 bp, Homo sapiens
gatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccatggtaggtcgggagtggcaaatgccggcgtagcagctgcccgagatttcttcccagatttctagttgttttgtttgttttttgtttgtttttggttcttggaggtttttcttttctgagtgttacgcagcagctgcgcttaaaggaggttgcattttggatttgcatctcggcgacctctgccagggagcttcatttattggttccccttggagctggacttggtcgtaggccgtccacgggcaggggctccggccgcaactgcagcgggggtttctgcatccaatccccctgccccccgcccagccccgcacccactgcatccactagcgccgcacccgggctgcctgcagcgcagcgtttcggcctgggagccgggcggggccgggcactagacccccccccccggcccgcccctccccaccccgcttctccgccggcgcgaaggtggcaggtcgggcgggcagtggagaatgaatgggctggagctggccggtggcgcacattgttccggccgggtgttgaggggcgcagtcagcgcccgccacctccccactttggccggccctgctgggcgccctccctcggtcgctctcccctccttcttcccggggggcgcggcgcgggcgtgggctgggaaggaaggagccggggaagggtggggttgggggcaggaaggcgaggggttgggggcggagagggcggaagcggcggccgggccgccctgcgcccgggcggggccctgcggtgtggccgtggcttgttcctgccgctttcgcaccctgcggccccccacccagtgcagcagtgcgggcgggcgtgagcctcggtgcaccaggaggcacttcccgcgggaggcgctgggctcgcgctaattggggcggggggggggggcggcgggggaggagggaactggcgcgcggcttggtttccattagagacgcaaagtttctgctccgggaggaggcggcggcgccgcgggctcgtcgcctgggggagcagaagcgggtgggaggtgcgggtggccttggcctcagccctggtgcgcgggggccgggggtggtgaccctcctggccgaggaggggcggcgtccagacgcccgctcgggggccgccttcccccccacgcctgcccccgggcacgcgccctgcccggtccctcgccccgcgccacttccagtccgcagagagatgccctccacgtttctgctttctctgcagcctctagattgccagatgcgactgtgcgcctcgctgggtgtgttttccacagccccttcctcctcggcgtgcagggctgacatcaccgactgcgtttctggtttggcgggtggggagatggttccccgcagggttctggtacacctttgcccccagggctagcgccatttgggggaggaggttttcgttgtcgagaaagttggatgctcctggtaacccctctaacaagagagttctgtagcgaggtgggactgttctccccataaggtgacagtttctcttgcgaggtgtggcagcgcttcctgttgtacaagacagatgttgccttggcgttacgtaaatcatcgtgtctccgtcatttaaagaaagccaatttttagtgattgaggtagaaagaaagatccgtttataatttgtaaaaacaaattttcacccagaatcaatatattggaacaccattcctactgttaaagttttcacttaagagtataaacttcatcagctttctattaggacttattttgtaattggcttcttaggcatccttctttaaaagagaaatccacgttagctctccttgaggtctcgagttccctcggctggaggcacaggttcagtggagaccaaataatgcaggtgaattaccttcgtggccattactgcctccaacgaagtgtgtttattaagaacagttcttatgtcattcttaaggtaggtagggttaatactctccagcaaatttagtagatactctttgccagaaaagagaggagtatatatagtttgataattattgtgtagttttctgtgtacttaatttttgcagttttgtaacacttcatttgtaagatggtaccattttttcctggcttctgaatcataggatagtttgacccagggcattagccattgtaatggtaggcttttaacaaataactgcctaatttaaaggattggaaagcatttgttacatggaaatgaagttggtggcgtacccagttgctgtatctttattttttctacttaattatttctcataaaatggatataaaagcctgttaatccaacccaatgccattatgtaacgccagtttggagatttcgagggcctggagcagtgcgcaaggtgcgctgaaagcctgcccctggatgagatccttatcctggctgtgatggcagtggcagtgggctgggtcccttgttgagtggaaagggggactgcggtgtccatggtgcagtaggtggcgctcttctgtcttagagcctgccgccactgcagctggtgccaaggggccttctgccactagaggtgccatttttcacatgatgaacttagcctagttagatcgcagagcaagctgtaagccatgggcccagaaaagaaaacttgaagtgagcagatgttgtcacttccttgtaatcctttgttaaaatagcataaggagttttctttattctatttactttcattaaatgaccgtgctacaggtttcaaaggattttaagattgatttttgaaagatcacaatattaaaagtataactggaaaacctatgttgaaatcaaccaaacatgtcgtggactgaatgataaccttttctttcttcatataggctgatcagctgaccgaagaacagattgctggtaagttgacaactccaaggagtccccagaaggccagaactaggcactgactcagttttggtgactcctctgttcctccccgctacagtctgggcagttttctaagaatttatttaaataagaacagtaagcagaaacactgaggtcagatgttattcttgccagtactttatagatgaggtgaaaggaagtaaaactaaggatgcccacatgttaaactctggagaatttgaccatgtttcacaatgtgcaaagtttgcgtatgattaattgtactgagcctgctactcagcggtttagtttacaattcttatgccatgggtctttcagtaatctgccacgaaagcttgtgctcgctatcctaaaataaatggaaatgggtgaatatgagtgttaggaccactgtagtaattgggaagaaagttacattagttaaactctgttgcccaggctggtctctaactcctgggctcaagcaatcctcctgcctcagcctcctgagtagctgggactacaggcatgtgccaccacgtctggcagattttagctttttaatattcctggaggacttgttttgagactgtttctcgttaggaaaccaggaatgcttctgaaatattctaaaagtcatgtggagagagtttacctgggaatgtacatttctagtaaccattttatttgttatgaaacaagggattcttatggctttagaaatgtaacaggaagggatttgaagggggcacatggaccaatcttgtcagattggatttagtcccttgaacctgggaggcaggggttgtagtgagctgagattgcaccactgcactccaatctcggtgacagagcgagactccattgtttaaaaaaaaaaaaaaagattggatttaggactaatttaagcatgttccagcttagccgccttgaaacctttgggaatattgtggtgtgtggcactgtttattgggagcagtgtttgctttatgggctgctgtatgaaggccagtccaacaggactattgtggtcattatttcagtagataaagaccagacttctgatacgttgcacaacttgaatggctggctttggcaagcccccggcaagtgtgtattgtgactgggttggataaagacattgattctaacgggtcaacttttgttttcagaattcaaggaagccttctccctatttgataaagatggcgatggcaccatcacaacaaaggaacttggaactgtcatgaggtcactgggtcagaacccaacagaagctgaattgcaggatatgatcaatgaagtggatgctgatggtaagagctttaaaaccatgaatgagggccattgttgtgtaattcaagttcagacatgttacaggattgtctttcaggtccccagagcaaagcaaatgtgcaaagatcctttctgtggttgccccagggccattgacaattaaaatagaagatgatgggccttgcgtccatcctgcttagtgtctagaatgttttctgcatgggatcactattgttttcttcctgcttggtgcgacctagagctcaaatctatttttttttttttttttggagacggagtctcgccctgtcgcccaggctggagtggcactggcgcgatctcggctcactgcaacctctgcctcttgggttccagcgattctcctgcgtcagccttctgagtagctggaattacaggcgtgtgtcgccacgcccagttagtgttttgtatctttagtagagatggggtttcaccatgttggccaggctggtctcaaactcctgacctcgtgatccgccctccccggcctcccaaagtgctgggattacaggcgtgaaccactgctcctggccgagctcaaagcttttatcaactggcccatgagtctgcactgagtcttgaggggggaggtgaaattaaatagccatagaaagtgctttttaacaaacttactgtgtttaaagaggaggaggaacccccagatgaagtaggtgacgagcactcttagaagttaccataaaagtgagtacagtgtgagctgtagatgtgtttgctgcagaggagcatgtgaggtttggaggcggatgtgtggtgactccaggggatagatttgcagaacctaacggaaagggaagctgtaaggtgcagggccagagggaaccagcagtaaccctgatagcggtctgtcatctgttcctctcgactctacagcagcggacaacagaactttgattgctgatttccatcagtaagcaggctttgaagcacacttccccacccctaaaaaaaaaccacgtattttggtaaatcctatatatattctaatgtactgtatgacagtatagaacatgatttttaaaagatgagttgggaggagaaaaggataaaagaaaaaataaaagaagcattaagaataaacaattcggatctagattttactttctagatgattgactcgagggtggtgtagtaaaatcgcttgtctggtcacaaacatttggcagcagagcttttgattaggttctttgacaaagccttcagcacgttagagtggttttcactaatagtgttttggaaagaaaaggttgtccatagttctctagtttgctaagatgatcagctacccaggaacgtggagtaacttcctcttgtttgtgggagccccgggaatctgtgcctggggaggggagaagtctgttaggctcttggattgtgtggaagaaggagaagttgtgccaggctacagaatcctgtgtttgcactgagaaaacaggatggtacctgaccttctctgcatggctgtgagatagcttaaaataatttcttttgtttttgatgaatatgaacaatatcttaaaatttttgaggctaaaaaagtcttgaagggatccctgaggtattttctttgaaaggtactggtgaaaatgagtaacttaacctaagggtttttctttctaattttatttccatttagttcaatgacactgttagtctggagtgcttgtctttgggggtattcatctcttagttttaaagaggagttgtttggagtactggccgtagaacagattgttctgacagttccctaagtgttactagtctgagctgtgagaatgctcctgagcttttcccttaatgggaaataaagatactgagttggaagaaaacaggtggctaaccatcatagcgtggccaagaaatgatcctggagaagacttggtaagacttcatggcccatgcatggcataacagaatcaatgttcctctctcataatcttttctcctctgaaacactttatacacttaacctgcagctcagttctaggccttttttgtgttactgctgtcactaaccaaggcagagtgagacctgagtgatttccctaactcagggatggcagtcgggggcgctttcttccctcggagtggaaagattcagcctgcggagtggtgtatgctatttttctcttgaactgtacagcccttcatgacccttccatgggcttgaatccagatgtgcagtttcctttgtataattaaatactatcctgggcactgatgatgagtttgaaattatgtgaaattgccctgtgaagtgtttgaacgttttagacctgcagatgattgaacctagtaagatagtctgcccctttgtcctagtacatgtttaccgttccgtacagtggttctgaaatgattactgcagagcagcgttaatggagtgcttactttacatgagctttttgttttttaattcgaggtaatggcaccattgacttccccgaatttttgactatgatggctagaaaaatgaaagatacagatagtgaagaagaaatccgtgaggcattccgagtctttgacaaggtaatccagcatctacatagcagatggtacttaagtatggcttcttccgctttcacttctaaaagctataataatgttatagacagaagacttaaatctaactgcctgagcctctgatctcactttcaaaaatcctccttatggtaaccgtatcaggggagggtaggcataataaataggaattttggaccatgtttcttgactgttactttgaattgttgtgagcttttgcaaatcctgttttctgcattagctgtttgcatgtatttagtaggttagaggtgggaactagagatcagagaattgtttatggcagcagagttagcagtaacttgagagggcatagctaagtcaaagacctacttccccacactacatcattagcaataacaattgctgaatgttcacaggatggcagtggttatatcagtgcagcagaactacgtcacgtcatgacaaacttaggagaaaaactaacagatgaagaagtagatgaaatgatcagagaagcagatattgatggagacggacaagtcaactatgaaggtaaaactaaattctctgagctcagtgtttcatagtcttacctttagatctgtaagcaagccaactgcttcactagacagcctttgactttattttatgtacagtaaagatgttgtgttcattaaagctgttttcaaagataaccaaaagttactattatatttgtcttttcagaattcgtacagatgatgactgcaaaatgaagacctactttcaactcctttttcccccctctagaagaatcaaattgaatcttttacttacctcttgcaaaaaaaagaaaaaagaaaaaagttcatttattcattctgtttctatatagcaaaactgaatgtcaaaagtaccttctgtccacacacacaaaatctgcatgtattggttggtggtcctgtcccctaaagatcaagctacacatcagttttacaatataaatacttgtactaccttaatgataaggactccttaaagttccatttgctaatgattaatacactgtttgggctggccagtttttcatgcatgcagcttgacgattgagcacagtcaggcctttgtattaaaaatgaaaaatgaaaaaacaaattcaaaacctattcaaatgggttctagttcaatttgtttagtataaattgtcatagctggtttactgaaaacaaacacatttaaaattggtttacctcaggatgacgtgcagaaaaatgggtgaaggataaaccgttgagacgtggccccactggtaggatggtcctcttgtacttcgtgtgctccgacccatggtgacgatgacacaccctggtggcatgcccgtgtatgttggtttagcgttgtctgcattgttctagagtgaaacaggtgtcaggctgtcactgttcacacaaatttttaataagaaacatttaccaagggagcatctttggactctctgtttttaaaaccttctgaaccatgacttggagccggcagagtaggctgtggctgtggacttcagcacaaccatcaacattgctgttcaaagaaattacagtttacgtccattccaagttgtaaatgctagtctttttttttttttttccaataaaaagaccattaacttaaagtggtgttaaatgctttgtaaagctgagatctaaatggggacaaggcaggtggaggggaggccagtgtacatgtaaatgcccacagcccagcattgggtttccctcccaaggccccagcaccaacctctgagcccaagaccttgcctgaaaacaagcagataccgattgcttcatcctatttatggacatgtaggtctagttgcattttcactggggggaggggggaaggtgaattatggtaacttttaatgatctattcaggcagtagagctcttaaggaaaaaaaaaaacccactttctctcaagcatgtatttaggggttgttctcaattgtgctgctgattacctgtcttatgtaactacttgagaccatctgcaagagacatgatttagtgtgtctgtaattcaatcttcgctgtgtgtggtagaagcagtagtcacttttgtaagccagtctcttcatgcctaaaagacactaccagtcacctttgattcgcgacttttaatttatgattatacttagcctcctcctcctttttttttttttcccaagttgacttgactttgcttttttccccccaagtagaactaatgctagcttccagcttgaaagtaaaactccagtgtggagtgaattttgtgtctaattataaacctgtaaccaaaactcagacatctggtactggtctttgcattgagattggtccctgtaaaaccccctttaaaagcatattgcatttagtacagagctcttttttgaaatgaaggctggagatgtgcatttttcacggtgttaactggttgtatcttattagcaaggagattggggttttgagtgtttgcgtgggtggtttcaatttgccagggaacagtggcaggctgctagcaaggcagtgagaagctcttggcagccaaatgggtgcattcagggctgatttatagagacccttggcttctccttctcctactccctgtctttctggcattttgtagcttgttagattttctgccagaggggtgggtcagagcagtggaggggagacatcgcccatgtgcttctgctactggtccttgggctgggtggttggtagaggagatgttgacactatgagctaagggttggcttttgtaattacctgaatctgaaaggaatgcctaaggttaccttggggtttctcttctggtgagatagggttcctggtttgagtaagttaatgtcctggatatttcttgtggcagggggtggtcaaagagcctgattgctgacccagtctcaggcctgtggtcgatgacctctcggtagtttcaaagggggctggagggggatatttgacttgttttttcgaaatgtagccttctaaccctcaagtctttagaagctgggtggactcttagtggtcctgcagcgtatcctaaaagactacctttgaaacaggattcttgtatggccaggatcctgtctgggaaccagaaaccctacaccctccccctccagggaatgctgagttccagttttgagcagaggtgaggcagaatccactgtagccttccgccctggtatttggggggatgaccagcccaggcgttgggtgttagtctgcatgagtttgtgagaggaaatagctgggtgtcctggcagtgcccttgaagttggttaggaccttcctgtaaactcttgcccctacttctaactactctataaatatatacatatatttatatataaagtgattagttgaactggcatcctgctttagcctgagacttgccataagaaactgctgagtacttggcaaaccctttcatagttttgttctccatctgtttggggtaggtgttgagcgaggcaaatggatctcgatatttcagatgggcttttgatgcactgttgccaaggaaggctttttctgattttttgacaaatgaatttttgcacactttcattggtgtctttcggcaacttacacacattgaaaatgagctattgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcagttactgtttttttaatcccctgaagttgtcctgtaggagacagaaattctttgctgtctgtatcccttggagtaagaaggtagtggcatgggtggagtgtgtgttctttctccaaatctattatgatgtttattaaacacttctgtagcaaagatggtggtagttcttttgttactgaagttgcccttcaccatggctatttgaaaaggagatgtacttggacgtttctgtaaatcttgagataaactgtttggagatttaaccacctctctgatgggggaccaactctatggaaattgtaaatacgttttatttataaacctggcactgtattcaataaacatttctgcagcctttcatctctaactgcgaa

SEQ ID NO: 12 - RNA, 4268 nt, Homo sapiens
gauacggcgcaccauauauauaucgcggggcgcagacucgcgcuccggcaguggugcugggagugucguggacgccgugccguuacucguagucaggcggcggcgcaggcggcggcggcggcauagcgcacagcgcgccuuagcagcagcagcagcagcagcggcaucggagguacccccgccgucgcagcccccgcgcuggugcagccacccucgcucccucugcucuuccucccuucgcucgcaccauggcugaucagcugaccgaagaacagauugcugaauucaaggaagccuucucccuauuugauaaagauggcgauggcaccaucacaacaaaggaacuuggaacugucaugaggucacugggucagaacccaacagaagcugaauugcaggauaugaucaaugaaguggaugcugaugguaauggcaccauugacuuccccgaauuuuugacuaugauggcuagaaaaaugaaagauacagauagugaagaagaaauccgugaggcauuccgagucuuugacaaggauggcagugguuauaucagugcagcagaacuacgucacgucaugacaaacuuaggagaaaaacuaacagaugaagaaguagaugaaaugaucagagaagcagauauugauggagacggacaagucaacuaugaagaauucguacagaugaugacugcaaaaugaagaccuacuuucaacuccuuuuuccccccucuagaagaaucaaauugaaucuuuuacuuaccucuugcaaaaaaaagaaaaaagaaaaaaguucauuuauucauucuguuucuauauagcaaaacugaaugucaaaaguaccuucuguccacacacacaaaaucugcauguauugguuggugguccuguccccuaaagaucaagcuacacaucaguuuuacaauauaaauacuuguacuaccuuaaugauaaggacuccuuaaaguuccauuugcuaaugauuaauacacuguuugggcuggccaguuuuucaugcaugcagcuugacgauugagcacagucaggccuuuguauuaaaaaugaaaaaugaaaaaacaaauucaaaaccuauucaaauggguucuaguucaauuuguuuaguauaaauugucauagcugguuuacugaaaacaaacacauuuaaaauugguuuaccucaggaugacgugcagaaaaaugggugaaggauaaaccguugagacguggccccacugguaggaugguccucuuguacuucgugugcuccgacccauggugacgaugacacacccugguggcaugcccguguauguugguuuagcguugucugcauuguucuagagugaaacaggugucaggcugucacuguucacacaaauuuuuaauaagaaacauuuaccaagggagcaucuuuggacucucuguuuuuaaaaccuucugaaccaugacuuggagccggcagaguaggcuguggcuguggacuucagcacaaccaucaacauugcuguucaaagaaauuacaguuuacguccauuccaaguuguaaaugcuagucuuuuuuuuuuuuuuuccaauaaaaagaccauuaacuuaaagugguguuaaaugcuuuguaaagcugagaucuaaauggggacaaggcagguggaggggaggccaguguacauguaaaugcccacagcccagcauuggguuucccucccaaggccccagcaccaaccucugagcccaagaccuugccugaaaacaagcagauaccgauugcuucauccuauuuauggacauguaggucuaguugcauuuucacuggggggaggggggaaggugaauuaugguaacuuuuaaugaucuauucaggcaguagagcucuuaaggaaaaaaaaaaacccacuuucucucaagcauguauuuagggguuguucucaauugugcugcugauuaccugucuuauguaacuacuugagaccaucugcaagagacaugauuuagugugucuguaauucaaucuucgcugugugugguagaagcaguagucacuuuuguaagccagucucuucaugccuaaaagacacuaccagucaccuuugauucgcgacuuuuaauuuaugauuauacuuagccuccuccuccuuuuuuuuuuuuucccaaguugacuugacuuugcuuuuuuccccccaaguagaacuaaugcuagcuuccagcuugaaaguaaaacuccaguguggagugaauuuugugucuaauuauaaaccuguaaccaaaacucagacaucugguacuggucuuugcauugagauuggucccuguaaaacccccuuuaaaagcauauugcauuuaguacagagcucuuuuuugaaaugaaggcuggagaugugcauuuuucacgguguuaacugguuguaucuuauuagcaaggagauugggguuuugaguguuugcgugggugguuucaauuugccagggaacaguggcaggcugcuagcaaggcagugagaagcucuuggcagccaaaugggugcauucagggcugauuuauagagacccuuggcuucuccuucuccuacucccugucuuucuggcauuuuguagcuuguuagauuuucugccagaggggugggucagagcaguggaggggagacaucgcccaugugcuucugcuacugguccuugggcugggugguugguagaggagauguugacacuaugagcuaaggguuggcuuuuguaauuaccugaaucugaaaggaaugccuaagguuaccuugggguuucucuucuggugagauaggguuccugguuugaguaaguuaauguccuggauauuucuuguggcaggggguggucaaagagccugauugcugacccagucucaggccuguggucgaugaccucucgguaguuucaaagggggcuggagggggauauuugacuuguuuuuucgaaauguagccuucuaacccucaagucuuuagaagcuggguggacucuuagugguccugcagcguauccuaaaagacuaccuuugaaacaggauucuuguauggccaggauccugucugggaaccagaaacccuacacccucccccuccagggaaugcugaguuccaguuuugagcagaggugaggcagaauccacuguagccuuccgcccugguauuuggggggaugaccagcccaggcguuggguguuagucugcaugaguuugugagaggaaauagcuggguguccuggcagugcccuugaaguugguuaggaccuuccuguaaacucuugccccuacuucuaacuacucuauaaauauauacauauauuuauauauaaagugauuaguugaacuggcauccugcuuuagccugagacuugccauaagaaacugcugaguacuuggcaaacccuuucauaguuuuguucuccaucuguuugggguagguguugagcgaggcaaauggaucucgauauuucagaugggcuuuugaugcacuguugccaaggaaggcuuuuucugauuuuuugacaaaugaauuuuugcacacuuucauuggugucuuucggcaacuuacacacauugaaaaugagcuauuguacauauuuuuauauucucuuuauaaaugcaugucugauuguacuuguaacaauauuguaaugaacggcugugcaguaggcccagcgcugcugugucucgucagaggaauagcuuaccacgaaccccucagcauacugggaaucucuuccugaacaacgaauguaaauuuggucaagucuacucuuccguucauucaauuauuuuaagcauuugaauuauuuauuguauauccuaaauauauuucuccuuuggcagugacuagauuuccacuaaugugucuuaaucuaucccuccagcuggcaguuacuguuuuuuuaauccccugaaguuguccuguaggagacagaaauucuuugcugucuguaucccuuggaguaagaagguaguggcauggguggaguguguguucuuucuccaaaucuauuaugauguuuauuaaacacuucuguagcaaagauggugguaguucuuuuguuacugaaguugcccuucaccauggcuauuugaaaaggagauguacuuggacguuucuguaaaucuugagauaaacuguuuggagauuuaaccaccucucugaugggggaccaacucuauggaaauuguaaauacguuuuauuuauaaaccuggcacuguauucaauaaacauuucugcagccuuucaucucuaacugcgaaaaaaaaaaaaaa

SEQ ID NO: 13 - protein, 148 aa, Homo sapiens
ADQLTEEQIAEFKEAFSLFDKDGDGTITTKELGTVMRSLGQNPTEAELQDMIIEVDADGNGTIDFPEFLTMMARKMKDTDSEEEIREAFRVFDKDGSGYISAAELRHVMTNLGEKLTDEEVDEMIREADIDGDGQVNYEEFVQMMTAK

SEQ ID NO: 14 - protein, 29 aa, Artificial Sequence
  RYR2 Calmodulin binding peptide
SKKAVWHKLLSKQRKRAVVACFRMAPLYN

SEQ ID NO: 15 - protein, 31 aa, Artificial Sequence
  RYR2p Calmodulin binding peptide
RSKKAVWHKLLSKQRKRAVVACFRMAPLYNL

SEQ ID NO: 16 - protein, 148 aa, Mouse; identical to SEQ ID NO: 4

SEQ ID NO: 17 - protein, 148 aa, Fish; identical to SEQ ID NO: 4

SEQ ID NO: 18 - protein, 148 aa, Round worm
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGFISAAELRHVMTNLGEKLTDEEVDEMIREADIDGDGQVNYEEFVTMMTTK

SEQ ID NO: 19 - protein, 148 aa, Plant
ADQLTDDQISEFKEAFSLFDKDGDGCITTKELGTVMRSLGQNPTEAELQDMINEVDADGNGTIDFPEFLTNLARKMKDTDSEEELKEAFRVFDKDQNGFISAAELRHVMTNLGEKLTDEEVDEMIKEADVDGDGQINYEEFKVMMMAK